(19) United States
(12) Patent Application Publication
(10) Pub. No.: US 2025/0089741 A1
(43) Pub. Date: Mar. 20, 2025

(54) POLYPEPTIDES HAVING LYSOZYME ACTIVITY, POLYNUCLEOTIDES ENCODING SAME AND USES AND COMPOSITIONS THEREOF

(71) Applicant: Novozymes A/S, Bagsvaerd (DK)

(72) Inventors: Mikkel KLAUSEN, Copenhagen (DK); Kirk Matthew SCHNORR, Holte (DK); Soeren NYMAND-GRARUP, Kobenhavn (DK); Peter Bjarke OLSEN, Kobenhavn (DK); Marianne Thorup COHN, Copenhagen (DK); Robert Piotr OLINSKI, Vaerloese (DK); Marc Dominique MORANT, Frederiksberg (DK); Ming LI, Beijing (CN); Ye LIU, Beijing (CN); Lars Kobbeeroee SKOV, Ballerup (DK); Dominique Aubert SKOVLUNDE, Vaerloese (DK); Han BUCONG, Beijing (CN)

(73) Assignee: Novozymes A/S, Bagsvaerd (DK)

(21) Appl. No.: 18/956,701

(22) Filed: Nov. 22, 2024

Related U.S. Application Data
(62) Division of application No. 18/469,388, filed on Sep. 18, 2023, now Pat. No. 12,207,671.
Application No. 18/469,388 is a division of application No. 16/462,764, filed on May 21, 2019, now Pat. No. 11,896,033.

(30) Foreign Application Priority Data
Dec. 21, 2016 (WO) PCT/CN2016/111317
Mar. 8, 2017 (WO) PCT/2017/075978

(51) Int. Cl. A23K 20/147; A23K 10/30; A23K 50/30; A23K 50/75; C12N 9/36; C12N 15/81

(52) U.S. Cl. CPC A23K 20/147; A23K 10/30; A23K 50/30; A23K 50/75; C12N 9/2462; C12N 15/815; C12Y 302/01017

(57) ABSTRACT

The present disclosure relates animal feed or animal feed additives comprising one or more polypeptides having lysozyme activity. The present disclosure also relates to polypeptides having lysozyme activity, polynucleotides encoding the polypeptides nucleic acid constructs, vectors, and host cells comprising the polynucleotides as well as methods of producing and using the polypeptides.

### CROSS-REFERENCE TO RELATED APPLICATIONS

This application is a divisional of Ser. No. 18/469,388, filed Sep. 18, 2023, which is a divisional of Ser. No. 16/462,764, filed May 21, 2019, and issued as U.S. Pat. No. 11,896,033 on Feb. 13, 2024, which is itself a 35 U.S.C. 371 national stage entry of PCT/CN2017/117765, filed Dec. 21, 2017, which claims priority to PCT/CN2016/111317, filed Dec. 21, 2016, and to PCT/CN2017/075978, filed Mar. 8, 2017. The contents of the aforementioned applications are fully incorporated herein by reference.

### REFERENCE TO A SEQUENCE LISTING

This application contains a Sequence Listing in computer readable form, which is incorporated herein by reference. The name of the file containing the Sequence Listing is SQ.xml, which contains 354, 119 bytes and which was created on Nov. 22, 2024.

### FIELD OF THE INVENTION

The present disclosure relates animal feed or animal feed additives comprising one or more polypeptides having lysozyme activity. The present disclosure also relates to polypeptides having lysozyme activity, polynucleotides encoding the polypeptides nucleic acid constructs, vectors, and host cells comprising the polynucleotides as well as methods of producing and using the polypeptides.

### BACKGROUND

Lysozyme is an O-glycosyl hydrolase produced as a defensive mechanism against bacteria by many organisms. The enzyme causes the hydrolysis of bacterial cell walls by cleaving the glycosidic bonds of peptidoglycan; an important structural molecule in bacteria. After having their cell walls weakened by lysozyme action, bacterial cells lyse as a result of unbalanced osmotic pressure.

Lysozyme naturally occurs in many organisms such as viruses, plants, insects, birds, reptiles and mammals. In mammals, Lysozyme has been isolated from nasal secretions, saliva, tears, intestinal content, urine and milk. The enzyme cleaves the glycosidic bond between carbon number 1 of N-acetylmuramic acid and carbon number 4 of N-acetyl-D-glucosamine. In vivo, these two carbohydrates are polymerized to form the cell wall polysaccharide of many microorganisms.

Lysozyme has been classified into five different glycoside hydrolase (GH) families (CAZy, www.cazy.org): hen egg-white lysozyme (GH22), goose egg-white lysozyme (GH23), bacteriophage T4 lysozyme (GH24), Sphingomonas flagellar protein (GH73) and Chalaropsis lysozymes (GH25). Lysozymes from the families GH23 and GH24 are primarily known from bacteriophages and have recently been identified in fungi. The lysozyme family GH25 has been found to be structurally unrelated to the other lysozyme families.

Lysozyme extracted from hen egg white is the primary product available on the commercial market, but does not cleave N,6-O-diacetylmuramic acid in e.g. Staphylococcus aureus cell walls and is thus unable to lyse this important human pathogen among others (Masschalck B, Deckers D, Michiels C W (2002), “Lytic and nonlytic mechanism of inactivation of gram-positive bacteria by lysozyme under atmospheric and high hydrostatic pressure”, J Food Prot. 65 (12): 1916-23).

Antimicrobial growth promoters (AGP's) have traditionally been used for growth promotion in animals, and probably work by preventing low level infections by pathogens such as Clostridium perfringens. However, AGP's are increasingly being banned worldwide and therefore new solutions to promote animal growth but which are not AGP's are of interest.

WO2013/076253 and WO2005/080559 disclose GH25 lysozymes for use in animal feed. However, said lysozymes are not highly active in degrading the cell wall from Micrococcus lysodeikticus (a typical lysozyme activity assay) and more active lysozymes would be desired. The object of the present disclosure is to provide new and more active lysozymes which could be suitable for animal health.

### SUMMARY

The present disclosure relates to polypeptides which have improved lysozyme activity against the peptidoglycans found in the cell walls of Micrococcus lysodeikticus. The lysozymes of the present disclosure are all GH25 lysozymes and all have improved ability to lyse bacterial cell walls of Micrococcus lysodeikticus, thereby rendering them suitable for use in animal feed to improve the animal health. The lysozymes of the present disclosure have improved activity compared to the lysozyme described in WO2013/076253 (it is described herein as SEQ ID NO 159). A large subset of the GH25 lysozymes have surprisingly been found to furthermore have lysozyme activity against Lactobacillus johnsonii. Lactobacillus johnsonii is an important bacterium of the intestinal flora of animals. Without being bound to a particular theory, it is believed that removal of dead Lactobacillus johnsonii cells from the intestinal flora, by means of enzymatic lyses of the partially degraded bacterial cell wall, to be an important contributor to intestinal health of an animal.

An aspect of the present disclosure is directed to an isolated GH25 polypeptide having lysozyme activity selected from the group consisting of:
(a) a polypeptide having at least 80% sequence identity to the polypeptide of SEQ ID NO: 3;
(b) a polypeptide having at least 84% sequence identity to the polypeptide of SEQ ID NO: 6;
(c) a polypeptide having at least 80% sequence identity to the polypeptide of SEQ ID NO: 9;
(d) a polypeptide having at least 80% sequence identity to the polypeptide of SEQ ID NO: 12;
(e) a polypeptide having at least 80% sequence identity to the polypeptide of SEQ ID NO: 15;
(f) a polypeptide having at least 80% sequence identity to the polypeptide of SEQ ID NO: 18;
(g) a polypeptide having at least 80% sequence identity to the polypeptide of SEQ ID NO: 21;
(h) a polypeptide having at least 80% sequence identity to the polypeptide of SEQ ID NO: 24;
(i) a polypeptide having at least 80% sequence identity to the polypeptide of SEQ ID NO: 27;
(j) a polypeptide having at least 84% sequence identity to the polypeptide of SEQ ID NO: 30;
(k) a polypeptide having at least 80% sequence identity to the polypeptide of SEQ ID NO: 33;
(l) a polypeptide having at least 80% sequence identity to the polypeptide of SEQ ID NO: 36;
(m) a polypeptide having at least 84% sequence identity to the polypeptide of SEQ ID NO: 39;
(n) a polypeptide having at least 80% sequence identity to the polypeptide of SEQ ID NO: 42;
(o) a polypeptide having at least 80% sequence identity to the polypeptide of SEQ ID NO: 45;
(p) a polypeptide having at least 80% sequence identity to the polypeptide of SEQ ID NO: 48;
(q) a polypeptide having at least 80% sequence identity to the polypeptide of SEQ ID NO: 51;
(r) a polypeptide having at least 80% sequence identity to the polypeptide of SEQ ID NO: 54;
(s) a polypeptide having at least 80% sequence identity to the polypeptide of SEQ ID NO: 57;
(t) a polypeptide having at least 80% sequence identity to the polypeptide of SEQ ID NO: 60;
(u) a polypeptide having at least 80% sequence identity to the polypeptide of SEQ ID NO: 63;
(v) a polypeptide having at least 80% sequence identity to the polypeptide of SEQ ID NO: 66;
(w) a polypeptide having at least 80% sequence identity to the polypeptide of SEQ ID NO: 69;
(x) a polypeptide having at least 84% sequence identity to the polypeptide of SEQ ID NO: 72;
(y) a polypeptide having at least 80% sequence identity to the polypeptide of SEQ ID NO: 75;
(z) a polypeptide having at least 80% sequence identity to the polypeptide of SEQ ID NO: 80;
(aa) a polypeptide having at least 80% sequence identity to the polypeptide of SEQ ID NO: 83;
(ab) a polypeptide having at least 80% sequence identity to the polypeptide of SEQ ID NO: 86;
(ac) a polypeptide having at least 86% sequence identity to the polypeptide of SEQ ID NO: 89;
(ad) a polypeptide having at least 82% sequence identity to the polypeptide of SEQ ID NO: 92;
(ae) a polypeptide having at least 82% sequence identity to the polypeptide of SEQ ID NO: 95;
(af) a polypeptide having at least 80% sequence identity to the polypeptide of SEQ ID NO: 98;
(ag) a polypeptide having at least 80% sequence identity to the polypeptide of SEQ ID NO: 101;
(ah) a polypeptide having at least 80% sequence identity to the polypeptide of SEQ ID NO: 104;
(ai) a polypeptide having at least 80% sequence identity to the polypeptide of SEQ ID NO: 107;
(aj) a polypeptide having at least 82% sequence identity to the polypeptide of SEQ ID NO: 110;
(ak) a polypeptide having at least 81% sequence identity to the polypeptide of SEQ ID NO: 113;
(al) a polypeptide having at least 80% sequence identity to the polypeptide of SEQ ID NO: 116;
(am) a polypeptide having at least 80% sequence identity to the polypeptide of SEQ ID NO: 119;
(an) a polypeptide having at least 80% sequence identity to the polypeptide of SEQ ID NO: 122;
(ao) a polypeptide having at least 80% sequence identity to the polypeptide of SEQ ID NO: 125;
(ap) a polypeptide having at least 80% sequence identity to the polypeptide of SEQ ID NO: 128;
(aq) a polypeptide having at least 80% sequence identity to the polypeptide of SEQ ID NO: 131;
(ar) a polypeptide having at least 80% sequence identity to the polypeptide of SEQ ID NO: 134;
(as) a polypeptide having at least 80% sequence identity to the polypeptide of SEQ ID NO: 137;
(at) a polypeptide having at least 80% sequence identity to the polypeptide of SEQ ID NO: 140;
(au) a polypeptide having at least 80% sequence identity to the polypeptide of SEQ ID NO: 143;
(av) a polypeptide having at least 80% sequence identity to the polypeptide of SEQ ID NO: 146;
(aw) a polypeptide having at least 80% sequence identity to the polypeptide of SEQ ID NO: 149;
(ax) a polypeptide having at least 80% sequence identity to the polypeptide of SEQ ID NO: 152;
(ay) a polypeptide having at least 80% sequence identity to the polypeptide of SEQ ID NO: 155;
(az) a polypeptide having at least 80% sequence identity to the polypeptide of SEQ ID NO: 158;
(ba) a polypeptide having at least 80% sequence identity to the polypeptide of SEQ ID NO: 221;
(bb) a polypeptide having at least 80% sequence identity to the polypeptide of SEQ ID NO: 224;
(bc) a polypeptide having at least 80% sequence identity to the polypeptide of SEQ ID NO: 227;
(bd) a polypeptide having at least 80% sequence identity to the polypeptide of SEQ ID NO: 230;
(be) a polypeptide having at least 80% sequence identity to the polypeptide of SEQ ID NO: 233;
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27 or 28 positions;
(bg) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal His-tag and/or HQ-tag;
(bh) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal extension of up to 10 amino acids, e.g. 1, 2, 3, 4, 5, 6, 7, 8, 9 or 10 amino acids; and
(bi) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) having lysozyme activity and having at least 90% of the length of the mature polypeptide.

A further aspect of the present disclosure is directed to an isolated GH25 polypeptide having lysozyme activity selected from the group consisting of:
(a) a polypeptide having at least 90% sequence identity to the polypeptide of SEQ ID NO: 3;
(b) a polypeptide having at least 90% sequence identity to the polypeptide of SEQ ID NO: 6;
(e) a polypeptide having at least 90% sequence identity to the polypeptide of SEQ ID NO: 15;
(f) a polypeptide having at least 90% sequence identity to the polypeptide of SEQ ID NO: 18;
(g) a polypeptide having at least 90% sequence identity to the polypeptide of SEQ ID NO: 21;
(h) a polypeptide having at least 90% sequence identity to the polypeptide of SEQ ID NO: 24;
(k) a polypeptide having at least 90% sequence identity to the polypeptide of SEQ ID NO: 33;
(l) a polypeptide having at least 90% sequence identity to the polypeptide of SEQ ID NO: 36;
(m) a polypeptide having at least 90% sequence identity to the polypeptide of SEQ ID NO: 39;
(o) a polypeptide having at least 90% sequence identity to the polypeptide of SEQ ID NO: 45;
(p) a polypeptide having at least 90% sequence identity to the polypeptide of SEQ ID NO: 48;
(q) a polypeptide having at least 90% sequence identity to the polypeptide of SEQ ID NO: 51;
(r) a polypeptide having at least 90% sequence identity to the polypeptide of SEQ ID NO: 54;
(s) a polypeptide having at least 90% sequence identity to the polypeptide of SEQ ID NO: 57;
(t) a polypeptide having at least 90% sequence identity to the polypeptide of SEQ ID NO: 60;
(x) a polypeptide having at least 90% sequence identity to the polypeptide of SEQ ID NO: 72;
(y) a polypeptide having at least 90% sequence identity to the polypeptide of SEQ ID NO: 75;
(z) a polypeptide having at least 90% sequence identity to the polypeptide of SEQ ID NO: 80;
(ac) a polypeptide having at least 90% sequence identity to the polypeptide of SEQ ID NO: 89;
(ae) a polypeptide having at least 90% sequence identity to the polypeptide of SEQ ID NO: 95;
(af) a polypeptide having at least 90% sequence identity to the polypeptide of SEQ ID NO: 98;
(ag) a polypeptide having at least 90% sequence identity to the polypeptide of SEQ ID NO: 101;
(ak) a polypeptide having at least 90% sequence identity to the polypeptide of SEQ ID NO: 113;
(al) a polypeptide having at least 90% sequence identity to the polypeptide of SEQ ID NO: 116;
(am) a polypeptide having at least 90% sequence identity to the polypeptide of SEQ ID NO: 119;
(an) a polypeptide having at least 90% sequence identity to the polypeptide of SEQ ID NO: 122;
(aq) a polypeptide having at least 90% sequence identity to the polypeptide of SEQ ID NO: 131;
(as) a polypeptide having at least 90% sequence identity to the polypeptide of SEQ ID NO: 137;
(au) a polypeptide having at least 90% sequence identity to the polypeptide of SEQ ID NO: 143;
(av) a polypeptide having at least 90% sequence identity to the polypeptide of SEQ ID NO: 146;
(aw) a polypeptide having at least 90% sequence identity to the polypeptide of SEQ ID NO: 149;
(ax) a polypeptide having at least 90% sequence identity to the polypeptide of SEQ ID NO: 152;
(ay) a polypeptide having at least 90% sequence identity to the polypeptide of SEQ ID NO: 155;
(ba) a polypeptide having at least 90% sequence identity to the polypeptide of SEQ ID NO: 221;
(bd) a polypeptide having at least 90% sequence identity to the polypeptide of SEQ ID NO: 230;
(be) a polypeptide having at least 90% sequence identity to the polypeptide of SEQ ID NO: 233; and
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions.

As stated, the polypeptides of the present disclosure have improved lysozyme activity. An interesting embodiment of the present disclosure relates to the polypeptides of the present disclosure having improved lysozyme activity a) compared to the lysozyme activity of hen eggwhite lysozyme (HEWL) or b) compared to the lysozyme activity of SEQ ID NO: 159 as determined by any one of i) Method for the Determination of Lysozyme Activity Against Micrococcus lysodeikticus and ii) Method for the Determination of Lysozyme Activity Against Lactobacillus johnsonii.

A further aspect of the present disclosure is directed to a method of hydrolysing peptidoglycan in bacterial cell walls comprising treating bacterial cells with one or more GH25 polypeptides having lysozyme activity, wherein the polypeptide is as defined above.

The present disclosure is furthermore directed to a method of increasing the digestibility of peptidoglycans in animal feed comprising the use of a polypeptide as described herein and to an animal feed additive or animal feed comprising the polypeptide as defined herein. A related aspect of the present disclosure is directed to a zootechnical additive for use in feed for poultry or swine, said additive comprising the polypeptide as defined herein.

An aspect of the present disclosure is directed to a method of improving the intestinal health in an animal comprising reducing the amount of dead Lactobacillus johnsonii cells, or cell wall debris therefrom, in the digestive tract of said animal, comprising feeding the animal with a feed or feed additive comprising a polypeptide as defined by the present disclosure.

An aspect of the present disclosure is directed to a method of improving the intestinal health in an animal comprising reducing the amount of dead Lactobacillus johnsonii cells, or cell wall debris therefrom, in the digestive tract of said animal, comprising feeding the animal with a feed or feed additive comprising a polypeptide selected from the group consisting of a polypeptide having at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO 159.

An aspect of the present disclosure is directed to a method of promoting the elimination of dead Lactobacillus johnsonii cells, or cell wall debris therefrom, from the digestive tract of an animal comprising feeding said animal a polypeptide or source of a polypeptide selected from the group consisting of a polpypeptide having at least 90% sequence identity to the polypeptide of SEQ ID NO: 15, a polpypeptide having at least 90% sequence identity to the polypeptide of SEQ ID NO: 36, a polpypeptide having at least 90% sequence identity to the polypeptide of SEQ ID NO: 75, a polpypeptide having at least 90% sequence identity to the polypeptide of SEQ ID NO: 80, and a polpypeptide having at least 90% sequence identity to the polypeptide of SEQ ID NO: 119 or a polypeptide or source of a polypeptide selected from the group consisting of a polypeptide having at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO 159.

The present disclosure also relates to a granule comprising one or more GH25 polypeptides as described above. The present disclosure further relates to an isolated polypeptide having lysozyme activity as described in the claims.

The present disclosure further relates to compositions comprising the polypeptide of the present disclosure, such as animal feed additives or animal feed; use of the polypeptide of the present disclosure in animal feed, in animal feed additives, in the preparation of a composition for use in animal feed, for improving one or more performance parameters in an animal; and isolated polynucleotides encoding the polypeptides of the present disclosure, recombinant host cells and method of producing the polypeptide of the present disclosure.

### Overview of Sequence Listing

SEQ ID NO: 1 is the cDNA sequence of a GH25 lysozyme as isolated from Sporormia fimetaria.

SEQ ID NO: 2 is the amino acid sequence as deduced from SEQ ID NO: 1.

SEQ ID NO: 3 is the amino acid sequence of the mature GH25 lysozyme from Sporormia fimetaria.

SEQ ID NO: 4 is the cDNA sequence of a GH25 lysozyme as isolated from Poronia punctata.

SEQ ID NO: 5 is the amino acid sequence as deduced from SEQ ID NO: 4.

SEQ ID NO: 6 is the amino acid sequence of the mature GH25 lysozyme from Poronia punctata.

SEQ ID NO: 7 is the cDNA sequence of a GH25 lysozyme as isolated from Poronia punctata.

SEQ ID NO: 8 is the amino acid sequence as deduced from SEQ ID NO: 7.

SEQ ID NO: 9 is the amino acid sequence of the mature GH25 lysozyme from Poronia punctata.

SEQ ID NO: 10 is the cDNA sequence of a GH25 lysozyme as isolated from Lecanicillium sp. WMM742.

SEQ ID NO: 11 is the amino acid sequence as deduced from SEQ ID NO: 10.

SEQ ID NO: 12 is the amino acid sequence of the mature GH25 lysozyme from Lecanicillium sp. WMM742.

SEQ ID NO: 13 is the cDNA sequence of a GH25 lysozyme as isolated from Lecanicillium sp. WMM742.

SEQ ID NO: 14 is the amino acid sequence as deduced from SEQ ID NO: 13.

SEQ ID NO: 15 is the amino acid sequence of the mature GH25 lysozyme from Lecanicillium sp. WMM742.

SEQ ID NO: 16 is the cDNA sequence of a GH25 lysozyme as isolated from Onygena equina.

SEQ ID NO: 17 is the amino acid sequence as deduced from SEQ ID NO: 16.

SEQ ID NO: 18 is the amino acid sequence of the mature GH25 lysozyme from Onygena equina.

SEQ ID NO: 19 is the cDNA sequence of a GH25 lysozyme as isolated from Purpureocillium lilacinum.

SEQ ID NO: 20 is the amino acid sequence as deduced from SEQ ID NO: 19.

SEQ ID NO: 21 is the amino acid sequence of the mature GH25 lysozyme from Purpureocillium lilacinum.

SEQ ID NO: 22 is the cDNA sequence of a GH25 lysozyme as isolated from Trichobolus zukalii.

SEQ ID NO: 23 is the amino acid sequence as deduced from SEQ ID NO: 22.

SEQ ID NO: 24 is the amino acid sequence of the mature GH25 lysozyme from Trichobolus zukalii.

SEQ ID NO: 25 is the cDNA sequence of a GH25 lysozyme as isolated from Penicillium citrinum.

SEQ ID NO: 26 is the amino acid sequence as deduced from SEQ ID NO: 25.

SEQ ID NO: 27 is the amino acid sequence of the mature GH25 lysozyme from Penicillium citrinum.

SEQ ID NO: 28 is the cDNA sequence of a GH25 lysozyme as isolated from Cladorrhinum bulbillosum.

SEQ ID NO: 29 is the amino acid sequence as deduced from SEQ ID NO: 28.

SEQ ID NO: 30 is the amino acid sequence of the mature GH25 lysozyme from Cladorrhinum bulbillosum.

SEQ ID NO: 31 is the cDNA sequence of a GH25 lysozyme as isolated from Umbelopsis westeae.

SEQ ID NO: 32 is the amino acid sequence as deduced from SEQ ID NO: 31.

SEQ ID NO: 33 is the amino acid sequence of the mature GH25 lysozyme from Umbelopsis westeae.

SEQ ID NO: 34 is the cDNA sequence of a GH25 lysozyme as isolated from Zygomycetes sp. XZ2655.

SEQ ID NO: 35 is the amino acid sequence as deduced from SEQ ID NO: 34.

SEQ ID NO: 36 is the amino acid sequence of the mature GH25 lysozyme from Zygomycetes sp. XZ2655.

SEQ ID NO: 37 is the cDNA sequence of a GH25 lysozyme as isolated from Chaetomium cupreum.

SEQ ID NO: 38 is the amino acid sequence as deduced from SEQ ID NO: 37.

SEQ ID NO: 39 is the amino acid sequence of the mature GH25 lysozyme from Chaetomium cupreum.

SEQ ID NO: 40 is the cDNA sequence of a GH25 lysozyme as isolated from Cordyceps cardinalis.

SEQ ID NO: 41 is the amino acid sequence as deduced from SEQ ID NO: 40.

SEQ ID NO: 42 is the amino acid sequence of the mature GH25 lysozyme from Cordyceps cardinalis.

SEQ ID NO: 43 is the cDNA sequence of a GH25 lysozyme as isolated from Penicillium sp. ‘qii’.

SEQ ID NO: 44 is the amino acid sequence as deduced from SEQ ID NO: 43.

SEQ ID NO: 45 is the amino acid sequence of the mature GH25 lysozyme from Penicillium sp. ‘qii’.

SEQ ID NO: 46 is the cDNA sequence of a GH25 lysozyme as isolated from Aspergillus sp. nov. XZ2609.

SEQ ID NO: 47 is the amino acid sequence as deduced from SEQ ID NO: 46.

SEQ ID NO: 48 is the amino acid sequence of the mature GH25 lysozyme from Aspergillus sp. nov. XZ2609.

SEQ ID NO: 49 is the cDNA sequence of a GH25 lysozyme as isolated from Paecilomyces sp. XZ2658.

SEQ ID NO: 50 is the amino acid sequence as deduced from SEQ ID NO: 49.

SEQ ID NO: 51 is the amino acid sequence of the mature GH25 lysozyme from Paecilomyces sp. XZ2658.

SEQ ID NO: 52 is the cDNA sequence of a GH25 lysozyme as isolated from Paecilomyces sp. XZ2658.

SEQ ID NO: 53 is the amino acid sequence as deduced from SEQ ID NO: 52.

SEQ ID NO: 54 is the amino acid sequence of the mature GH25 lysozyme from Paecilomyces sp. XZ2658.

SEQ ID NO: 55 is the cDNA sequence of a GH25 lysozyme as isolated from Rhizomucor pusillus.

SEQ ID NO: 56 is the amino acid sequence as deduced from SEQ ID NO: 55.

SEQ ID NO: 57 is the amino acid sequence of the mature GH25 lysozyme from Rhizomucor pusillus.

SEQ ID NO: 58 is the cDNA sequence of a GH25 lysozyme as isolated from Pycnidiophora cf. dispera.

SEQ ID NO: 59 is the amino acid sequence as deduced from SEQ ID NO: 58.

SEQ ID NO: 60 is the amino acid sequence of the mature GH25 lysozyme from Pycnidiophora cf. dispera.

SEQ ID NO: 61 is the cDNA sequence of a GH25 lysozyme as isolated from Thermomucor indicae-seudaticae.

SEQ ID NO: 62 is the amino acid sequence as deduced from SEQ ID NO: 61.

SEQ ID NO: 63 is the amino acid sequence of the mature GH25 lysozyme from Thermomucor indicae-seudaticae.

SEQ ID NO: 64 is the cDNA sequence of a GH25 lysozyme as isolated from Isaria farinosa.

SEQ ID NO: 65 is the amino acid sequence as deduced from SEQ ID NO: 64.

SEQ ID NO: 66 is the amino acid sequence of the mature GH25 lysozyme from Isaria farinosa.

SEQ ID NO: 67 is the cDNA sequence of a GH25 lysozyme as isolated from Lecanicillium sp. WMM742.

SEQ ID NO: 68 is the amino acid sequence as deduced from SEQ ID NO: 67.

SEQ ID NO: 69 is the amino acid sequence of the mature GH25 lysozyme from Lecanicillium sp. WMM742.

SEQ ID NO: 70 is the cDNA sequence of a GH25 lysozyme as isolated from Zopfiella sp. t180-6.

SEQ ID NO: 71 is the amino acid sequence as deduced from SEQ ID NO: 70.

SEQ ID NO: 72 is the amino acid sequence of the mature GH25 lysozyme from Zopfiella sp. t180-6.

SEQ ID NO: 73 is the cDNA sequence of a GH25 lysozyme as isolated from Malbranchea flava.

SEQ ID NO: 74 is the amino acid sequence as deduced from SEQ ID NO: 73.

SEQ ID NO: 75 is the amino acid sequence of the mature GH25 lysozyme from Malbranchea flava.

SEQ ID NO: 76 is the cDNA sequence of a GH25 lysozyme as isolated from Hypholoma polytrichi.

SEQ ID NO: 77 is the amino acid sequence as deduced from SEQ ID NO: 76.

SEQ ID NO: 78 is the codon optimised DNA the GH25 lysozyme as isolated from Hypholoma polytrichi.

SEQ ID NO: 79 is the amino acid sequence as deduced from SEQ ID NO: 78.

SEQ ID NO: 80 is the amino acid sequence of the mature GH25 lysozyme from Hypholoma polytrichi.

SEQ ID NO: 81 is the cDNA sequence of a GH25 lysozyme as isolated from Aspergillus deflectus.

SEQ ID NO: 82 is the amino acid sequence as deduced from SEQ ID NO: 81.

SEQ ID NO: 83 is the amino acid sequence of the mature GH25 lysozyme from Aspergillus deflectus.

SEQ ID NO: 84 is the cDNA sequence of a GH25 lysozyme as isolated from Ascobolus stictoideus.

SEQ ID NO: 85 is the amino acid sequence as deduced from SEQ ID NO: 84.

SEQ ID NO: 86 is the amino acid sequence of the mature GH25 lysozyme from Ascobolus stictoideus.

SEQ ID NO: 87 is the cDNA sequence of a GH25 lysozyme as isolated from Coniochaeta sp.

SEQ ID NO: 88 is the amino acid sequence as deduced from SEQ ID NO: 87.

SEQ ID NO: 89 is the amino acid sequence of the mature GH25 lysozyme from Coniochaeta sp.

SEQ ID NO: 90 is the cDNA sequence of a GH25 lysozyme as isolated from Daldinia fissa

SEQ ID NO: 91 is the amino acid sequence as deduced from SEQ ID NO: 90.

SEQ ID NO: 92 is the amino acid sequence of the mature GH25 lysozyme from Daldinia fissa.

SEQ ID NO: 93 is the cDNA sequence of a GH25 lysozyme as isolated from Rosellinia sp.

SEQ ID NO: 94 is the amino acid sequence as deduced from SEQ ID NO: 93.

SEQ ID NO: 95 is the amino acid sequence of the mature GH25 lysozyme from Rosellinia sp.

SEQ ID NO: 96 is the cDNA sequence of a GH25 lysozyme as isolated from Ascobolus sp. ZY179.

SEQ ID NO: 97 is the amino acid sequence as deduced from SEQ ID NO: 96.

SEQ ID NO: 98 is the amino acid sequence of the mature GH25 lysozyme from Ascobolus sp. ZY179.

SEQ ID NO: 99 is the cDNA sequence of a GH25 lysozyme as isolated from Curreya sp. XZ2623.

SEQ ID NO: 100 is the amino acid sequence as deduced from SEQ ID NO: 99.

SEQ ID NO: 101 is the amino acid sequence of the mature GH25 lysozyme from Curreya sp. XZ2623.

SEQ ID NO: 102 is the cDNA sequence of a GH25 lysozyme as isolated from Coniothyrium sp.

SEQ ID NO: 103 is the amino acid sequence as deduced from SEQ ID NO: 102.

SEQ ID NO: 104 is the amino acid sequence of the mature GH25 lysozyme from Coniothyrium sp.

SEQ ID NO: 105 is the cDNA sequence of a GH25 lysozyme as isolated from Hypoxylon sp.

SEQ ID NO: 106 is the amino acid sequence as deduced from SEQ ID NO: 105.

SEQ ID NO: 107 is the amino acid sequence of the mature GH25 lysozyme from Hypoxylon sp.

SEQ ID NO: 108 is the cDNA sequence of a GH25 lysozyme as isolated from Xylariaceae sp. 1653h.

SEQ ID NO: 109 is the amino acid sequence as deduced from SEQ ID NO: 108.

SEQ ID NO: 110 is the amino acid sequence of the mature GH25 lysozyme from Xylariaceae sp. 1653h.

SEQ ID NO: 111 is the cDNA sequence of a GH25 lysozyme as isolated from Hypoxylon sp.

SEQ ID NO: 112 is the amino acid sequence as deduced from SEQ ID NO: 111.

SEQ ID NO: 113 is the amino acid sequence of the mature GH25 lysozyme from Hypoxylon sp.

SEQ ID NO: 114 is the cDNA sequence of a GH25 lysozyme as isolated from Yunnania penicillata.

SEQ ID NO: 115 is the amino acid sequence as deduced from SEQ ID NO: 114.

SEQ ID NO: 116 is the amino acid sequence of the mature GH25 lysozyme from Yunnania penicillata.

SEQ ID NO: 117 is the cDNA sequence of a GH25 lysozyme as isolated from Engyodontium album.

SEQ ID NO: 118 is the amino acid sequence as deduced from SEQ ID NO: 117.

SEQ ID NO: 119 is the amino acid sequence of the mature GH25 lysozyme from Engyodontium album.

SEQ ID NO: 120 is the cDNA sequence of a GH25 lysozyme as isolated from Metapochonia bulbillosa.

SEQ ID NO: 121 is the amino acid sequence as deduced from SEQ ID NO: 120.

SEQ ID NO: 122 is the amino acid sequence of the mature GH25 lysozyme from Metapochonia bulbillosa.

SEQ ID NO: 123 is the cDNA sequence of a GH25 lysozyme as isolated from Hamigera paravellanea.

SEQ ID NO: 124 is the amino acid sequence as deduced from SEQ ID NO: 123.

SEQ ID NO: 125 is the amino acid sequence of the mature GH25 lysozyme from Hamigera paravellanea.

SEQ ID NO: 126 is the cDNA sequence of a GH25 lysozyme as isolated from Metarhizium iadini.

SEQ ID NO: 127 is the amino acid sequence as deduced from SEQ ID NO: 126.

SEQ ID NO: 128 is the amino acid sequence of the mature GH25 lysozyme from Metarhizium iadini.

SEQ ID NO: 129 is the cDNA sequence of a GH25 lysozyme as isolated from Thermoascus aurantiacus.

SEQ ID NO: 130 is the amino acid sequence as deduced from SEQ ID NO: 129.

SEQ ID NO: 131 is the amino acid sequence of the mature GH25 lysozyme from Thermoascus aurantiacus.

SEQ ID NO: 132 is the cDNA sequence of a GH25 lysozyme as isolated from Clonostachys rossmaniae.

SEQ ID NO: 133 is the amino acid sequence as deduced from SEQ ID NO: 132.

SEQ ID NO: 134 is the amino acid sequence of the mature GH25 lysozyme from Clonostachys rossmaniae.

SEQ ID NO: 135 is the cDNA sequence of a GH25 lysozyme as isolated from Simplicillium obclavatum.

SEQ ID NO: 136 is the amino acid sequence as deduced from SEQ ID NO: 135.

SEQ ID NO: 137 is the amino acid sequence of the mature GH25 lysozyme from Simplicillium obclavatum.

SEQ ID NO: 138 is the cDNA sequence of a GH25 lysozyme as isolated from Aspergillus inflatus.

SEQ ID NO: 139 is the amino acid sequence as deduced from SEQ ID NO: 138.

SEQ ID NO: 140 is the amino acid sequence of the mature GH25 lysozyme from Aspergillus inflatus.

SEQ ID NO: 141 is the cDNA sequence of a GH25 lysozyme as isolated from Paracremonium inflatum.

SEQ ID NO: 142 is the amino acid sequence as deduced from SEQ ID NO: 141.

SEQ ID NO: 143 is the amino acid sequence of the mature GH25 lysozyme from Paracremonium inflatum.

SEQ ID NO: 144 is the cDNA sequence of a GH25 lysozyme as isolated from Westerdykella sp.

SEQ ID NO: 145 is the amino acid sequence as deduced from SEQ ID NO: 144.

SEQ ID NO: 146 is the amino acid sequence of the mature GH25 lysozyme from Westerdykella sp.

SEQ ID NO: 147 is the cDNA sequence of a GH25 lysozyme as isolated from Stropharia semiglobata.

SEQ ID NO: 148 is the amino acid sequence as deduced from SEQ ID NO: 147.

SEQ ID NO: 149 is the amino acid sequence of the mature GH25 lysozyme from Stropharia semiglobata.

SEQ ID NO: 150 is the cDNA sequence of a GH25 lysozyme as isolated from Stropharia semiglobata.

SEQ ID NO: 151 is the amino acid sequence as deduced from SEQ ID NO: 150.

SEQ ID NO: 152 is the amino acid sequence of the mature GH25 lysozyme from Stropharia semiglobata.

SEQ ID NO: 153 is the cDNA sequence of a GH25 lysozyme as isolated from Stropharia semiglobata.

SEQ ID NO: 154 is the amino acid sequence as deduced from SEQ ID NO: 153.

SEQ ID NO: 155 is the amino acid sequence of the mature GH25 lysozyme from Stropharia semiglobata.

SEQ ID NO: 156 is the cDNA sequence of a GH25 lysozyme as isolated from Gelasinospora cratophora.

SEQ ID NO: 157 is the amino acid sequence as deduced from SEQ ID NO: 156.

SEQ ID NO: 158 is the amino acid sequence of the mature GH25 lysozyme from Gelasinospora cratophora.

SEQ ID NO: 159 is the mature amino acid sequence of a wild type GH25 lysozyme from Acremonium alcalophilum as described in WO 2013/076253.

SEQ ID NO: 160 is the conserved motif YKNA.

SEQ ID NO: 161 is the conserved motif FGGW[S/T].

SEQ ID NO: 162 is the forward primer C8VRJ-F.

SEQ ID NO: 163 is the reverse primer C8VRJ-R.

SEQ ID NO: 164 is the forward primer C8VRQ-F.

SEQ ID NO: 165 is the reverse primer C8VRQ-R.

SEQ ID NO: 166 is the forward primer C8VS9-F.

SEQ ID NO: 167 is the reverse primer C8VS9-R.

SEQ ID NO: 168 is the forward primer C8VSA-F.

SEQ ID NO: 169 is the reverse primer C8VSA-R.

SEQ ID NO: 170 is the forward primer C8VSC-F.

SEQ ID NO: 171 is the reverse primer C8VSC-R.

SEQ ID NO: 172 is the forward primer C8VRT-F.

SEQ ID NO: 173 is the reverse primer C8VRT-R.

SEQ ID NO: 174 is the forward primer C8VS8-F.

SEQ ID NO: 175 is the reverse primer C8VS8-R.

SEQ ID NO: 176 is the forward primer C122P5-F.

SEQ ID NO: 177 is the reverse primer C122P5-R.

SEQ ID NO: 178 is the forward primer C6ZGC-F.

SEQ ID NO: 179 is the reverse primer C6ZGC-R.

SEQ ID NO: 180 is the forward primer C6ZG7-FF.

SEQ ID NO: 181 is the reverse primer C6ZG7-R.

SEQ ID NO: 182 is the forward primer C122PC-F.

SEQ ID NO: 183 is the reverse primer C122PC-R.

SEQ ID NO: 184 is the forward primer C122PA-F.

SEQ ID NO: 185 is the reverse primer C122PA-R.

SEQ ID NO: 186 is the forward primer C122P9-F.

SEQ ID NO: 187 is the reverse primer C122P9-R.

SEQ ID NO: 188 is the forward primer WIN1054-F.

SEQ ID NO: 189 is the reverse primer WIN1054-R.

SEQ ID NO: 190 is the forward primer WIN1065-F.

SEQ ID NO: 191 is the reverse primer WIN1065-R.

SEQ ID NO: 192 is the forward primer WIN1057-F.

SEQ ID NO: 193 is the reverse primer WIN1057-R.

SEQ ID NO: 194 is the forward primer WIN1058-F.

SEQ ID NO: 195 is the reverse primer WIN1058-R.

SEQ ID NO: 196 is the forward primer KKSC0132-F.

SEQ ID NO: 197 is the reverse primer KKSC0132-R.

SEQ ID NO: 198 is the forward primer KKSC0133-F.

SEQ ID NO: 199 is the reverse primer KKSC0133-R.

SEQ ID NO: 200 is the forward primer KKSC0311-F.

SEQ ID NO: 201 is the reverse primer KKSC0311-R.

SEQ ID NO: 202 is the forward primer KKSC0314-F.

SEQ ID NO: 203 is the reverse primer KKSC0314-R.

SEQ ID NO: 204 is the forward primer KKSC0862-F.

SEQ ID NO: 205 is the reverse primer KKSC0862-R.

SEQ ID NO: 206 is the forward primer KKSC0819-F.

SEQ ID NO: 207 is the reverse primer KKSC0819-R.

SEQ ID NO: 208 is the forward primer KKSC0317-F.

SEQ ID NO: 209 is the reverse primer KKSC0317-F.

SEQ ID NO: 210 is forward primer F1.

SEQ ID NO: 211 is reverse primer F1.

SEQ ID NO: 212 is forward primer F3.

SEQ ID NO: 213 is reverse primer F3.

SEQ ID NO: 214 is primer bind forward.

SEQ ID NO: 215 is primer bind reverse.

SEQ ID NO: 216 is the synthetic DNA construct of plasmid pDAu770.

SEQ ID NO: 217 is the cDNA sequence of a GH25 lysozyme as isolated from Flammulina velutipes.

SEQ ID NO: 218 is the amino acid sequence as deduced from SEQ ID NO: 217.

SEQ ID NO: 219 is the codon optimised DNA the GH25 lysozyme as isolated from Flammulina velutipes.

SEQ ID NO: 220 is the amino acid sequence as deduced from SEQ ID NO: 219.

SEQ ID NO: 221 is the amino acid sequence of the mature GH25 lysozyme from Flammulina velutipes.

SEQ ID NO: 222 is the cDNA sequence of a GH25 lysozyme as isolated from Deconica coprophila.

SEQ ID NO: 223 is the amino acid sequence as deduced from SEQ ID NO: 222.

SEQ ID NO: 224 is the amino acid sequence of the mature GH25 lysozyme from Deconica coprophila.

SEQ ID NO: 225 is the cDNA sequence of a GH25 lysozyme as isolated from Rhizomucor pusillus.

SEQ ID NO: 226 is the amino acid sequence as deduced from SEQ ID NO: 225.

SEQ ID NO: 227 is the amino acid sequence of the mature GH25 lysozyme from Rhizomucor pusillus.

SEQ ID NO: 228 is the cDNA sequence of a GH25 lysozyme as isolated from Stropharia semiglobata.

SEQ ID NO: 229 is the amino acid sequence as deduced from SEQ ID NO: 228.

SEQ ID NO: 230 is the amino acid sequence of the mature GH25 lysozyme from Stropharia semiglobata.

SEQ ID NO: 231 is the cDNA sequence of a GH25 lysozyme as isolated from Stropharia semiglobata.

SEQ ID NO: 232 is the amino acid sequence as deduced from SEQ ID NO: 231.

SEQ ID NO: 233 is the amino acid sequence of the mature GH25 lysozyme from Stropharia semiglobata.

SEQ ID NO: 234 is the forward primer C8VSE-F.

SEQ ID NO: 235 is the reverse primer C8VSE-R.

### BRIEF DESCRIPTION OF THE FIGURES

FIG. 1 represents the map of the different DNA features included on the plasmid pDAu770. The amy2 locus flanking regions (3′ and 5′) are indicated by white boxes. Promoter regions are indicated by green boxes for the promoter region of the pyrG, tef1 and tpi gene. The purple boxes indicate the selection cassette (ampR for ampicillin resistance and amdS for acetamide selection). The terminator regions are indicated by blue boxes for the terminator region of the niaD and amg genes. The coding region of the FLPase (sFLP) and the first exon of the pyrG gene are indicated in orange. The 5′ region of the pyrG intron is indicated in grey. The origin of replication of the plasmid is indicated by ORI.

FIG. 2 is the schematic representation of transformation of the host strain DAu785 by the transforming DNA (either plasmid pDAu724 or derivatives or OverlapExtension PCR products.

The top panel represents the locus amy2 with the integration of the FLP landing pad composed of FRT-F and FRT-F3 the FLPase recognition site, as well as the amdS (acetamide) selection marker and the FLPase expression cassette. A split PyrG marker has been used and at the amy2 locus 5′ end of the pyrG marker is inserted.

The middle panel represents the transforming DNA, in particular the region that is integrated at the FLP landing pad by site specific recombination mediated by FLPase. The palsmid or PCR product must contain FRT-F and F3 sites as well as the remaining 3′ part of the pyrG marker.

The bottom panel represents the resulting amy2 locus after site specific integration of the transformaing DNA between the FRT sites. The amdS and FLP cassettes have been exchanged with the GOI expression cassette and 3′ part of the pyrG marker reconstituting a fully functional selection marker.

FIGS. 3 and 4 are a series of panels each illustrating the results of Example 56: Method for the Determination of Lysozyme Activity (OD405) Against Lactobacillus johnsonii at concentrations of the enzymes of 0.625 ppm, 1.25 ppm, 2.5 ppm and 5 ppm. Each OD measurement represents the difference after the original (background) reading was subtracted and represents the average of two OD measurements.

The Figures illustrate that a number of the enzymes that have improved lysozyme activity against the peptidoglycans found in the cell walls of Micrococcus lysodeikticus also have excellent lysozyme activity against the peptidoglycans found in the cell walls of Lactobacillus johnsonii even at very low doses.

FIGS. 5 and 6 represents a series of panels each illustrating the results of Example 56: Method for the Determination of Lysozyme Activity (OD405) Against Lactobacillus johnsonii at concentrations of the enzymes of 0.625 ppm, 1.25 ppm, 2.5 ppm, 5 ppm, 10 and 20 ppm. Each OD measurement represents the difference after the original (background) reading was subtracted and represents the average of two OD measurements.

The Figures illustrate that a number of the enzymes that have improved lysozyme activity against the peptidoglycans found in the cell walls of Micrococcus lysodeikticus also have excellent lysozyme activity against the peptidoglycans found in the cell walls of Lactobacillus johnsonii, even at very low doses.

### DETAILED DESCRIPTION

Animal: The term “animal” refers to any animal except humans. Examples of animals are monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer hens (referred to herein as layers)); horses (including but not limited to hotbloods, coldbloods and warm bloods) crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

Animal feed: The term “animal feed” refers to any compound, preparation, or mixture suitable for, or intended for intake by an animal. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Antimicrobial activity: The term “antimicrobial activity” is defined herein as an activity that kills or inhibits the growth of microorganisms, such as, algae, archea, bacteria, fungi and/or protozoans. The antimicrobial activity can, for example, be bactericidal meaning the killing of bacteria or bacteriostatic meaning the prevention of bacterial growth. The antimicrobial activity can include catalyzing the hydrolysis of 1,4-beta-linkages between N-acetylmuramic acid and

N-acetyl-D-glucosamine residues in a peptidoglycan and between N-acetyl-D-glucosamine residues in chitodextrins. Antimicrobial activity can also include the lysozyme binding to the surface of the microorganism and inhibiting its growth. The antimicrobial effect can also include the use of the lysozymes of the present disclosure for activation of bacterial autolysins, as an immunostimulator, by inhibiting or reducing bacterial toxins and by an opsonin effect.

Body Weight Gain: The term “body weight gain” means an increase in live weight of an animal during a given period of time e.g. the increase in weight from day 1 to day 21.

CDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Concentrates: The term “concentrates” means feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Control sequences: The term “control sequences” means nucleic acid sequences necessary for expression of a polynucleotide encoding a mature polypeptide of the present disclosure. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

European Production Efficacy Factor (EPEF): The “European Production Efficacy Factor” is a way of comparing the performance of animals. This single-figure facilitates comparison of performance within and among farms and can be used to assess environmental, climatic and managemental variables. The EPEF is calculated as [(liveability (%)×Liveweight (kg))/(Age at depletion (days)×FCR)]×100, wherein livability is the percentage of animals alive at slaughter, Liveweight is the average weight of the animals at slaughter, age of depletion is the age of the animals at slaughter and FCR is the feed conversion ratio at slaughter.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Feed Conversion Ratio (FCR): FCR is a measure of an animal's efficiency in converting feed mass into increases of the desired output. Animals raised for meat—such as swine, poultry and fish—the output is the mass gained by the animal. Specifically, FCR is calculated as feed intake divided by weight gain, all over a specified period. Improvement in FCR means reduction of the FCR value. A FCR improvement of 2% means that the FCR was reduced by 2%.

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present disclosure in feed results in an increased weight gain per unit of feed intake compared with an animal fed without said feed additive composition being present.

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Fragment: The term “fragment” means a polypeptide having one or more (e.g., several) amino acids absent from the amino and/or carboxyl terminus of a mature polypeptide or domain; wherein the fragment has lysozyme activity.

In one aspect, the fragment comprises at least 90% of the length of the mature polypeptide, such as at least 186 amino acids of SEQ ID NO: 2, at least 186 amino acids of SEQ ID NO: 3, at least 180 amino acids of SEQ ID NO: 5, at least 180 amino acids of SEQ ID NO: 6, at least 180 amino acids of SEQ ID NO: 8, at least 180 amino acids of SEQ ID NO: 9, at least 182 amino acids of SEQ ID NO: 11, at least 182 amino acids of SEQ ID NO: 12, at least 187 amino acids of SEQ ID NO: 14, at least 187 amino acids of SEQ ID NO: 15, at least 186 amino acids of SEQ ID NO: 17, at least 186 amino acids of SEQ ID NO: 18, at least 187 amino acids of SEQ ID NO: 20, at least 187 amino acids of SEQ ID NO: 21, at least 186 amino acids of SEQ ID NO: 23, at least 186 amino acids of SEQ ID NO: 24, at least 186 amino acids of SEQ ID NO: 26, at least 186 amino acids of SEQ ID NO: 27, at least 186 amino acids of SEQ ID NO: 29, at least 186 amino acids of SEQ ID NO: 30, at least 187 amino acids of SEQ ID NO: 32, at least 187 amino acids of SEQ ID NO: 33, at least 187 amino acids of SEQ ID NO: 35, at least 187 amino acids of SEQ ID NO: 36, at least 185 amino acids of SEQ ID NO: 38, at least 185 amino acids of SEQ ID NO: 39, at least 186 amino acids of SEQ ID NO: 41, at least 186 amino acids of SEQ ID NO: 42, at least 194 amino acids of SEQ ID NO: 44, at least 194 amino acids of SEQ ID NO: 45, at least 196 amino acids of SEQ ID NO: 47, at least 196 amino acids of SEQ ID NO: 48, at least 183 amino acids of SEQ ID NO: 50, at least 183 amino acids of SEQ ID NO: 51, at least 182 amino acids of SEQ ID NO: 53, at least 182 amino acids of SEQ ID NO: 54, at least 188 amino acids of SEQ ID NO: 56, at least 188 amino acids of SEQ ID NO: 57, at least 187 amino acids of SEQ ID NO: 59, at least 187 amino acids of SEQ ID NO: 60, at least 189 amino acids of SEQ ID NO: 62, at least 189 amino acids of SEQ ID NO: 63, at least 186 amino acids of SEQ ID NO: 65, at least 186 amino acids of SEQ ID NO: 66, at least 186 amino acids of SEQ ID NO: 68, at least 186 amino acids of SEQ ID NO: 69, at least 187 amino acids of SEQ ID NO: 71, at least 187 amino acids of SEQ ID NO: 72, at least 195 amino acids of SEQ ID NO: 74, at least 195 amino acids of SEQ ID NO: 75, at least 187 amino acids of SEQ ID NO: 77, at least 187 amino acids of SEQ ID NO: 79, at least 187 amino acids of SEQ ID NO: 80, at least 180 amino acids of SEQ ID NO: 82, at least 180 amino acids of SEQ ID NO: 83, at least 181 amino acids of SEQ ID NO: 85, at least 181 amino acids of SEQ ID NO: 86, at least 186 amino acids of SEQ ID NO: 88, at least 186 amino acids of SEQ ID NO: 89, at least 181 amino acids of SEQ ID NO: 91, at least 181 amino acids of SEQ ID NO: 92, at least 180 amino acids of SEQ ID NO: 94, at least 180 amino acids of SEQ ID NO: 95, at least 181 amino acids of SEQ ID NO: 97, at least 181 amino acids of SEQ ID NO: 98, at least 185 amino acids of SEQ ID NO: 100, at least 185 amino acids of SEQ ID NO: 101, at least 181 amino acids of SEQ ID NO: 103, at least 181 amino acids of SEQ ID NO: 104, at least 181 amino acids of SEQ ID NO: 106, at least 181 amino acids of SEQ ID NO: 107, at least 181 amino acids of SEQ ID NO: 109, at least 181 amino acids of SEQ ID NO: 110, at least 181 amino acids of SEQ ID NO: 112, at least 181 amino acids of SEQ ID NO: 113, at least 185 amino acids of SEQ ID NO: 115, at least 185 amino acids of SEQ ID NO: 116, at least 186 amino acids of SEQ ID NO: 118, at least 186 amino acids of SEQ ID NO: 119, at least 187 amino acids of SEQ ID NO: 121, at least 187 amino acids of SEQ ID NO: 122, at least 193 amino acids of SEQ ID NO: 124, at least 193 amino acids of SEQ ID NO: 125, at least 195 amino acids of SEQ ID NO: 127, at least 195 amino acids of SEQ ID NO: 128, at least 192 amino acids of SEQ ID NO: 130, at least 192 amino acids of SEQ ID NO: 131, at least 187 amino acids of SEQ ID NO: 133, at least 187 amino acids of SEQ ID NO: 134, at least 182 amino acids of SEQ ID NO: 136, at least 182 amino acids of SEQ ID NO: 137, at least 194 amino acids of SEQ ID NO: 139, at least 194 amino acids of SEQ ID NO: 140, at least 186 amino acids of SEQ ID NO: 142, at least 186 amino acids of SEQ ID NO: 143, at least 187 amino acids of SEQ ID NO: 145, at least 187 amino acids of SEQ ID NO: 146, at least 186 amino acids of SEQ ID NO: 148, at least 186 amino acids of SEQ ID NO: 149, at least 186 amino acids of SEQ ID NO: 151, at least 186 amino acids of SEQ ID NO: 152, at least 186 amino acids of SEQ ID NO: 154, at least 186 amino acids of SEQ ID NO: 155, at least 187 amino acids of SEQ ID NO: 157, at least 187 amino acids of SEQ ID NO: 158, at least 186 amino acids of SEQ ID NO: 218, at least 186 amino acids of SEQ ID NO: 220, at least 186 amino acids of SEQ ID NO: 221, at least 186 amino acids of SEQ ID NO: 223, at least 186 amino acids of SEQ ID NO: 224, at least 187 amino acids of SEQ ID NO: 226, at least 187 amino acids of SEQ ID NO: 227, at least 186 amino acids of SEQ ID NO: 229, at least 186 amino acids of SEQ ID NO: 230, at least 186 amino acids of SEQ ID NO: 232, or at least 186 amino acids of SEQ ID NO: 233.

In one aspect, the fragment comprises at least 92% of the length of the mature polypeptide, such as at least 190 amino acids of SEQ ID NO: 2, at least 190 amino acids of SEQ ID NO: 3, at least 184 amino acids of SEQ ID NO: 5, at least 184 amino acids of SEQ ID NO: 6, at least 184 amino acids of SEQ ID NO: 8, at least 184 amino acids of SEQ ID NO: 9, at least 186 amino acids of SEQ ID NO: 11, at least 186 amino acids of SEQ ID NO: 12, at least 191 amino acids of SEQ ID NO: 14, at least 191 amino acids of SEQ ID NO: 15, at least 190 amino acids of SEQ ID NO: 17, at least 190 amino acids of SEQ ID NO: 18, at least 191 amino acids of SEQ ID NO: 20, at least 191 amino acids of SEQ ID NO: 21, at least 190 amino acids of SEQ ID NO: 23, at least 190 amino acids of SEQ ID NO: 24, at least 190 amino acids of SEQ ID NO: 26, at least 190 amino acids of SEQ ID NO: 27, at least 190 amino acids of SEQ ID NO: 29, at least 190 amino acids of SEQ ID NO: 30, at least 191 amino acids of SEQ ID NO: 32, at least 191 amino acids of SEQ ID NO: 33, at least 191 amino acids of SEQ ID NO: 35, at least 191 amino acids of SEQ ID NO: 36, at least 189 amino acids of SEQ ID NO: 38, at least 189 amino acids of SEQ ID NO: 39, at least 190 amino acids of SEQ ID NO: 41, at least 190 amino acids of SEQ ID NO: 42, at least 198 amino acids of SEQ ID NO: 44, at least 198 amino acids of SEQ ID NO: 45, at least 200 amino acids of SEQ ID NO: 47, at least 200 amino acids of SEQ ID NO: 48, at least 187 amino acids of SEQ ID NO: 50, at least 187 amino acids of SEQ ID NO: 51, at least 186 amino acids of SEQ ID NO: 53, at least 186 amino acids of SEQ ID NO: 54, at least 192 amino acids of SEQ ID NO: 56, at least 192 amino acids of SEQ ID NO: 57, at least 191 amino acids of SEQ ID NO: 59, at least 191 amino acids of SEQ ID NO: 60, at least 193 amino acids of SEQ ID NO: 62, at least 193 amino acids of SEQ ID NO: 63, at least 190 amino acids of SEQ ID NO: 65, at least 190 amino acids of SEQ ID NO: 66, at least 190 amino acids of SEQ ID NO: 68, at least 190 amino acids of SEQ ID NO: 69, at least 191 amino acids of SEQ ID NO: 71, at least 191 amino acids of SEQ ID NO: 72, at least 199 amino acids of SEQ ID NO: 74, at least 199 amino acids of SEQ ID NO: 75, at least 191 amino acids of SEQ ID NO: 77, at least 191 amino acids of SEQ ID NO: 79, at least 191 amino acids of SEQ ID NO: 80, at least 184 amino acids of SEQ ID NO: 82, at least 184 amino acids of SEQ ID NO: 83, at least 185 amino acids of SEQ ID NO: 85, at least 185 amino acids of SEQ ID NO: 86, at least 190 amino acids of SEQ ID NO: 88, at least 190 amino acids of SEQ ID NO: 89, at least 185 amino acids of SEQ ID NO: 91, at least 185 amino acids of SEQ ID NO: 92, at least 184 amino acids of SEQ ID NO: 94, at least 184 amino acids of SEQ ID NO: 95, at least 185 amino acids of SEQ ID NO: 97, at least 185 amino acids of SEQ ID NO: 98, at least 189 amino acids of SEQ ID NO: 100, at least 189 amino acids of SEQ ID NO: 101, at least 185 amino acids of SEQ ID NO: 103, at least 185 amino acids of SEQ ID NO: 104, at least 185 amino acids of SEQ ID NO: 106, at least 185 amino acids of SEQ ID NO: 107, at least 185 amino acids of SEQ ID NO: 109, at least 185 amino acids of SEQ ID NO: 110, at least 185 amino acids of SEQ ID NO: 112, at least 185 amino acids of SEQ ID NO: 113, at least 189 amino acids of SEQ ID NO: 115, at least 189 amino acids of SEQ ID NO: 116, at least 190 amino acids of SEQ ID NO: 118, at least 190 amino acids of SEQ ID NO: 119, at least 191 amino acids of SEQ ID NO: 121, at least 191 amino acids of SEQ ID NO: 122, at least 197 amino acids of SEQ ID NO: 124, at least 197 amino acids of SEQ ID NO: 125, at least 199 amino acids of SEQ ID NO: 127, at least 199 amino acids of SEQ ID NO: 128, at least 196 amino acids of SEQ ID NO: 130, at least 196 amino acids of SEQ ID NO: 131, at least 191 amino acids of SEQ ID NO: 133, at least 191 amino acids of SEQ ID NO: 134, at least 186 amino acids of SEQ ID NO: 136, at least 186 amino acids of SEQ ID NO: 137, at least 198 amino acids of SEQ ID NO: 139, at least 198 amino acids of SEQ ID NO: 140, at least 190 amino acids of SEQ ID NO: 142, at least 190 amino acids of SEQ ID NO: 143, at least 191 amino acids of SEQ ID NO: 145, at least 191 amino acids of SEQ ID NO: 146, at least 190 amino acids of SEQ ID NO: 148, at least 190 amino acids of SEQ ID NO: 149, at least 190 amino acids of SEQ ID NO: 151, at least 190 amino acids of SEQ ID NO: 152, at least 190 amino acids of SEQ ID NO: 154, at least 190 amino acids of SEQ ID NO: 155, at least 191 amino acids of SEQ ID NO: 157, at least 191 amino acids of SEQ ID NO: 158, at least 190 amino acids of SEQ ID NO: 218, at least 190 amino acids of SEQ ID NO: 220, at least 190 amino acids of SEQ ID NO: 221, at least 190 amino acids of SEQ ID NO: 223, at least 190 amino acids of SEQ ID NO: 224, at least 191 amino acids of SEQ ID NO: 226, at least 191 amino acids of SEQ ID NO: 227, at least 190 amino acids of SEQ ID NO: 229, at least 190 amino acids of SEQ ID NO: 230, at least 190 amino acids of SEQ ID NO: 232, or at least 190 amino acids of SEQ ID NO: 233.

In one aspect, the fragment comprises at least 94% of the length of the mature polypeptide, such as at least 194 amino acids of SEQ ID NO: 2, at least 194 amino acids of SEQ ID NO: 3, at least 188 amino acids of SEQ ID NO: 5, at least 188 amino acids of SEQ ID NO: 6, at least 188 amino acids of SEQ ID NO: 8, at least 188 amino acids of SEQ ID NO: 9, at least 190 amino acids of SEQ ID NO: 11, at least 190 amino acids of SEQ ID NO: 12, at least 195 amino acids of SEQ ID NO: 14, at least 195 amino acids of SEQ ID NO: 15, at least 194 amino acids of SEQ ID NO: 17, at least 194 amino acids of SEQ ID NO: 18, at least 195 amino acids of SEQ ID NO: 20, at least 195 amino acids of SEQ ID NO: 21, at least 194 amino acids of SEQ ID NO: 23, at least 194 amino acids of SEQ ID NO: 24, at least 194 amino acids of SEQ ID NO: 26, at least 194 amino acids of SEQ ID NO: 27, at least 194 amino acids of SEQ ID NO: 29, at least 194 amino acids of SEQ ID NO: 30, at least 195 amino acids of SEQ ID NO: 32, at least 195 amino acids of SEQ ID NO: 33, at least 195 amino acids of SEQ ID NO: 35, at least 195 amino acids of SEQ ID NO: 36, at least 193 amino acids of SEQ ID NO: 38, at least 193 amino acids of SEQ ID NO: 39, at least 194 amino acids of SEQ ID NO: 41, at least 194 amino acids of SEQ ID NO: 42, at least 203 amino acids of SEQ ID NO: 44, at least 203 amino acids of SEQ ID NO: 45, at least 204 amino acids of SEQ ID NO: 47, at least 204 amino acids of SEQ ID NO: 48, at least 191 amino acids of SEQ ID NO: 50, at least 191 amino acids of SEQ ID NO: 51, at least 190 amino acids of SEQ ID NO: 53, at least 190 amino acids of SEQ ID NO: 54, at least 196 amino acids of SEQ ID NO: 56, at least 196 amino acids of SEQ ID NO: 57, at least 195 amino acids of SEQ ID NO: 59, at least 195 amino acids of SEQ ID NO: 60, at least 197 amino acids of SEQ ID NO: 62, at least 197 amino acids of SEQ ID NO: 63, at least 194 amino acids of SEQ ID NO: 65, at least 194 amino acids of SEQ ID NO: 66, at least 194 amino acids of SEQ ID NO: 68, at least 194 amino acids of SEQ ID NO: 69, at least 195 amino acids of SEQ ID NO: 71, at least 195 amino acids of SEQ ID NO: 72, at least 203 amino acids of SEQ ID NO: 74, at least 203 amino acids of SEQ ID NO: 75, at least 195 amino acids of SEQ ID NO: 77, at least 195 amino acids of SEQ ID NO: 79, at least 195 amino acids of SEQ ID NO: 80, at least 188 amino acids of SEQ ID NO: 82, at least 188 amino acids of SEQ ID NO: 83, at least 189 amino acids of SEQ ID NO: 85, at least 189 amino acids of SEQ ID NO: 86, at least 194 amino acids of SEQ ID NO: 88, at least 194 amino acids of SEQ ID NO: 89, at least 189 amino acids of SEQ ID NO: 91, at least 189 amino acids of SEQ ID NO: 92, at least 188 amino acids of SEQ ID NO: 94, at least 188 amino acids of SEQ ID NO: 95, at least 189 amino acids of SEQ ID NO: 97, at least 189 amino acids of SEQ ID NO: 98, at least 193 amino acids of SEQ ID NO: 100, at least 193 amino acids of SEQ ID NO: 101, at least 189 amino acids of SEQ ID NO: 103, at least 189 amino acids of SEQ ID NO: 104, at least 189 amino acids of SEQ ID NO: 106, at least 189 amino acids of SEQ ID NO: 107, at least 189 amino acids of SEQ ID NO: 109, at least 189 amino acids of SEQ ID NO: 110, at least 189 amino acids of SEQ ID NO: 112, at least 189 amino acids of SEQ ID NO: 113, at least 193 amino acids of SEQ ID NO: 115, at least 193 amino acids of SEQ ID NO: 116, at least 194 amino acids of SEQ ID NO: 118, at least 194 amino acids of SEQ ID NO: 119, at least 195 amino acids of SEQ ID NO: 121, at least 195 amino acids of SEQ ID NO: 122, at least 202 amino acids of SEQ ID NO: 124, at least 202 amino acids of SEQ ID NO: 125, at least 203 amino acids of SEQ ID NO: 127, at least 203 amino acids of SEQ ID NO: 128, at least 201 amino acids of SEQ ID NO: 130, at least 201 amino acids of SEQ ID NO: 131, at least 195 amino acids of SEQ ID NO: 133, at least 195 amino acids of SEQ ID NO: 134, at least 190 amino acids of SEQ ID NO: 136, at least 190 amino acids of SEQ ID NO: 137, at least 203 amino acids of SEQ ID NO: 139, at least 203 amino acids of SEQ ID NO: 140, at least 194 amino acids of SEQ ID NO: 142, at least 194 amino acids of SEQ ID NO: 143, at least 195 amino acids of SEQ ID NO: 145, at least 195 amino acids of SEQ ID NO: 146, at least 194 amino acids of SEQ ID NO: 148, at least 194 amino acids of SEQ ID NO: 149, at least 194 amino acids of SEQ ID NO: 151, at least 194 amino acids of SEQ ID NO: 152, at least 194 amino acids of SEQ ID NO: 154, at least 194 amino acids of SEQ ID NO: 155, at least 195 amino acids of SEQ ID NO: 157, at least 195 amino acids of SEQ ID NO: 158, at least 194 amino acids of SEQ ID NO: 218, at least 194 amino acids of SEQ ID NO: 220, at least 194 amino acids of SEQ ID NO: 221, at least 194 amino acids of SEQ ID NO: 223, at least 194 amino acids of SEQ ID NO: 224, at least 195 amino acids of SEQ ID NO: 226, at least 195 amino acids of SEQ ID NO: 227, at least 194 amino acids of SEQ ID NO: 229, at least 194 amino acids of SEQ ID NO: 230, at least 194 amino acids of SEQ ID NO: 232, or at least 194 amino acids of SEQ ID NO: 233.

In one aspect, the fragment comprises at least 96% of the length of the mature polypeptide, such as at least 198 amino acids of SEQ ID NO: 2, at least 198 amino acids of SEQ ID NO: 3, at least 192 amino acids of SEQ ID NO: 5, at least 192 amino acids of SEQ ID NO: 6, at least 192 amino acids of SEQ ID NO: 8, at least 192 amino acids of SEQ ID NO: 9, at least 194 amino acids of SEQ ID NO: 11, at least 194 amino acids of SEQ ID NO: 12, at least 199 amino acids of SEQ ID NO: 14, at least 199 amino acids of SEQ ID NO: 15, at least 198 amino acids of SEQ ID NO: 17, at least 198 amino acids of SEQ ID NO: 18, at least 199 amino acids of SEQ ID NO: 20, at least 199 amino acids of SEQ ID NO: 21, at least 198 amino acids of SEQ ID NO: 23, at least 198 amino acids of SEQ ID NO: 24, at least 198 amino acids of SEQ ID NO: 26, at least 198 amino acids of SEQ ID NO: 27, at least 198 amino acids of SEQ ID NO: 29, at least 198 amino acids of SEQ ID NO: 30, at least 199 amino acids of SEQ ID NO: 32, at least 199 amino acids of SEQ ID NO: 33, at least 199 amino acids of SEQ ID NO: 35, at least 199 amino acids of SEQ ID NO: 36, at least 197 amino acids of SEQ ID NO: 38, at least 197 amino acids of SEQ ID NO: 39, at least 198 amino acids of SEQ ID NO: 41, at least 198 amino acids of SEQ ID NO: 42, at least 207 amino acids of SEQ ID NO: 44, at least 207 amino acids of SEQ ID NO: 45, at least 209 amino acids of SEQ ID NO: 47, at least 209 amino acids of SEQ ID NO: 48, at least 195 amino acids of SEQ ID NO: 50, at least 195 amino acids of SEQ ID NO: 51, at least 194 amino acids of SEQ ID NO: 53, at least 194 amino acids of SEQ ID NO: 54, at least 200 amino acids of SEQ ID NO: 56, at least 200 amino acids of SEQ ID NO: 57, at least 199 amino acids of SEQ ID NO: 59, at least 199 amino acids of SEQ ID NO: 60, at least 201 amino acids of SEQ ID NO: 62, at least 201 amino acids of SEQ ID NO: 63, at least 198 amino acids of SEQ ID NO: 65, at least 198 amino acids of SEQ ID NO: 66, at least 198 amino acids of SEQ ID NO: 68, at least 198 amino acids of SEQ ID NO: 69, at least 199 amino acids of SEQ ID NO: 71, at least 199 amino acids of SEQ ID NO: 72, at least 208 amino acids of SEQ ID NO: 74, at least 208 amino acids of SEQ ID NO: 75, at least 199 amino acids of SEQ ID NO: 77, at least 199 amino acids of SEQ ID NO: 79, at least 199 amino acids of SEQ ID NO: 80, at least 192 amino acids of SEQ ID NO: 82, at least 192 amino acids of SEQ ID NO: 83, at least 193 amino acids of SEQ ID NO: 85, at least 193 amino acids of SEQ ID NO: 86, at least 198 amino acids of SEQ ID NO: 88, at least 198 amino acids of SEQ ID NO: 89, at least 193 amino acids of SEQ ID NO: 91, at least 193 amino acids of SEQ ID NO: 92, at least 192 amino acids of SEQ ID NO: 94, at least 192 amino acids of SEQ ID NO: 95, at least 193 amino acids of SEQ ID NO: 97, at least 193 amino acids of SEQ ID NO: 98, at least 197 amino acids of SEQ ID NO: 100, at least 197 amino acids of SEQ ID NO: 101, at least 193 amino acids of SEQ ID NO: 103, at least 193 amino acids of SEQ ID NO: 104, at least 193 amino acids of SEQ ID NO: 106, at least 193 amino acids of SEQ ID NO: 107, at least 193 amino acids of SEQ ID NO: 109, at least 193 amino acids of SEQ ID NO: 110, at least 193 amino acids of SEQ ID NO: 112, at least 193 amino acids of SEQ ID NO: 113, at least 197 amino acids of SEQ ID NO: 115, at least 197 amino acids of SEQ ID NO: 116, at least 198 amino acids of SEQ ID NO: 118, at least 198 amino acids of SEQ ID NO: 119, at least 199 amino acids of SEQ ID NO: 121, at least 199 amino acids of SEQ ID NO: 122, at least 206 amino acids of SEQ ID NO: 124, at least 206 amino acids of SEQ ID NO: 125, at least 208 amino acids of SEQ ID NO: 127, at least 208 amino acids of SEQ ID NO: 128, at least 205 amino acids of SEQ ID NO: 130, at least 205 amino acids of SEQ ID NO: 131, at least 199 amino acids of SEQ ID NO: 133, at least 199 amino acids of SEQ ID NO: 134, at least 194 amino acids of SEQ ID NO: 136, at least 194 amino acids of SEQ ID NO: 137, at least 207 amino acids of SEQ ID NO: 139, at least 207 amino acids of SEQ ID NO: 140, at least 198 amino acids of SEQ ID NO: 142, at least 198 amino acids of SEQ ID NO: 143, at least 199 amino acids of SEQ ID NO: 145, at least 199 amino acids of SEQ ID NO: 146, at least 198 amino acids of SEQ ID NO: 148, at least 198 amino acids of SEQ ID NO: 149, at least 198 amino acids of SEQ ID NO: 151, at least 198 amino acids of SEQ ID NO: 152, at least 198 amino acids of SEQ ID NO: 154, at least 198 amino acids of SEQ ID NO: 155, at least 199 amino acids of SEQ ID NO: 157, at least 199 amino acids of SEQ ID NO: 158, at least 198 amino acids of SEQ ID NO: 218, at least 198 amino acids of SEQ ID NO: 220, at least 198 amino acids of SEQ ID NO: 221, at least 198 amino acids of SEQ ID NO: 223, at least 198 amino acids of SEQ ID NO: 224, at least 199 amino acids of SEQ ID NO: 226, at least 199 amino acids of SEQ ID NO: 227, at least 198 amino acids of SEQ ID NO: 229, at least 198 amino acids of SEQ ID NO: 230, at least 198 amino acids of SEQ ID NO: 232, or at least 198 amino acids of SEQ ID NO: 233.

In one aspect, the fragment comprises at least 98% of the length of the mature polypeptide, such as at least 202 amino acids of SEQ ID NO: 2, at least 202 amino acids of SEQ ID NO: 3, at least 196 amino acids of SEQ ID NO: 5, at least 196 amino acids of SEQ ID NO: 6, at least 196 amino acids of SEQ ID NO: 8, at least 196 amino acids of SEQ ID NO: 9, at least 198 amino acids of SEQ ID NO: 11, at least 198 amino acids of SEQ ID NO: 12, at least 203 amino acids of SEQ ID NO: 14, at least 203 amino acids of SEQ ID NO: 15, at least 202 amino acids of SEQ ID NO: 17, at least 202 amino acids of SEQ ID NO: 18, at least 203 amino acids of SEQ ID NO: 20, at least 203 amino acids of SEQ ID NO: 21, at least 202 amino acids of SEQ ID NO: 23, at least 202 amino acids of SEQ ID NO: 24, at least 202 amino acids of SEQ ID NO: 26, at least 202 amino acids of SEQ ID NO: 27, at least 202 amino acids of SEQ ID NO: 29, at least 202 amino acids of SEQ ID NO: 30, at least 203 amino acids of SEQ ID NO: 32, at least 203 amino acids of SEQ ID NO: 33, at least 203 amino acids of SEQ ID NO: 35, at least 203 amino acids of SEQ ID NO: 36, at least 201 amino acids of SEQ ID NO: 38, at least 201 amino acids of SEQ ID NO: 39, at least 202 amino acids of SEQ ID NO: 41, at least 202 amino acids of SEQ ID NO: 42, at least 211 amino acids of SEQ ID NO: 44, at least 211 amino acids of SEQ ID NO: 45, at least 213 amino acids of SEQ ID NO: 47, at least 213 amino acids of SEQ ID NO: 48, at least 199 amino acids of SEQ ID NO: 50, at least 199 amino acids of SEQ ID NO: 51, at least 198 amino acids of SEQ ID NO: 53, at least 198 amino acids of SEQ ID NO: 54, at least 204 amino acids of SEQ ID NO: 56, at least 204 amino acids of SEQ ID NO: 57, at least 203 amino acids of SEQ ID NO: 59, at least 203 amino acids of SEQ ID NO: 60, at least 205 amino acids of SEQ ID NO: 62, at least 205 amino acids of SEQ ID NO: 63, at least 202 amino acids of SEQ ID NO: 65, at least 202 amino acids of SEQ ID NO: 66, at least 202 amino acids of SEQ ID NO: 68, at least 202 amino acids of SEQ ID NO: 69, at least 203 amino acids of SEQ ID NO: 71, at least 203 amino acids of SEQ ID NO: 72, at least 212 amino acids of SEQ ID NO: 74, at least 212 amino acids of SEQ ID NO: 75, at least 203 amino acids of SEQ ID NO: 77, at least 203 amino acids of SEQ ID NO: 79, at least 203 amino acids of SEQ ID NO: 80, at least 196 amino acids of SEQ ID NO: 82, at least 196 amino acids of SEQ ID NO: 83, at least 197 amino acids of SEQ ID NO: 85, at least 197 amino acids of SEQ ID NO: 86, at least 202 amino acids of SEQ ID NO: 88, at least 202 amino acids of SEQ ID NO: 89, at least 197 amino acids of SEQ ID NO: 91, at least 197 amino acids of SEQ ID NO: 92, at least 196 amino acids of SEQ ID NO: 94, at least 196 amino acids of SEQ ID NO: 95, at least 197 amino acids of SEQ ID NO: 97, at least 197 amino acids of SEQ ID NO: 98, at least 201 amino acids of SEQ ID NO: 100, at least 201 amino acids of SEQ ID NO: 101, at least 197 amino acids of SEQ ID NO: 103, at least 197 amino acids of SEQ ID NO: 104, at least 197 amino acids of SEQ ID NO: 106, at least 197 amino acids of SEQ ID NO: 107, at least 197 amino acids of SEQ ID NO: 109, at least 197 amino acids of SEQ ID NO: 110, at least 197 amino acids of SEQ ID NO: 112, at least 197 amino acids of SEQ ID NO: 113, at least 201 amino acids of SEQ ID NO: 115, at least 201 amino acids of SEQ ID NO: 116, at least 202 amino acids of SEQ ID NO: 118, at least 202 amino acids of SEQ ID NO: 119, at least 203 amino acids of SEQ ID NO: 121, at least 203 amino acids of SEQ ID NO: 122, at least 210 amino acids of SEQ ID NO: 124, at least 210 amino acids of SEQ ID NO: 125, at least 212 amino acids of SEQ ID NO: 127, at least 212 amino acids of SEQ ID NO: 128, at least 209 amino acids of SEQ ID NO: 130, at least 209 amino acids of SEQ ID NO: 131, at least 203 amino acids of SEQ ID NO: 133, at least 203 amino acids of SEQ ID NO: 134, at least 198 amino acids of SEQ ID NO: 136, at least 198 amino acids of SEQ ID NO: 137, at least 211 amino acids of SEQ ID NO: 139, at least 211 amino acids of SEQ ID NO: 140, at least 202 amino acids of SEQ ID NO: 142, at least 202 amino acids of SEQ ID NO: 143, at least 203 amino acids of SEQ ID NO: 145, at least 203 amino acids of SEQ ID NO: 146, at least 202 amino acids of SEQ ID NO: 148, at least 202 amino acids of SEQ ID NO: 149, at least 202 amino acids of SEQ ID NO: 151, at least 202 amino acids of SEQ ID NO: 152, at least 202 amino acids of SEQ ID NO: 154, at least 202 amino acids of SEQ ID NO: 155, at least 203 amino acids of SEQ ID NO: 157, at least 203 amino acids of SEQ ID NO: 158, at least 202 amino acids of SEQ ID NO: 218, at least 202 amino acids of SEQ ID NO: 220, at least 202 amino acids of SEQ ID NO: 221, at least 202 amino acids of SEQ ID NO: 223, at least 202 amino acids of SEQ ID NO: 224, at least 203 amino acids of SEQ ID NO: 226, at least 203 amino acids of SEQ ID NO: 227, at least 202 amino acids of SEQ ID NO: 229, at least 202 amino acids of SEQ ID NO: 230, at least 202 amino acids of SEQ ID NO: 232, or at least 202 amino acids of SEQ ID NO: 233.

In one aspect, the fragment comprises at least 99% of the length of the mature polypeptide, such as at least 204 amino acids of SEQ ID NO: 2, at least 204 amino acids of SEQ ID NO: 3, at least 198 amino acids of SEQ ID NO: 5, at least 198 amino acids of SEQ ID NO: 6, at least 198 amino acids of SEQ ID NO: 8, at least 198 amino acids of SEQ ID NO: 9, at least 200 amino acids of SEQ ID NO: 11, at least 200 amino acids of SEQ ID NO: 12, at least 205 amino acids of SEQ ID NO: 14, at least 205 amino acids of SEQ ID NO: 15, at least 204 amino acids of SEQ ID NO: 17, at least 204 amino acids of SEQ ID NO: 18, at least 205 amino acids of SEQ ID NO: 20, at least 205 amino acids of SEQ ID NO: 21, at least 204 amino acids of SEQ ID NO: 23, at least 204 amino acids of SEQ ID NO: 24, at least 204 amino acids of SEQ ID NO: 26, at least 204 amino acids of SEQ ID NO: 27, at least 204 amino acids of SEQ ID NO: 29, at least 204 amino acids of SEQ ID NO: 30, at least 205 amino acids of SEQ ID NO: 32, at least 205 amino acids of SEQ ID NO: 33, at least 205 amino acids of SEQ ID NO: 35, at least 205 amino acids of SEQ ID NO: 36, at least 203 amino acids of SEQ ID NO: 38, at least 203 amino acids of SEQ ID NO: 39, at least 204 amino acids of SEQ ID NO: 41, at least 204 amino acids of SEQ ID NO: 42, at least 213 amino acids of SEQ ID NO: 44, at least 213 amino acids of SEQ ID NO: 45, at least 215 amino acids of SEQ ID NO: 47, at least 215 amino acids of SEQ ID NO: 48, at least 201 amino acids of SEQ ID NO: 50, at least 201 amino acids of SEQ ID NO: 51, at least 200 amino acids of SEQ ID NO: 53, at least 200 amino acids of SEQ ID NO: 54, at least 206 amino acids of SEQ ID NO: 56, at least 206 amino acids of SEQ ID NO: 57, at least 205 amino acids of SEQ ID NO: 59, at least 205 amino acids of SEQ ID NO: 60, at least 207 amino acids of SEQ ID NO: 62, at least 207 amino acids of SEQ ID NO: 63, at least 204 amino acids of SEQ ID NO: 65, at least 204 amino acids of SEQ ID NO: 66, at least 204 amino acids of SEQ ID NO: 68, at least 204 amino acids of SEQ ID NO: 69, at least 205 amino acids of SEQ ID NO: 71, at least 205 amino acids of SEQ ID NO: 72, at least 214 amino acids of SEQ ID NO: 74, at least 214 amino acids of SEQ ID NO: 75, at least 205 amino acids of SEQ ID NO: 77, at least 205 amino acids of SEQ ID NO: 79, at least 205 amino acids of SEQ ID NO: 80, at least 198 amino acids of SEQ ID NO: 82, at least 198 amino acids of SEQ ID NO: 83, at least 199 amino acids of SEQ ID NO: 85, at least 199 amino acids of SEQ ID NO: 86, at least 204 amino acids of SEQ ID NO: 88, at least 204 amino acids of SEQ ID NO: 89, at least 199 amino acids of SEQ ID NO: 91, at least 199 amino acids of SEQ ID NO: 92, at least 198 amino acids of SEQ ID NO: 94, at least 198 amino acids of SEQ ID NO: 95, at least 199 amino acids of SEQ ID NO: 97, at least 199 amino acids of SEQ ID NO: 98, at least 203 amino acids of SEQ ID NO: 100, at least 203 amino acids of SEQ ID NO: 101, at least 199 amino acids of SEQ ID NO: 103, at least 199 amino acids of SEQ ID NO: 104, at least 199 amino acids of SEQ ID NO: 106, at least 199 amino acids of SEQ ID NO: 107, at least 199 amino acids of SEQ ID NO: 109, at least 199 amino acids of SEQ ID NO: 110, at least 199 amino acids of SEQ ID NO: 112, at least 199 amino acids of SEQ ID NO: 113, at least 203 amino acids of SEQ ID NO: 115, at least 203 amino acids of SEQ ID NO: 116, at least 204 amino acids of SEQ ID NO: 118, at least 204 amino acids of SEQ ID NO: 119, at least 205 amino acids of SEQ ID NO: 121, at least 205 amino acids of SEQ ID NO: 122, at least 212 amino acids of SEQ ID NO: 124, at least 212 amino acids of SEQ ID NO: 125, at least 214 amino acids of SEQ ID NO: 127, at least 214 amino acids of SEQ ID NO: 128, at least 211 amino acids of SEQ ID NO: 130, at least 211 amino acids of SEQ ID NO: 131, at least 205 amino acids of SEQ ID NO: 133, at least 205 amino acids of SEQ ID NO: 134, at least 200 amino acids of SEQ ID NO: 136, at least 200 amino acids of SEQ ID NO: 137, at least 213 amino acids of SEQ ID NO: 139, at least 213 amino acids of SEQ ID NO: 140, at least 204 amino acids of SEQ ID NO: 142, at least 204 amino acids of SEQ ID NO: 143, at least 205 amino acids of SEQ ID NO: 145, at least 205 amino acids of SEQ ID NO: 146, at least 204 amino acids of SEQ ID NO: 148, at least 204 amino acids of SEQ ID NO: 149, at least 204 amino acids of SEQ ID NO: 151, at least 204 amino acids of SEQ ID NO: 152, at least 204 amino acids of SEQ ID NO: 154, at least 204 amino acids of SEQ ID NO: 155, at least 205 amino acids of SEQ ID NO: 157, at least 205 amino acids of SEQ ID NO: 158, at least 204 amino acids of SEQ ID NO: 218, at least 204 amino acids of SEQ ID NO: 220, at least 204 amino acids of SEQ ID NO: 221, at least 204 amino acids of SEQ ID NO: 223, at least 204 amino acids of SEQ ID NO: 224, at least 205 amino acids of SEQ ID NO: 226, at least 205 amino acids of SEQ ID NO: 227, at least 204 amino acids of SEQ ID NO: 229, at least 204 amino acids of SEQ ID NO: 230, at least 204 amino acids of SEQ ID NO: 232, or at least 204 amino acids of SEQ ID NO: 233.

Fusion polypeptide: The term “fusion polypeptide” is a polypeptide in which one polypeptide is fused at the N-terminus or the C-terminus of the polypeptide of the present disclosure. A fusion polypeptide is produced by fusing a polynucleotide encoding another polypeptide to a polynucleotide of the present disclosure. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

Host cell: The term “host cell” means any cell type that is susceptible to transformation, transfection, transduction, or the like with a nucleic acid construct or expression vector comprising a polynucleotide of the present disclosure. The term “host cell” encompasses any progeny of a parent cell that is not identical to the parent cell due to mutations that occur during replication.

Hybrid polypeptide: The term “hybrid polypeptide” means a polypeptide comprising domains from two or more polypeptides, e.g., a binding domain from one polypeptide and a catalytic domain from another polypeptide. The domains may be fused at the N-terminus or the C-terminus.

Isolated: The term “isolated” means a substance in a form that does not occur in nature or in an environment in which the substance does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Lysozyme activity: The term “lysozyme activity” means the hydrolysis of the 1,4-beta-linkages between N-acetylmuramic acid and N-acetyl-D-glucosamine residues in a peptidoglycan or between N-acetyl-D-glucosamine residues in chitodextrins, resulting in bacteriolysis. Lysozyme belongs to the enzyme class EC 3.2.1.17. Lysozyme activity is typically measured by the lytic action of the lysozyme on Micrococcus luteus ATCC 4698. In appropriate experimental conditions these changes are proportional to the amount of lysozyme in the medium (c.f. INS 1105 of the Combined Compendium of Food Additive Specifications of the Food and Agriculture Organisation of the UN (www.fao.org)). For the purpose of the present disclosure, lysozyme activity is determined according to the reducing-ends assay described in example 1 (“Determination of Lysozyme Activity”). The polypeptide has lysozyme activity if it shows activity against Micrococcus luteus ATCC 4698, and specifically the lysozymes of the present disclosure exhibit improved activity compared to the prior art lysozyme of SEQ ID NO: 159 wherein lysozyme activity is determined as described in example 1.

In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159. In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as follows: lysozyme (50 μL of 0.7 μg/mL lysozyme in phosphate buffer (5 mM citrate, 5 mM K₂HPO₄, 0.01% TritonX-100, pH 5.0)) is mixed with Micrococcus lysodeikticus solution (450 μL of 1% lyophilized Micrococcus lysodeikticus ATCC No. 4698 in milli-Q water) and incubated at 40° C. with shaking (500 rpm) for 45 min; the sample is centrifuged (4000 g, 5 min); supernatant (100 μL) is mixed with HCl (50 μL 3.2M) and incubated at 95° C. for 80 min; NaOH (50 μL, 3.5 M) is added and 150 μL of the sample is added to 4-hydroxybenzhydrazide in K—Na tartrate/NaOH buffer (75 μL of 50 g/L K—Na tartrate+20 g/L NaOH); the mixture is incubated at 95° C. for 10 min; and the optical density is measured at 405 nm.

Mature polypeptide: The term “mature polypeptide” means a polypeptide in its final form following translation and any post-translational modifications, such as N-terminal processing, C-terminal truncation, glycosylation, phosphorylation, etc.

In one aspect, the mature polypeptide is amino acids 1 to 207 of SEQ ID NO: 2 and amino acids −17 to −1 of SEQ ID NO: 2 are a signal peptide. In another aspect, the mature polypeptide is amino acids 1 to 207 of SEQ ID NO: 3.

In one aspect, the mature polypeptide is amino acids 1 to 201 of SEQ ID NO: 5 and amino acids −17 to −1 of SEQ ID NO: 5 are a signal peptide. In another aspect, the mature polypeptide is amino acids 1 to 201 of SEQ ID NO: 6.

In one aspect, the mature polypeptide is amino acids 1 to 201 of SEQ ID NO: 8 and amino acids −16 to −1 of SEQ ID NO: 8 are a signal peptide. In another aspect, the mature polypeptide is amino acids 1 to 201 of SEQ ID NO: 9.

In one aspect, the mature polypeptide is amino acids 1 to 203 of SEQ ID NO: 11 and amino acids −21 to −1 of SEQ ID NO: 11 are a signal peptide. In another aspect, the mature polypeptide is amino acids 1 to 203 of SEQ ID NO: 12.

In one aspect, the mature polypeptide is amino acids 1 to 208 of SEQ ID NO: 14 and amino acids −20 to −1 of SEQ ID NO: 14 are a signal peptide. In another aspect, the mature polypeptide is amino acids 1 to 208 of SEQ ID NO: 15.

In one aspect, the mature polypeptide is amino acids 1 to 207 of SEQ ID NO: 17 and amino acids −18 to −1 of SEQ ID NO: 17 are a signal peptide. In another aspect, the mature polypeptide is amino acids 1 to 207 of SEQ ID NO: 18.

In one aspect, the mature polypeptide is amino acids 1 to 208 of SEQ ID NO: 20 and amino acids −19 to −1 of SEQ ID NO: 20 are a signal peptide. In another aspect, the mature polypeptide is amino acids 1 to 208 of SEQ ID NO: 21.

In one aspect, the mature polypeptide is amino acids 1 to 207 of SEQ ID NO: 23 and amino acids −18 to −1 of SEQ ID NO: 23 are a signal peptide. In another aspect, the mature polypeptide is amino acids 1 to 207 of SEQ ID NO: 24.

In one aspect, the mature polypeptide is amino acids 1 to 207 of SEQ ID NO: 26 and amino acids −16 to −1 of SEQ ID NO: 26 are a signal peptide. In another aspect, the mature polypeptide is amino acids 1 to 207 of SEQ ID NO: 27.

In one aspect, the mature polypeptide is amino acids 1 to 207 of SEQ ID NO: 29 and amino acids −22 to −1 of SEQ ID NO: 29 are a signal peptide. In another aspect, the mature polypeptide is amino acids 1 to 207 of SEQ ID NO: 30.

In one aspect, the mature polypeptide is amino acids 1 to 208 of SEQ ID NO: 32 and amino acids −20 to −1 of SEQ ID NO: 32 are a signal peptide. In another aspect, the mature polypeptide is amino acids 1 to 208 of SEQ ID NO: 33.

In one aspect, the mature polypeptide is amino acids 1 to 208 of SEQ ID NO: 35 and amino acids −19 to −1 of SEQ ID NO: 35 are a signal peptide. In another aspect, the mature polypeptide is amino acids 1 to 208 of SEQ ID NO: 36.

In one aspect, the mature polypeptide is amino acids 1 to 206 of SEQ ID NO: 38 and amino acids −17 to −1 of SEQ ID NO: 38 are a signal peptide. In another aspect, the mature polypeptide is amino acids 1 to 206 of SEQ ID NO: 39.

In one aspect, the mature polypeptide is amino acids 1 to 207 of SEQ ID NO: 41 and amino acids −17 to −1 of SEQ ID NO: 41 are a signal peptide. In another aspect, the mature polypeptide is amino acids 1 to 207 of SEQ ID NO: 42.

In one aspect, the mature polypeptide is amino acids 1 to 216 of SEQ ID NO: 44 and amino acids −17 to −1 of SEQ ID NO: 44 are a signal peptide. In another aspect, the mature polypeptide is amino acids 1 to 216 of SEQ ID NO: 45.

In one aspect, the mature polypeptide is amino acids 1 to 218 of SEQ ID NO: 47 and amino acids −18 to −1 of SEQ ID NO: 47 are a signal peptide. In another aspect, the mature polypeptide is amino acids 1 to 218 of SEQ ID NO: 48.

In one aspect, the mature polypeptide is amino acids 1 to 204 of SEQ ID NO: 50 and amino acids −19 to −1 of SEQ ID NO: 50 are a signal peptide. In another aspect, the mature polypeptide is amino acids 1 to 204 of SEQ ID NO: 51.

In one aspect, the mature polypeptide is amino acids 1 to 203 of SEQ ID NO: 53 and amino acids −19 to −1 of SEQ ID NO: 53 are a signal peptide. In another aspect, the mature polypeptide is amino acids 1 to 203 of SEQ ID NO: 54.

In one aspect, the mature polypeptide is amino acids 1 to 209 of SEQ ID NO: 56 and amino acids −20 to −1 of SEQ ID NO: 56 are a signal peptide. In another aspect, the mature polypeptide is amino acids 1 to 209 of SEQ ID NO: 57.

In one aspect, the mature polypeptide is amino acids 1 to 208 of SEQ ID NO: 59 and amino acids −17 to −1 of SEQ ID NO: 59 are a signal peptide. In another aspect, the mature polypeptide is amino acids 1 to 208 of SEQ ID NO: 60.

In one aspect, the mature polypeptide is amino acids 1 to 210 of SEQ ID NO: 62 and amino acids −22 to −1 of SEQ ID NO: 62 are a signal peptide. In another aspect, the mature polypeptide is amino acids 1 to 210 of SEQ ID NO: 63.

In one aspect, the mature polypeptide is amino acids 1 to 207 of SEQ ID NO: 65 and amino acids −17 to −1 of SEQ ID NO: 65 are a signal peptide. In another aspect, the mature polypeptide is amino acids 1 to 207 of SEQ ID NO: 66.

In one aspect, the mature polypeptide is amino acids 1 to 207 of SEQ ID NO: 68 and amino acids −19 to −1 of SEQ ID NO: 68 are a signal peptide. In another aspect, the mature polypeptide is amino acids 1 to 207 of SEQ ID NO: 69.

In one aspect, the mature polypeptide is amino acids 1 to 208 of SEQ ID NO: 71 and amino acids −17 to −1 of SEQ ID NO: 71 are a signal peptide. In another aspect, the mature polypeptide is amino acids 1 to 208 of SEQ ID NO: 72.

In one aspect, the mature polypeptide is amino acids 1 to 217 of SEQ ID NO: 74 and amino acids −18 to −1 of SEQ ID NO: 74 are a signal peptide. In another aspect, the mature polypeptide is amino acids 1 to 217 of SEQ ID NO: 75.

In one aspect, the mature polypeptide is amino acids 1 to 208 of SEQ ID NO: 77 and amino acids −20 to −1 of SEQ ID NO: 77 are a signal peptide. In another aspect, the mature polypeptide is amino acids 1 to 208 of SEQ ID NO: 80.

In one aspect, the mature polypeptide is amino acids 1 to 201 of SEQ ID NO: 82 and amino acids −15 to −1 of SEQ ID NO: 82 are a signal peptide. In another aspect, the mature polypeptide is amino acids 1 to 201 of SEQ ID NO: 83.

In one aspect, the mature polypeptide is amino acids 1 to 202 of SEQ ID NO: 85 and amino acids −16 to −1 of SEQ ID NO: 85 are a signal peptide. In another aspect, the mature polypeptide is amino acids 1 to 202 of SEQ ID NO: 86.

In one aspect, the mature polypeptide is amino acids 1 to 207 of SEQ ID NO: 88 and amino acids −17 to −1 of SEQ ID NO: 88 are a signal peptide. In another aspect, the mature polypeptide is amino acids 1 to 207 of SEQ ID NO: 89.

In one aspect, the mature polypeptide is amino acids 1 to 202 of SEQ ID NO: 91 and amino acids −20 to −1 of SEQ ID NO: 91 are a signal peptide. In another aspect, the mature polypeptide is amino acids 1 to 202 of SEQ ID NO: 92.

In one aspect, the mature polypeptide is amino acids 1 to 201 of SEQ ID NO: 94 and amino acids −17 to −1 of SEQ ID NO: 94 are a signal peptide. In another aspect, the mature polypeptide is amino acids 1 to 201 of SEQ ID NO: 95.

In one aspect, the mature polypeptide is amino acids 1 to 202 of SEQ ID NO: 97 and amino acids −16 to −1 of SEQ ID NO: 97 are a signal peptide. In another aspect, the mature polypeptide is amino acids 1 to 202 of SEQ ID NO: 98.

In one aspect, the mature polypeptide is amino acids 1 to 206 of SEQ ID NO: 100 and amino acids −18 to −1 of SEQ ID NO: 100 are a signal peptide. In another aspect, the mature polypeptide is amino acids 1 to 206 of SEQ ID NO: 101.

In one aspect, the mature polypeptide is amino acids 1 to 202 of SEQ ID NO: 103 and amino acids −16 to −1 of SEQ ID NO: 103 are a signal peptide. In another aspect, the mature polypeptide is amino acids 1 to 202 of SEQ ID NO: 104.

In one aspect, the mature polypeptide is amino acids 1 to 202 of SEQ ID NO: 106 and amino acids −17 to −1 of SEQ ID NO: 106 are a signal peptide. In another aspect, the mature polypeptide is amino acids 1 to 202 of SEQ ID NO: 107.

In one aspect, the mature polypeptide is amino acids 1 to 202 of SEQ ID NO: 109 and amino acids −17 to −1 of SEQ ID NO: 109 are a signal peptide. In another aspect, the mature polypeptide is amino acids 1 to 202 of SEQ ID NO: 110.

In one aspect, the mature polypeptide is amino acids 1 to 202 of SEQ ID NO: 112 and amino acids −17 to −1 of SEQ ID NO: 112 are a signal peptide. In another aspect, the mature polypeptide is amino acids 1 to 202 of SEQ ID NO: 113.

In one aspect, the mature polypeptide is amino acids 1 to 206 of SEQ ID NO: 115 and amino acids −18 to −1 of SEQ ID NO: 115 are a signal peptide. In another aspect, the mature polypeptide is amino acids 1 to 206 of SEQ ID NO: 116.

In one aspect, the mature polypeptide is amino acids 1 to 207 of SEQ ID NO: 118 and amino acids −20 to −1 of SEQ ID NO: 118 are a signal peptide. In another aspect, the mature polypeptide is amino acids 1 to 207 of SEQ ID NO: 119.

In one aspect, the mature polypeptide is amino acids 1 to 208 of SEQ ID NO: 121 and amino acids −19 to −1 of SEQ ID NO: 121 are a signal peptide. In another aspect, the mature polypeptide is amino acids 1 to 208 of SEQ ID NO: 122.

In one aspect, the mature polypeptide is amino acids 1 to 215 of SEQ ID NO: 124 and amino acids −18 to −1 of SEQ ID NO: 124 are a signal peptide. In another aspect, the mature polypeptide is amino acids 1 to 215 of SEQ ID NO: 125.

In one aspect, the mature polypeptide is amino acids 1 to 217 of SEQ ID NO: 127 and amino acids −20 to −1 of SEQ ID NO: 127 are a signal peptide. In another aspect, the mature polypeptide is amino acids 1 to 217 of SEQ ID NO: 128.

In one aspect, the mature polypeptide is amino acids 1 to 214 of SEQ ID NO: 130 and amino acids −16 to −1 of SEQ ID NO: 130 are a signal peptide. In another aspect, the mature polypeptide is amino acids 1 to 214 of SEQ ID NO: 131.

In one aspect, the mature polypeptide is amino acids 1 to 208 of SEQ ID NO: 133 and amino acids −18 to −1 of SEQ ID NO: 133 are a signal peptide. In another aspect, the mature polypeptide is amino acids 1 to 208 of SEQ ID NO: 134.

In one aspect, the mature polypeptide is amino acids 1 to 203 of SEQ ID NO: 136 and amino acids −19 to −1 of SEQ ID NO: 136 are a signal peptide. In another aspect, the mature polypeptide is amino acids 1 to 203 of SEQ ID NO: 137.

In one aspect, the mature polypeptide is amino acids 1 to 216 of SEQ ID NO: 139 and amino acids −17 to −1 of SEQ ID NO: 139 are a signal peptide. In another aspect, the mature polypeptide is amino acids 1 to 216 of SEQ ID NO: 140.

In one aspect, the mature polypeptide is amino acids 1 to 207 of SEQ ID NO: 142 and amino acids −21 to −1 of SEQ ID NO: 142 are a signal peptide. In another aspect, the mature polypeptide is amino acids 1 to 207 of SEQ ID NO: 143.

In one aspect, the mature polypeptide is amino acids 1 to 208 of SEQ ID NO: 145 and amino acids −17 to −1 of SEQ ID NO: 145 are a signal peptide. In another aspect, the mature polypeptide is amino acids 1 to 208 of SEQ ID NO: 146.

In one aspect, the mature polypeptide is amino acids 1 to 207 of SEQ ID NO: 148 and amino acids −20 to −1 of SEQ ID NO: 148 are a signal peptide. In another aspect, the mature polypeptide is amino acids 1 to 207 of SEQ ID NO: 149.

In one aspect, the mature polypeptide is amino acids 1 to 207 of SEQ ID NO: 151 and amino acids −20 to −1 of SEQ ID NO: 151 are a signal peptide. In another aspect, the mature polypeptide is amino acids 1 to 207 of SEQ ID NO: 152.

In one aspect, the mature polypeptide is amino acids 1 to 207 of SEQ ID NO: 154 and amino acids −19 to −1 of SEQ ID NO: 154 are a signal peptide. In another aspect, the mature polypeptide is amino acids 1 to 207 of SEQ ID NO: 155.

In one aspect, the mature polypeptide is amino acids 1 to 208 of SEQ ID NO: 157 and amino acids −18 to −1 of SEQ ID NO: 157 are a signal peptide. In another aspect, the mature polypeptide is amino acids 1 to 208 of SEQ ID NO: 158.

In one aspect, the mature polypeptide is amino acids 1 to 207 of SEQ ID NO: 218 and amino acids −17 to −1 of SEQ ID NO: 218 are a signal peptide. In another aspect, the mature polypeptide is amino acids 1 to 207 of SEQ ID NO: 221.

In one aspect, the mature polypeptide is amino acids 1 to 207 of SEQ ID NO: 223 and amino acids −19 to −1 of SEQ ID NO: 223 are a signal peptide. In another aspect, the mature polypeptide is amino acids 1 to 207 of SEQ ID NO: 224.

In one aspect, the mature polypeptide is amino acids 1 to 208 of SEQ ID NO: 226 and amino acids −20 to −1 of SEQ ID NO: 226 are a signal peptide. In another aspect, the mature polypeptide is amino acids 1 to 208 of SEQ ID NO: 227.

In one aspect, the mature polypeptide is amino acids 1 to 207 of SEQ ID NO: 229 and amino acids −20 to −1 of SEQ ID NO: 229 are a signal peptide. In another aspect, the mature polypeptide is amino acids 1 to 207 of SEQ ID NO: 230.

In one aspect, the mature polypeptide is amino acids 1 to 207 of SEQ ID NO: 232 and amino acids −20 to −1 of SEQ ID NO: 232 are a signal peptide. In another aspect, the mature polypeptide is amino acids 1 to 207 of SEQ ID NO: 233.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having lysozyme activity.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Obtained or obtainable from: The term “obtained or obtainable from” means that the polypeptide may be found in an organism from a specific taxonomic rank. In one embodiment, the polypeptide is obtained or obtainable from the kingdom Fungi, wherein the term kingdom is the taxonomic rank. In a preferred embodiment, the polypeptide is obtained or obtainable from the phylum Ascomycota, wherein the term phylum is the taxonomic rank. In another preferred embodiment, the polypeptide is obtained or obtainable from the subphylum Pezizomycotina, wherein the term subphylum is the taxonomic rank.

If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Operably linked: The term “operably linked” means a configuration in which a control sequence is placed at an appropriate position relative to the coding sequence of a polynucleotide such that the control sequence directs expression of the coding sequence.

Roughage: The term “roughage” means dry plant material with high levels of fiber, such as fiber, bran, husks from seeds and grains and crop residues (such as stover, copra, straw, chaff, sugar beet waste).

Sequence identity: The relatedness between two amino acid sequences or between two nucleotide sequences is described by the parameter “sequence identity”.

For purposes of the present disclosure, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labelled “longest identity” (obtained using the −nobrief option) is used as the percent identity and is calculated as follows:

(Identical Residues×100)/(Length of Alignment−Total Number of Gaps in Alignment)

For purposes of the present disclosure, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labelled “longest identity” (obtained using the −nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having lysozyme activity.

Substantially pure polypeptide: The term “substantially pure polypeptide” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the polypeptide is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The polypeptides of the present disclosure are preferably in a substantially pure form. This can be accomplished, for example, by preparing the polypeptide by well known recombinant methods or by classical purification methods.

Variant: The term “variant” means a polypeptide having lysozyme activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In one aspect, a lysozyme variant according to the present disclosure may comprise from 1 to 5; from 1 to 10; from 1 to 15; from 1 to 20; from 1 to 25; from 1 to 30; from 1 to 35; from 1 to 40; from 1 to 45; or from 1-50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 alterations and have at least 150%, e.g., at least 175%, at least 200%, at least 225%, at least 250%, at least 275%, at least 300%, at least 350%, or at least 400% of the lysozyme activity of SEQ ID NO: 159.

In one aspect, the variant of the present disclosure has at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In one aspect, the variant of the present disclosure has at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as follows: lysozyme (50 μL of 0.7 μg/mL lysozyme in phosphate buffer (5 mM citrate, 5 mM K₂HPO₄, 0.01% TritonX-100, pH 5.0)) is mixed with Micrococcus lysodeikticus solution (450 μL of 1% lyophilized Micrococcus lysodeikticus ATCC No. 4698 in milli-Q water) and incubated at 40° C. with shaking (500 rpm) for 45 min; the sample is centrifuged (4000 g, 5 min); supernatant (100 μL) is mixed with HCl (50 μL 3.2M) and incubated at 95° C. for 80 min; NaOH (50 μL, 3.5 M) is added and 150 μL of the sample is added to 4-hydroxybenzhydrazide in K—Na tartrate/NaOH buffer (75 μL of 50 g/L K—Na tartrate+20 g/L NaOH); the mixture is incubated at 95° C. for 10 min; and the optical density is measured at 405 nm.

For purposes of the present disclosure, the nomenclature [E/Q] means that the amino acid at this position may be a glutamic acid (Glu, E) or a glutamine (Gln, Q). Likewise the nomenclature [V/G/A/I] means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

In the first aspect, the present disclosure relates to a method of hydrolysing peptidoglycan in bacterial cell walls comprising treating bacterial cells with one or more GH25 polypeptides having lysozyme activity, wherein the polypeptide is selected from the group consisting of:
(a) a polypeptide having at least 80%, such as at least 85%, at least 90% or at least 95% sequence identity to the polypeptide of SEQ ID NO: 3;
(b) a polypeptide having at least 84%, such as at least 85%, at least 90% or at least 95% sequence identity to the polypeptide of SEQ ID NO: 6;
(c) a polypeptide having at least 80%, such as at least 85%, at least 90% or at least 95% sequence identity to the polypeptide of SEQ ID NO: 9;
(d) a polypeptide having at least 80%, such as at least 85%, at least 90% or at least 95% sequence identity to the polypeptide of SEQ ID NO: 12;
(e) a polypeptide having at least 80%, such as at least 85%, at least 90% or at least 95% sequence identity to the polypeptide of SEQ ID NO: 15;
(f) a polypeptide having at least 80%, such as at least 85%, at least 90% or at least 95% sequence identity to the polypeptide of SEQ ID NO: 18;
(g) a polypeptide having at least 80%, such as at least 85%, at least 90% or at least 95% sequence identity to the polypeptide of SEQ ID NO: 21;
(h) a polypeptide having at least 80%, such as at least 85%, at least 90% or at least 95% sequence identity to the polypeptide of SEQ ID NO: 24;
(i) a polypeptide having at least 80%, such as at least 85%, at least 90% or at least 95% sequence identity to the polypeptide of SEQ ID NO: 27;
(j) a polypeptide having at least 84%, such as at least 85%, at least 90% or at least 95% sequence identity to the polypeptide of SEQ ID NO: 30;
(k) a polypeptide having at least 80%, such as at least 85%, at least 90% or at least 95% sequence identity to the polypeptide of SEQ ID NO: 33;
(l) a polypeptide having at least 80%, such as at least 85%, at least 90% or at least 95% sequence identity to the polypeptide of SEQ ID NO: 36;
(m) a polypeptide having at least 84%, such as at least 85%, at least 90% or at least 95% sequence identity to the polypeptide of SEQ ID NO: 39;
(n) a polypeptide having at least 80%, such as at least 85%, at least 90% or at least 95% sequence identity to the polypeptide of SEQ ID NO: 42;
(o) a polypeptide having at least 80%, such as at least 85%, at least 90% or at least 95% sequence identity to the polypeptide of SEQ ID NO: 45;
(p) a polypeptide having at least 80%, such as at least 85%, at least 90% or at least 95% sequence identity to the polypeptide of SEQ ID NO: 48;
(q) a polypeptide having at least 80%, such as at least 85%, at least 90% or at least 95% sequence identity to the polypeptide of SEQ ID NO: 51;
(r) a polypeptide having at least 80%, such as at least 85%, at least 90% or at least 95% sequence identity to the polypeptide of SEQ ID NO: 54;
(s) a polypeptide having at least 80%, such as at least 85%, at least 90% or at least 95% sequence identity to the polypeptide of SEQ ID NO: 57;
(t) a polypeptide having at least 80%, such as at least 85%, at least 90% or at least 95% sequence identity to the polypeptide of SEQ ID NO: 60;
(u) a polypeptide having at least 80%, such as at least 85%, at least 90% or at least 95% sequence identity to the polypeptide of SEQ ID NO: 63;
(v) a polypeptide having at least 80%, such as at least 85%, at least 90% or at least 95% sequence identity to the polypeptide of SEQ ID NO: 66;
(w) a polypeptide having at least 80%, such as at least 85%, at least 90% or at least 95% sequence identity to the polypeptide of SEQ ID NO: 69;
(x) a polypeptide having at least 84%, such as at least 85%, at least 90% or at least 95% sequence identity to the polypeptide of SEQ ID NO: 72;
(y) a polypeptide having at least 80%, such as at least 85%, at least 90% or at least 95% sequence identity to the polypeptide of SEQ ID NO: 75;
(z) a polypeptide having at least 80%, such as at least 85%, at least 90% or at least 95% sequence identity to the polypeptide of SEQ ID NO: 80;
(aa) a polypeptide having at least 80%, such as at least 85%, at least 90% or at least 95% sequence identity to the polypeptide of SEQ ID NO: 83;
(ab) a polypeptide having at least 80%, such as at least 85%, at least 90% or at least 95% sequence identity to the polypeptide of SEQ ID NO: 86;
(ac) a polypeptide having at least 86%, such as at least 90% or at least 95% sequence identity to the polypeptide of SEQ ID NO: 89;
(ad) a polypeptide having at least 82%, such as at least 85%, at least 90% or at least 95% sequence identity to the polypeptide of SEQ ID NO: 92;
(ae) a polypeptide having at least 82%, such as at least 85%, at least 90% or at least 95% sequence identity to the polypeptide of SEQ ID NO: 95;
(af) a polypeptide having at least 80%, such as at least 85%, at least 90% or at least 95% sequence identity to the polypeptide of SEQ ID NO: 98;
(ag) a polypeptide having at least 80%, such as at least 85%, at least 90% or at least 95% sequence identity to the polypeptide of SEQ ID NO: 101;
(ah) a polypeptide having at least 80%, such as at least 85%, at least 90% or at least 95% sequence identity to the polypeptide of SEQ ID NO: 104;
(ai) a polypeptide having at least 80%, such as at least 85%, at least 90% or at least 95% sequence identity to the polypeptide of SEQ ID NO: 107;
(aj) a polypeptide having at least 82%, such as at least 85%, at least 90% or at least 95% sequence identity to the polypeptide of SEQ ID NO: 110;
(ak) a polypeptide having at least 81%, such as at least 85%, at least 90% or at least 95% sequence identity to the polypeptide of SEQ ID NO: 113;
(al) a polypeptide having at least 80%, such as at least 85%, at least 90% or at least 95% sequence identity to the polypeptide of SEQ ID NO: 116;
(am) a polypeptide having at least 80%, such as at least 85%, at least 90% or at least 95% sequence identity to the polypeptide of SEQ ID NO: 119;
(an) a polypeptide having at least 80%, such as at least 85%, at least 90% or at least 95% sequence identity to the polypeptide of SEQ ID NO: 122;
(ao) a polypeptide having at least 80%, such as at least 85%, at least 90% or at least 95% sequence identity to the polypeptide of SEQ ID NO: 125;
(ap) a polypeptide having at least 80%, such as at least 85%, at least 90% or at least 95% sequence identity to the polypeptide of SEQ ID NO: 128;
(aq) a polypeptide having at least 80%, such as at least 85%, at least 90% or at least 95% sequence identity to the polypeptide of SEQ ID NO: 131;
(ar) a polypeptide having at least 80%, such as at least 85%, at least 90% or at least 95% sequence identity to the polypeptide of SEQ ID NO: 134;
(as) a polypeptide having at least 80%, such as at least 85%, at least 90% or at least 95% sequence identity to the polypeptide of SEQ ID NO: 137;
(at) a polypeptide having at least 80%, such as at least 85%, at least 90% or at least 95% sequence identity to the polypeptide of SEQ ID NO: 140;
(au) a polypeptide having at least 80%, such as at least 85%, at least 90% or at least 95% sequence identity to the polypeptide of SEQ ID NO: 143;
(av) a polypeptide having at least 80%, such as at least 85%, at least 90% or at least 95% sequence identity to the polypeptide of SEQ ID NO: 146;
(aw) a polypeptide having at least 80%, such as at least 85%, at least 90% or at least 95% sequence identity to the polypeptide of SEQ ID NO: 149;
(ax) a polypeptide having at least 80%, such as at least 85%, at least 90% or at least 95% sequence identity to the polypeptide of SEQ ID NO: 152;
(ay) a polypeptide having at least 80%, such as at least 85%, at least 90% or at least 95% sequence identity to the polypeptide of SEQ ID NO: 155;
(az) a polypeptide having at least 80%, such as at least 85%, at least 90% or at least 95% sequence identity to the polypeptide of SEQ ID NO: 158;
(ba) a polypeptide having at least 80%, such as at least 85%, at least 90% or at least 95% sequence identity to the polypeptide of SEQ ID NO: 221;
(bb) a polypeptide having at least 80%, such as at least 85%, at least 90% or at least 95% sequence identity to the polypeptide of SEQ ID NO: 224;
(bc) a polypeptide having at least 80%, such as at least 85%, at least 90% or at least 95% sequence identity to the polypeptide of SEQ ID NO: 227;
(bd) a polypeptide having at least 80%, such as at least 85%, at least 90% or at least 95% sequence identity to the polypeptide of SEQ ID NO: 230;
(be) a polypeptide having at least 80%, such as at least 85%, at least 90% or at least 95% sequence identity to the polypeptide of SEQ ID NO: 233;
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27 or 28 positions;
(bg) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal His-tag and/or HQ-tag;
(bh) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal extension of up to 10 amino acids, e.g. 1, 2, 3, 4, 5, 6, 7, 8, 9 or 10 amino acids; and
(bi) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) having lysozyme activity and having at least 90% of the length of the mature polypeptide.

In an embodiment, the one or more GH25 polypeptides comprises the motif YKNA (SEQ ID NO: 160) and/or the motif FGGW[S/T] (SEQ ID NO: 161), and preferably is selected from the group selected from SEQ ID NO: 12, SEQ ID NO: 27, SEQ ID NO: 42, SEQ ID NO: 57, SEQ ID NO: 63, SEQ ID NO: 66, SEQ ID NO: 80, SEQ ID NO: 149, SEQ ID NO: 152, SEQ ID NO: 155, SEQ ID NO: 224, SEQ ID NO: 227, SEQ ID NO: 230 and SEQ ID NO: 233.

In an embodiment, the polypeptide comprises or consists of amino acids 1 to 207 of SEQ ID NO: 3, amino acids 1 to 201 of SEQ ID NO: 6, amino acids 1 to 201 of SEQ ID NO: 9, amino acids 1 to 203 of SEQ ID NO: 12, amino acids 1 to 208 of SEQ ID NO: 15, amino acids 1 to 207 of SEQ ID NO: 18, amino acids 1 to 208 of SEQ ID NO: 21, amino acids 1 to 207 of SEQ ID NO: 24, amino acids 1 to 207 of SEQ ID NO: 27, amino acids 1 to 207 of SEQ ID NO: 30, amino acids 1 to 208 of SEQ ID NO: 33, amino acids 1 to 208 of SEQ ID NO: 36, amino acids 1 to 206 of SEQ ID NO: 39, amino acids 1 to 207 of SEQ ID NO: 42, amino acids 1 to 216 of SEQ ID NO: 45, amino acids 1 to 218 of SEQ ID NO: 48, amino acids 1 to 204 of SEQ ID NO: 51, amino acids 1 to 203 of SEQ ID NO: 54, amino acids 1 to 209 of SEQ ID NO: 57, amino acids 1 to 208 of SEQ ID NO: 60, amino acids 1 to 210 of SEQ ID NO: 63, amino acids 1 to 207 of SEQ ID NO: 66, amino acids 1 to 207 of SEQ ID NO: 69, amino acids 1 to 208 of SEQ ID NO: 72, amino acids 1 to 217 of SEQ ID NO: 75, amino acids 1 to 208 of SEQ ID NO: 80, amino acids 1 to 201 of SEQ ID NO: 83, amino acids 1 to 202 of SEQ ID NO: 86, amino acids 1 to 207 of SEQ ID NO: 89, amino acids 1 to 202 of SEQ ID NO: 92, amino acids 1 to 201 of SEQ ID NO: 95, amino acids 1 to 202 of SEQ ID NO: 98, amino acids 1 to 206 of SEQ ID NO: 101, amino acids 1 to 202 of SEQ ID NO: 104, amino acids 1 to 202 of SEQ ID NO: 107, amino acids 1 to 202 of SEQ ID NO: 110, amino acids 1 to 202 of SEQ ID NO: 113, amino acids 1 to 206 of SEQ ID NO: 116, amino acids 1 to 207 of SEQ ID NO: 119, amino acids 1 to 208 of SEQ ID NO: 122, amino acids 1 to 215 of SEQ ID NO: 125, amino acids 1 to 217 of SEQ ID NO: 128, amino acids 1 to 214 of SEQ ID NO: 131, amino acids 1 to 208 of SEQ ID NO: 134, amino acids 1 to 203 of SEQ ID NO: 137, amino acids 1 to 216 of SEQ ID NO: 140, amino acids 1 to 207 of SEQ ID NO: 143, amino acids 1 to 208 of SEQ ID NO: 146, amino acids 1 to 207 of SEQ ID NO: 149, amino acids 1 to 207 of SEQ ID NO: 152, amino acids 1 to 207 of SEQ ID NO: 155, amino acids 1 to 208 of SEQ ID NO: 158, amino acids 1 to 207 of SEQ ID NO: 221, amino acids 1 to 207 of SEQ ID NO: 224, amino acids 1 to 208 of SEQ ID NO: 227, amino acids 1 to 207 of SEQ ID NO: 230 or amino acids 1 to 207 of SEQ ID NO: 233.

In a preferred embodiment, the polypeptide has at least 150%, such as at least 175%, at least 200%, at least 225%, at least 250%, at least 275%, at least 300%, at least 350%, or at least 400% of the lysozyme activity of SEQ ID NO: 159. In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as follows: lysozyme (50 μL of 0.7 μg/mL lysozyme in phosphate buffer (5 mM citrate, 5 mM K₂HPO₄, 0.01% TritonX-100, pH 5.0)) is mixed with Micrococcus lysodeikticus solution (450 μL of 1% lyophilized Micrococcus lysodeikticus ATCC No. 4698 in milli-Q water) and incubated at 40° C. with shaking (500 rpm) for 45 min; the sample is centrifuged (4000 g, 5 min); supernatant (100 μL) is mixed with HCl (50 μL 3.2M) and incubated at 95° C. for 80 min; NaOH (50 μL, 3.5 M) is added and 150 L of the sample is added to 4-hydroxybenzhydrazide in K—Na tartrate/NaOH buffer (75 μL of 50 g/L K—Na tartrate+20 g/L NaOH); the mixture is incubated at 95° C. for 10 min; and the optical density is measured at 405 nm.

In a second aspect, the present disclosure relates to a granule comprising one or more GH25 polypeptides having lysozyme activity, wherein the polypeptide is selected from the group consisting of:
(a) a polypeptide having at least 80%, such as at least 85%, at least 90% or at least 95% sequence identity to the polypeptide of SEQ ID NO: 3;
(b) a polypeptide having at least 84%, such as at least 85%, at least 90% or at least 95% sequence identity to the polypeptide of SEQ ID NO: 6;
(c) a polypeptide having at least 80%, such as at least 85%, at least 90% or at least 95% sequence identity to the polypeptide of SEQ ID NO: 9;
(d) a polypeptide having at least 80%, such as at least 85%, at least 90% or at least 95% sequence identity to the polypeptide of SEQ ID NO: 12;
(e) a polypeptide having at least 80%, such as at least 85%, at least 90% or at least 95% sequence identity to the polypeptide of SEQ ID NO: 15;
(f) a polypeptide having at least 80%, such as at least 85%, at least 90% or at least 95% sequence identity to the polypeptide of SEQ ID NO: 18;
(g) a polypeptide having at least 80%, such as at least 85%, at least 90% or at least 95% sequence identity to the polypeptide of SEQ ID NO: 21;
(h) a polypeptide having at least 80%, such as at least 85%, at least 90% or at least 95% sequence identity to the polypeptide of SEQ ID NO: 24;
(i) a polypeptide having at least 80%, such as at least 85%, at least 90% or at least 95% sequence identity to the polypeptide of SEQ ID NO: 27;
(j) a polypeptide having at least 84%, such as at least 85%, at least 90% or at least 95% sequence identity to the polypeptide of SEQ ID NO: 30;
(k) a polypeptide having at least 80%, such as at least 85%, at least 90% or at least 95% sequence identity to the polypeptide of SEQ ID NO: 33;
(l) a polypeptide having at least 80%, such as at least 85%, at least 90% or at least 95% sequence identity to the polypeptide of SEQ ID NO: 36;
(m) a polypeptide having at least 84%, such as at least 85%, at least 90% or at least 95% sequence identity to the polypeptide of SEQ ID NO: 39;
(n) a polypeptide having at least 80%, such as at least 85%, at least 90% or at least 95% sequence identity to the polypeptide of SEQ ID NO: 42;
(o) a polypeptide having at least 80%, such as at least 85%, at least 90% or at least 95% sequence identity to the polypeptide of SEQ ID NO: 45;
(p) a polypeptide having at least 80%, such as at least 85%, at least 90% or at least 95% sequence identity to the polypeptide of SEQ ID NO: 48;
(q) a polypeptide having at least 80%, such as at least 85%, at least 90% or at least 95% sequence identity to the polypeptide of SEQ ID NO: 51;
(r) a polypeptide having at least 80%, such as at least 85%, at least 90% or at least 95% sequence identity to the polypeptide of SEQ ID NO: 54;
(s) a polypeptide having at least 80%, such as at least 85%, at least 90% or at least 95% sequence identity to the polypeptide of SEQ ID NO: 57;
(t) a polypeptide having at least 80%, such as at least 85%, at least 90% or at least 95% sequence identity to the polypeptide of SEQ ID NO: 60;
(u) a polypeptide having at least 80%, such as at least 85%, at least 90% or at least 95% sequence identity to the polypeptide of SEQ ID NO: 63;
(v) a polypeptide having at least 80%, such as at least 85%, at least 90% or at least 95% sequence identity to the polypeptide of SEQ ID NO: 66;
(w) a polypeptide having at least 80%, such as at least 85%, at least 90% or at least 95% sequence identity to the polypeptide of SEQ ID NO: 69;
(x) a polypeptide having at least 84%, such as at least 85%, at least 90% or at least 95% sequence identity to the polypeptide of SEQ ID NO: 72;
(y) a polypeptide having at least 80%, such as at least 85%, at least 90% or at least 95% sequence identity to the polypeptide of SEQ ID NO: 75;
(z) a polypeptide having at least 80%, such as at least 85%, at least 90% or at least 95% sequence identity to the polypeptide of SEQ ID NO: 80;
(aa) a polypeptide having at least 80%, such as at least 85%, at least 90% or at least 95% sequence identity to the polypeptide of SEQ ID NO: 83;
(ab) a polypeptide having at least 80%, such as at least 85%, at least 90% or at least 95% sequence identity to the polypeptide of SEQ ID NO: 86;
(ac) a polypeptide having at least 86%, such as at least 90% or at least 95% sequence identity to the polypeptide of SEQ ID NO: 89;
(ad) a polypeptide having at least 82%, such as at least 85%, at least 90% or at least 95% sequence identity to the polypeptide of SEQ ID NO: 92;
(ae) a polypeptide having at least 82%, such as at least 85%, at least 90% or at least 95% sequence identity to the polypeptide of SEQ ID NO: 95;
(af) a polypeptide having at least 80%, such as at least 85%, at least 90% or at least 95% sequence identity to the polypeptide of SEQ ID NO: 98;
(ag) a polypeptide having at least 80%, such as at least 85%, at least 90% or at least 95% sequence identity to the polypeptide of SEQ ID NO: 101;
(ah) a polypeptide having at least 80%, such as at least 85%, at least 90% or at least 95% sequence identity to the polypeptide of SEQ ID NO: 104;
(ai) a polypeptide having at least 80%, such as at least 85%, at least 90% or at least 95% sequence identity to the polypeptide of SEQ ID NO: 107;
(aj) a polypeptide having at least 82%, such as at least 85%, at least 90% or at least 95% sequence identity to the polypeptide of SEQ ID NO: 110;
(ak) a polypeptide having at least 81%, such as at least 85%, at least 90% or at least 95% sequence identity to the polypeptide of SEQ ID NO: 113;
(al) a polypeptide having at least 80%, such as at least 85%, at least 90% or at least 95% sequence identity to the polypeptide of SEQ ID NO: 116;
(am) a polypeptide having at least 80%, such as at least 85%, at least 90% or at least 95% sequence identity to the polypeptide of SEQ ID NO: 119;
(an) a polypeptide having at least 80%, such as at least 85%, at least 90% or at least 95% sequence identity to the polypeptide of SEQ ID NO: 122;
(ao) a polypeptide having at least 80%, such as at least 85%, at least 90% or at least 95% sequence identity to the polypeptide of SEQ ID NO: 125;
(ap) a polypeptide having at least 80%, such as at least 85%, at least 90% or at least 95% sequence identity to the polypeptide of SEQ ID NO: 128;
(aq) a polypeptide having at least 80%, such as at least 85%, at least 90% or at least 95% sequence identity to the polypeptide of SEQ ID NO: 131;
(ar) a polypeptide having at least 80%, such as at least 85%, at least 90% or at least 95% sequence identity to the polypeptide of SEQ ID NO: 134;
(as) a polypeptide having at least 80%, such as at least 85%, at least 90% or at least 95% sequence identity to the polypeptide of SEQ ID NO: 137;
(at) a polypeptide having at least 80%, such as at least 85%, at least 90% or at least 95% sequence identity to the polypeptide of SEQ ID NO: 140;
(au) a polypeptide having at least 80%, such as at least 85%, at least 90% or at least 95% sequence identity to the polypeptide of SEQ ID NO: 143;
(av) a polypeptide having at least 80%, such as at least 85%, at least 90% or at least 95% sequence identity to the polypeptide of SEQ ID NO: 146;
(aw) a polypeptide having at least 80%, such as at least 85%, at least 90% or at least 95% sequence identity to the polypeptide of SEQ ID NO: 149;
(ax) a polypeptide having at least 80%, such as at least 85%, at least 90% or at least 95% sequence identity to the polypeptide of SEQ ID NO: 152;
(ay) a polypeptide having at least 80%, such as at least 85%, at least 90% or at least 95% sequence identity to the polypeptide of SEQ ID NO: 155;
(az) a polypeptide having at least 80%, such as at least 85%, at least 90% or at least 95% sequence identity to the polypeptide of SEQ ID NO: 158;
(ba) a polypeptide having at least 80%, such as at least 85%, at least 90% or at least 95% sequence identity to the polypeptide of SEQ ID NO: 221;
(bb) a polypeptide having at least 80%, such as at least 85%, at least 90% or at least 95% sequence identity to the polypeptide of SEQ ID NO: 224;
(bc) a polypeptide having at least 80%, such as at least 85%, at least 90% or at least 95% sequence identity to the polypeptide of SEQ ID NO: 227;
(bd) a polypeptide having at least 80%, such as at least 85%, at least 90% or at least 95% sequence identity to the polypeptide of SEQ ID NO: 230;
(be) a polypeptide having at least 80%, such as at least 85%, at least 90% or at least 95% sequence identity to the polypeptide of SEQ ID NO: 233;
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27 or 28 positions;
(bg) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal His-tag and/or HQ-tag;
(bh) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal extension of up to 10 amino acids, e.g. 1, 2, 3, 4, 5, 6, 7, 8, 9 or 10 amino acids; and
(bi) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) having lysozyme activity and having at least 90% of the length of the mature polypeptide.

In an embodiment, the one or more GH25 polypeptides comprises the motif YKNA (SEQ ID NO: 160) and/or the motif FGGW[S/T] (SEQ ID NO: 161), and preferably is selected from the group selected from SEQ ID NO: 12, SEQ ID NO: 27, SEQ ID NO: 42, SEQ ID NO: 57, SEQ ID NO: 63, SEQ ID NO: 66, SEQ ID NO: 80, SEQ ID NO: 149, SEQ ID NO:152, SEQ ID NO: 155, SEQ ID NO: 224, SEQ ID NO: 227, SEQ ID NO: 230 and SEQ ID NO: 233.

In an embodiment, the polypeptide comprises or consists of amino acids 1 to 207 of SEQ ID NO: 3, amino acids 1 to 201 of SEQ ID NO: 6, amino acids 1 to 201 of SEQ ID NO: 9, amino acids 1 to 203 of SEQ ID NO: 12, amino acids 1 to 208 of SEQ ID NO: 15, amino acids 1 to 207 of SEQ ID NO: 18, amino acids 1 to 208 of SEQ ID NO: 21, amino acids 1 to 207 of SEQ ID NO: 24, amino acids 1 to 207 of SEQ ID NO: 27, amino acids 1 to 207 of SEQ ID NO: 30, amino acids 1 to 208 of SEQ ID NO: 33, amino acids 1 to 208 of SEQ ID NO: 36, amino acids 1 to 206 of SEQ ID NO: 39, amino acids 1 to 207 of SEQ ID NO: 42, amino acids 1 to 216 of SEQ ID NO: 45, amino acids 1 to 218 of SEQ ID NO: 48, amino acids 1 to 204 of SEQ ID NO: 51, amino acids 1 to 203 of SEQ ID NO: 54, amino acids 1 to 209 of SEQ ID NO: 57, amino acids 1 to 208 of SEQ ID NO: 60, amino acids 1 to 210 of SEQ ID NO: 63, amino acids 1 to 207 of SEQ ID NO: 66, amino acids 1 to 207 of SEQ ID NO: 69, amino acids 1 to 208 of SEQ ID NO: 72, amino acids 1 to 217 of SEQ ID NO: 75, amino acids 1 to 208 of SEQ ID NO: 80, amino acids 1 to 201 of SEQ ID NO: 83, amino acids 1 to 202 of SEQ ID NO: 86, amino acids 1 to 207 of SEQ ID NO: 89, amino acids 1 to 202 of SEQ ID NO: 92, amino acids 1 to 201 of SEQ ID NO: 95, amino acids 1 to 202 of SEQ ID NO: 98, amino acids 1 to 206 of SEQ ID NO: 101, amino acids 1 to 202 of SEQ ID NO: 104, amino acids 1 to 202 of SEQ ID NO: 107, amino acids 1 to 202 of SEQ ID NO: 110, amino acids 1 to 202 of SEQ ID NO: 113, amino acids 1 to 206 of SEQ ID NO: 116, amino acids 1 to 207 of SEQ ID NO: 119, amino acids 1 to 208 of SEQ ID NO: 122, amino acids 1 to 215 of SEQ ID NO: 125, amino acids 1 to 217 of SEQ ID NO: 128, amino acids 1 to 214 of SEQ ID NO: 131, amino acids 1 to 208 of SEQ ID NO: 134, amino acids 1 to 203 of SEQ ID NO: 137, amino acids 1 to 216 of SEQ ID NO: 140, amino acids 1 to 207 of SEQ ID NO: 143, amino acids 1 to 208 of SEQ ID NO: 146, amino acids 1 to 207 of SEQ ID NO: 149, amino acids 1 to 207 of SEQ ID NO: 152, amino acids 1 to 207 of SEQ ID NO: 155, amino acids 1 to 208 of SEQ ID NO: 158, amino acids 1 to 207 of SEQ ID NO: 221, amino acids 1 to 207 of SEQ ID NO: 224, amino acids 1 to 208 of SEQ ID NO: 227, amino acids 1 to 207 of SEQ ID NO: 230 or amino acids 1 to 207 of SEQ ID NO: 233.

In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159. In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as follows: lysozyme (50 μL of 0.7 μg/mL lysozyme in phosphate buffer (5 mM citrate, 5 mM K₂HPO₄, 0.01% TritonX-100, pH 5.0)) is mixed with Micrococcus lysodeikticus solution (450 μL of 1% lyophilized Micrococcus lysodeikticus ATCC No. 4698 in milli-Q water) and incubated at 40° C. with shaking (500 rpm) for 45 min; the sample is centrifuged (4000 g, 5 min); supernatant (100 μL) is mixed with HCl (50 μL 3.2M) and incubated at 95° C. for 80 min; NaOH (50 μL, 3.5 M) is added and 150 μL of the sample is added to 4-hydroxybenzhydrazide in K—Na tartrate/NaOH buffer (75 μL of 50 g/L K—Na tartrate+20 g/L NaOH); the mixture is incubated at 95° C. for 10 min; and the optical density is measured at 405 nm.

In one embodiment of the second aspect, the granule comprises one or more formulating agents (such as those described herein), preferably a formulating agent selected from the list consisting of glycerol, ethylene glycol, 1,2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose, preferably selected from the list consisting of 1, 2-propylene glycol, 1,3-propylene glycol, sodium sulfate, dextrin, cellulose, sodium thiosulfate, kaolin and calcium carbonate.

In a further embodiment to any part of the second aspect, the granule comprises a core particle and one or more coatings. In a preferred embodiment, the coating comprises salt and/or wax and/or flour.

In one embodiment, the granule comprises one or more additional enzymes. The one or more additional enzymes is preferably selected from the group consisting of phytase, xylanase, galactanase, alpha-galactosidase, beta-galactosidase, protease, phospholipase A1, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.

In one embodiment, the granule comprises one or more probiotics. The one or more probiotics is preferably selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococsus acidilactici, Pediococcus sp., Propionibacterium thoenii, Propionibacterium sp. and Streptococcus sp. or any combination thereof.

In a third aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 2. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 2.

In a continuation of the third aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 3.

In one embodiment, the present disclosure relates to polypeptides having lysozyme activity and having a sequence identity to SEQ ID NO: 3 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 3 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 3 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 3 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 2. In one embodiment, the polypeptide preferably comprises or consists of the amino acid sequence of SEQ ID NO: 3; comprises the amino acid sequence of SEQ ID NO: 3 and a N-terminal and/or C-terminal His-tag and/or HQ-tag; comprises the amino acid sequence of SEQ ID NO: 3 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 3. In one embodiment, the polypeptide comprises or consists of amino acids 1 to 207 of SEQ ID NO: 3. In an embodiment, the polypeptide has been isolated.

In a continuation of the third aspect, the present disclosure relates to a polypeptide having lysozyme activity encoded by a polynucleotide having a sequence identity to the mature polypeptide coding sequence of SEQ ID NO: 1 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the third aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the third aspect, the present disclosure relates to variants of SEQ ID NO: 3 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 3 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 3 is not more than 10, e.g., 1, 2, 3, 4, 5, 6, 7, 8, 9 or 10. In one embodiment, the number of substitutions and/or deletions and/or insertions in SEQ ID NO: 3 is not more than 10, e.g., 1, 2, 3, 4, 5, 6, 7, 8, 9 or 10. In a further embodiment, the number of substitutions, preferably conservative substitutions, in SEQ ID NO: 3 is not more than 10, e.g., 1, 2, 3, 4, 5, 6, 7, 8, 9 or 10.

In an embodiment of the third aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly. Other examples of conservative substitutions are G to A; A to G, S; V to I, L, A, T, S; I to V, L, M; L to I, M, V; M to L, I, V; P to A, S, N; F to Y, W, H; Y to F, W, H; W to Y, F, H; R to K, E, D; K to R, E, D; H to Q, N, S; D to N, E, K, R, Q; E to Q, D, K, R, N; S to T, A; T to S, V, A; C to S, T, A; N to D, Q, H, S; Q to E, N, H, K, R.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant mutant molecules are tested for lysozyme activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labelling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g., Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide or a fusion polypeptide.

In a fourth aspect the present disclosure relates to polypeptides having lysozyme activity having at least 89%, e.g., at least 90%, at least 91%, at least 92%, at least 93%, at least 94%, at least 95%, at least 96%, at least 97%, at least 98%, at least 99%, or 100% sequence identity to the mature polypeptide of SEQ ID NO: 5. In one embodiment, the polypeptides differ by up to 22 amino acids, e.g., 1, 2, 3, 4, 5, 6, 7, 8, 9, 10, 11, 12, 13, 14, 15, 16, 17, 18, 19, 20, 21 or 22 amino acids from the mature polypeptide of SEQ ID NO: 5.

In a continuation of the fourth aspect, the present disclosure relates to polypeptides having lysozyme activity having at least 89%, e.g., at least 90%, at least 91%, at least 92%, at least 93%, at least 94%, at least 95%, at least 96%, at least 97%, at least 98%, at least 99%, or 100% sequence identity to SEQ ID NO: 6. In one embodiment, the polypeptides differ by up to 22 amino acids, e.g., 1, 2, 3, 4, 5, 6, 7, 8, 9, 10, 11, 12, 13, 14, 15, 16, 17, 18, 19, 20, 21 or 22 amino acids from the mature polypeptide of SEQ ID NO: 6.

In one embodiment, the present disclosure relates to polypeptides having lysozyme activity and having a sequence identity to SEQ ID NO: 6 of at least 89% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 6 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 6 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 5. In one embodiment, the polypeptide preferably comprises or consists of the amino acid sequence of SEQ ID NO: 6; comprises the amino acid sequence of SEQ ID NO: 6 and a N-terminal and/or C-terminal His-tag and/or HQ-tag; comprises the amino acid sequence of SEQ ID NO: 6 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 6. In one embodiment, the polypeptide comprises or consists of amino acids 1 to 201 of SEQ ID NO: 6. In an embodiment, the polypeptide has been isolated.

In a continuation of the fourth aspect, the present disclosure relates to a polypeptide having lysozyme activity encoded by a polynucleotide having a sequence identity to the mature polypeptide coding sequence of SEQ ID NO: 4 of at least 89%, e.g., at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fourth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fourth aspect, the present disclosure relates to variants of SEQ ID NO: 6 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 6 is not more than 22, e.g., 1, 2, 3, 4, 5, 6, 7, 8, 9, 10, 11, 12, 13, 14, 15, 16, 17, 18, 19, 20, 21 or 22. In an embodiment, the number of positions comprising one or more amino acid substitutions, and/or one or more amino acid deletions, and/or one or more amino acid insertions or any combination thereof in SEQ ID NO: 6 is not more than 10, e.g., 1, 2, 3, 4, 5, 6, 7, 8, 9 or 10. In one embodiment, the number of substitutions and/or deletions and/or insertions in SEQ ID NO: 6 is not more than 10, e.g., 1, 2, 3, 4, 5, 6, 7, 8, 9 or 10. In a further embodiment, the number of substitutions, preferably conservative substitutions, in SEQ ID NO: 6 is not more than 10, e.g., 1, 2, 3, 4, 5, 6, 7, 8, 9 or 10. Examples of amino acid changes and conservative substitutions are described in the third aspect of the present disclosure.

In an embodiment of the fourth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a fifth aspect the present disclosure relates to polypeptides having lysozyme activity having at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 8. In one embodiment, the polypeptides differ by up to 38 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 acids from the mature polypeptide of SEQ ID NO: 8.

In a continuation of the fifth aspect, the present disclosure relates to polypeptides having lysozyme activity having at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9. In one embodiment, the polypeptides differ by up to 38 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 amino acids from the mature polypeptide of SEQ ID NO: 9.

In one embodiment, the present disclosure relates to polypeptides having lysozyme activity and having a sequence identity to SEQ ID NO: 9 of at least 81% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 9 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 9 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 9 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 8. In one embodiment, the polypeptide preferably comprises or consists of the amino acid sequence of SEQ ID NO: 9; comprises the amino acid sequence of SEQ ID NO: 9 and a N-terminal and/or C-terminal His-tag and/or HQ-tag; comprises the amino acid sequence of SEQ ID NO: 9 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 9. In one embodiment, the polypeptide comprises or consists of amino acids 1 to 201 of SEQ ID NO: 9. In an embodiment, the polypeptide has been isolated.

In a continuation of the fifth aspect, the present disclosure relates to a polypeptide having lysozyme activity encoded by a polynucleotide having a sequence identity to the mature polypeptide coding sequence of SEQ ID NO: 7 of at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fifth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fifth aspect, the present disclosure relates to variants of SEQ ID NO: 9 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 9 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 9 is not more than 10, e.g., 1, 2, 3, 4, 5, 6, 7, 8, 9 or 10. In one embodiment, the number of substitutions and/or deletions and/or insertions in SEQ ID NO: 9 is not more than 10, e.g., 1, 2, 3, 4, 5, 6, 7, 8, 9 or 10. In a further embodiment, the number of substitutions, preferably conservative substitutions, in SEQ ID NO: 9 is not more than 10, e.g., 1, 2, 3, 4, 5, 6, 7, 8, 9 or 10. Examples of amino acid changes and conservative substitutions are described in the third aspect of the present disclosure.

In an embodiment of the fifth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a sixth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 11. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 11.

In a continuation of the sixth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 12.

In one embodiment, the present disclosure relates to polypeptides having lysozyme activity and having a sequence identity to SEQ ID NO: 12 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 12 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 12 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 12 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 11. In one embodiment, the polypeptide preferably comprises or consists of the amino acid sequence of SEQ ID NO: 12; comprises the amino acid sequence of SEQ ID NO: 12 and a N-terminal and/or C-terminal His-tag and/or HQ-tag; comprises the amino acid sequence of SEQ ID NO: 12 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 12. In one embodiment, the polypeptide comprises or consists of amino acids 1 to 203 of SEQ ID NO: 12. In an embodiment, the polypeptide has been isolated.

In a continuation of the sixth aspect, the present disclosure relates to a polypeptide having lysozyme activity encoded by a polynucleotide having a sequence identity to the mature polypeptide coding sequence of SEQ ID NO: 10 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the sixth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the sixth aspect, the present disclosure relates to variants of SEQ ID NO: 12 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 12 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 12 is not more than 10, e.g., 1, 2, 3, 4, 5, 6, 7, 8, 9 or 10. In one embodiment, the number of substitutions and/or deletions and/or insertions in SEQ ID NO: 12 is not more than 10, e.g., 1, 2, 3, 4, 5, 6, 7, 8, 9 or 10. In a further embodiment, the number of substitutions, preferably conservative substitutions, in SEQ ID NO: 12 is not more than 10, e.g., 1, 2, 3, 4, 5, 6, 7, 8, 9 or 10. Examples of amino acid changes and conservative substitutions are described in the third aspect of the present disclosure.

In an embodiment of the sixth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the sixth aspect comprises the motif YKNA (SEQ ID NO: 160) and/or the motif FGGW[S/T] (SEQ ID NO: 161).

In a seventh aspect the present disclosure relates to polypeptides having lysozyme activity having at least 97%, e.g., at least 97.5%, at least 98%, at least 98.5%, at least 99%, at least 99.5%, or 100% sequence identity to the mature polypeptide of SEQ ID NO: 14. In one embodiment, the polypeptides differ by up to 6 amino acids, e.g., 1, 2, 3, 4, 5 or 6 amino acids from the mature polypeptide of SEQ ID NO: 14.

In a continuation of the seventh aspect, the present disclosure relates to polypeptides having lysozyme activity having at least 97%, e.g., at least 97.5%, at least 98%, at least 98.5%, at least 99%, at least 99.5%, or 100% sequence identity to SEQ ID NO: 15. In one embodiment, the polypeptides differ by up to 6 amino acids, e.g., 1, 2, 3, 4, 5 or 6 amino acids from the mature polypeptide of SEQ ID NO: 15.

In one embodiment, the present disclosure relates to polypeptides having lysozyme activity and having a sequence identity to SEQ ID NO: 15 of at least 97% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 15 of at least 98% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 15 of at least 99% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 14. In one embodiment, the polypeptide preferably comprises or consists of the amino acid sequence of SEQ ID NO: 15; comprises the amino acid sequence of SEQ ID NO: 15 and a N-terminal and/or C-terminal His-tag and/or HQ-tag; comprises the amino acid sequence of SEQ ID NO: 15 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 15. In one embodiment, the polypeptide comprises or consists of amino acids 1 to 208 of SEQ ID NO: 15. In an embodiment, the polypeptide has been isolated.

In a continuation of the seventh aspect, the present disclosure relates to a polypeptide having lysozyme activity encoded by a polynucleotide having a sequence identity to the mature polypeptide coding sequence of SEQ ID NO: 13 of at least 97%, e.g., at least 97.5%, at least 98%, at least 98.5%, at least 99%, at least 99.5%, or 100%. In a further embodiment, the polypeptide has been isolated.

In an embodiment of the seventh aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the seventh aspect, the present disclosure relates to variants of SEQ ID NO: 15 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 15 is not more than 6, e.g., 1, 2, 3, 4, 5 or 6. In an embodiment, the number of positions comprising one or more amino acid substitutions, and/or one or more amino acid deletions, and/or one or more amino acid insertions or any combination thereof in SEQ ID NO: 15 is not more than 6, e.g., 1, 2, 3, 4, 5 or 6. In one embodiment, the number of substitutions and/or deletions and/or insertions in SEQ ID NO: 15 is not more than 6, e.g., 1, 2, 3, 4, 5 or 6. In a further embodiment, the number of substitutions, preferably conservative substitutions, in SEQ ID NO: 15 is not more than 6, e.g., 1, 2, 3, 4, 5 or 6. Examples of amino acid changes and conservative substitutions are described in the third aspect of the present disclosure.

In an embodiment of the seventh aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an eighth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 17. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 17.

In a continuation of the eighth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 18. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 18.

In one embodiment, the present disclosure relates to polypeptides having lysozyme activity and having a sequence identity to SEQ ID NO: 18 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 18 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 18 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 18 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 17. In one embodiment, the polypeptide preferably comprises or consists of the amino acid sequence of SEQ ID NO: 18; comprises the amino acid sequence of SEQ ID NO: 18 and a N-terminal and/or C-terminal His-tag and/or HQ-tag; comprises the amino acid sequence of SEQ ID NO: 18 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 18. In one embodiment, the polypeptide comprises or consists of amino acids 1 to 207 of SEQ ID NO: 18. In an embodiment, the polypeptide has been isolated.

In a continuation of the eighth aspect, the present disclosure relates to a polypeptide having lysozyme activity encoded by a polynucleotide having a sequence identity to the mature polypeptide coding sequence of SEQ ID NO: 16 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the eighth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the eighth aspect, the present disclosure relates to variants of SEQ ID NO: 18 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 18 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 18 is not more than 10, e.g., 1, 2, 3, 4, 5, 6, 7, 8, 9 or 10. In one embodiment, the number of substitutions and/or deletions and/or insertions in SEQ ID NO: 18 is not more than 10, e.g., 1, 2, 3, 4, 5, 6, 7, 8, 9 or 10. In a further embodiment, the number of substitutions, preferably conservative substitutions, in SEQ ID NO: 18 is not more than 10, e.g., 1, 2, 3, 4, 5, 6, 7, 8, 9 or 10. Examples of amino acid changes and conservative substitutions are described in the third aspect of the present disclosure.

In an embodiment of the eighth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a ninth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 23. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 23.

In a continuation of the ninth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 24.

In one embodiment, the present disclosure relates to polypeptides having lysozyme activity and having a sequence identity to SEQ ID NO: 24 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 24 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 24 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 24 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 23. In one embodiment, the polypeptide preferably comprises or consists of the amino acid sequence of SEQ ID NO: 24; comprises the amino acid sequence of SEQ ID NO: 24 and a N-terminal and/or C-terminal His-tag and/or HQ-tag; comprises the amino acid sequence of SEQ ID NO: 24 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 24. In one embodiment, the polypeptide comprises or consists of amino acids 1 to 207 of SEQ ID NO: 24. In an embodiment, the polypeptide has been isolated.

In a continuation of the ninth aspect, the present disclosure relates to a polypeptide having lysozyme activity encoded by a polynucleotide having a sequence identity to the mature polypeptide coding sequence of SEQ ID NO: 22 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the ninth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the ninth aspect, the present disclosure relates to variants of SEQ ID NO: 24 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 24 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 24 is not more than 10, e.g., 1, 2, 3, 4, 5, 6, 7, 8, 9 or 10. In one embodiment, the number of substitutions and/or deletions and/or insertions in SEQ ID NO: 24 is not more than 10, e.g., 1, 2, 3, 4, 5, 6, 7, 8, 9 or 10. In a further embodiment, the number of substitutions, preferably conservative substitutions, in SEQ ID NO: 24 is not more than 10, e.g., 1, 2, 3, 4, 5, 6, 7, 8, 9 or 10. Examples of amino acid changes and conservative substitutions are described in the third aspect of the present disclosure.

In an embodiment of the ninth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a tenth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 26. In one embodiment, the polypeptides differ by up to 40 amino acids, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 26.

In a continuation of the tenth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 27.

In one embodiment, the present disclosure relates to polypeptides having lysozyme activity and having a sequence identity to SEQ ID NO: 27 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 27 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 27 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 27 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 26. In one embodiment, the polypeptide preferably comprises or consists of the amino acid sequence of SEQ ID NO: 27; comprises the amino acid sequence of SEQ ID NO: 27 and a N-terminal and/or C-terminal His-tag and/or HQ-tag; comprises the amino acid sequence of SEQ ID NO: 27 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 27. In one embodiment, the polypeptide comprises or consists of amino acids 1 to 207 of SEQ ID NO: 27. In an embodiment, the polypeptide has been isolated.

In a continuation of the tenth aspect, the present disclosure relates to a polypeptide having lysozyme activity encoded by a polynucleotide having a sequence identity to the mature polypeptide coding sequence of SEQ ID NO: 25 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the tenth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the tenth aspect, the present disclosure relates to variants of SEQ ID NO: 27 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 27 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 27 is not more than 10, e.g., 1, 2, 3, 4, 5, 6, 7, 8, 9 or 10. In one embodiment, the number of substitutions and/or deletions and/or insertions in SEQ ID NO: 27 is not more than 10, e.g., 1, 2, 3, 4, 5, 6, 7, 8, 9 or 10. In a further embodiment, the number of substitutions, preferably conservative substitutions, in SEQ ID NO: 27 is not more than 10, e.g., 1, 2, 3, 4, 5, 6, 7, 8, 9 or 10. Examples of amino acid changes and conservative substitutions are described in the third aspect of the present disclosure.

In an embodiment of the tenth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the tenth aspect comprises the motif YKNA (SEQ ID NO: 160) and/or the motif FGGW[S/T] (SEQ ID NO: 161).

In an eleventh aspect the present disclosure relates to polypeptides having lysozyme activity having at least 83%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 29. In one embodiment, the polypeptides differ by up to 35 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids from the mature polypeptide of SEQ ID NO: 29.

In a continuation of the eleventh aspect, the present disclosure relates to polypeptides having lysozyme activity having at least 83%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30. In one embodiment, the polypeptides differ by up to 35 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids from the mature polypeptide of SEQ ID NO: 30.

In one embodiment, the present disclosure relates to polypeptides having lysozyme activity and having a sequence identity to SEQ ID NO: 30 of at least 83% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 30 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 30 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 30 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 29. In one embodiment, the polypeptide preferably comprises or consists of the amino acid sequence of SEQ ID NO: 30; comprises the amino acid sequence of SEQ ID NO: 30 and a N-terminal and/or C-terminal His-tag and/or HQ-tag; comprises the amino acid sequence of SEQ ID NO: 30 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 30. In one embodiment, the polypeptide comprises or consists of amino acids 1 to 207 of SEQ ID NO: 30. In an embodiment, the polypeptide has been isolated.

In a continuation of the eleventh aspect, the present disclosure relates to a polypeptide having lysozyme activity encoded by a polynucleotide having a sequence identity to the mature polypeptide coding sequence of SEQ ID NO: 28 of at least 83%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the eleventh aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the eleventh aspect, the present disclosure relates to variants of SEQ ID NO: 30 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 30 is not more than 33, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33. In an embodiment, the number of positions comprising one or more amino acid substitutions, and/or one or more amino acid deletions, and/or one or more amino acid insertions or any combination thereof in SEQ ID NO: 30 is not more than 10, e.g., 1, 2, 3, 4, 5, 6, 7, 8, 9 or 10. In one embodiment, the number of substitutions and/or deletions and/or insertions in SEQ ID NO: 30 is not more than 10, e.g., 1, 2, 3, 4, 5, 6, 7, 8, 9 or 10. In a further embodiment, the number of substitutions, preferably conservative substitutions, in SEQ ID NO: 30 is not more than 10, e.g., 1, 2, 3, 4, 5, 6, 7, 8, 9 or 10. Examples of amino acid changes and conservative substitutions are described in the third aspect of the present disclosure.

In an embodiment of the eleventh aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a twelfth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 32. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 32.

In a continuation of the twelfth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 33.

In one embodiment, the present disclosure relates to polypeptides having lysozyme activity and having a sequence identity to SEQ ID NO: 33 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 33 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 33 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 33 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 32. In one embodiment, the polypeptide preferably comprises or consists of the amino acid sequence of SEQ ID NO: 33; comprises the amino acid sequence of SEQ ID NO: 33 and a N-terminal and/or C-terminal His-tag and/or HQ-tag; comprises the amino acid sequence of SEQ ID NO: 33 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 33. In one embodiment, the polypeptide comprises or consists of amino acids 1 to 208 of SEQ ID NO: 33. In an embodiment, the polypeptide has been isolated.

In a continuation of the twelfth aspect, the present disclosure relates to a polypeptide having lysozyme activity encoded by a polynucleotide having a sequence identity to the mature polypeptide coding sequence of SEQ ID NO: 31 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twelfth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twelfth aspect, the present disclosure relates to variants of SEQ ID NO: 33 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 33 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 33 is not more than 10, e.g., 1, 2, 3, 4, 5, 6, 7, 8, 9 or 10. In one embodiment, the number of substitutions and/or deletions and/or insertions in SEQ ID NO: 33 is not more than 10, e.g., 1, 2, 3, 4, 5, 6, 7, 8, 9 or 10. In a further embodiment, the number of substitutions, preferably conservative substitutions, in SEQ ID NO: 33 is not more than 10, e.g., 1, 2, 3, 4, 5, 6, 7, 8, 9 or 10. Examples of amino acid changes and conservative substitutions are described in the third aspect of the present disclosure.

In an embodiment of the twelfth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirteenth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 35. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 35.

In a continuation of the thirteenth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 36.

In one embodiment, the present disclosure relates to polypeptides having lysozyme activity and having a sequence identity to SEQ ID NO: 36 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 36 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 36 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 36 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 35. In one embodiment, the polypeptide preferably comprises or consists of the amino acid sequence of SEQ ID NO: 36; comprises the amino acid sequence of SEQ ID NO: 36 and a N-terminal and/or C-terminal His-tag and/or HQ-tag; comprises the amino acid sequence of SEQ ID NO: 36 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 36. In one embodiment, the polypeptide comprises or consists of amino acids 1 to 208 of SEQ ID NO: 36. In an embodiment, the polypeptide has been isolated.

In a continuation of the thirteenth aspect, the present disclosure relates to a polypeptide having lysozyme activity encoded by a polynucleotide having a sequence identity to the mature polypeptide coding sequence of SEQ ID NO: 34 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the thirteenth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirteenth aspect, the present disclosure relates to variants of SEQ ID NO: 36 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 36 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 36 is not more than 10, e.g., 1, 2, 3, 4, 5, 6, 7, 8, 9 or 10. In one embodiment, the number of substitutions and/or deletions and/or insertions in SEQ ID NO: 36 is not more than 10, e.g., 1, 2, 3, 4, 5, 6, 7, 8, 9 or 10. In a further embodiment, the number of substitutions, preferably conservative substitutions, in SEQ ID NO: 36 is not more than 10, e.g., 1, 2, 3, 4, 5, 6, 7, 8, 9 or 10. Examples of amino acid changes and conservative substitutions are described in the third aspect of the present disclosure.

In an embodiment of the thirteenth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a fourteenth aspect the present disclosure relates to polypeptides having lysozyme activity having at least 83%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 38. In one embodiment, the polypeptides differ by up to 35 amino 26, 27, 28, 29, 30, 31, 32, 33, 34 or 35 amino acids from the mature polypeptide of SEQ ID NO: 38.

In a continuation of the fourteenth aspect, the present disclosure relates to polypeptides having lysozyme activity having at least 83%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39. In one embodiment, the polypeptides differ by up to 35 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids from the mature polypeptide of SEQ ID NO: 39.

In one embodiment, the present disclosure relates to polypeptides having lysozyme activity and having a sequence identity to SEQ ID NO: 39 of at least 83% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 39 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 39 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 39 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 38. In one embodiment, the polypeptide preferably comprises or consists of the amino acid sequence of SEQ ID NO: 39; comprises the amino acid sequence of SEQ ID NO: 39 and a N-terminal and/or C-terminal His-tag and/or HQ-tag; comprises the amino acid sequence of SEQ ID NO: 39 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 39. In one embodiment, the polypeptide comprises or consists of amino acids 1 to 206 of SEQ ID NO: 39. In an embodiment, the polypeptide has been isolated.

In a continuation of the fourteenth aspect, the present disclosure relates to a polypeptide having lysozyme activity encoded by a polynucleotide having a sequence identity to the mature polypeptide coding sequence of SEQ ID NO: 37 of at least 83%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fourteenth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fourteenth aspect, the present disclosure relates to variants of SEQ ID NO: 39 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 39 is not more than 33, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33. In an embodiment, the number of positions comprising one or more amino acid substitutions, and/or one or more amino acid deletions, and/or one or more amino acid insertions or any combination thereof in SEQ ID NO: 39 is not more than 10, e.g., 1, 2, 3, 4, 5, 6, 7, 8, 9 or 10. In one embodiment, the number of substitutions and/or deletions and/or insertions in SEQ ID NO: 39 is not more than 10, e.g., 1, 2, 3, 4, 5, 6, 7, 8, 9 or 10. In a further embodiment, the number of substitutions, preferably conservative substitutions, in SEQ ID NO: 39 is not more than 10, e.g., 1, 2, 3, 4, 5, 6, 7, 8, 9 or 10. Examples of amino acid changes and conservative substitutions are described in the third aspect of the present disclosure.

In an embodiment of the fourteenth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a fifteenth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 41. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 41.

In a continuation of the fifteenth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 42.

In one embodiment, the present disclosure relates to polypeptides having lysozyme activity and having a sequence identity to SEQ ID NO: 42 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 42 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 42 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 42 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 41. In one embodiment, the polypeptide preferably comprises or consists of the amino acid sequence of SEQ ID NO: 42; comprises the amino acid sequence of SEQ ID NO: 42 and a N-terminal and/or C-terminal His-tag and/or HQ-tag; comprises the amino acid sequence of SEQ ID NO: 42 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 42. In one embodiment, the polypeptide comprises or consists of amino acids 1 to 207 of SEQ ID NO: 42. In an embodiment, the polypeptide has been isolated.

In a continuation of the fifteenth aspect, the present disclosure relates to a polypeptide having lysozyme activity encoded by a polynucleotide having a sequence identity to the mature polypeptide coding sequence of SEQ ID NO: 40 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fifteenth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fifteenth aspect, the present disclosure relates to variants of SEQ ID NO: 42 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 42 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 42 is not more than 10, e.g., 1, 2, 3, 4, 5, 6, 7, 8, 9 or 10. In one embodiment, the number of substitutions and/or deletions and/or insertions in SEQ ID NO: 42 is not more than 10, e.g., 1, 2, 3, 4, 5, 6, 7, 8, 9 or 10. In a further embodiment, the number of substitutions, preferably conservative substitutions, in SEQ ID NO: 42 is not more than 10, e.g., 1, 2, 3, 4, 5, 6, 7, 8, 9 or 10. Examples of amino acid changes and conservative substitutions are described in the third aspect of the present disclosure.

In an embodiment of the fifteenth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the fifteenth aspect comprises the motif YKNA (SEQ ID NO: 160) and/or the motif FGGW[S/T] (SEQ ID NO: 161).

In a sixteenth aspect the present disclosure relates to polypeptides having lysozyme activity having at least 86%, e.g., at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 44. In one embodiment, the polypeptides differ by up to 30 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids from the mature polypeptide of SEQ ID NO: 44.

In a continuation of the sixteenth aspect, the present disclosure relates to polypeptides having lysozyme activity having at least 86%, e.g., at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45. In one embodiment, the polypeptides differ by up to 30 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids from the mature polypeptide of SEQ ID NO: 45.

In one embodiment, the present disclosure relates to polypeptides having lysozyme activity and having a sequence identity to SEQ ID NO: 45 of at least 86% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 45 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 45 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 44. In one embodiment, the polypeptide preferably comprises or consists of the amino acid sequence of SEQ ID NO: 45; comprises the amino acid sequence of SEQ ID NO: 45 and a N-terminal and/or C-terminal His-tag and/or HQ-tag; comprises the amino acid sequence of SEQ ID NO: 45 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 45. In one embodiment, the polypeptide comprises or consists of amino acids 1 to 216 of SEQ ID NO: 45. In an embodiment, the polypeptide has been isolated.

In a continuation of the sixteenth aspect, the present disclosure relates to a polypeptide having lysozyme activity encoded by a polynucleotide having a sequence identity to the mature polypeptide coding sequence of SEQ ID NO: 43 of at least 86%, e.g., at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the sixteenth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the sixteenth aspect, the present disclosure relates to variants of SEQ ID NO: 45 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 45 is not more than 30, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. In an embodiment, the number of positions comprising one or more amino acid substitutions, and/or one or more amino acid deletions, and/or one or more amino acid insertions or any combination thereof in SEQ ID NO: 45 is not more than 10, e.g., 1, 2, 3, 4, 5, 6, 7, 8, 9 or 10. In one embodiment, the number of substitutions and/or deletions and/or insertions in SEQ ID NO: 45 is not more than 10, e.g., 1, 2, 3, 4, 5, 6, 7, 8, 9 or 10. In a further embodiment, the number of substitutions, preferably conservative substitutions, in SEQ ID NO: 45 is not more than 10, e.g., 1, 2, 3, 4, 5, 6, 7, 8, 9 or 10. Examples of amino acid changes and conservative substitutions are described in the third aspect of the present disclosure.

In an embodiment of the sixteenth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a seventeenth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 47. In one embodiment, the polypeptides differ by up to 40 amino acids, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 47.

In a continuation of the seventeenth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 48.

In one embodiment, the present disclosure relates to polypeptides having lysozyme activity and having a sequence identity to SEQ ID NO: 48 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 48 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 48 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 48 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 47. In one embodiment, the polypeptide preferably comprises or consists of the amino acid sequence of SEQ ID NO: 48; comprises the amino acid sequence of SEQ ID NO: 48 and a N-terminal and/or C-terminal His-tag and/or HQ-tag; comprises the amino acid sequence of SEQ ID NO: 48 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 48. In one embodiment, the polypeptide comprises or consists of amino acids 1 to 218 of SEQ ID NO: 48. In an embodiment, the polypeptide has been isolated.

In a continuation of the seventeenth aspect, the present disclosure relates to a polypeptide having lysozyme activity encoded by a polynucleotide having a sequence identity to the mature polypeptide coding sequence of SEQ ID NO: 46 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the seventeenth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the seventeenth aspect, the present disclosure relates to variants of SEQ ID NO: 48 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 48 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 48 is not more than 10, e.g., 1, 2, 3, 4, 5, 6, 7, 8, 9 or 10. In one embodiment, the number of substitutions and/or deletions and/or insertions in SEQ ID NO: 48 is not more than 10, e.g., 1, 2, 3, 4, 5, 6, 7, 8, 9 or 10. In a further embodiment, the number of substitutions, preferably conservative substitutions, in SEQ ID NO: 48 is not more than 10, e.g., 1, 2, 3, 4, 5, 6, 7, 8, 9 or 10. Examples of amino acid changes and conservative substitutions are described in the third aspect of the present disclosure.

In an embodiment of the seventeenth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an eighteenth aspect, the present disclosure relates to polypeptides having lysozyme activity having at least 92%, e.g., at least 93%, at least 94%, at least 95%, at least 96%, at least 97%, at least 98%, at least 99%, or 100% sequence identity to the mature polypeptide of SEQ ID NO: 50. In one embodiment, the polypeptides differ by up to 16 amino acids, e.g., 1, 2, 3, 4, 5, 6, 7, 8, 9, 10, 11, 12, 13, 14, 15 or 16 amino acids from the mature polypeptide of SEQ ID NO: 50.

In a continuation of the eighteenth aspect, the present disclosure relates to polypeptides having lysozyme activity having at least 92%, e.g., at least 93%, at least 94%, at least 95%, at least 96%, at least 97%, at least 98%, at least 99%, or 100% sequence identity to SEQ ID NO: 51. In one embodiment, the polypeptides differ by up to 16 amino acids, e.g., 1, 2, 3, 4, 5, 6, 7, 8, 9, 10, 11, 12, 13, 14, 15 or 16 amino acids from the mature polypeptide of SEQ ID NO: 51.

In one embodiment, the present disclosure relates to polypeptides having lysozyme activity and having a sequence identity to SEQ ID NO: 51 of at least 92% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 51 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 50. In one embodiment, the polypeptide preferably comprises or consists of the amino acid sequence of SEQ ID NO: 51; comprises the amino acid sequence of SEQ ID NO: 51 and a N-terminal and/or C-terminal His-tag and/or HQ-tag; comprises the amino acid sequence of SEQ ID NO: 51 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 51. In one embodiment, the polypeptide comprises or consists of amino acids 1 to 204 of SEQ ID NO: 51. In an embodiment, the polypeptide has been isolated.

In a continuation of the eighteenth aspect, the present disclosure relates to a polypeptide having lysozyme activity encoded by a polynucleotide having a sequence identity to the mature polypeptide coding sequence of SEQ ID NO: 49 of at least 92%, e.g., at least 93%, at least 94%, at least 95%, at least 96%, at least 97%, at least 98%, at least 99%, or 100%. In a further embodiment, the polypeptide has been isolated.

In an embodiment of the eighteenth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the eighteenth aspect, the present disclosure relates to variants of SEQ ID NO: 51 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 51 is not more than 16, e.g., 1, 2, 3, 4, 5, 6, 7, 8, 9, 10, 11, 12, 13, 14, 15 or 16. In an embodiment, the number of positions comprising one or more amino acid substitutions, and/or one or more amino acid deletions, and/or one or more amino acid insertions or any combination thereof in SEQ ID NO: 51 is not more than 10, e.g., 1, 2, 3, 4, 5, 6, 7, 8, 9 or 10. In one embodiment, the number of substitutions and/or deletions and/or insertions in SEQ ID NO: 51 is not more than 10, e.g., 1, 2, 3, 4, 5, 6, 7, 8, 9 or 10. In a further embodiment, the number of substitutions, preferably conservative substitutions, in SEQ ID NO: 51 is not more than 10, e.g., 1, 2, 3, 4, 5, 6, 7, 8, 9 or 10. Examples of amino acid changes and conservative substitutions are described in the third aspect of the present disclosure.

In an embodiment of the eighteenth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a nineteenth aspect the present disclosure relates to polypeptides having lysozyme activity having at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 53. In one embodiment, the polypeptides differ by up to 38 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 amino acids from the mature polypeptide of SEQ ID NO: 53.

In a continuation of the nineteenth aspect, the present disclosure relates to polypeptides having lysozyme activity having at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 54. In one embodiment, the polypeptides differ by up to 38 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 amino acids from the mature polypeptide of SEQ ID NO: 54.

In one embodiment, the present disclosure relates to polypeptides having lysozyme activity and having a sequence identity to SEQ ID NO: 54 of at least 81% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 54 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 54 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 54 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 53. In one embodiment, the polypeptide preferably comprises or consists of the amino acid sequence of SEQ ID NO: 54; comprises the amino acid sequence of SEQ ID NO: 54 and a N-terminal and/or C-terminal His-tag and/or HQ-tag; comprises the amino acid sequence of SEQ ID NO: 54 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 54. In one embodiment, the polypeptide comprises or consists of amino acids 1 to 203 of SEQ ID NO: 54. In an embodiment, the polypeptide has been isolated.

In a continuation of the nineteenth aspect, the present disclosure relates to a polypeptide having lysozyme activity encoded by a polynucleotide having a sequence identity to the mature polypeptide coding sequence of SEQ ID NO: 52 of at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the nineteenth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the nineteenth aspect, the present disclosure relates to variants of SEQ ID NO: 54 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 54 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 54 is not more than 10, e.g., 1, 2, 3, 4, 5, 6, 7, 8, 9 or 10. In one embodiment, the number of substitutions and/or deletions and/or insertions in SEQ ID NO: 54 is not more than 10, e.g., 1, 2, 3, 4, 5, 6, 7, 8, 9 or 10. In a further embodiment, the number of substitutions, preferably conservative substitutions, in SEQ ID NO: 54 is not more than 10, e.g., 1, 2, 3, 4, 5, 6, 7, 8, 9 or 10. Examples of amino acid changes and conservative substitutions are described in the third aspect of the present disclosure.

In an embodiment of the nineteenth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a twentieth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 56. In one embodiment, the polypeptides differ by up to 40 amino 26, 27, 28, 29, 30, 31, 32, 33, 34, 35, 36, 37, 38, 39 or 40 amino acids from the mature polypeptide of SEQ ID NO: 56.

In a continuation of the twentieth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 57. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 57.

In one embodiment, the present disclosure relates to polypeptides having lysozyme activity and having a sequence identity to SEQ ID NO: 57 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 57 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 57 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 57 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 56. In one embodiment, the polypeptide preferably comprises or consists of the amino acid sequence of SEQ ID NO: 57; comprises the amino acid sequence of SEQ ID NO: 57 and a N-terminal and/or C-terminal His-tag and/or HQ-tag; comprises the amino acid sequence of SEQ ID NO: 57 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 57. In one embodiment, the polypeptide comprises or consists of amino acids 1 to 209 of SEQ ID NO: 57. In an embodiment, the polypeptide has been isolated.

In a continuation of the twentieth aspect, the present disclosure relates to a polypeptide having lysozyme activity encoded by a polynucleotide having a sequence identity to the mature polypeptide coding sequence of SEQ ID NO: 55 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twentieth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twentieth aspect, the present disclosure relates to variants of SEQ ID NO: 57 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 57 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 57 is not more than 10, e.g., 1, 2, 3, 4, 5, 6, 7, 8, 9 or 10. In one embodiment, the number of substitutions and/or deletions and/or insertions in SEQ ID NO: 57 is not more than 10, e.g., 1, 2, 3, 4, 5, 6, 7, 8, 9 or 10. In a further embodiment, the number of substitutions, preferably conservative substitutions, in SEQ ID NO: 57 is not more than 10, e.g., 1, 2, 3, 4, 5, 6, 7, 8, 9 or 10. Examples of amino acid changes and conservative substitutions are described in the third aspect of the present disclosure.

In an embodiment of the twentieth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the twentieth aspect comprises the motif YKNA (SEQ ID NO: 160) and/or the motif FGGW[S/T] (SEQ ID NO: 161).

In a twenty-first aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 59. In one embodiment, the polypeptides differ by up to 40 amino 26, 27, 28, 29, 30, 31, 32, 33, 34, 35, 36, 37, 38, 39 or 40 amino acids from the mature polypeptide of SEQ ID NO: 59.

In a continuation of the twenty-first aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 60.

In one embodiment, the present disclosure relates to polypeptides having lysozyme activity and having a sequence identity to SEQ ID NO: 60 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 60 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 60 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 60 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 59. In one embodiment, the polypeptide preferably comprises or consists of the amino acid sequence of SEQ ID NO: 60; comprises the amino acid sequence of SEQ ID NO: 60 and a N-terminal and/or C-terminal His-tag and/or HQ-tag; comprises the amino acid sequence of SEQ ID NO: 60 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 60. In one embodiment, the polypeptide comprises or consists of amino acids 1 to 208 of SEQ ID NO: 60. In an embodiment, the polypeptide has been isolated.

In a continuation of the twenty-first aspect, the present disclosure relates to a polypeptide having lysozyme activity encoded by a polynucleotide having a sequence identity to the mature polypeptide coding sequence of SEQ ID NO: 58 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twenty-first aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twenty-first aspect, the present disclosure relates to variants of SEQ ID NO: 60 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 60 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 60 is not more than 10, e.g., 1, 2, 3, 4, 5, 6, 7, 8, 9 or 10. In one embodiment, the number of substitutions and/or deletions and/or insertions in SEQ ID NO: 60 is not more than 10, e.g., 1, 2, 3, 4, 5, 6, 7, 8, 9 or 10. In a further embodiment, the number of substitutions, preferably conservative substitutions, in SEQ ID NO: 60 is not more than 10, e.g., 1, 2, 3, 4, 5, 6, 7, 8, 9 or 10. Examples of amino acid changes and conservative substitutions are described in the third aspect of the present disclosure.

In an embodiment of the twenty-first aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a twenty-second aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 62. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 62.

In a continuation of the twenty-second aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 63. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 63.

In one embodiment, the present disclosure relates to polypeptides having lysozyme activity and having a sequence identity to SEQ ID NO: 63 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 63 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 63 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 63 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 62. In one embodiment, the polypeptide preferably comprises or consists of the amino acid sequence of SEQ ID NO: 63; comprises the amino acid sequence of SEQ ID NO: 63 and a N-terminal and/or C-terminal His-tag and/or HQ-tag; comprises the amino acid sequence of SEQ ID NO: 63 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 63. In one embodiment, the polypeptide comprises or consists of amino acids 1 to 210 of SEQ ID NO: 63. In an embodiment, the polypeptide has been isolated.

In a continuation of the twenty-second aspect, the present disclosure relates to a polypeptide having lysozyme activity encoded by a polynucleotide having a sequence identity to the mature polypeptide coding sequence of SEQ ID NO: 61 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twenty-second aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twenty-second aspect, the present disclosure relates to variants of SEQ ID NO: 63 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 63 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 63 is not more than 10, e.g., 1, 2, 3, 4, 5, 6, 7, 8, 9 or 10. In one embodiment, the number of substitutions and/or deletions and/or insertions in SEQ ID NO: 63 is not more than 10, e.g., 1, 2, 3, 4, 5, 6, 7, 8, 9 or 10. In a further embodiment, the number of substitutions, preferably conservative substitutions, in SEQ ID NO: 63 is not more than 10, e.g., 1, 2, 3, 4, 5, 6, 7, 8, 9 or 10. Examples of amino acid changes and conservative substitutions are described in the third aspect of the present disclosure.

In an embodiment of the twenty-second aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the twenty-second aspect comprises the motif YKNA (SEQ ID NO: 160) and/or the motif FGGW[S/T] (SEQ ID NO: 161).

In a twenty-third aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 65. In one embodiment, the polypeptides differ by up to 40 amino 26, 27, 28, 29, 30, 31, 32, 33, 34, 35, 36, 37, 38, 39 or 40 amino acids from the mature polypeptide of SEQ ID NO: 65.

In a continuation of the twenty-third aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 66. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 66.

In one embodiment, the present disclosure relates to polypeptides having lysozyme activity and having a sequence identity to SEQ ID NO: 66 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 66 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 66 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 66 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 65. In one embodiment, the polypeptide preferably comprises or consists of the amino acid sequence of SEQ ID NO: 66; comprises the amino acid sequence of SEQ ID NO: 66 and a N-terminal and/or C-terminal His-tag and/or HQ-tag; comprises the amino acid sequence of SEQ ID NO: 66 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 66. In one embodiment, the polypeptide comprises or consists of amino acids 1 to 207 of SEQ ID NO: 66. In an embodiment, the polypeptide has been isolated.

In a continuation of the twenty-third aspect, the present disclosure relates to a polypeptide having lysozyme activity encoded by a polynucleotide having a sequence identity to the mature polypeptide coding sequence of SEQ ID NO: 64 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twenty-third aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twenty-third aspect, the present disclosure relates to variants of SEQ ID NO: 66 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 66 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 66 is not more than 10, e.g., 1, 2, 3, 4, 5, 6, 7, 8, 9 or 10. In one embodiment, the number of substitutions and/or deletions and/or insertions in SEQ ID NO: 66 is not more than 10, e.g., 1, 2, 3, 4, 5, 6, 7, 8, 9 or 10. In a further embodiment, the number of substitutions, preferably conservative substitutions, in SEQ ID NO: 66 is not more than 10, e.g., 1, 2, 3, 4, 5, 6, 7, 8, 9 or 10. Examples of amino acid changes and conservative substitutions are described in the third aspect of the present disclosure.

In an embodiment of the twenty-third aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the twenty-third aspect comprises the motif YKNA (SEQ ID NO: 160) and/or the motif FGGW[S/T] (SEQ ID NO: 161).

In a twenty-fourth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 68. In one embodiment, the polypeptides differ by up to 40 amino 26, 27, 28, 29, 30, 31, 32, 33, 34, 35, 36, 37, 38, 39 or 40 amino acids from the mature polypeptide of SEQ ID NO: 68.

In a continuation of the twenty-fourth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 69. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 69.

In one embodiment, the present disclosure relates to polypeptides having lysozyme activity and having a sequence identity to SEQ ID NO: 69 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 69 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 69 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 69 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 68. In one embodiment, the polypeptide preferably comprises or consists of the amino acid sequence of SEQ ID NO: 69; comprises the amino acid sequence of SEQ ID NO: 69 and a N-terminal and/or C-terminal His-tag and/or HQ-tag; comprises the amino acid sequence of SEQ ID NO: 69 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 69. In one embodiment, the polypeptide comprises or consists of amino acids 1 to 207 of SEQ ID NO: 69. In an embodiment, the polypeptide has been isolated.

In a continuation of the twenty-fourth aspect, the present disclosure relates to a polypeptide having lysozyme activity encoded by a polynucleotide having a sequence identity to the mature polypeptide coding sequence of SEQ ID NO: 67 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twenty-fourth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twenty-fourth aspect, the present disclosure relates to variants of SEQ ID NO: 69 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 69 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 69 is not more than 10, e.g., 1, 2, 3, 4, 5, 6, 7, 8, 9 or 10. In one embodiment, the number of substitutions and/or deletions and/or insertions in SEQ ID NO: 69 is not more than 10, e.g., 1, 2, 3, 4, 5, 6, 7, 8, 9 or 10. In a further embodiment, the number of substitutions, preferably conservative substitutions, in SEQ ID NO: 69 is not more than 10, e.g., 1, 2, 3, 4, 5, 6, 7, 8, 9 or 10. Examples of amino acid changes and conservative substitutions are described in the third aspect of the present disclosure.

In an embodiment of the twenty-fourth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a twenty-fifth aspect the present disclosure relates to polypeptides having lysozyme activity having at least 84%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 71. In one embodiment, the polypeptides differ by up to 33 amino 26, 27, 28, 29, 30, 31, 32 or 33 amino acids from the mature polypeptide of SEQ ID NO: 71.

In a continuation of the twenty-fifth aspect, the present disclosure relates to polypeptides having lysozyme activity having at least 84%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 72. In one embodiment, the polypeptides differ by up to 33 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 amino acids from the mature polypeptide of SEQ ID NO: 72.

In one embodiment, the present disclosure relates to polypeptides having lysozyme activity and having a sequence identity to SEQ ID NO: 72 of at least 84% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 72 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 72 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 72 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 71. In one embodiment, the polypeptide preferably comprises or consists of the amino acid sequence of SEQ ID NO: 72; comprises the amino acid sequence of SEQ ID NO: 72 and a N-terminal and/or C-terminal His-tag and/or HQ-tag; comprises the amino acid sequence of SEQ ID NO: 72 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 72. In one embodiment, the polypeptide comprises or consists of amino acids 1 to 208 of SEQ ID NO: 72. In an embodiment, the polypeptide has been isolated.

In a continuation of the twenty-fifth aspect, the present disclosure relates to a polypeptide having lysozyme activity encoded by a polynucleotide having a sequence identity to the mature polypeptide coding sequence of SEQ ID NO: 70 of at least 84%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twenty-fifth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twenty-fifth aspect, the present disclosure relates to variants of SEQ ID NO: 72 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 72 is not more than 33, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33. In an embodiment, the number of positions comprising one or more amino acid substitutions, and/or one or more amino acid deletions, and/or one or more amino acid insertions or any combination thereof in SEQ ID NO: 72 is not more than 10, e.g., 1, 2, 3, 4, 5, 6, 7, 8, 9 or 10. In one embodiment, the number of substitutions and/or deletions and/or insertions in SEQ ID NO: 72 is not more than 10, e.g., 1, 2, 3, 4, 5, 6, 7, 8, 9 or 10. In a further embodiment, the number of substitutions, preferably conservative substitutions, in SEQ ID NO: 72 is not more than 10, e.g., 1, 2, 3, 4, 5, 6, 7, 8, 9 or 10. Examples of amino acid changes and conservative substitutions are described in the third aspect of the present disclosure.

In an embodiment of the twenty-fifth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a twenty-sixth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 74. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 74.

In a continuation of the twenty-sixth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 75. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 75.

In one embodiment, the present disclosure relates to polypeptides having lysozyme activity and having a sequence identity to SEQ ID NO: 75 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 75 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 75 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 75 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 74. In one embodiment, the polypeptide preferably comprises or consists of the amino acid sequence of SEQ ID NO: 75; comprises the amino acid sequence of SEQ ID NO: 75 and a N-terminal and/or C-terminal His-tag and/or HQ-tag; comprises the amino acid sequence of SEQ ID NO: 75 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 75. In one embodiment, the polypeptide comprises or consists of amino acids 1 to 217 of SEQ ID NO: 75. In an embodiment, the polypeptide has been isolated.

In a continuation of the twenty-sixth aspect, the present disclosure relates to a polypeptide having lysozyme activity encoded by a polynucleotide having a sequence identity to the mature polypeptide coding sequence of SEQ ID NO: 73 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twenty-sixth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twenty-sixth aspect, the present disclosure relates to variants of SEQ ID NO: 75 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 75 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 75 is not more than 10, e.g., 1, 2, 3, 4, 5, 6, 7, 8, 9 or 10. In one embodiment, the number of substitutions and/or deletions and/or insertions in SEQ ID NO: 75 is not more than 10, e.g., 1, 2, 3, 4, 5, 6, 7, 8, 9 or 10. In a further embodiment, the number of substitutions, preferably conservative substitutions, in SEQ ID NO: 75 is not more than 10, e.g., 1, 2, 3, 4, 5, 6, 7, 8, 9 or 10. Examples of amino acid changes and conservative substitutions are described in the third aspect of the present disclosure.

In an embodiment of the twenty-sixth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a twenty-seventh aspect the present disclosure relates to polypeptides having lysozyme activity having at least 84%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 77. In one embodiment, the polypeptides differ by up to 33 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 amino acids from the mature polypeptide of SEQ ID NO: 77.

In a continuation of the twenty-seventh aspect, the present disclosure relates to polypeptides having lysozyme activity having at least 84%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 79. In one embodiment, the polypeptides differ by up to 33 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 amino acids from the mature polypeptide of SEQ ID NO: 79.

In a continuation of the twenty-seventh aspect, the present disclosure relates to polypeptides having lysozyme activity having at least 84%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 80. In one embodiment, the polypeptides differ by up to 33 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 amino acids from SEQ ID NO: 80.

In one embodiment, the present disclosure relates to polypeptides having lysozyme activity and having a sequence identity to SEQ ID NO: 80 of at least 84% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 80 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 80 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 80 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 77. In one embodiment, the polypeptide comprises or consists of the mature polypeptide of SEQ ID NO: 79. In one embodiment, the polypeptide preferably comprises or consists of the amino acid sequence of SEQ ID NO: 80; comprises the amino acid sequence of SEQ ID NO: 80 and a N-terminal and/or C-terminal His-tag and/or HQ-tag; comprises the amino acid sequence of SEQ ID NO: 80 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 80. In one embodiment, the polypeptide comprises or consists of amino acids 1 to 208 of SEQ ID NO: 80. In an embodiment, the polypeptide has been isolated.

In a continuation of the twenty-seventh aspect, the present disclosure relates to a polypeptide having lysozyme activity encoded by a polynucleotide having a sequence identity to the mature polypeptide coding sequence of SEQ ID NO: 76 of at least 84%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twenty-seventh aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twenty-seventh aspect, the present disclosure relates to variants of SEQ ID NO: 80 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 80 is not more than 33, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33. In an embodiment, the number of positions comprising one or more amino acid substitutions, and/or one or more amino acid deletions, and/or one or more amino acid insertions or any combination thereof in SEQ ID NO: 80 is not more than 10, e.g., 1, 2, 3, 4, 5, 6, 7, 8, 9 or 10. In one embodiment, the number of substitutions and/or deletions and/or insertions in SEQ ID NO: 80 is not more than 10, e.g., 1, 2, 3, 4, 5, 6, 7, 8, 9 or 10. In a further embodiment, the number of substitutions, preferably conservative substitutions, in SEQ ID NO: 80 is not more than 10, e.g., 1, 2, 3, 4, 5, 6, 7, 8, 9 or 10. Examples of amino acid changes and conservative substitutions are described in the third aspect of the present disclosure.

In an embodiment of the twenty-seventh aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the twenty-seventh aspect comprises the motif YKNA (SEQ ID NO: 160) and/or the motif FGGW[S/T] (SEQ ID NO: 161).

In a twenty-eighth aspect the present disclosure relates to polypeptides having lysozyme activity having at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 82. In one embodiment, the polypeptides differ by up to 36 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 amino acids from the mature polypeptide of SEQ ID NO: 82.

In a continuation of the twenty-eighth aspect, the present disclosure relates to polypeptides having lysozyme activity having at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 83. In one embodiment, the polypeptides differ by up to 36 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 amino acids from the mature polypeptide of SEQ ID NO: 83.

In one embodiment, the present disclosure relates to polypeptides having lysozyme activity and having a sequence identity to SEQ ID NO: 83 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 83 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 83 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 83 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 82. In one embodiment, the polypeptide preferably comprises or consists of the amino acid sequence of SEQ ID NO: 83; comprises the amino acid sequence of SEQ ID NO: 83 and a N-terminal and/or C-terminal His-tag and/or HQ-tag; comprises the amino acid sequence of SEQ ID NO: 83 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 83. In one embodiment, the polypeptide comprises or consists of amino acids 1 to 201 of SEQ ID NO: 83. In an embodiment, the polypeptide has been isolated.

In a continuation of the twenty-eighth aspect, the present disclosure relates to a polypeptide having lysozyme activity encoded by a polynucleotide having a sequence identity to the mature polypeptide coding sequence of SEQ ID NO: 81 of at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twenty-eighth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twenty-eighth aspect, the present disclosure relates to variants of SEQ ID NO: 83 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 83 is not more than 36 e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36. In an embodiment, the number of positions comprising one or more amino acid substitutions, and/or one or more amino acid deletions, and/or one or more amino acid insertions or any combination thereof in SEQ ID NO: 83 is not more than 10, e.g., 1, 2, 3, 4, 5, 6, 7, 8, 9 or 10. In one embodiment, the number of substitutions and/or deletions and/or insertions in SEQ ID NO: 83 is not more than 10, e.g., 1, 2, 3, 4, 5, 6, 7, 8, 9 or 10. In a further embodiment, the number of substitutions, preferably conservative substitutions, in SEQ ID NO: 83 is not more than 10, e.g., 1, 2, 3, 4, 5, 6, 7, 8, 9 or 10. Examples of amino acid changes and conservative substitutions are described in the third aspect of the present disclosure.

In an embodiment of the twenty-eighth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a twenty-ninth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 85. In one embodiment, the polypeptides differ by up to 40 amino 26, 27, 28, 29, 30, 31, 32, 33, 34, 35, 36, 37, 38, 39 or 40 amino acids from the mature polypeptide of SEQ ID NO: 85.

In a continuation of the twenty-ninth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 86. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 86.

In one embodiment, the present disclosure relates to polypeptides having lysozyme activity and having a sequence identity to SEQ ID NO: 86 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 86 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 86 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 86 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 85. In one embodiment, the polypeptide preferably comprises or consists of the amino acid sequence of SEQ ID NO: 86; comprises the amino acid sequence of SEQ ID NO: 86 and a N-terminal and/or C-terminal His-tag and/or HQ-tag; comprises the amino acid sequence of SEQ ID NO: 86 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 86. In one embodiment, the polypeptide comprises or consists of amino acids 1 to 202 of SEQ ID NO: 86. In an embodiment, the polypeptide has been isolated.

In a continuation of the twenty-ninth aspect, the present disclosure relates to a polypeptide having lysozyme activity encoded by a polynucleotide having a sequence identity to the mature polypeptide coding sequence of SEQ ID NO: 84 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the twenty-ninth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the twenty-ninth aspect, the present disclosure relates to variants of SEQ ID NO: 86 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 86 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 86 is not more than 10, e.g., 1, 2, 3, 4, 5, 6, 7, 8, 9 or 10. In one embodiment, the number of substitutions and/or deletions and/or insertions in SEQ ID NO: 86 is not more than 10, e.g., 1, 2, 3, 4, 5, 6, 7, 8, 9 or 10. In a further embodiment, the number of substitutions, preferably conservative substitutions, in SEQ ID NO: 86 is not more than 10, e.g., 1, 2, 3, 4, 5, 6, 7, 8, 9 or 10. Examples of amino acid changes and conservative substitutions are described in the third aspect of the present disclosure.

In an embodiment of the twenty-ninth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirtieth aspect the present disclosure relates to polypeptides having lysozyme activity having at least 86%, e.g., at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 88. In one embodiment, the polypeptides differ by up to 28 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27 or 28 amino acids from the mature polypeptide of SEQ ID NO: 88.

In a continuation of the thirtieth aspect, the present disclosure relates to polypeptides having lysozyme activity having at least 86%, e.g., at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 89. In one embodiment, the polypeptides differ by up to 28 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27 or 28 amino acids from the mature polypeptide of SEQ ID NO: 89.

In one embodiment, the present disclosure relates to polypeptides having lysozyme activity and having a sequence identity to SEQ ID NO: 89 of at least 86% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 89 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 89 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 88. In one embodiment, the polypeptide preferably comprises or consists of the amino acid sequence of SEQ ID NO: 89; comprises the amino acid sequence of SEQ ID NO: 89 and a N-terminal and/or C-terminal His-tag and/or HQ-tag; comprises the amino acid sequence of SEQ ID NO: 89 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 89. In one embodiment, the polypeptide comprises or consists of amino acids 1 to 207 of SEQ ID NO: 89. In an embodiment, the polypeptide has been isolated.

In a continuation of the thirtieth aspect, the present disclosure relates to a polypeptide having lysozyme activity encoded by a polynucleotide having a sequence identity to the mature polypeptide coding sequence of SEQ ID NO: 87 of at least 86%, e.g., at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the thirtieth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirtieth aspect, the present disclosure relates to variants of SEQ ID NO: 89 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 89 is not more than 28, e.g., 1, 2, 3, 4, 5, 6, 7, 8, 9, 10, 11, 12, 13, 14, 15, 16, 17, 18, 19, 20, 21, 22, 23, 24, 25, 26, 27 or 28. In an embodiment, the number of positions comprising one or more amino acid substitutions, and/or one or more amino acid deletions, and/or one or more amino acid insertions or any combination thereof in SEQ ID NO: 89 is not more than 10, e.g., 1, 2, 3, 4, 5, 6, 7, 8, 9 or 10. In one embodiment, the number of substitutions and/or deletions and/or insertions in SEQ ID NO: 89 is not more than 10, e.g., 1, 2, 3, 4, 5, 6, 7, 8, 9 or 10. In a further embodiment, the number of substitutions, preferably conservative substitutions, in SEQ ID NO: 89 is not more than 10, e.g., 1, 2, 3, 4, 5, 6, 7, 8, 9 or 10. Examples of amino acid changes and conservative substitutions are described in the third aspect of the present disclosure.

In an embodiment of the thirtieth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirty-first aspect the present disclosure relates to polypeptides having lysozyme activity having at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 91. In one embodiment, the polypeptides differ by up to 36 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 amino acids from the mature polypeptide of SEQ ID NO: 91.

In a continuation of the thirty-first aspect, the present disclosure relates to polypeptides having lysozyme activity having at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 92. In one embodiment, the polypeptides differ by up to 36 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 amino acids from the mature polypeptide of SEQ ID NO: 92.

In one embodiment, the present disclosure relates to polypeptides having lysozyme activity and having a sequence identity to SEQ ID NO: 92 of at least 82% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 92 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 92 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 92 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 91. In one embodiment, the polypeptide preferably comprises or consists of the amino acid sequence of SEQ ID NO: 92; comprises the amino acid sequence of SEQ ID NO: 92 and a N-terminal and/or C-terminal His-tag and/or HQ-tag; comprises the amino acid sequence of SEQ ID NO: 92 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 92. In one embodiment, the polypeptide comprises or consists of amino acids 1 to 202 of SEQ ID NO: 92. In an embodiment, the polypeptide has been isolated.

In a continuation of the thirty-first aspect, the present disclosure relates to a polypeptide having lysozyme activity encoded by a polynucleotide having a sequence identity to the mature polypeptide coding sequence of SEQ ID NO: 90 of at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the thirty-first aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirty-first aspect, the present disclosure relates to variants of SEQ ID NO: 92 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 92 is not more than 36, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36. In an embodiment, the number of positions comprising one or more amino acid substitutions, and/or one or more amino acid deletions, and/or one or more amino acid insertions or any combination thereof in SEQ ID NO: 92 is not more than 10, e.g., 1, 2, 3, 4, 5, 6, 7, 8, 9 or 10. In one embodiment, the number of substitutions and/or deletions and/or insertions in SEQ ID NO: 92 is not more than 10, e.g., 1, 2, 3, 4, 5, 6, 7, 8, 9 or 10. In a further embodiment, the number of substitutions, preferably conservative substitutions, in SEQ ID NO: 92 is not more than 10, e.g., 1, 2, 3, 4, 5, 6, 7, 8, 9 or 10. Examples of amino acid changes and conservative substitutions are described in the third aspect of the present disclosure.

In an embodiment of the thirty-first aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirty-second aspect the present disclosure relates to polypeptides having lysozyme activity having at least 94%, e.g., at least 95%, at least 96%, at least 97%, at least 98%, at least 99%, or 100% sequence identity to the mature polypeptide of SEQ ID NO: 94. In one embodiment, the polypeptides differ by up to 12 amino acids, e.g., 1, 2, 3, 4, 5, 6, 7, 8, 9, 10, 11 or 12 amino acids from the mature polypeptide of SEQ ID NO: 94.

In a continuation of the thirty-second aspect, the present disclosure relates to polypeptides having lysozyme activity having at least 94%, e.g., at least 95%, at least 96%, at least 97%, at least 98%, at least 99%, or 100% sequence identity to SEQ ID NO: 95. In one embodiment, the polypeptides differ by up to 12 amino acids, e.g., 1, 2, 3, 4, 5, 6, 7, 8, 9, 10, 11 or 12 amino acids from the mature polypeptide of SEQ ID NO: 95.

In one embodiment, the present disclosure relates to polypeptides having lysozyme activity and having a sequence identity to SEQ ID NO: 95 of at least 94% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 95 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 94. In one embodiment, the polypeptide preferably comprises or consists of the amino acid sequence of SEQ ID NO: 95; comprises the amino acid sequence of SEQ ID NO: 95 and a N-terminal and/or C-terminal His-tag and/or HQ-tag; comprises the amino acid sequence of SEQ ID NO: 95 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 95. In one embodiment, the polypeptide comprises or consists of amino acids 1 to 201 of SEQ ID NO: 95. In an embodiment, the polypeptide has been isolated.

In a continuation of the thirty-second aspect, the present disclosure relates to a polypeptide having lysozyme activity encoded by a polynucleotide having a sequence identity to the mature polypeptide coding sequence of SEQ ID NO: 93 of at least 94%, e.g., at least 95%, at least 96%, at least 97%, at least 98%, at least 99%, or 100%. In a further embodiment, the polypeptide has been isolated.

In an embodiment of the thirty-second aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirty-second aspect, the present disclosure relates to variants of SEQ ID NO: 95 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 95 is not more than 12, e.g., 1, 2, 3, 4, 5, 6, 7, 8, 9, 10, 11 or 12. In an embodiment, the number of positions comprising one or more amino acid substitutions, and/or one or more amino acid deletions, and/or one or more amino acid insertions or any combination thereof in SEQ ID NO: 95 is not more than 10, e.g., 1, 2, 3, 4, 5, 6, 7, 8, 9 or 10. In one embodiment, the number of substitutions and/or deletions and/or insertions in SEQ ID NO: 95 is not more than 10, e.g., 1, 2, 3, 4, 5, 6, 7, 8, 9 or 10. In a further embodiment, the number of substitutions, preferably conservative substitutions, in SEQ ID NO: 95 is not more than 10, e.g., 1, 2, 3, 4, 5, 6, 7, 8, 9 or 10. Examples of amino acid changes and conservative substitutions are described in the third aspect of the present disclosure.

In an embodiment of the thirty-second aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirty-third aspect the present disclosure relates to polypeptides having lysozyme activity having at least 94%, e.g., at least 95%, at least 96%, at least 97%, at least 98%, at least 99%, or 100% sequence identity to the mature polypeptide of SEQ ID NO: 97. In one embodiment, the polypeptides differ by up to 12 amino acids, e.g., 1, 2, 3, 4, 5, 6, 7, 8, 9, 10, 11 or 12 amino acids from the mature polypeptide of SEQ ID NO: 97.

In a continuation of the thirty-third aspect, the present disclosure relates to polypeptides having lysozyme activity having at least 94%, e.g., at least 95%, at least 96%, at least 97%, at least 98%, at least 99%, or 100% sequence identity to SEQ ID NO: 98. In one embodiment, the polypeptides differ by up to 12 amino acids, e.g., 1, 2, 3, 4, 5, 6, 7, 8, 9, 10, 11 or 12 amino acids from the mature polypeptide of SEQ ID NO: 98.

In one embodiment, the present disclosure relates to polypeptides having lysozyme activity and having a sequence identity to SEQ ID NO: 98 of at least 94% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 98 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 97. In one embodiment, the polypeptide preferably comprises or consists of the amino acid sequence of SEQ ID NO: 98; comprises the amino acid sequence of SEQ ID NO: 98 and a N-terminal and/or C-terminal His-tag and/or HQ-tag; comprises the amino acid sequence of SEQ ID NO: 98 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 98. In one embodiment, the polypeptide comprises or consists of amino acids 1 to 202 of SEQ ID NO: 98. In an embodiment, the polypeptide has been isolated.

In a continuation of the thirty-third aspect, the present disclosure relates to a polypeptide having lysozyme activity encoded by a polynucleotide having a sequence identity to the mature polypeptide coding sequence of SEQ ID NO: 96 of at least 94%, e.g., at least 95%, at least 96%, at least 97%, at least 98%, at least 99%, or 100%. In a further embodiment, the polypeptide has been isolated.

In an embodiment of the thirty-third aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirty-third aspect, the present disclosure relates to variants of SEQ ID NO: 98 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 98 is not more than 12, e.g., 1, 2, 3, 4, 5, 6, 7, 8, 9, 10, 11 or 12. In an embodiment, the number of positions comprising one or more amino acid substitutions, and/or one or more amino acid deletions, and/or one or more amino acid insertions or any combination thereof in SEQ ID NO: 98 is not more than 10, e.g., 1, 2, 3, 4, 5, 6, 7, 8, 9 or 10. In one embodiment, the number of substitutions and/or deletions and/or insertions in SEQ ID NO: 98 is not more than 10, e.g., 1, 2, 3, 4, 5, 6, 7, 8, 9 or 10. In a further embodiment, the number of substitutions, preferably conservative substitutions, in SEQ ID NO: 98 is not more than 10, e.g., 1, 2, 3, 4, 5, 6, 7, 8, 9 or 10. Examples of amino acid changes and conservative substitutions are described in the third aspect of the present disclosure.

In an embodiment of the thirty-third aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirty-fourth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 100. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 100.

In a continuation of the thirty-fourth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 101. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 101.

In one embodiment, the present disclosure relates to polypeptides having lysozyme activity and having a sequence identity to SEQ ID NO: 101 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 101 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 101 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 101 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 100. In one embodiment, the polypeptide preferably comprises or consists of the amino acid sequence of SEQ ID NO: 101; comprises the amino acid sequence of SEQ ID NO: 101 and a N-terminal and/or C-terminal His-tag and/or HQ-tag; comprises the amino acid sequence of SEQ ID NO: 101 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 101. In one embodiment, the polypeptide comprises or consists of amino acids 1 to 206 of SEQ ID NO: 101. In an embodiment, the polypeptide has been isolated.

In a continuation of the thirty-fourth aspect, the present disclosure relates to a polypeptide having lysozyme activity encoded by a polynucleotide having a sequence identity to the mature polypeptide coding sequence of SEQ ID NO: 99 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the thirty-fourth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirty-fourth aspect, the present disclosure relates to variants of SEQ ID NO: 101 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 101 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 101 is not more than 10, e.g., 1, 2, 3, 4, 5, 6, 7, 8, 9 or 10. In one embodiment, the number of substitutions and/or deletions and/or insertions in SEQ ID NO: 101 is not more than 10, e.g., 1, 2, 3, 4, 5, 6, 7, 8, 9 or 10. In a further embodiment, the number of substitutions, preferably conservative substitutions, in SEQ ID NO: 101 is not more than 10, e.g., 1, 2, 3, 4, 5, 6, 7, 8, 9 or 10. Examples of amino acid changes and conservative substitutions are described in the third aspect of the present disclosure.

In an embodiment of the thirty-fourth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirty-fifth aspect the present disclosure relates to polypeptides having lysozyme activity having at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 103. In one embodiment, the polypeptides differ by up to 38 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 amino acids from the mature polypeptide of SEQ ID NO: 103.

In a continuation of the thirty-fifth aspect, the present disclosure relates to polypeptides having lysozyme activity having at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 104. In one embodiment, the polypeptides differ by up to 38 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 amino acids from the mature polypeptide of SEQ ID NO: 104.

In one embodiment, the present disclosure relates to polypeptides having lysozyme activity and having a sequence identity to SEQ ID NO: 104 of at least 81% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 104 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 104 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 104 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 103. In one embodiment, the polypeptide preferably comprises or consists of the amino acid sequence of SEQ ID NO: 104; comprises the amino acid sequence of SEQ ID NO: 104 and a N-terminal and/or C-terminal His-tag and/or HQ-tag; comprises the amino acid sequence of SEQ ID NO: 104 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 104. In one embodiment, the polypeptide comprises or consists of amino acids 1 to 202 of SEQ ID NO: 104. In an embodiment, the polypeptide has been isolated.

In a continuation of the thirty-fifth aspect, the present disclosure relates to a polypeptide having lysozyme activity encoded by a polynucleotide having a sequence identity to the mature polypeptide coding sequence of SEQ ID NO: 102 of at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the thirty-fifth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirty-fifth aspect, the present disclosure relates to variants of SEQ ID NO: 104 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 104 is not more than 38, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38. In an embodiment, the number of positions comprising one or more amino acid substitutions, and/or one or more amino acid deletions, and/or one or more amino acid insertions or any combination thereof in SEQ ID NO: 104 is not more than 10, e.g., 1, 2, 3, 4, 5, 6, 7, 8, 9 or 10. In one embodiment, the number of substitutions and/or deletions and/or insertions in SEQ ID NO: 104 is not more than 10, e.g., 1, 2, 3, 4, 5, 6, 7, 8, 9 or 10. In a further embodiment, the number of substitutions, preferably conservative substitutions, in SEQ ID NO: 104 is not more than 10, e.g., 1, 2, 3, 4, 5, 6, 7, 8, 9 or 10. Examples of amino acid changes and conservative substitutions are described in the third aspect of the present disclosure.

In an embodiment of the thirty-fifth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirty-sixth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 106. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 106.

In a continuation of the thirty-sixth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 107. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 107.

In one embodiment, the present disclosure relates to polypeptides having lysozyme activity and having a sequence identity to SEQ ID NO: 107 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 107 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 107 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 107 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 106. In one embodiment, the polypeptide preferably comprises or consists of the amino acid sequence of SEQ ID NO: 107; comprises the amino acid sequence of SEQ ID NO: 107 and a N-terminal and/or C-terminal His-tag and/or HQ-tag; comprises the amino acid sequence of SEQ ID NO: 107 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 107. In one embodiment, the polypeptide comprises or consists of amino acids 1 to 202 of SEQ ID NO: 107. In an embodiment, the polypeptide has been isolated.

In a continuation of the thirty-sixth aspect, the present disclosure relates to a polypeptide having lysozyme activity encoded by a polynucleotide having a sequence identity to the mature polypeptide coding sequence of SEQ ID NO: 105 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the thirty-sixth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirty-sixth aspect, the present disclosure relates to variants of SEQ ID NO: 107 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 107 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 107 is not more than 10, e.g., 1, 2, 3, 4, 5, 6, 7, 8, 9 or 10. In one embodiment, the number of substitutions and/or deletions and/or insertions in SEQ ID NO: 107 is not more than 10, e.g., 1, 2, 3, 4, 5, 6, 7, 8, 9 or 10. In a further embodiment, the number of substitutions, preferably conservative substitutions, in SEQ ID NO: 107 is not more than 10, e.g., 1, 2, 3, 4, 5, 6, 7, 8, 9 or 10. Examples of amino acid changes and conservative substitutions are described in the third aspect of the present disclosure.

In an embodiment of the thirty-sixth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirty-seventh aspect the present disclosure relates to polypeptides having lysozyme activity having at least 88%, e.g., at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 109. In one embodiment, the polypeptides differ by up to 24 amino acids, e.g., 1, 2, 3, 4, 5, 6, 7, 8, 9, 10, 11, 12, 13, 14, 15, 16, 17, 18, 19, 20, 21, 22, 23 or 24 amino acids from the mature polypeptide of SEQ ID NO: 109.

In a continuation of the thirty-seventh aspect, the present disclosure relates to polypeptides having lysozyme activity having at least 88%, e.g., at least 89%, at least 90%, at least 91%, at least 92%, at least 93%, at least 94%, at least 95%, at least 96%, at least 97%, at least 98%, at least 99%, or 100% sequence identity to SEQ ID NO: 110. In one embodiment, the polypeptides differ by up to 24 amino acids, e.g., 1, 2, 3, 4, 5, 6, 7, 8, 9, 10, 11, 12, 13, 14, 15, 16, 17, 18, 19, 20, 21, 22, 23 or 24 amino acids from the mature polypeptide of SEQ ID NO: 110.

In one embodiment, the present disclosure relates to polypeptides having lysozyme activity and having a sequence identity to SEQ ID NO: 110 of at least 88% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 110 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 110 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 109. In one embodiment, the polypeptide preferably comprises or consists of the amino acid sequence of SEQ ID NO: 110; comprises the amino acid sequence of SEQ ID NO: 110 and a N-terminal and/or C-terminal His-tag and/or HQ-tag; comprises the amino acid sequence of SEQ ID NO: 110 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 110. In one embodiment, the polypeptide comprises or consists of amino acids 1 to 202 of SEQ ID NO: 110. In an embodiment, the polypeptide has been isolated.

In a continuation of the thirty-seventh aspect, the present disclosure relates to a polypeptide having lysozyme activity encoded by a polynucleotide having a sequence identity to the mature polypeptide coding sequence of SEQ ID NO: 108 of at least 88%, e.g., at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the thirty-seventh aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirty-seventh aspect, the present disclosure relates to variants of SEQ ID NO: 110 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 110 is not more than 24, e.g., 1, 2, 3, 4, 5, 6, 7, 8, 9, 10, 11, 12, 13, 14, 15, 16, 17, 18, 19, 20, 21, 22, 23 or 24. In an embodiment, the number of positions comprising one or more amino acid substitutions, and/or one or more amino acid deletions, and/or one or more amino acid insertions or any combination thereof in SEQ ID NO: 110 is not more than 10, e.g., 1, 2, 3, 4, 5, 6, 7, 8, 9 or 10. In one embodiment, the number of substitutions and/or deletions and/or insertions in SEQ ID NO: 110 is not more than 10, e.g., 1, 2, 3, 4, 5, 6, 7, 8, 9 or 10. In a further embodiment, the number of substitutions, preferably conservative substitutions, in SEQ ID NO: 110 is not more than 10, e.g., 1, 2, 3, 4, 5, 6, 7, 8, 9 or 10. Examples of amino acid changes and conservative substitutions are described in the third aspect of the present disclosure.

In an embodiment of the thirty-seventh aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirty-eighth aspect the present disclosure relates to polypeptides having lysozyme activity having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 112. In one embodiment, the polypeptides differ by up to 30 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids from the mature polypeptide of SEQ ID NO: 112.

In a continuation of the thirty-eighth aspect, the present disclosure relates to polypeptides having lysozyme activity having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 113. In one embodiment, the polypeptides differ by up to 30 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids from the mature polypeptide of SEQ ID NO: 113.

In one embodiment, the present disclosure relates to polypeptides having lysozyme activity and having a sequence identity to SEQ ID NO: 113 of at least 85% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 113 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 113 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 112. In one embodiment, the polypeptide preferably comprises or consists of the amino acid sequence of SEQ ID NO: 113; comprises the amino acid sequence of SEQ ID NO: 113 and a N-terminal and/or C-terminal His-tag and/or HQ-tag; comprises the amino acid sequence of SEQ ID NO: 113 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 113. In one embodiment, the polypeptide comprises or consists of amino acids 1 to 202 of SEQ ID NO: 113. In an embodiment, the polypeptide has been isolated.

In a continuation of the thirty-eighth aspect, the present disclosure relates to a polypeptide having lysozyme activity encoded by a polynucleotide having a sequence identity to the mature polypeptide coding sequence of SEQ ID NO: 111 of at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the thirty-eighth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirty-eighth aspect, the present disclosure relates to variants of SEQ ID NO: 113 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 113 is not more than 30, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. In an embodiment, the number of positions comprising one or more amino acid substitutions, and/or one or more amino acid deletions, and/or one or more amino acid insertions or any combination thereof in SEQ ID NO: 113 is not more than 10, e.g., 1, 2, 3, 4, 5, 6, 7, 8, 9 or 10. In one embodiment, the number of substitutions and/or deletions and/or insertions in SEQ ID NO: 113 is not more than 10, e.g., 1, 2, 3, 4, 5, 6, 7, 8, 9 or 10. In a further embodiment, the number of substitutions, preferably conservative substitutions, in SEQ ID NO: 113 is not more than 10, e.g., 1, 2, 3, 4, 5, 6, 7, 8, 9 or 10. Examples of amino acid changes and conservative substitutions are described in the third aspect of the present disclosure.

In an embodiment of the thirty-eighth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a thirty-ninth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 115. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 115.

In a continuation of the thirty-ninth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 116. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 116.

In one embodiment, the present disclosure relates to polypeptides having lysozyme activity and having a sequence identity to SEQ ID NO: 116 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 116 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 116 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 116 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 115. In one embodiment, the polypeptide preferably comprises or consists of the amino acid sequence of SEQ ID NO: 116; comprises the amino acid sequence of SEQ ID NO: 116 and a N-terminal and/or C-terminal His-tag and/or HQ-tag; comprises the amino acid sequence of SEQ ID NO: 116 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 116. In one embodiment, the polypeptide comprises or consists of amino acids 1 to 206 of SEQ ID NO: 116. In an embodiment, the polypeptide has been isolated.

In a continuation of the thirty-ninth aspect, the present disclosure relates to a polypeptide having lysozyme activity encoded by a polynucleotide having a sequence identity to the mature polypeptide coding sequence of SEQ ID NO: 114 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the thirty-ninth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the thirty-ninth aspect, the present disclosure relates to variants of SEQ ID NO: 116 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 116 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 116 is not more than 10, e.g., 1, 2, 3, 4, 5, 6, 7, 8, 9 or 10. In one embodiment, the number of substitutions and/or deletions and/or insertions in SEQ ID NO: 116 is not more than 10, e.g., 1, 2, 3, 4, 5, 6, 7, 8, 9 or 10. In a further embodiment, the number of substitutions, preferably conservative substitutions, in SEQ ID NO: 116 is not more than 10, e.g., 1, 2, 3, 4, 5, 6, 7, 8, 9 or 10. Examples of amino acid changes and conservative substitutions are described in the third aspect of the present disclosure.

In an embodiment of the thirty-ninth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a fourteenth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 118. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 118.

In a continuation of the fourteenth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 119. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 119.

In one embodiment, the present disclosure relates to polypeptides having lysozyme activity and having a sequence identity to SEQ ID NO: 119 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 119 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 119 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 119 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 118. In one embodiment, the polypeptide preferably comprises or consists of the amino acid sequence of SEQ ID NO: 119; comprises the amino acid sequence of SEQ ID NO: 119 and a N-terminal and/or C-terminal His-tag and/or HQ-tag; comprises the amino acid sequence of SEQ ID NO: 119 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 119. In one embodiment, the polypeptide comprises or consists of amino acids 1 to 207 of SEQ ID NO: 119. In an embodiment, the polypeptide has been isolated.

In a continuation of the fourteenth aspect, the present disclosure relates to a polypeptide having lysozyme activity encoded by a polynucleotide having a sequence identity to the mature polypeptide coding sequence of SEQ ID NO: 117 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fourteenth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fourteenth aspect, the present disclosure relates to variants of SEQ ID NO: 119 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 119 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 119 is not more than 10, e.g., 1, 2, 3, 4, 5, 6, 7, 8, 9 or 10. In one embodiment, the number of substitutions and/or deletions and/or insertions in SEQ ID NO: 119 is not more than 10, e.g., 1, 2, 3, 4, 5, 6, 7, 8, 9 or 10. In a further embodiment, the number of substitutions, preferably conservative substitutions, in SEQ ID NO: 119 is not more than 10, e.g., 1, 2, 3, 4, 5, 6, 7, 8, 9 or 10. Examples of amino acid changes and conservative substitutions are described in the third aspect of the present disclosure.

In an embodiment of the fourteenth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a forty-first aspect the present disclosure relates to polypeptides having lysozyme activity having at least 96.5%, e.g., at least 97%, at least 97.5%, at least 98%, at least 98.5%, at least 99%, at least 99.5%, or 100% sequence identity to the mature polypeptide of SEQ ID NO: 121. In one embodiment, the polypeptides differ by up to 7 amino acids, e.g., 1, 2, 3, 4, 5, 6 or 7 amino acids from the mature polypeptide of SEQ ID NO: 121.

In a continuation of the forty-first aspect, the present disclosure relates to polypeptides having lysozyme activity having at least 96.5%, e.g., at least 97%, at least 97.5%, at least 98%, at least 98.5%, at least 99%, at least 99.5%, or 100% sequence identity to SEQ ID NO: 122. In one embodiment, the polypeptides differ by up to 7 amino acids, e.g., 1, 2, 3, 4, 5, 6 or 7 amino acids from the mature polypeptide of SEQ ID NO: 122.

In one embodiment, the present disclosure relates to polypeptides having lysozyme activity and having a sequence identity to SEQ ID NO: 122 of at least 96.5% and wherein the polypeptide has at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 121. In one embodiment, the polypeptide preferably comprises or consists of the amino acid sequence of SEQ ID NO: 122; comprises the amino acid sequence of SEQ ID NO: 122 and a N-terminal and/or C-terminal His-tag and/or HQ-tag; comprises the amino acid sequence of SEQ ID NO: 122 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 122. In one embodiment, the polypeptide comprises or consists of amino acids 1 to 208 of SEQ ID NO: 122. In an embodiment, the polypeptide has been isolated.

In a continuation of the forty-first aspect, the present disclosure relates to a polypeptide having lysozyme activity encoded by a polynucleotide having a sequence identity to the mature polypeptide coding sequence of SEQ ID NO: 120 of at least 96.5%, e.g., at least 97%, at least 97.5%, at least 98%, at least 98.5%, at least 99%, at least 99.5%, or 100%. In a further embodiment, the polypeptide has been isolated.

In an embodiment of the forty-first aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the forty-first aspect, the present disclosure relates to variants of SEQ ID NO: 122 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 122 is not more than 7, e.g., 1, 2, 3, 4, 5, 6 or 7. In one embodiment, the number of substitutions and/or deletions and/or insertions in SEQ ID NO: 122 is not more than 7, e.g., 1, 2, 3, 4, 5, 6 or 7. In a further embodiment, the number of substitutions, preferably conservative substitutions, in SEQ ID NO: 122 is not more than 7, e.g., 1, 2, 3, 4, 5, 6 or 7. Examples of amino acid changes and conservative substitutions are described in the third aspect of the present disclosure.

In an embodiment of the forty-first aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a forty-second aspect the present disclosure relates to polypeptides having lysozyme activity having at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 124. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 124.

In a continuation of the forty-second aspect, the present disclosure relates to polypeptides having lysozyme activity having at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 125. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 125.

In one embodiment, the present disclosure relates to polypeptides having lysozyme activity and having a sequence identity to SEQ ID NO: 125 of at least 81% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 125 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 125 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 125 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 124. In one embodiment, the polypeptide preferably comprises or consists of the amino acid sequence of SEQ ID NO: 125; comprises the amino acid sequence of SEQ ID NO: 125 and a N-terminal and/or C-terminal His-tag and/or HQ-tag; comprises the amino acid sequence of SEQ ID NO: 125 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 125. In one embodiment, the polypeptide comprises or consists of amino acids 1 to 215 of SEQ ID NO: 125. In an embodiment, the polypeptide has been isolated.

In a continuation of the forty-second aspect, the present disclosure relates to a polypeptide having lysozyme activity encoded by a polynucleotide having a sequence identity to the mature polypeptide coding sequence of SEQ ID NO: 123 of at least 81%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the forty-second aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the forty-second aspect, the present disclosure relates to variants of SEQ ID NO: 125 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 125 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 125 is not more than 10, e.g., 1, 2, 3, 4, 5, 6, 7, 8, 9 or 10. In one embodiment, the number of substitutions and/or deletions and/or insertions in SEQ ID NO: 125 is not more than 10, e.g., 1, 2, 3, 4, 5, 6, 7, 8, 9 or 10. In a further embodiment, the number of substitutions, preferably conservative substitutions, in SEQ ID NO: 125 is not more than 10, e.g., 1, 2, 3, 4, 5, 6, 7, 8, 9 or 10. Examples of amino acid changes and conservative substitutions are described in the third aspect of the present disclosure.

In an embodiment of the forty-second aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a forty-third aspect the present disclosure relates to polypeptides having lysozyme activity having at least 89%, e.g., at least 90%, at least 91%, at least 92%, at least 93%, at least 94%, at least 95%, at least 96%, at least 97%, at least 98%, at least 99%, or 100% sequence identity to the mature polypeptide of SEQ ID NO: 127. In one embodiment, the polypeptides differ by up to 23 amino acids, e.g., 1, 2, 3, 4, 5, 6, 7, 8, 9, 10, 11, 12, 13, 14, 15, 16, 17, 18, 19, 20, 21, 22 or 23 amino acids from the mature polypeptide of SEQ ID NO: 127.

In a continuation of the forty-third aspect, the present disclosure relates to polypeptides having lysozyme activity having at least 89%, e.g., at least 90%, at least 91%, at least 92%, at least 93%, at least 94%, at least 95%, at least 96%, at least 97%, at least 98%, at least 99%, or 100% sequence identity to SEQ ID NO: 128. In one embodiment, the polypeptides differ by up to 23 amino acids, e.g., 1, 2, 3, 4, 5, 6, 7, 8, 9, 10, 11, 12, 13, 14, 15, 16, 17, 18, 19, 20, 21, 22 or 23 amino acids from the mature polypeptide of SEQ ID NO: 128.

In one embodiment, the present disclosure relates to polypeptides having lysozyme activity and having a sequence identity to SEQ ID NO: 128 of at least 89% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 128 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 128 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 127. In one embodiment, the polypeptide preferably comprises or consists of the amino acid sequence of SEQ ID NO: 128; comprises the amino acid sequence of SEQ ID NO: 128 and a N-terminal and/or C-terminal His-tag and/or HQ-tag; comprises the amino acid sequence of SEQ ID NO: 128 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 128. In one embodiment, the polypeptide comprises or consists of amino acids 1 to 217 of SEQ ID NO: 128. In an embodiment, the polypeptide has been isolated.

In a continuation of the forty-third aspect, the present disclosure relates to a polypeptide having lysozyme activity encoded by a polynucleotide having a sequence identity to the mature polypeptide coding sequence of SEQ ID NO: 126 of at least 89%, e.g., at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the forty-third aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the forty-third aspect, the present disclosure relates to variants of SEQ ID NO: 128 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 128 is not more than 23, e.g., 1, 2, 3, 4, 5, 6, 7, 8, 9, 10, 11, 12, 13, 14, 15, 16, 17, 18, 19, 20, 21, 22 or 23. In an embodiment, the number of positions comprising one or more amino acid substitutions, and/or one or more amino acid deletions, and/or one or more amino acid insertions or any combination thereof in SEQ ID NO: 128 is not more than 10, e.g., 1, 2, 3, 4, 5, 6, 7, 8, 9 or 10. In one embodiment, the number of substitutions and/or deletions and/or insertions in SEQ ID NO: 128 is not more than 10, e.g., 1, 2, 3, 4, 5, 6, 7, 8, 9 or 10. In a further embodiment, the number of substitutions, preferably conservative substitutions, in SEQ ID NO: 128 is not more than 10, e.g., 1, 2, 3, 4, 5, 6, 7, 8, 9 or 10. Examples of amino acid changes and conservative substitutions are described in the third aspect of the present disclosure.

In an embodiment of the forty-third aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a forty-fourth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 130. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 130.

In a continuation of the forty-fourth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 131. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 131.

In one embodiment, the present disclosure relates to polypeptides having lysozyme activity and having a sequence identity to SEQ ID NO: 131 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 131 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 131 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 131 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 130. In one embodiment, the polypeptide preferably comprises or consists of the amino acid sequence of SEQ ID NO: 131; comprises the amino acid sequence of SEQ ID NO: 131 and a N-terminal and/or C-terminal His-tag and/or HQ-tag; comprises the amino acid sequence of SEQ ID NO: 131 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 131. In one embodiment, the polypeptide comprises or consists of amino acids 1 to 214 of SEQ ID NO: 131. In an embodiment, the polypeptide has been isolated.

In a continuation of the forty-fourth aspect, the present disclosure relates to a polypeptide having lysozyme activity encoded by a polynucleotide having a sequence identity to the mature polypeptide coding sequence of SEQ ID NO: 129 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the forty-fourth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the forty-fourth aspect, the present disclosure relates to variants of SEQ ID NO: 131 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 131 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 131 is not more than 10, e.g., 1, 2, 3, 4, 5, 6, 7, 8, 9 or 10. In one embodiment, the number of substitutions and/or deletions and/or insertions in SEQ ID NO: 131 is not more than 10, e.g., 1, 2, 3, 4, 5, 6, 7, 8, 9 or 10. In a further embodiment, the number of substitutions, preferably conservative substitutions, in SEQ ID NO: 131 is not more than 10, e.g., 1, 2, 3, 4, 5, 6, 7, 8, 9 or 10. Examples of amino acid changes and conservative substitutions are described in the third aspect of the present disclosure.

In an embodiment of the forty-fourth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a forty-fifth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 133. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 133.

In a continuation of the forty-fifth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 134. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 134.

In one embodiment, the present disclosure relates to polypeptides having lysozyme activity and having a sequence identity to SEQ ID NO: 134 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 134 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 134 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 134 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 133. In one embodiment, the polypeptide preferably comprises or consists of the amino acid sequence of SEQ ID NO: 134; comprises the amino acid sequence of SEQ ID NO: 134 and a N-terminal and/or C-terminal His-tag and/or HQ-tag; comprises the amino acid sequence of SEQ ID NO: 134 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 134. In one embodiment, the polypeptide comprises or consists of amino acids 1 to 208 of SEQ ID NO: 134. In an embodiment, the polypeptide has been isolated.

In a continuation of the forty-fifth aspect, the present disclosure relates to a polypeptide having lysozyme activity encoded by a polynucleotide having a sequence identity to the mature polypeptide coding sequence of SEQ ID NO: 132 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the forty-fifth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the forty-fifth aspect, the present disclosure relates to variants of SEQ ID NO: 134 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 134 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 134 is not more than 10, e.g., 1, 2, 3, 4, 5, 6, 7, 8, 9 or 10. In one embodiment, the number of substitutions and/or deletions and/or insertions in SEQ ID NO: 134 is not more than 10, e.g., 1, 2, 3, 4, 5, 6, 7, 8, 9 or 10. In a further embodiment, the number of substitutions, preferably conservative substitutions, in SEQ ID NO: 134 is not more than 10, e.g., 1, 2, 3, 4, 5, 6, 7, 8, 9 or 10. Examples of amino acid changes and conservative substitutions are described in the third aspect of the present disclosure.

In an embodiment of the forty-fifth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a forty-sixth aspect the present disclosure relates to polypeptides having lysozyme activity having at least 90%, e.g., at least 91%, at least 92%, at least 93%, at least 94%, at least 95%, at least 96%, at least 97%, at least 98%, at least 99%, or 100% sequence identity to the mature polypeptide of SEQ ID NO: 136. In one embodiment, the polypeptides differ by up to 20 amino acids, e.g., 1, 2, 3, 4, 5, 6, 7, 8, 9, 10, 11, 12, 13, 14, 15, 16, 17, 18, 19 or 20 amino acids from the mature polypeptide of SEQ ID NO: 136.

In a continuation of the forty-sixth aspect, the present disclosure relates to polypeptides having lysozyme activity having at least 90%, e.g., at least 91%, at least 92%, at least 93%, at least 94%, at least 95%, at least 96%, at least 97%, at least 98%, at least 99%, or 100% sequence identity to SEQ ID NO: 137. In one embodiment, the polypeptides differ by up to 20 amino acids, e.g., 1, 2, 3, 4, 5, 6, 7, 8, 9, 10, 11, 12, 13, 14, 15, 16, 17, 18, 19 or 20 amino acids from the mature polypeptide of SEQ ID NO: 137.

In one embodiment, the present disclosure relates to polypeptides having lysozyme activity and having a sequence identity to SEQ ID NO: 137 of at least 90% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 137 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 136. In one embodiment, the polypeptide preferably comprises or consists of the amino acid sequence of SEQ ID NO: 137; comprises the amino acid sequence of SEQ ID NO: 137 and a N-terminal and/or C-terminal His-tag and/or HQ-tag; comprises the amino acid sequence of SEQ ID NO: 137 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 137. In one embodiment, the polypeptide comprises or consists of amino acids 1 to 203 of SEQ ID NO: 137. In an embodiment, the polypeptide has been isolated.

In a continuation of the forty-sixth aspect, the present disclosure relates to a polypeptide having lysozyme activity encoded by a polynucleotide having a sequence identity to the mature polypeptide coding sequence of SEQ ID NO: 135 of at least 90%, e.g., at least 91%, at least 92%, at least 93%, at least 94%, at least 95%, at least 96%, at least 97%, at least 98%, at least 99%, or 100%. In a further embodiment, the polypeptide has been isolated.

In an embodiment of the forty-sixth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the forty-sixth aspect, the present disclosure relates to variants of SEQ ID NO: 137 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 137 is not more than 20, e.g., 1, 2, 3, 4, 5, 6, 7, 8, 9, 10, 11, 12, 13, 14, 15, 16, 17, 18, 19 or 20. In an embodiment, the number of positions comprising one or more amino acid substitutions, and/or one or more amino acid deletions, and/or one or more amino acid insertions or any combination thereof in SEQ ID NO: 137 is not more than 10, e.g., 1, 2, 3, 4, 5, 6, 7, 8, 9 or 10.

In one embodiment, the number of substitutions and/or deletions and/or insertions in SEQ ID NO: 137 is not more than 10, e.g., 1, 2, 3, 4, 5, 6, 7, 8, 9 or 10. In a further embodiment, the number of substitutions, preferably conservative substitutions, in SEQ ID NO: 137 is not more than 10, e.g., 1, 2, 3, 4, 5, 6, 7, 8, 9 or 10. Examples of amino acid changes and conservative substitutions are described in the third aspect of the present disclosure.

In an embodiment of the forty-sixth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a forty-seventh aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 139. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 139.

In a continuation of the forty-seventh aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 140. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 140.

In one embodiment, the present disclosure relates to polypeptides having lysozyme activity and having a sequence identity to SEQ ID NO: 140 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 140 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 140 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 140 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 139. In one embodiment, the polypeptide preferably comprises or consists of the amino acid sequence of SEQ ID NO: 140; comprises the amino acid sequence of SEQ ID NO: 140 and a N-terminal and/or C-terminal His-tag and/or HQ-tag; comprises the amino acid sequence of SEQ ID NO: 140 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 140. In one embodiment, the polypeptide comprises or consists of amino acids 1 to 216 of SEQ ID NO: 140. In an embodiment, the polypeptide has been isolated.

In a continuation of the forty-seventh aspect, the present disclosure relates to a polypeptide having lysozyme activity encoded by a polynucleotide having a sequence identity to the mature polypeptide coding sequence of SEQ ID NO: 138 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the forty-seventh aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the forty-seventh aspect, the present disclosure relates to variants of SEQ ID NO: 140 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 140 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 140 is not more than 10, e.g., 1, 2, 3, 4, 5, 6, 7, 8, 9 or 10. In one embodiment, the number of substitutions and/or deletions and/or insertions in SEQ ID NO: 140 is not more than 10, e.g., 1, 2, 3, 4, 5, 6, 7, 8, 9 or 10. In a further embodiment, the number of substitutions, preferably conservative substitutions, in SEQ ID NO: 140 is not more than 10, e.g., 1, 2, 3, 4, 5, 6, 7, 8, 9 or 10. Examples of amino acid changes and conservative substitutions are described in the third aspect of the present disclosure.

In an embodiment of the forty-seventh aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a forty-eighth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 142. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 142.

In a continuation of the forty-eighth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 143. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 143.

In one embodiment, the present disclosure relates to polypeptides having lysozyme activity and having a sequence identity to SEQ ID NO: 143 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 143 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 143 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 143 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 142. In one embodiment, the polypeptide preferably comprises or consists of the amino acid sequence of SEQ ID NO: 143; comprises the amino acid sequence of SEQ ID NO: 143 and a N-terminal and/or C-terminal His-tag and/or HQ-tag; comprises the amino acid sequence of SEQ ID NO: 143 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 143. In one embodiment, the polypeptide comprises or consists of amino acids 1 to 207 of SEQ ID NO: 143. In an embodiment, the polypeptide has been isolated.

In a continuation of the forty-eighth aspect, the present disclosure relates to a polypeptide having lysozyme activity encoded by a polynucleotide having a sequence identity to the mature polypeptide coding sequence of SEQ ID NO: 141 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the forty-eighth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the forty-eighth aspect, the present disclosure relates to variants of SEQ ID NO: 143 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 143 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 143 is not more than 10, e.g., 1, 2, 3, 4, 5, 6, 7, 8, 9 or 10. In one embodiment, the number of substitutions and/or deletions and/or insertions in SEQ ID NO: 143 is not more than 10, e.g., 1, 2, 3, 4, 5, 6, 7, 8, 9 or 10. In a further embodiment, the number of substitutions, preferably conservative substitutions, in SEQ ID NO: 143 is not more than 10, e.g., 1, 2, 3, 4, 5, 6, 7, 8, 9 or 10. Examples of amino acid changes and conservative substitutions are described in the third aspect of the present disclosure.

In an embodiment of the forty-eighth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a forty-ninth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 145. In one embodiment, the polypeptides differ by up to 40 amino 26, 27, 28, 29, 30, 31, 32, 33, 34, 35, 36, 37, 38, 39 or 40 amino acids from the mature polypeptide of SEQ ID NO: 145.

In a continuation of the forty-ninth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 146. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 146.

In one embodiment, the present disclosure relates to polypeptides having lysozyme activity and having a sequence identity to SEQ ID NO: 146 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 146 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 146 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 146 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 145. In one embodiment, the polypeptide preferably comprises or consists of the amino acid sequence of SEQ ID NO: 146; comprises the amino acid sequence of SEQ ID NO: 146 and a N-terminal and/or C-terminal His-tag and/or HQ-tag; comprises the amino acid sequence of SEQ ID NO: 146 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 146. In one embodiment, the polypeptide comprises or consists of amino acids 1 to 208 of SEQ ID NO: 146. In an embodiment, the polypeptide has been isolated.

In a continuation of the forty-ninth aspect, the present disclosure relates to a polypeptide having lysozyme activity encoded by a polynucleotide having a sequence identity to the mature polypeptide coding sequence of SEQ ID NO: 144 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the forty-ninth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the forty-ninth aspect, the present disclosure relates to variants of SEQ ID NO: 146 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 146 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 146 is not more than 10, e.g., 1, 2, 3, 4, 5, 6, 7, 8, 9 or 10. In one embodiment, the number of substitutions and/or deletions and/or insertions in SEQ ID NO: 146 is not more than 10, e.g., 1, 2, 3, 4, 5, 6, 7, 8, 9 or 10. In a further embodiment, the number of substitutions, preferably conservative substitutions, in SEQ ID NO: 146 is not more than 10, e.g., 1, 2, 3, 4, 5, 6, 7, 8, 9 or 10. Examples of amino acid changes and conservative substitutions are described in the third aspect of the present disclosure.

In an embodiment of the forty-ninth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a fiftieth aspect the present disclosure relates to polypeptides having lysozyme activity having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 148. In one embodiment, the polypeptides differ by up to 31 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acids from the mature polypeptide of SEQ ID NO: 148.

In a continuation of the fiftieth aspect, the present disclosure relates to polypeptides having lysozyme activity having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 149. In one embodiment, the polypeptides differ by up to 31 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acids from the mature polypeptide of SEQ ID NO: 149.

In one embodiment, the present disclosure relates to polypeptides having lysozyme activity and having a sequence identity to SEQ ID NO: 149 of at least 85% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 149 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 149 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 148. In one embodiment, the polypeptide preferably comprises or consists of the amino acid sequence of SEQ ID NO: 149; comprises the amino acid sequence of SEQ ID NO: 149 and a N-terminal and/or C-terminal His-tag and/or HQ-tag; comprises the amino acid sequence of SEQ ID NO: 149 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 149. In one embodiment, the polypeptide comprises or consists of amino acids 1 to 207 of SEQ ID NO: 149. In an embodiment, the polypeptide has been isolated.

In a continuation of the fiftieth aspect, the present disclosure relates to a polypeptide having lysozyme activity encoded by a polynucleotide having a sequence identity to the mature polypeptide coding sequence of SEQ ID NO: 147 of at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fiftieth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fiftieth aspect, the present disclosure relates to variants of SEQ ID NO: 149 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 149 is not more than 31, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31. In an embodiment, the number of positions comprising one or more amino acid substitutions, and/or one or more amino acid deletions, and/or one or more amino acid insertions or any combination thereof in SEQ ID NO: 149 is not more than 10, e.g., 1, 2, 3, 4, 5, 6, 7, 8, 9 or 10. In one embodiment, the number of substitutions and/or deletions and/or insertions in SEQ ID NO: 149 is not more than 10, e.g., 1, 2, 3, 4, 5, 6, 7, 8, 9 or 10. In a further embodiment, the number of substitutions, preferably conservative substitutions, in SEQ ID NO: 149 is not more than 10, e.g., 1, 2, 3, 4, 5, 6, 7, 8, 9 or 10. Examples of amino acid changes and conservative substitutions are described in the third aspect of the present disclosure.

In an embodiment of the fiftieth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the fiftieth aspect comprises the motif YKNA (SEQ ID NO: 160) and/or the motif FGGW[S/T] (SEQ ID NO: 161).

In a fifty-first aspect the present disclosure relates to polypeptides having lysozyme activity having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 151. In one embodiment, the polypeptides differ by up to 31 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acids from the mature polypeptide of SEQ ID NO: 151.

In a continuation of the fifty-first aspect, the present disclosure relates to polypeptides having lysozyme activity having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 152. In one embodiment, the polypeptides differ by up to 31 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acids from the mature polypeptide of SEQ ID NO: 152.

In one embodiment, the present disclosure relates to polypeptides having lysozyme activity and having a sequence identity to SEQ ID NO: 152 of at least 85% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 152 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 152 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 151. In one embodiment, the polypeptide preferably comprises or consists of the amino acid sequence of SEQ ID NO: 152; comprises the amino acid sequence of SEQ ID NO: 152 and a N-terminal and/or C-terminal His-tag and/or HQ-tag; comprises the amino acid sequence of SEQ ID NO: 152 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 152. In one embodiment, the polypeptide comprises or consists of amino acids 1 to 207 of SEQ ID NO: 152. In an embodiment, the polypeptide has been isolated.

In a continuation of the fifty-first aspect, the present disclosure relates to a polypeptide having lysozyme activity encoded by a polynucleotide having a sequence identity to the mature polypeptide coding sequence of SEQ ID NO: 150 of at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fifty-first aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fifty-first aspect, the present disclosure relates to variants of SEQ ID NO: 152 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 152 is not more than 31, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31. In an embodiment, the number of positions comprising one or more amino acid substitutions, and/or one or more amino acid deletions, and/or one or more amino acid insertions or any combination thereof in SEQ ID NO: 152 is not more than 10, e.g., 1, 2, 3, 4, 5, 6, 7, 8, 9 or 10. In one embodiment, the number of substitutions and/or deletions and/or insertions in SEQ ID NO: 152 is not more than 10, e.g., 1, 2, 3, 4, 5, 6, 7, 8, 9 or 10. In a further embodiment, the number of substitutions, preferably conservative substitutions, in SEQ ID NO: 152 is not more than 10, e.g., 1, 2, 3, 4, 5, 6, 7, 8, 9 or 10. Examples of amino acid changes and conservative substitutions are described in the third aspect of the present disclosure.

In an embodiment of the fifty-first aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the fifty-first aspect comprises the motif YKNA (SEQ ID NO: 160) and/or the motif FGGW[S/T] (SEQ ID NO: 161).

In a fifty-second aspect the present disclosure relates to polypeptides having lysozyme activity having at least 84%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 154. In one embodiment, the polypeptides differ by up to 33 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 amino acids from the mature polypeptide of SEQ ID NO: 154.

In a continuation of the fifty-second aspect, the present disclosure relates to polypeptides having lysozyme activity having at least 84%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 155. In one embodiment, the polypeptides differ by up to 33 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 amino acids from the mature polypeptide of SEQ ID NO: 155.

In one embodiment, the present disclosure relates to polypeptides having lysozyme activity and having a sequence identity to SEQ ID NO: 155 of at least 84% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 155 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 155 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 155 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 154. In one embodiment, the polypeptide preferably comprises or consists of the amino acid sequence of SEQ ID NO: 155; comprises the amino acid sequence of SEQ ID NO: 155 and a N-terminal and/or C-terminal His-tag and/or HQ-tag; comprises the amino acid sequence of SEQ ID NO: 155 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 155. In one embodiment, the polypeptide comprises or consists of amino acids 1 to 207 of SEQ ID NO: 155. In an embodiment, the polypeptide has been isolated.

In a continuation of the fifty-second aspect, the present disclosure relates to a polypeptide having lysozyme activity encoded by a polynucleotide having a sequence identity to the mature polypeptide coding sequence of SEQ ID NO: 153 of at least 84%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fifty-second aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fifty-second aspect, the present disclosure relates to variants of SEQ ID NO: 155 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 155 is not more than 33, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33. In an embodiment, the number of positions comprising one or more amino acid substitutions, and/or one or more amino acid deletions, and/or one or more amino acid insertions or any combination thereof in SEQ ID NO: 155 is not more than 10, e.g., 1, 2, 3, 4, 5, 6, 7, 8, 9 or 10. In one embodiment, the number of substitutions and/or deletions and/or insertions in SEQ ID NO: 155 is not more than 10, e.g., 1, 2, 3, 4, 5, 6, 7, 8, 9 or 10. In a further embodiment, the number of substitutions, preferably conservative substitutions, in SEQ ID NO: 155 is not more than 10, e.g., 1, 2, 3, 4, 5, 6, 7, 8, 9 or 10. Examples of amino acid changes and conservative substitutions are described in the third aspect of the present disclosure.

In an embodiment of the fifty-second aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the fifty-second aspect comprises the motif YKNA (SEQ ID NO: 160) and/or the motif FGGW[S/T] (SEQ ID NO: 161).

In a fifty-third aspect the present disclosure relates to polypeptides having lysozyme activity having at least 95%, e.g., at least 96%, at least 97%, at least 98%, at least 99%, or 100% sequence identity to the mature polypeptide of SEQ ID NO: 157. In one embodiment, the polypeptides differ by up to 10 amino acids, e.g., 1, 2, 3, 4, 5, 6, 7, 8, 9 or 10 amino acids from the mature polypeptide of SEQ ID NO: 157.

In a continuation of the fifty-third aspect, the present disclosure relates to polypeptides having lysozyme activity having at least 95%, e.g., at least 96%, at least 97%, at least 98%, at least 99%, or 100% sequence identity to SEQ ID NO: 158. In one embodiment, the polypeptides differ by up to 10 amino acids, e.g., 1, 2, 3, 4, 5, 6, 7, 8, 9 or 10 amino acids from the mature polypeptide of SEQ ID NO: 158.

In one embodiment, the present disclosure relates to polypeptides having lysozyme activity and having a sequence identity to SEQ ID NO: 158 of at least 95% and wherein the polypeptide has at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 157. In one embodiment, the polypeptide preferably comprises or consists of the amino acid sequence of SEQ ID NO: 158; comprises the amino acid sequence of SEQ ID NO: 158 and a N-terminal and/or C-terminal His-tag and/or HQ-tag; comprises the amino acid sequence of SEQ ID NO: 158 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 158. In one embodiment, the polypeptide comprises or consists of amino acids 1 to 208 of SEQ ID NO: 158. In an embodiment, the polypeptide has been isolated.

In a continuation of the fifty-third aspect, the present disclosure relates to a polypeptide having lysozyme activity encoded by a polynucleotide having a sequence identity to the mature polypeptide coding sequence of SEQ ID NO: 156 of at least 95%, e.g., at least 96%, at least 97%, at least 98%, at least 99%, or 100%. In a further embodiment, the polypeptide has been isolated.

In an embodiment of the fifty-third aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fifty-third aspect, the present disclosure relates to variants of SEQ ID NO: 158 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 158 is not more than 10, e.g., 1, 2, 3, 4, 5, 6, 7, 8, 9 or 10. In one embodiment, the number of substitutions and/or deletions and/or insertions in SEQ ID NO: 158 is not more than 10, e.g., 1, 2, 3, 4, 5, 6, 7, 8, 9 or 10. In a further embodiment, the number of substitutions, preferably conservative substitutions, in SEQ ID NO: 158 is not more than 10, e.g., 1, 2, 3, 4, 5, 6, 7, 8, 9 or 10. Examples of amino acid changes and conservative substitutions are described in the third aspect of the present disclosure.

In an embodiment of the fifty-third aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a fifty-fourth aspect the present disclosure relates to polypeptides having lysozyme activity having at least 83%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 218. In one embodiment, the polypeptides differ by up to 35 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids from the mature polypeptide of SEQ ID NO: 218.

In a continuation of the fifty-fourth aspect, the present disclosure relates to polypeptides having lysozyme activity having at least 83%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 220. In one embodiment, the polypeptides differ by up to 35 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids from the mature polypeptide of SEQ ID NO: 220.

In a continuation of the fifty-fourth aspect, the present disclosure relates to polypeptides having lysozyme activity having at least 83%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 221. In one embodiment, the polypeptides differ by up to 35 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids from SEQ ID NO: 221.

In one embodiment, the present disclosure relates to polypeptides having lysozyme activity and having a sequence identity to SEQ ID NO: 221 of at least 83% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 221 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 221 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 221 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 218. In one embodiment, the polypeptide comprises or consists of the mature polypeptide of SEQ ID NO: 220. In one embodiment, the polypeptide preferably comprises or consists of the amino acid sequence of SEQ ID NO: 221; comprises the amino acid sequence of SEQ ID NO: 221 and a N-terminal and/or C-terminal His-tag and/or HQ-tag; comprises the amino acid sequence of SEQ ID NO: 221 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 221. In one embodiment, the polypeptide comprises or consists of amino acids 1 to 207 of SEQ ID NO: 221. In an embodiment, the polypeptide has been isolated.

In a continuation of the fifty-fourth aspect, the present disclosure relates to a polypeptide having lysozyme activity encoded by a polynucleotide having a sequence identity to the mature polypeptide coding sequence of SEQ ID NO: 217 of at least 83%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fifty-fourth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fifty-fourth aspect, the present disclosure relates to variants of SEQ ID NO: 221 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 221 is not more than 35, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35. In an embodiment, the number of positions comprising one or more amino acid substitutions, and/or one or more amino acid deletions, and/or one or more amino acid insertions or any combination thereof in SEQ ID NO: 221 is not more than 10, e.g., 1, 2, 3, 4, 5, 6, 7, 8, 9 or 10. In one embodiment, the number of substitutions and/or deletions and/or insertions in SEQ ID NO: 221 is not more than 10, e.g., 1, 2, 3, 4, 5, 6, 7, 8, 9 or 10. In a further embodiment, the number of substitutions, preferably conservative substitutions, in SEQ ID NO: 221 is not more than 10, e.g., 1, 2, 3, 4, 5, 6, 7, 8, 9 or 10. Examples of amino acid changes and conservative substitutions are described in the third aspect of the present disclosure.

In an embodiment of the fifty-fourth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a forty-sixth aspect the present disclosure relates to polypeptides having lysozyme activity having at least 90%, e.g., at least 91%, at least 92%, at least 93%, at least 94%, at least 95%, at least 96%, at least 97%, at least 98%, at least 99%, or 100% sequence identity to the mature polypeptide of SEQ ID NO: 136. In one embodiment, the polypeptides differ by up to 20 amino acids, e.g., 1, 2, 3, 4, 5, 6, 7, 8, 9, 10, 11, 12, 13, 14, 15, 16, 17, 18, 19 or 20 amino acids from the mature polypeptide of SEQ ID NO: 136.

In a continuation of the forty-sixth aspect, the present disclosure relates to polypeptides having lysozyme activity having at least 90%, e.g., at least 91%, at least 92%, at least 93%, at least 94%, at least 95%, at least 96%, at least 97%, at least 98%, at least 99%, or 100% sequence identity to SEQ ID NO: 137. In one embodiment, the polypeptides differ by up to 20 amino acids, e.g., 1, 2, 3, 4, 5, 6, 7, 8, 9, 10, 11, 12, 13, 14, 15, 16, 17, 18, 19 or 20 amino acids from the mature polypeptide of SEQ ID NO: 137.

In one embodiment, the present disclosure relates to polypeptides having lysozyme activity and having a sequence identity to SEQ ID NO: 137 of at least 90% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 137 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 136. In one embodiment, the polypeptide preferably comprises or consists of the amino acid sequence of SEQ ID NO: 137; comprises the amino acid sequence of SEQ ID NO: 137 and a N-terminal and/or C-terminal His-tag and/or HQ-tag; comprises the amino acid sequence of SEQ ID NO: 137 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 137. In one embodiment, the polypeptide comprises or consists of amino acids 1 to 203 of SEQ ID NO: 137. In an embodiment, the polypeptide has been isolated.

In a continuation of the forty-sixth aspect, the present disclosure relates to a polypeptide having lysozyme activity encoded by a polynucleotide having a sequence identity to the mature polypeptide coding sequence of SEQ ID NO: 135 of at least 90%, e.g., at least 91%, at least 92%, at least 93%, at least 94%, at least 95%, at least 96%, at least 97%, at least 98%, at least 99%, or 100%. In a further embodiment, the polypeptide has been isolated.

In an embodiment of the forty-sixth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the forty-sixth aspect, the present disclosure relates to variants of SEQ ID NO: 137 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 137 is not more than 20, e.g., 1, 2, 3, 4, 5, 6, 7, 8, 9, 10, 11, 12, 13, 14, 15, 16, 17, 18, 19 or 20. In an embodiment, the number of positions comprising one or more amino acid substitutions, and/or one or more amino acid deletions, and/or one or more amino acid insertions or any combination thereof in SEQ ID NO: 137 is not more than 10, e.g., 1, 2, 3, 4, 5, 6, 7, 8, 9 or 10. In one embodiment, the number of substitutions and/or deletions and/or insertions in SEQ ID NO: 137 is not more than 10, e.g., 1, 2, 3, 4, 5, 6, 7, 8, 9 or 10. In a further embodiment, the number of substitutions, preferably conservative substitutions, in SEQ ID NO: 137 is not more than 10, e.g., 1, 2, 3, 4, 5, 6, 7, 8, 9 or 10. Examples of amino acid changes and conservative substitutions are described in the third aspect of the present disclosure.

In an embodiment of the forty-sixth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a fifty-fifth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 223. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 223.

In a continuation of the fifty-fifth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 224. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 224.

In one embodiment, the present disclosure relates to polypeptides having lysozyme activity and having a sequence identity to SEQ ID NO: 224 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 224 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 224 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 224 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 223. In one embodiment, the polypeptide preferably comprises or consists of the amino acid sequence of SEQ ID NO: 224; comprises the amino acid sequence of SEQ ID NO: 224 and a N-terminal and/or C-terminal His-tag and/or HQ-tag; comprises the amino acid sequence of SEQ ID NO: 224 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 224. In one embodiment, the polypeptide comprises or consists of amino acids 1 to 207 of SEQ ID NO: 224. In an embodiment, the polypeptide has been isolated.

In a continuation of the fifty-fifth aspect, the present disclosure relates to a polypeptide having lysozyme activity encoded by a polynucleotide having a sequence identity to the mature polypeptide coding sequence of SEQ ID NO: 222 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fifty-fifth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fifty-fifth aspect, the present disclosure relates to variants of SEQ ID NO: 224 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 224 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 224 is not more than 10, e.g., 1, 2, 3, 4, 5, 6, 7, 8, 9 or 10. In one embodiment, the number of substitutions and/or deletions and/or insertions in SEQ ID NO: 224 is not more than 10, e.g., 1, 2, 3, 4, 5, 6, 7, 8, 9 or 10. In a further embodiment, the number of substitutions, preferably conservative substitutions, in SEQ ID NO: 224 is not more than 10, e.g., 1, 2, 3, 4, 5, 6, 7, 8, 9 or 10. Examples of amino acid changes and conservative substitutions are described in the third aspect of the present disclosure.

In an embodiment of the fifty-fifth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the fifty-fifth aspect comprises the motif YKNA (SEQ ID NO: 160) and/or the motif FGGW[S/T] (SEQ ID NO: 161).

In a fifty-sixth aspect the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 226. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 226.

In a continuation of the fifty-sixth aspect, the present disclosure relates to polypeptides having lysozyme activity having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 227. In one embodiment, the polypeptides differ by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids from the mature polypeptide of SEQ ID NO: 227.

In one embodiment, the present disclosure relates to polypeptides having lysozyme activity and having a sequence identity to SEQ ID NO: 227 of at least 80% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 227 of at least 85% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 227 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 227 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 226. In one embodiment, the polypeptide preferably comprises or consists of the amino acid sequence of SEQ ID NO: 227; comprises the amino acid sequence of SEQ ID NO: 227 and a N-terminal and/or C-terminal His-tag and/or HQ-tag; comprises the amino acid sequence of SEQ ID NO: 227 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 227. In one embodiment, the polypeptide comprises or consists of amino acids 1 to 208 of SEQ ID NO: 227. In an embodiment, the polypeptide has been isolated.

In a continuation of the fifty-sixth aspect, the present disclosure relates to a polypeptide having lysozyme activity encoded by a polynucleotide having a sequence identity to the mature polypeptide coding sequence of SEQ ID NO: 225 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fifty-sixth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fifty-sixth aspect, the present disclosure relates to variants of SEQ ID NO: 227 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 227 is not more than 40, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In an embodiment, the number of positions comprising one or more amino acid substitutions, and/or one or more amino acid deletions, and/or one or more amino acid insertions or any combination thereof in SEQ ID NO: 227 is not more than 10, e.g., 1, 2, 3, 4, 5, 6, 7, 8, 9 or 10. In one embodiment, the number of substitutions and/or deletions and/or insertions in SEQ ID NO: 227 is not more than 10, e.g., 1, 2, 3, 4, 5, 6, 7, 8, 9 or 10. In a further embodiment, the number of substitutions, preferably conservative substitutions, in SEQ ID NO: 227 is not more than 10, e.g., 1, 2, 3, 4, 5, 6, 7, 8, 9 or 10. Examples of amino acid changes and conservative substitutions are described in the third aspect of the present disclosure.

In an embodiment of the fifty-sixth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the fifty-sixth aspect comprises the motif YKNA (SEQ ID NO: 160) and/or the motif FGGW[S/T] (SEQ ID NO: 161).

In a fifty-seventh aspect the present disclosure relates to polypeptides having lysozyme activity having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 229. In one embodiment, the polypeptides differ by up to 31 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acids from the mature polypeptide of SEQ ID NO: 229.

In a continuation of the fifty-seventh aspect, the present disclosure relates to polypeptides having lysozyme activity having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 230. In one embodiment, the polypeptides differ by up to 31 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acids from the mature polypeptide of SEQ ID NO: 230.

In one embodiment, the present disclosure relates to polypeptides having lysozyme activity and having a sequence identity to SEQ ID NO: 230 of at least 85% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 230 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 230 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 229. In one embodiment, the polypeptide preferably comprises or consists of the amino acid sequence of SEQ ID NO: 230; comprises the amino acid sequence of SEQ ID NO: 230 and a N-terminal and/or C-terminal His-tag and/or HQ-tag; comprises the amino acid sequence of SEQ ID NO: 230 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 230. In one embodiment, the polypeptide comprises or consists of amino acids 1 to 207 of SEQ ID NO: 230. In an embodiment, the polypeptide has been isolated.

In a continuation of the fifty-seventh aspect, the present disclosure relates to a polypeptide having lysozyme activity encoded by a polynucleotide having a sequence identity to the mature polypeptide coding sequence of SEQ ID NO: 228 of at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fifty-seventh aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fifty-seventh aspect, the present disclosure relates to variants of SEQ ID NO: 230 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 230 is not more than 31, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31. In an embodiment, the number of positions comprising one or more amino acid substitutions, and/or one or more amino acid deletions, and/or one or more amino acid insertions or any combination thereof in SEQ ID NO: 230 is not more than 10, e.g., 1, 2, 3, 4, 5, 6, 7, 8, 9 or 10. In one embodiment, the number of substitutions and/or deletions and/or insertions in SEQ ID NO: 230 is not more than 10, e.g., 1, 2, 3, 4, 5, 6, 7, 8, 9 or 10. In a further embodiment, the number of substitutions, preferably conservative substitutions, in SEQ ID NO: 230 is not more than 10, e.g., 1, 2, 3, 4, 5, 6, 7, 8, 9 or 10. Examples of amino acid changes and conservative substitutions are described in the third aspect of the present disclosure.

In an embodiment of the fifty-seventh aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the fifty-seventh aspect comprises the motif YKNA (SEQ ID NO: 160) and/or the motif FGGW[S/T] (SEQ ID NO: 161).

In a fifty-eighth aspect the present disclosure relates to polypeptides having lysozyme activity having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 232. In one embodiment, the polypeptides differ by up to 31 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acids from the mature polypeptide of SEQ ID NO: 232.

In a continuation of the fifty-eighth aspect, the present disclosure relates to polypeptides having lysozyme activity having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 233. In one embodiment, the polypeptides differ by up to 31 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acids from the mature polypeptide of SEQ ID NO: 233.

In one embodiment, the present disclosure relates to polypeptides having lysozyme activity and having a sequence identity to SEQ ID NO: 233 of at least 85% and wherein the polypeptide has at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 233 of at least 90% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159. In one embodiment, the present disclosure relates to polypeptides having lysozyme activity and having a sequence identity to SEQ ID NO: 233 of at least 95% and wherein the polypeptide has at least at least 150%, such as at least 175%, at least 200%, at least 225%, at least 250%, at least 275%, at least 300%, at least 350%, or at least 400% of the lysozyme activity of SEQ ID NO: 159.

In one embodiment, the polypeptide comprises or consists of the mature polypeptide of SEQ ID NO: 232. In one embodiment, the polypeptide preferably comprises or consists of the amino acid sequence of SEQ ID NO: 233; comprises the amino acid sequence of SEQ ID NO: 233 and a N-terminal and/or C-terminal His-tag and/or HQ-tag; comprises the amino acid sequence of SEQ ID NO: 233 and a N-terminal and/or C-terminal extension of up to 10 amino acids, e.g. 1, 2, 3, 4, 5, 6, 7, 8, 9 or 10 amino acids; or is a fragment thereof having lysozyme activity and having at least 90% such as at least 91%, at least 92%, at least 93%, at least 94%, at least 95%, at least 96%, at least 97%, at least 98% or at least 99% of the length of SEQ ID NO: 233. In one embodiment, the polypeptide comprises or consists of amino acids 1 to 207 of SEQ ID NO: 233. In an embodiment, the polypeptide has been isolated.

In a continuation of the fifty-eighth aspect, the present disclosure relates to a polypeptide having lysozyme activity encoded by a polynucleotide having a sequence identity to the mature polypeptide coding sequence of SEQ ID NO: 231 of at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In a further embodiment, the polypeptide has been isolated.

In an embodiment of the fifty-eighth aspect, the polypeptide has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In a continuation of the fifty-eighth aspect, the present disclosure relates to variants of SEQ ID NO: 233 having lysozyme activity comprising one or more amino acid substitutions, and/or one or more amino acid deletions, and/or one or more amino acid insertions or any combination thereof at one or more (e.g., several) positions. In an embodiment, the number of positions comprising one or more amino acid substitutions, and/or one or more amino acid deletions, and/or one or more amino acid insertions or any combination thereof in SEQ ID NO: 233 is not more than 31, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31. In an embodiment, the number of positions comprising one or more amino acid substitutions, and/or one or more amino acid deletions, and/or one or more amino acid insertions or any combination thereof in SEQ ID NO: 233 is not more than 10, e.g., 1, 2, 3, 4, 5, 6, 7, 8, 9 or 10. In one embodiment, the number of substitutions and/or deletions and/or insertions in SEQ ID NO: 233 is not more than 10, e.g., 1, 2, 3, 4, 5, 6, 7, 8, 9 or 10. In a further embodiment, the number of substitutions, preferably conservative substitutions, in SEQ ID NO: 233 is not more than 10, e.g., 1, 2, 3, 4, 5, 6, 7, 8, 9 or 10. Examples of amino acid changes and conservative substitutions are described in the third aspect of the present disclosure.

In an embodiment of the fifty-eighth aspect, the variant has at least at least 150%, such as at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In an embodiment, the polypeptide of the fifty-eighth aspect comprises the motif YKNA (SEQ ID NO: 160) and/or the motif FGGW[S/T] (SEQ ID NO: 161).

In one embodiment, the GH25 polypeptide comprises the motif YKNA (SEQ ID NO: 160) and/or the motif FGGW[S/T] (SEQ ID NO: 161), and preferably is selected from the group selected from SEQ ID NO: 12, SEQ ID NO: 27, SEQ ID NO: 42, SEQ ID NO: 57, SEQ ID NO: 63, SEQ ID NO: 66, SEQ ID NO: 80, SEQ ID NO: 149, SEQ ID NO:152, SEQ ID NO: 155, SEQ ID NO: 224, SEQ ID NO: 227, SEQ ID NO: 230 and SEQ ID NO: 233.

In one embodiment, the GH25 polypeptide is obtained or is obtainable from the taxonomic order Agaricales and is preferably is selected from the group selected from SEQ ID NO: 80, SEQ ID NO: 149, SEQ ID NO:152, SEQ ID NO: 155, SEQ ID NO: 221, SEQ ID NO: 224, SEQ ID NO: 230 and SEQ ID NO: 233.

In one embodiment, the GH25 polypeptide is obtained or is obtainable from the taxonomic order Agaricales, preferably the taxonomic family Strophariaceae. In one embodiment, the GH25 polypeptide is obtained or is obtainable from the taxonomic family Strophariaceae and is selected from the group selected from SEQ ID NO: 80, SEQ ID NO: 155, SEQ ID NO: 224, SEQ ID NO: 230 and SEQ ID NO: 233.

In one embodiment, the GH25 polypeptide is obtained or is obtainable from the taxonomic class Dothideomycetes, preferably the taxonomic order Pleosporales. In one embodiment, the GH25 polypeptide is obtained or is obtainable from the taxonomic class Dothideomycetes, preferably the taxonomic order Pleosporales and is selected from the group selected from SEQ ID NO: 3, SEQ ID NO: 60, SEQ ID NO: 95, SEQ ID NO:101, SEQ ID NO: 104 and SEQ ID NO: 146.

In one embodiment, the GH25 polypeptide is obtained or is obtainable from the taxonomic class Eurotiomycetes but not from the taxonomic genus Aspergillus fumigatus. In one embodiment, the GH25 polypeptide is obtained or is obtainable from the taxonomic class Eurotiomycetes but not from the taxonomic genus Aspergillus fumigatus and is selected from the group selected from SEQ ID NO: 18, SEQ ID NO: 27, SEQ ID NO: 45, SEQ ID NO: 48, SEQ ID NO: 75, SEQ ID NO: 82, SEQ ID NO: 125, SEQ ID NO: 131 and SEQ ID NO: 140.

In one embodiment, the GH25 polypeptide is obtained or is obtainable from the taxonomic order Eurotiales but not from the taxonomic genus Aspergillus fumigatus. In one embodiment, the GH25 polypeptide is obtained or is obtainable from the taxonomic order Eurotiales but not from the taxonomic genus Aspergillus fumigatus and is selected from the group selected from SEQ ID NO: 27, SEQ ID NO: 45, SEQ ID NO: 48, SEQ ID NO: 82, SEQ ID NO: 125, SEQ ID NO: 131 and SEQ ID NO: 140.

In one embodiment, the GH25 polypeptide is obtained or is obtainable from the taxonomic family Aspergillaceae but not from the taxonomic genus Aspergillus fumigatus. In one embodiment, the GH25 polypeptide is obtained or is obtainable from the taxonomic family Aspergillaceae but not from the taxonomic genus Aspergillus fumigatus and is selected from the group selected from SEQ ID NO: 27, SEQ ID NO: 45, SEQ ID NO: 48, SEQ ID NO: 82, SEQ ID NO: 125 and SEQ ID NO: 140.

In one embodiment, the GH25 polypeptide is obtained or is obtainable from the taxonomic class Pezizomycetes, preferably the taxonomic order Pezizales, more preferably the taxonomic family Ascobolaceae. In one embodiment, the GH25 polypeptide is obtained or is obtainable from the taxonomic class Pezizomycetes, preferably the taxonomic order Pezizales, more preferably the taxonomic family Ascobolaceae and is selected from the group selected from SEQ ID NO: 86 and SEQ ID NO: 98.

In one embodiment, the GH25 polypeptide is obtained or is obtainable from the taxonomic order Coniochaetales. In one embodiment, the GH25 polypeptide is obtained or is obtainable from the taxonomic order Coniochaetales and is selected from the group selected from SEQ ID NO: 89.

In one embodiment, the GH25 polypeptide is obtained or is obtainable from the taxonomic order Hypocreales but not from the taxonomic genus Trichoderma reesei. In one embodiment, the GH25 polypeptide is obtained or is obtainable from the taxonomic order Hypocreales but not from the taxonomic genus Trichoderma reesei and is selected from the group selected from SEQ ID NO: 12, SEQ ID NO: 15, SEQ ID NO: 21, SEQ ID NO: 42, SEQ ID NO: 51, SEQ ID NO: 54, SEQ ID NO: 66, SEQ ID NO: 69, SEQ ID NO: 122, SEQ ID NO: 128, SEQ ID NO: 134, SEQ ID NO: 137 and SEQ ID NO: 143.

In one embodiment, the GH25 polypeptide is obtained or is obtainable from the taxonomic family Clavicipitaceae. In one embodiment, the GH25 polypeptide is obtained or is obtainable from the taxonomic family Clavicipitaceae and is selected from the group selected from SEQ ID NO: 51, SEQ ID NO: 54, SEQ ID NO: 122 and SEQ ID NO: 128.

In one embodiment, the GH25 polypeptide is obtained or is obtainable from the taxonomic family Cordycipitaceae. In one embodiment, the GH25 polypeptide is obtained or is obtainable from the taxonomic family Cordycipitaceae and is selected from the group selected from SEQ ID NO: 12, SEQ ID NO: 15, SEQ ID NO: 42, SEQ ID NO: 66, SEQ ID NO: 69 and SEQ ID NO: 137.

In one embodiment, the GH25 polypeptide is obtained or is obtainable from the taxonomic order Microascales. In one embodiment, the GH25 polypeptide is obtained or is obtainable from the taxonomic order Microascales and is selected from the group selected from SEQ ID NO: 16.

In one embodiment, the GH25 polypeptide is obtained or is obtainable from the taxonomic order Sordariales. In one embodiment, the GH25 polypeptide is obtained or is obtainable from the taxonomic order Sordariales and is selected from the group selected from SEQ ID NO: 30, SEQ ID NO: 39, SEQ ID NO: 7, and SEQ ID NO: 158.

In one embodiment, the GH25 polypeptide is obtained or is obtainable from the taxonomic order Sordariales. In one embodiment, the GH25 polypeptide is obtained or is obtainable from the taxonomic order Sordariales and is selected from the group selected from SEQ ID NO: 30, SEQ ID NO: 39, SEQ ID NO: 7, and SEQ ID NO: 158.

In one embodiment, the GH25 polypeptide is obtained or is obtainable from the taxonomic order Xylariales. In one embodiment, the GH25 polypeptide is obtained or is obtainable from the taxonomic order Xylariales and is selected from the group selected from SEQ ID NO: 6, SEQ ID NO: 9, SEQ ID NO: 92, SEQ ID NO: 107, SEQ ID NO: 110 and SEQ ID NO: 113.

A polypeptide having lysozyme activity of the present disclosure may be obtained from microorganisms of any genus. For purposes of the present disclosure, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a fungal polypeptide. In one aspect, the polypeptide is a polypeptide having lysozyme activity from a fungus of the class Eurotiomycetes, such as from the order Eurotiales, or from the family Aspergillaceae, or from the genus Aspergillus or from the species Aspergillus sp. nov. XZ2609.

In another aspect, the polypeptide may be a fungal polypeptide. In one aspect, the polypeptide is a polypeptide having lysozyme activity from a fungus of the class Sordariomycetes, such as from the order Sordariales, or from the family Chaetomiaceae, or from the genus Chaetomium or from the species Chaetomium cupreum.

In another aspect, the polypeptide may be a fungal polypeptide. In one aspect, the polypeptide is a polypeptide having lysozyme activity from a fungus of the class Sordariomycetes, such as from the order Sordariales, or from the family Lasiosphaeriaceae, or from the genus Cladorrhinum or from the species Cladorrhinum bulbillosum.

In another aspect, the polypeptide may be a fungal polypeptide. In one aspect, the polypeptide is a polypeptide having lysozyme activity from a fungus of the class Sordariomycetes, such as from the order Hypocreales, or from the family Cordycipitaceae, or from the genus Cordyceps or from the species Cordyceps cardinalis.

In another aspect, the polypeptide may be a fungal polypeptide. In one aspect, the polypeptide is a polypeptide having lysozyme activity from a fungus of the class Agaricomycetes, such as from the order Agaricales, or from the family Strophariaceae, or from the genus Hypholoma or from the species Hypholoma polytrichi.

In another aspect, the polypeptide may be a fungal polypeptide. In one aspect, the polypeptide is a polypeptide having lysozyme activity from a fungus of the class Sordariomycetes, such as from the order Hypocreales, or from the family Cordycipitaceae, or from the genus Isaria or from the species Isaria farinosa.

In another aspect, the polypeptide may be a fungal polypeptide. In one aspect, the polypeptide is a polypeptide having lysozyme activity from a fungus of the class Sordariomycetes, such as from the order Hypocreales, or from the family Cordycipitaceae, or from the genus Lecanicillium or from the species Lecanicillium sp. WMM742.

In another aspect, the polypeptide may be a fungal polypeptide. In one aspect, the polypeptide is a polypeptide having lysozyme activity from a fungus of the class Eurotiomycetes, such as from the order Onygenales, or from the genus Malbranchea or from the species Malbranchea flava.

In another aspect, the polypeptide may be a fungal polypeptide. In one aspect, the polypeptide is a polypeptide having lysozyme activity from a fungus of the class Eurotiomycetes, such as from the order Onygenales, or from the family Onygenaceae, or from the genus Onygena or from the species Onygena equina.

In another aspect, the polypeptide may be a fungal polypeptide. In one aspect, the polypeptide is a polypeptide having lysozyme activity from a fungus of the class Sordariomycetes, such as from the order Hypocreales, or from the family Clavicipitaceae, or from the genus Paecilomyces or from the species Paecilomyces sp. XZ2658.

In another aspect, the polypeptide may be a fungal polypeptide. In one aspect, the polypeptide is a polypeptide having lysozyme activity from a fungus of the class Eurotiomycetes, such as from the order Eurotiales, or from the family Aspergillaceae, or from the genus Penicillium or from the species Penicillium citrinum.

In another aspect, the polypeptide may be a fungal polypeptide. In one aspect, the polypeptide is a polypeptide having lysozyme activity from a fungus of the class Eurotiomycetes, such as from the order Eurotiales, or from the family Aspergillaceae, or from the genus Penicillium or from the species Penicillium citrinum or Penicillium sp. ‘qii’.

In another aspect, the polypeptide may be a fungal polypeptide. In one aspect, the polypeptide is a polypeptide having lysozyme activity from a fungus of the class Sordariomycetes, such as from the order Xylariales, or from the family Xylariaceae, or from the genus Poronia or from the species Poronia punctata.

In another aspect, the polypeptide may be a fungal polypeptide. In one aspect, the polypeptide is a polypeptide having lysozyme activity from a fungus of the class Dothideomycetes, such as from the order Pleosporales, or from the family Sporormiaceae, or from the genus Pycnidiophora or from the species Pycnidiophora cf. dispera.

In another aspect, the polypeptide may be a fungal polypeptide. In one aspect, the polypeptide is a polypeptide having lysozyme activity from a fungus of the order Mucorales, such as from the family Lichtheimiaceae, or from the genus Rhizomucor or from the species Rhizomucor pusillus.

In another aspect, the polypeptide may be a fungal polypeptide. In one aspect, the polypeptide is a polypeptide having lysozyme activity from a fungus of the class Dothideomycetes, such as from the order Pleosporales, or from the family Sporormiaceae, or from the genus Sporormia or from the species Sporormia fimetaria.

In another aspect, the polypeptide may be a fungal polypeptide. In one aspect, the polypeptide is a polypeptide having lysozyme activity from a fungus of the order Mucorales, such as from the family Lichtheimiaceae, or from the genus Thermomucor or from the species Thermomucor indicae-seudaticae.

In another aspect, the polypeptide may be a fungal polypeptide. In one aspect, the polypeptide is a polypeptide having lysozyme activity from a fungus of the genus Trichobolus or from the species Trichobolus zukalii.

In another aspect, the polypeptide may be a fungal polypeptide. In one aspect, the polypeptide is a polypeptide having lysozyme activity from a fungus of the order Mucorales, such as from the family Umbelopsidaceae, or from the genus Umbelopsis or from the species Umbelopsis westeae.

In another aspect, the polypeptide may be a fungal polypeptide. In one aspect, the polypeptide is a polypeptide having lysozyme activity from a fungus of the class Sordariomycetes, such as from the order Sordariales, or from the family Chaetomiaceae, or from the genus Zopfiella or from the species Zopfiella sp. t180-6.

In another aspect, the polypeptide may be a fungal polypeptide. In one aspect, the polypeptide is a polypeptide having lysozyme activity from a fungus of the genus Zygomycetes or from the species Zygomycetes sp. XZ2655.

In another aspect, the polypeptide may be a fungal polypeptide. In one aspect, the polypeptide is a polypeptide having lysozyme activity from a fungus of the class Sordariomycetes, such as from the order Sordariales, or from the family Sordariaceae, or from the genus Gelasinospora, or from the species Gelasinospora cratophora.

In another aspect, the polypeptide may be a fungal polypeptide. In one aspect, the polypeptide is a polypeptide having lysozyme activity from a fungus of the class Agaricomycetes, such as from the order Agaricales, or from the family Strophariaceae, or from the genus Stropharia, or from the species Stropharia semiglobata.

In another aspect, the polypeptide may be a fungal polypeptide. In one aspect, the polypeptide is a polypeptide having lysozyme activity from a fungus of the class Sordariomycetes, such as from the order Hypocreales, or from the family Bionectriaceae, or from the genus Clonostachys, or from the species Clonostachys rossmaniae.

In another aspect, the polypeptide may be a fungal polypeptide. In one aspect, the polypeptide is a polypeptide having lysozyme activity from a fungus of the class Sordariomycetes, such as from the order Hypocreales, or from the family Nectriaceae, or from the genus Paracremonium, or from the species Paracremonium inflatum.

In another aspect, the polypeptide may be a fungal polypeptide. In one aspect, the polypeptide is a polypeptide having lysozyme activity from a fungus of the class Dothideomycetes, such as from the order Pleosporales, or from the family Cucurbitariaceae, or from the genus Curreya, or from the species Curreya sp. XZ2623.

In another aspect, the polypeptide may be a fungal polypeptide. In one aspect, the polypeptide is a polypeptide having lysozyme activity from a fungus of the class Sordariomycetes, such as from the order Hypocreales, or from the family Clavicipitaceae, or from the genus Metapochonia, or from the species Metapochonia bulbillosa.

In another aspect, the polypeptide may be a fungal polypeptide. In one aspect, the polypeptide is a polypeptide having lysozyme activity from a fungus of the class Eurotiomycetes, such as from the order Eurotiales, or from the family Aspergillaceae, or from the genus Aspergillus, or from the species Aspergillus inflatus.

In another aspect, the polypeptide may be a fungal polypeptide. In one aspect, the polypeptide is a polypeptide having lysozyme activity from a fungus of the class Pezizomycetes, such as from the order Pezizales, or from the family Ascobolaceae, or from the genus Ascobolus, or from the species Ascobolus sp. ZY179.

In another aspect, the polypeptide may be a fungal polypeptide. In one aspect, the polypeptide is a polypeptide having lysozyme activity from a fungus of the class Sordariomycetes, such as from the order Xylariales, or from the family Xylariaceae, or from the genus Daldinia, or from the species Daldinia fissa.

In another aspect, the polypeptide may be a fungal polypeptide. In one aspect, the polypeptide is a polypeptide having lysozyme activity from a fungus of the class Sordariomycetes, such as from the order Coniochaetales, or from the family Coniochaetaceae, or from the genus Coniochaeta, or from the species Coniochaeta sp.

In another aspect, the polypeptide may be a fungal polypeptide. In one aspect, the polypeptide is a polypeptide having lysozyme activity from a fungus of the class Eurotiomycetes, such as from the order Eurotiales, or from the family Aspergillaceae, or from the genus Aspergillus, or from the species Aspergillus deflectus.

In another aspect, the polypeptide may be a fungal polypeptide. In one aspect, the polypeptide is a polypeptide having lysozyme activity from a fungus of the class Dothideomycetes, such as from the order Pleosporales, or from the family Delitschiaceae, or from the genus Delitschia, or from the species Delitschia sp.

In another aspect, the polypeptide may be a fungal polypeptide. In one aspect, the polypeptide is a polypeptide having lysozyme activity from a fungus of the class Sordariomycetes, such as from the order Hypocreales, or from the family Clavicipitaceae, or from the genus Metarhizium, or from the species Metarhizium iadini.

In another aspect, the polypeptide may be a fungal polypeptide. In one aspect, the polypeptide is a polypeptide having lysozyme activity from a fungus of the class Sordariomycetes, such as from the order Xylariales, or from the family Xylariaceae, or from the genus Hypoxylon, or from the species Hypoxylon sp.

In another aspect, the polypeptide may be a fungal polypeptide. In one aspect, the polypeptide is a polypeptide having lysozyme activity from a fungus of the class Sordariomycetes, such as from the order Xylariales, or from the family Xylariaceae, or from the genus Hypoxylon, or from the species Hypoxylon sp.

In another aspect, the polypeptide may be a fungal polypeptide. In one aspect, the polypeptide is a polypeptide having lysozyme activity from a fungus of the class Sordariomycetes, such as from the order Hypocreales, or from the family Cordycipitaceae, or from the genus Simplicillium, or from the species Simplicillium obclavatum.

In another aspect, the polypeptide may be a fungal polypeptide. In one aspect, the polypeptide is a polypeptide having lysozyme activity from a fungus of the class Sordariomycetes, such as from the order Microascales, or from the family Microascaceae, or from the genus Yunnania, or from the species Yunnania penicillata.

In another aspect, the polypeptide may be a fungal polypeptide. In one aspect, the polypeptide is a polypeptide having lysozyme activity from a fungus of the class Dothideomycetes, such as from the order Pleosporales, or from the family Sporormiaceae, or from the genus Westerdykella, or from the species Westerdykella sp.

In another aspect, the polypeptide may be a fungal polypeptide. In one aspect, the polypeptide is a polypeptide having lysozyme activity from a fungus of the class Eurotiomycetes, such as from the order Eurotiales, or from the family Thermoascaceae, or from the genus Thermoascus, or from the species Thermoascus aurantiacus.

In another aspect, the polypeptide may be a fungal polypeptide. In one aspect, the polypeptide is a polypeptide having lysozyme activity from a fungus of the class Dothideomycetes, such as from the order Pleosporales, or from the family Coniothyriaceae, or from the genus Coniothyrium, or from the species Coniothyrium sp.

In another aspect, the polypeptide may be a fungal polypeptide. In one aspect, the polypeptide is a polypeptide having lysozyme activity from a fungus of the class Pezizomycetes, such as from the order Pezizales, or from the family Ascobolaceae, or from the genus Ascobolus, or from the species Ascobolus stictoideus.

In another aspect, the polypeptide may be a fungal polypeptide. In one aspect, the polypeptide is a polypeptide having lysozyme activity from a fungus of the class Eurotiomycetes, such as from the order Eurotiales, or from the family Aspergillaceae, or from the genus Hamigera, or from the species Hamigera paravellanea.

In another aspect, the polypeptide may be a fungal polypeptide. In one aspect, the polypeptide is a polypeptide having lysozyme activity from a fungus of the phylum Ascomycota, such as from the genus Engyodontium, or from the species Engyodontium album.

In another aspect, the polypeptide may be a fungal polypeptide. In one aspect, the polypeptide is a polypeptide having lysozyme activity from a fungus of the class Sordariomycetes, such as from the order Xylariales, or from the genus Xylariaceae, or from the species Xylariaceae sp. 1653h.

In another aspect, the polypeptide may be a fungal polypeptide. In one aspect, the polypeptide is a polypeptide having lysozyme activity from a fungus of the class Agaricomycetes, such as from the order Agaricales, or from the family Strophariaceae, or from the genus Deconica or from the species Deconica coprophila.

In another aspect, the polypeptide may be a fungal polypeptide. In one aspect, the polypeptide is a polypeptide having lysozyme activity from a fungus of the class Agaricomycetes, such as from the order Agaricales, or from the family Physalacriaceae, or from the genus Flammulina or from the species Flammulina velutipes.

It will be understood that for the aforementioned species, the present disclosure encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

The present disclosure also relates to polynucleotides encoding a polypeptide of the present disclosure, as described herein. In an embodiment, the polynucleotide encoding the polypeptide of the present disclosure has been isolated.

The techniques used to isolate or clone a polynucleotide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Trichophaea or a strain of Trichoderma, or a related organism and thus, for example, may be an allelic or species variant of the polypeptide encoding region of the polynucleotide.

Modification of a polynucleotide encoding a polypeptide of the present disclosure may be necessary for synthesizing polypeptides substantially similar to the polypeptide. The term “substantially similar” to the polypeptide refers to non-naturally occurring forms of the polypeptide.

The present disclosure also relates to nucleic acid constructs comprising a polynucleotide of the present disclosure operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present disclosure. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present disclosure in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13:97-107), E. coli lac operon, E. coli trc promoter (Egon et al., 1988, Gene 69:301-315), Streptomyces coelicolor agarase gene (dagA), and prokaryotic beta-lactamase gene (Villa-Kamaroff et al., 1978, Proc. Natl. Acad. Sci. USA 75:3727-3731), as well as the tac promoter (DeBoer et al., 1983, Proc. Natl. Acad. Sci. USA 80:21-25). Further promoters are described in “Useful proteins from recombinant bacteria” in Gilbert et al., 1980, Scientific American 242:74-94; and in Sambrook et al., 1989, supra. Examples of tandem promoters are disclosed in WO 99/43835.

Examples of suitable promoters for directing transcription of the nucleic acid constructs of the present disclosure in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8:423-488.

The control sequence may also be a transcription terminator, which is recognized by a host cell to terminate transcription. The terminator is operably linked to 3′-terminus of the polynucleotide encoding the polypeptide. Any terminator that is functional in the host cell may be used in the present disclosure.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177:3465-3471).

The control sequence may also be a leader, a nontranslated region of an mRNA that is important for translation by the host cell. The leader is operably linked to 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15:5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57:109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

The present disclosure also relates to recombinant expression vectors comprising a polynucleotide of the present disclosure, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMB1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98:61-67; Cullen et al., 1987, Nucleic Acids Res. 15:9163-9175; WO 00/24883). Isolation of the AMA1 gene and construction of plasmids or vectors comprising the gene can be accomplished according to the methods disclosed in WO 00/24883.

More than one copy of a polynucleotide of the present disclosure may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present disclosure are well known to one skilled in the art (see, e.g., Sambrook et al., 1989, supra).

The present disclosure also relates to recombinant host cells, comprising a polynucleotide of the present disclosure operably linked to one or more control sequences that direct the production of a polypeptide of the present disclosure. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

In some embodiments, the polypeptide is heterologous to the recombinant host cell.

In some embodiments, at least one of the one or more control sequences is heterologous to the polynucleotide encoding the polypeptide.

In some embodiments, the recombinant host cell comprises at least two copies, e.g., three, four, or five, of the polynucleotide of the present disclosure.

The host cell may be any cell useful in the recombinant production of a polypeptide of the present disclosure, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168:111-115), competent cell transformation (see, e.g., Young and Spizizen, 1961, J. Bacteriol. 81:823-829, or Dubnau and Davidoff-Abelson, 1971, J. Mol. Biol. 56:209-221), electroporation (see, e.g., Shigekawa and Dower, 1988, Biotechniques 6:742-751), or conjugation (see, e.g., Koehler and Thorne, 1987, J. Bacteriol. 169:5271-5278). The introduction of DNA into an E. coli cell may be effected by protoplast transformation (see, e.g., Hanahan, 1983, J. Mol. Biol. 166:557-580) or electroporation (see, e.g., Dower et al., 1988, Nucleic Acids Res. 16:6127-6145). The introduction of DNA into a Streptomyces cell may be effected by protoplast transformation, electroporation (see, e.g., Gong et al., 2004, Folia Microbiol. (Praha) 49:399-405), conjugation (see, e.g., Mazodier et al., 1989, J. Bacteriol. 171:3583-3585), or transduction (see, e.g., Burke et al., 2001, Proc. Natl. Acad. Sci. USA 98:6289-6294). The introduction of DNA into a Pseudomonas cell may be effected by electroporation (see, e.g., Choi et al., 2006, J. Microbiol. Methods 64:391-397) or conjugation (see, e.g., Pinedo and Smets, 2005, Appl. Environ. Microbiol. 71:51-57). The introduction of DNA into a Streptococcus cell may be effected by natural competence (see, e.g., Perry and Kuramitsu, 1981, Infect. Immun. 32:1295-1297), protoplast transformation (see, e.g., Catt and Jollick, 1991, Microbios 68:189-207), electroporation (see, e.g., Buckley et al., 1999, Appl. Environ. Microbiol. 65:3800-3804), or conjugation (see, e.g., Clewell, 1981, Microbiol. Rev. 45:409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this disclosure, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolismmay be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81:1470-1474, and Christensen et al., 1988, Bio/Technology 6:1419-1422. Suitable methods for transforming Fusarium species are described by Malardier et al., 1989, Gene 78:147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153:163; and Hinnen et al., 1978, Proc. Natl. Acad. Sci. USA 75:1920.

The present disclosure also relates to methods of producing a polypeptide of the present disclosure, comprising (a) cultivating a cell, which in its wild-type form produces the polypeptide, under conditions conducive for production of the polypeptide; and optionally, (b) recovering the polypeptide. In one aspect, the cell is an Aspergillus sp. nov. XZ2609 cell. In one aspect, the cell is a Chaetomium cupreum cell. In one aspect, the cell is a Cladorrhinum bulbillosum cell. In one aspect, the cell is a Cordyceps cardinalis cell. In one aspect, the cell is a Hypholoma polytrichi cell. In one aspect, the cell is a Isaria farinosa cell. In one aspect, the cell is a Lecanicillium sp. WMM742 cell. In one aspect, the cell is a Malbranchea flava cell. In one aspect, the cell is a Onygena equina cell. In one aspect, the cell is a Paecilomyces sp. XZ2658 cell. In one aspect, the cell is a Penicillium citrinum cell. In one aspect, the cell is a Penicillium sp. ‘qii’ cell. In one aspect, the cell is a Poronia punctata cell. In one aspect, the cell is a Pycnidiophora cf. dispera cell. In one aspect, the cell is a Rhizomucor pusillus cell. In one aspect, the cell is a Sporormia fimetaria cell. In one aspect, the cell is a Thermomucor indicae-seudaticae cell. In one aspect, the cell is a Trichobolus zukalii cell. In one aspect, the cell is a Umbelopsis westeae cell. In one aspect, the cell is a Zopfiella sp. t180-6 cell. In one aspect, the cell is a Zygomycetes sp. XZ2655 cell.

In one aspect, the cell is a Gelasinospora cratophora cell. In one aspect, the cell is a Stropharia semiglobata cell. In one aspect, the cell is a Clonostachys rossmaniae cell. In one aspect, the cell is a Paracremonium inflatum cell. In one aspect, the cell is a Curreya sp. XZ2623 cell. In one aspect, the cell is a Metapochonia bulbillosa cell. In one aspect, the cell is a Aspergillus inflatus cell. In one aspect, the cell is a Ascobolus sp. ZY179 cell. In one aspect, the cell is a Daldinia fissa cell. In one aspect, the cell is a Coniochaeta sp. cell. In one aspect, the cell is a Aspergillus deflectus cell. In one aspect, the cell is a Delitschia sp. cell. In one aspect, the cell is a Metarhizium iadini cell. In one aspect, the cell is a Hypoxylon sp. cell. In one aspect, the cell is a Hypoxylon sp. cell. In one aspect, the cell is a Simplicillium obclavatum cell. In one aspect, the cell is a Yunnania penicillata cell. In one aspect, the cell is a Westerdykella sp. cell. In one aspect, the cell is a Thermoascus aurantiacus cell. In one aspect, the cell is a Coniothyrium sp. cell. In one aspect, the cell is a Ascobolus stictoideus cell. In one aspect, the cell is a Hamigera paravellanea cell. In one aspect, the cell is a Engyodontium album cell. In one aspect, the cell is a Xylariaceae sp. 1653h cell. In one aspect, the cell is a Deconica coprophila cell. In one aspect, the cell is a Flammulina velutipes cell.

The present disclosure also relates to methods of producing a polypeptide of the present disclosure, comprising (a) cultivating a recombinant host cell of the present disclosure under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

The present disclosure also relates to isolated plants, e.g., a transgenic plant, plant part, or plant cell, comprising a polynucleotide of the present disclosure so as to express and produce a polypeptide or domain in recoverable quantities. The polypeptide or domain may be recovered from the plant or plant part. Alternatively, the plant or plant part containing the polypeptide or domain may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems.

Plant cells and specific plant cell compartments, such as chloroplasts, apoplasts, mitochondria, vacuoles, peroxisomes and cytoplasm are also considered to be a plant part.

Also included within the scope of the present disclosure are the progeny of such plants, plant parts, and plant cells.

The transgenic plant or plant cell expressing the polypeptide or domain may be constructed in accordance with methods known in the art.

The present disclosure also relates to methods of producing a polypeptide or domain of the present disclosure comprising (a) cultivating a transgenic plant or a plant cell comprising a polynucleotide encoding the polypeptide or domain under conditions conducive for production of the polypeptide or domain; and (b) recovering the polypeptide or domain.

### Fermentation Broth Formulations or Cell Compositions

The present disclosure also relates to a fermentation broth formulation or a cell composition comprising a polypeptide of the present disclosure. The fermentation broth product further comprises additional ingredients used in the fermentation process, such as, for example, cells (including, the host cells containing the gene encoding the polypeptide of the present disclosure which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present disclosure may be produced by a method described in WO 90/15861 or WO 2010/096673.

The present disclosure also relates to compositions comprising a polypeptide of the present disclosure. Preferably, the compositions are enriched in the polypeptide of the present disclosure. The term “enriched” indicates that the lysozyme activity of the composition has been increased, e.g., with an enrichment factor of at least 1.1, such as at least 1.2, at least 1.3, at least 1.4, at least 1.5, at least 2.0, at least 3.0, at least 4.0, at least 5.0, at least 10.

In a preferred embodiment, the composition comprises one or more lysozymes selected from the list consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233.

In an embodiment, the composition comprises the polypeptide of the present disclosure and one or more formulating agents, as described below.

The compositions may further comprise multiple enzymatic activities, such as one or more (e.g., several) enzymes selected from the group consisting of phytase, xylanase, galactanase, alpha-galactosidase, beta-galactosidase, protease, phospholipase A1, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.

The compositions may further comprise one or more probiotics. In an embodiment, the probiotic is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococsus acidilactici, Pediococcus sp., Propionibacterium thoenii, Propionibacterium sp. and Streptococcus sp. or any combination thereof.

In an embodiment, the composition comprises one or more formulating agents as disclosed herein, preferably one or more of the compounds selected from the list consisting of glycerol, ethylene glycol, 1,2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose.

In an embodiment, the composition comprises one or more components selected from the list consisting of vitamins, minerals and amino acids.

Enzymes of the present disclosure may be formulated as a liquid or a solid. For a liquid formulation, the formulating agent may comprise a polyol (such as e.g. glycerol, ethylene glycol or propylene glycol), a salt (such as e.g. sodium chloride, sodium benzoate, potassium sorbate) or a sugar or sugar derivative (such as e.g. dextrin, glucose, sucrose, and sorbitol). Thus in one embodiment, the composition is a liquid composition comprising the polypeptide of the present disclosure and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol. The liquid formulation may be sprayed onto the feed after it has been pelleted or may be added to drinking water given to the animals.

For a solid formulation, the formulation may be for example as a granule, spray dried powder or agglomerate (e.g. as disclosed in WO 00/70034). The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

In one embodiment, the composition is a solid composition, such as a spray dried composition, comprising the lysozyme of the present disclosure and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: sodium sulfate, dextrin, cellulose, sodium thiosulfate, magnesium sulfate and calcium carbonate.

The present disclosure also relates to enzyme granules/particles comprising the lysozyme of the present disclosure optionally combined with one or more additional enzymes. The granule is composed of a core, and optionally one or more coatings (outer layers) surrounding the core.

Typically the granule/particle size, measured as equivalent spherical diameter (volume based average particle size), of the granule is 20-2000 μm, particularly 50-1500 μm, 100-1500 μm or 250-1200 μm.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier. Preparation methods include known feed and granule formulation technologies, e.g.:
a) spray dried products, wherein a liquid enzyme-containing solution is atomized in a spray drying tower to form small droplets which during their way down the drying tower dry to form an enzyme-containing particulate material;
b) layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606;
c) absorbed core particles, wherein rather than coating the enzyme as a layer around the core, the enzyme is absorbed onto and/or into the surface of the core. Such a process is described in WO 97/39116.
d) extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme;
e) prilled products, wherein an enzyme-containing powder is suspended in molten wax and the suspension is sprayed, e.g., through a rotating disk atomiser, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. Also U.S. Pat. Nos. 4,016,040 and 4,713,245 are documents relating to this technique;
f) mixer granulation products, wherein a liquid is added to a dry powder composition of, e.g., conventional granulating components, the enzyme being introduced either via the liquid or the powder or both. The liquid and the powder are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process wherein various high-shear mixers can be used as granulators, granulates consisting of enzyme as enzyme, fillers and binders etc. are mixed with cellulose fibres to reinforce the particles to give the so-called T-granulate. Reinforced particles, being more robust, release less enzymatic dust.
g) size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in (Martin Rhodes (editor); Principles of Powder Technology; 1990; Chapter 10; John Wiley & Sons);
h) fluid bed granulation, which involves suspending particulates in an air stream and spraying a liquid onto the fluidized particles via nozzles. Particles hit by spray droplets get wetted and become tacky. The tacky particles collide with other particles and adhere to them and form a granule;
i) the cores may be subjected to drying, such as in a fluid bed drier. Other known methods for drying granules in the feed or detergent industry can be used by the skilled person. The drying preferably takes place at a product temperature of from 25 to 90° C. For some enzymes it is important the cores comprising the enzyme contain a low amount of water before coating. If water sensitive enzymes are coated before excessive water is removed, it will be trapped within the core and it may affect the activity of the enzyme negatively. After drying, the cores preferably contain 0.1-10% w/w water.

The core may include additional materials such as fillers, fibre materials (cellulose or synthetic fibres), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

In one embodiment, the core comprises a material selected from the group consisting of salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals and clay minerals (also known as hydrous aluminium phyllosilicates). In one embodiment, the core comprises a clay mineral such as kaolinite or kaolin.

The core may include an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt and/or wax and/or flour coating, or other suitable coating materials.

The coating may be applied in an amount of at least 0.1% by weight of the core, e.g., at least 0.5%, 1% or 5%. The amount may be at most 100%, 70%, 50%, 40% or 30%.

The coating is preferably at least 0.1 μm thick, particularly at least 0.5 μm, at least 1 μm or at least 5 μm. In some embodiments the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit is encapsulated or enclosed with few or no uncoated areas. The layer or coating should in particular be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, sorbate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO1997/05245, WO1998/54980, WO1998/55599, WO2000/70034, WO2006/034710, WO2008/017661, WO2008/017659, WO2000/020569, WO2001/004279, WO1997/05245, WO2000/01793, WO2003/059086, WO2003/059087, WO2007/031483, WO2007/031485, WO2007/044968, WO2013/192043, WO2014/014647 and WO2015/197719 or polymer coating such as described in WO 2001/00042.

Specific examples of suitable salts are NaCl (CH20° C.=76%), Na2CO3 (CH20° C.=92%), NaNO3 (CH20° C.=73%), Na2HPO4 (CH20° C.=95%), Na3PO4 (CH25° C.=92%), NH4Cl (CH20° C.=79.5%), (NH4)2HPO4 (CH20° C.=93.0%), NH4H2PO4 (CH20° C.=93.1%), (NH4)2SO4 (CH20° C.=81.1%), KCl (CH20° C.=85%), K2HPO4 (CH20° C.=92%), KH2PO4 (CH20° C.=96.5%), KNO3 (CH20° C.=93.5%), Na2SO4 (CH20° C.=93%), K2SO4 (CH20° C.=98%), KHSO4 (CH20° C.=86%), MgSO4 (CH20° C.=90%), ZnSO4 (CH20° C.=90%) and sodium citrate (CH25° C.=86%). Other examples include NaH2PO4, (NH4) H2PO4, CuSO4, Mg (NO3) 2, magnesium acetate, calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, sodium acetate, sodium benzoate, sodium citrate, sodium sulfate, zinc acetate, zinc benzoate, zinc carbonate, zinc chloride, zinc citrate and zinc sorbate.

The salt may be in anhydrous form, or it may be a hydrated salt, i.e. a crystalline salt hydrate with bound water(s) of crystallization, such as described in WO 99/32595. Specific examples include anhydrous sodium sulfate (Na2SO4), anhydrous magnesium sulfate (MgSO4), magnesium sulfate heptahydrate (MgSO4.γH2O), zinc sulfate heptahydrate (ZnSO4·7H2O), sodium phosphate dibasic heptahydrate (Na2HPO4·7H2O), magnesium nitrate hexahydrate (Mg (NO3) 2 (6H2O)), sodium citrate dihydrate and magnesium acetate tetrahydrate.

Preferably the salt is applied as a solution of the salt, e.g., using a fluid bed.

A wax coating may comprise at least 60% by weight of a wax, e.g., at least 65%, at least 70%, at least 75%, at least 80%, at least 85%, at least 90%, at least 95% or at least 99% by weight.

Specific examples of waxes are polyethylene glycols; polypropylenes; Carnauba wax; Candelilla wax; bees wax; hydrogenated plant oil or animal tallow such as polyethylene glycol (PEG), methyl hydroxy-propyl cellulose (MHPC), polyvinyl alcohol (PVA), hydrogenated ox tallow, hydrogenated palm oil, hydrogenated cotton seeds and/or hydrogenated soy bean oil; fatty acid alcohols; mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil.

The granule may comprise a core comprising the lysozyme of the present disclosure, one or more salt coatings and one or more wax coatings. Examples of enzyme granules with multiple coatings are shown in WO1993/07263, WO1997/23606 and WO2016/149636.

Non-dusting granulates may be produced, e.g., as disclosed in U.S. Pat. Nos. 4,106,991 and 4,661,452 and may optionally be coated by methods known in the art. The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granulate may further comprise one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of enzymes by the use of co-granulates is disclosed in WO 2013/188331.

The present disclosure also relates to protected enzymes prepared according to the method disclosed in EP 238,216.

Thus, in a further aspect, the present disclosure provides a granule, which comprises:
(a) a core comprising a lysozyme according to the present disclosure, and
(b) a coating consisting of one or more layer(s) surrounding the core.

In one embodiment, the coating comprises a salt coating as described herein. In one embodiment, the coating comprises a wax coating as described herein. In one embodiment, the coating comprises a salt coating followed by a wax coating as described herein.

The present disclosure also relates to animal feed additives comprising one or more lysozymes of the present disclosure. Thus in one embodiment, the present disclosure relates to an animal feed additive comprising one or more GH25 polypeptides having lysozyme activity, wherein the polypeptide is selected from the group consisting of:
(a) a polypeptide having at least 80%, such as at least 85%, at least 90% or at least 95% sequence identity to the polypeptide of SEQ ID NO: 3;
(b) a polypeptide having at least 84%, such as at least 85%, at least 90% or at least 95% sequence identity to the polypeptide of SEQ ID NO: 6;
(c) a polypeptide having at least 80%, such as at least 85%, at least 90% or at least 95% sequence identity to the polypeptide of SEQ ID NO: 9;
(d) a polypeptide having at least 80%, such as at least 85%, at least 90% or at least 95% sequence identity to the polypeptide of SEQ ID NO: 12;
(e) a polypeptide having at least 80%, such as at least 85%, at least 90% or at least 95% sequence identity to the polypeptide of SEQ ID NO: 15;
(f) a polypeptide having at least 80%, such as at least 85%, at least 90% or at least 95% sequence identity to the polypeptide of SEQ ID NO: 18;
(g) a polypeptide having at least 80%, such as at least 85%, at least 90% or at least 95% sequence identity to the polypeptide of SEQ ID NO: 21;
(h) a polypeptide having at least 80%, such as at least 85%, at least 90% or at least 95% sequence identity to the polypeptide of SEQ ID NO: 24;
(i) a polypeptide having at least 80%, such as at least 85%, at least 90% or at least 95% sequence identity to the polypeptide of SEQ ID NO: 27;
(j) a polypeptide having at least 84%, such as at least 85%, at least 90% or at least 95% sequence identity to the polypeptide of SEQ ID NO: 30;
(k) a polypeptide having at least 80%, such as at least 85%, at least 90% or at least 95% sequence identity to the polypeptide of SEQ ID NO: 33;
(l) a polypeptide having at least 80%, such as at least 85%, at least 90% or at least 95% sequence identity to the polypeptide of SEQ ID NO: 36;
(m) a polypeptide having at least 84%, such as at least 85%, at least 90% or at least 95% sequence identity to the polypeptide of SEQ ID NO: 39;
(n) a polypeptide having at least 80%, such as at least 85%, at least 90% or at least 95% sequence identity to the polypeptide of SEQ ID NO: 42;
(0) a polypeptide having at least 80%, such as at least 85%, at least 90% or at least 95% sequence identity to the polypeptide of SEQ ID NO: 45;
(p) a polypeptide having at least 80%, such as at least 85%, at least 90% or at least 95% sequence identity to the polypeptide of SEQ ID NO: 48;
(q) a polypeptide having at least 80%, such as at least 85%, at least 90% or at least 95% sequence identity to the polypeptide of SEQ ID NO: 51;
(r) a polypeptide having at least 80%, such as at least 85%, at least 90% or at least 95% sequence identity to the polypeptide of SEQ ID NO: 54;
(s) a polypeptide having at least 80%, such as at least 85%, at least 90% or at least 95% sequence identity to the polypeptide of SEQ ID NO: 57;
(t) a polypeptide having at least 80%, such as at least 85%, at least 90% or at least 95% sequence identity to the polypeptide of SEQ ID NO: 60;
(u) a polypeptide having at least 80%, such as at least 85%, at least 90% or at least 95% sequence identity to the polypeptide of SEQ ID NO: 63;
(v) a polypeptide having at least 80%, such as at least 85%, at least 90% or at least 95% sequence identity to the polypeptide of SEQ ID NO: 66;
(w) a polypeptide having at least 80%, such as at least 85%, at least 90% or at least 95% sequence identity to the polypeptide of SEQ ID NO: 69;
(x) a polypeptide having at least 84%, such as at least 85%, at least 90% or at least 95% sequence identity to the polypeptide of SEQ ID NO: 72;
(y) a polypeptide having at least 80%, such as at least 85%, at least 90% or at least 95% sequence identity to the polypeptide of SEQ ID NO: 75;
(z) a polypeptide having at least 80%, such as at least 85%, at least 90% or at least 95% sequence identity to the polypeptide of SEQ ID NO: 80;
(aa) a polypeptide having at least 80%, such as at least 85%, at least 90% or at least 95% sequence identity to the polypeptide of SEQ ID NO: 83;
(ab) a polypeptide having at least 80%, such as at least 85%, at least 90% or at least 95% sequence identity to the polypeptide of SEQ ID NO: 86;
(ac) a polypeptide having at least 86%, such as at least 90% or at least 95% sequence identity to the polypeptide of SEQ ID NO: 89;
(ad) a polypeptide having at least 82%, such as at least 85%, at least 90% or at least 95% sequence identity to the polypeptide of SEQ ID NO: 92;
(ae) a polypeptide having at least 82%, such as at least 85%, at least 90% or at least 95% sequence identity to the polypeptide of SEQ ID NO: 95;
(af) a polypeptide having at least 80%, such as at least 85%, at least 90% or at least 95% sequence identity to the polypeptide of SEQ ID NO: 98;
(ag) a polypeptide having at least 80%, such as at least 85%, at least 90% or at least 95% sequence identity to the polypeptide of SEQ ID NO: 101;
(ah) a polypeptide having at least 80%, such as at least 85%, at least 90% or at least 95% sequence identity to the polypeptide of SEQ ID NO: 104;
(ai) a polypeptide having at least 80%, such as at least 85%, at least 90% or at least 95% sequence identity to the polypeptide of SEQ ID NO: 107;
(aj) a polypeptide having at least 82%, such as at least 85%, at least 90% or at least 95% sequence identity to the polypeptide of SEQ ID NO: 110;
(ak) a polypeptide having at least 81%, such as at least 85%, at least 90% or at least 95% sequence identity to the polypeptide of SEQ ID NO: 113;
(al) a polypeptide having at least 80%, such as at least 85%, at least 90% or at least 95% sequence identity to the polypeptide of SEQ ID NO: 116;
(am) a polypeptide having at least 80%, such as at least 85%, at least 90% or at least 95% sequence identity to the polypeptide of SEQ ID NO: 119;
(an) a polypeptide having at least 80%, such as at least 85%, at least 90% or at least 95% sequence identity to the polypeptide of SEQ ID NO: 122;
(ao) a polypeptide having at least 80%, such as at least 85%, at least 90% or at least 95% sequence identity to the polypeptide of SEQ ID NO: 125;
(ap) a polypeptide having at least 80%, such as at least 85%, at least 90% or at least 95% sequence identity to the polypeptide of SEQ ID NO: 128;
(aq) a polypeptide having at least 80%, such as at least 85%, at least 90% or at least 95% sequence identity to the polypeptide of SEQ ID NO: 131;
(ar) a polypeptide having at least 80%, such as at least 85%, at least 90% or at least 95% sequence identity to the polypeptide of SEQ ID NO: 134;
(as) a polypeptide having at least 80%, such as at least 85%, at least 90% or at least 95% sequence identity to the polypeptide of SEQ ID NO: 137;
(at) a polypeptide having at least 80%, such as at least 85%, at least 90% or at least 95% sequence identity to the polypeptide of SEQ ID NO: 140;
(au) a polypeptide having at least 80%, such as at least 85%, at least 90% or at least 95% sequence identity to the polypeptide of SEQ ID NO: 143;
(av) a polypeptide having at least 80%, such as at least 85%, at least 90% or at least 95% sequence identity to the polypeptide of SEQ ID NO: 146;
(aw) a polypeptide having at least 80%, such as at least 85%, at least 90% or at least 95% sequence identity to the polypeptide of SEQ ID NO: 149;
(ax) a polypeptide having at least 80%, such as at least 85%, at least 90% or at least 95% sequence identity to the polypeptide of SEQ ID NO: 152;
(ay) a polypeptide having at least 80%, such as at least 85%, at least 90% or at least 95% sequence identity to the polypeptide of SEQ ID NO: 155;
(az) a polypeptide having at least 80%, such as at least 85%, at least 90% or at least 95% sequence identity to the polypeptide of SEQ ID NO: 158;
(ba) a polypeptide having at least 80%, such as at least 85%, at least 90% or at least 95% sequence identity to the polypeptide of SEQ ID NO: 221;
(bb) a polypeptide having at least 80%, such as at least 85%, at least 90% or at least 95% sequence identity to the polypeptide of SEQ ID NO: 224;
(bc) a polypeptide having at least 80%, such as at least 85%, at least 90% or at least 95% sequence identity to the polypeptide of SEQ ID NO: 227;
(bd) a polypeptide having at least 80%, such as at least 85%, at least 90% or at least 95% sequence identity to the polypeptide of SEQ ID NO: 230;
(be) a polypeptide having at least 80%, such as at least 85%, at least 90% or at least 95% sequence identity to the polypeptide of SEQ ID NO: 233;
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27 or 28 positions;
(bg) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal His-tag and/or HQ-tag;
(bh) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal extension of up to 10 amino acids, e.g. 1, 2, 3, 4, 5, 6, 7, 8, 9 or 10 amino acids; and
(bi) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) having lysozyme activity and having at least 90% of the length of the mature polypeptide.

In an embodiment, the one or more GH25 polypeptides comprises the motif YKNA (SEQ ID NO: 160) and/or the motif FGGW[S/T] (SEQ ID NO: 161), and preferably is selected from the group selected from SEQ ID NO: 12, SEQ ID NO: 27, SEQ ID NO: 42, SEQ ID NO: 57, SEQ ID NO: 63, SEQ ID NO: 66, SEQ ID NO: 80, SEQ ID NO: 149, SEQ ID NO: 152, SEQ ID NO: 155, SEQ ID NO: 224, SEQ ID NO: 227, SEQ ID NO: 230 and SEQ ID NO: 233.

In an embodiment, the polypeptide comprises or consists of amino acids 1 to 207 of SEQ ID NO: 3, amino acids 1 to 201 of SEQ ID NO: 6, amino acids 1 to 201 of SEQ ID NO: 9, amino acids 1 to 203 of SEQ ID NO: 12, amino acids 1 to 208 of SEQ ID NO: 15, amino acids 1 to 207 of SEQ ID NO: 18, amino acids 1 to 208 of SEQ ID NO: 21, amino acids 1 to 207 of SEQ ID NO: 24, amino acids 1 to 207 of SEQ ID NO: 27, amino acids 1 to 207 of SEQ ID NO: 30, amino acids 1 to 208 of SEQ ID NO: 33, amino acids 1 to 208 of SEQ ID NO: 36, amino acids 1 to 206 of SEQ ID NO: 39, amino acids 1 to 207 of SEQ ID NO: 42, amino acids 1 to 216 of SEQ ID NO: 45, amino acids 1 to 218 of SEQ ID NO: 48, amino acids 1 to 204 of SEQ ID NO: 51, amino acids 1 to 203 of SEQ ID NO: 54, amino acids 1 to 209 of SEQ ID NO: 57, amino acids 1 to 208 of SEQ ID NO: 60, amino acids 1 to 210 of SEQ ID NO: 63, amino acids 1 to 207 of SEQ ID NO: 66, amino acids 1 to 207 of SEQ ID NO: 69, amino acids 1 to 208 of SEQ ID NO: 72, amino acids 1 to 217 of SEQ ID NO: 75, amino acids 1 to 208 of SEQ ID NO: 80, amino acids 1 to 201 of SEQ ID NO: 83, amino acids 1 to 202 of SEQ ID NO: 86, amino acids 1 to 207 of SEQ ID NO: 89, amino acids 1 to 202 of SEQ ID NO: 92, amino acids 1 to 201 of SEQ ID NO: 95, amino acids 1 to 202 of SEQ ID NO: 98, amino acids 1 to 206 of SEQ ID NO: 101, amino acids 1 to 202 of SEQ ID NO: 104, amino acids 1 to 202 of SEQ ID NO: 107, amino acids 1 to 202 of SEQ ID NO: 110, amino acids 1 to 202 of SEQ ID NO: 113, amino acids 1 to 206 of SEQ ID NO: 116, amino acids 1 to 207 of SEQ ID NO: 119, amino acids 1 to 208 of SEQ ID NO: 122, amino acids 1 to 215 of SEQ ID NO: 125, amino acids 1 to 217 of SEQ ID NO: 128, amino acids 1 to 214 of SEQ ID NO: 131, amino acids 1 to 208 of SEQ ID NO: 134, amino acids 1 to 203 of SEQ ID NO: 137, amino acids 1 to 216 of SEQ ID NO: 140, amino acids 1 to 207 of SEQ ID NO: 143, amino acids 1 to 208 of SEQ ID NO: 146, amino acids 1 to 207 of SEQ ID NO: 149, amino acids 1 to 207 of SEQ ID NO: 152, amino acids 1 to 207 of SEQ ID NO: 155, amino acids 1 to 208 of SEQ ID NO: 158, amino acids 1 to 207 of SEQ ID NO: 221, amino acids 1 to 207 of SEQ ID NO: 224, amino acids 1 to 208 of SEQ ID NO: 227, amino acids 1 to 207 of SEQ ID NO: 230 or amino acids 1 to 207 of SEQ ID NO: 233.

In a preferred embodiment, the polypeptide has at least 150%, such as at least 175%, at least 200%, at least 225%, at least 250%, at least 275%, at least 300%, at least 350%, or at least 400% of the lysozyme activity of SEQ ID NO: 159. In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as follows: lysozyme (50 μL of 0.7 μg/mL lysozyme in phosphate buffer (5 mM citrate, 5 mM K₂HPO₄, 0.01% TritonX-100, pH 5.0)) is mixed with Micrococcus lysodeikticus solution (450 μL of 1% lyophilized Micrococcus lysodeikticus ATCC No. 4698 in milli-Q water) and incubated at 40° C. with shaking (500 rpm) for 45 min; the sample is centrifuged (4000 g, 5 min); supernatant (100 μL) is mixed with HCl (50 μL 3.2M) and incubated at 95° C. for 80 min; NaOH (50 μL, 3.5 M) is added and 150 μL of the sample is added to 4-hydroxybenzhydrazide in K—Na tartrate/NaOH buffer (75 μL of 50 g/L K—Na tartrate+20 g/L NaOH); the mixture is incubated at 95° C. for 10 min; and the optical density is measured at 405 nm.

In an embodiment, the animal feed additive comprises one or more formulating agents, preferably as described herein above.

In an embodiment, the animal feed additive comprises one or more additional enzymes, preferably as described herein below.

In an embodiment, the animal feed additive comprises one or more probiotics, preferably as described herein below.

In an embodiment, the animal feed additive comprises one or more vitamins, preferably as described herein below.

In an embodiment, the animal feed additive comprises one or more minerals, preferably as described herein below.

In an embodiment, the animal feed additive comprises one or more amino acids, preferably as described herein below.

In an embodiment, the animal feed additive comprises one or more prebiotics, preferably as described herein below.

In an embodiment, the animal feed additive comprises one or more organic acids, preferably as described herein below.

In an embodiment, the animal feed additive comprises one or more phytogenics, preferably as described herein below.

The present disclosure also relates to animal feed compositions comprising one or more lysozymes of the present disclosure. In one embodiment, the present disclosure relates to an animal feed comprising the granule as described herein and plant based material. In one embodiment, the present disclosure relates to an animal feed comprising the animal feed additive as described herein and plant based material.

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg.

An animal feed composition according to the present disclosure has a crude protein content of 50-800 g/kg, and furthermore comprises at least one lysozyme as claimed herein.

Furthermore, or in the alternative (to the crude protein content indicated above), the animal feed composition of the present disclosure has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington DC).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen bv, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the present disclosure contains at least one vegetable protein as defined above.

The animal feed composition of the present disclosure may also contain animal protein, such as Meat and Bone Meal, Feather meal, and/or Fish Meal, typically in an amount of 0-25%. The animal feed composition of the present disclosure may also comprise Dried Distillers Grains with Solubles (DDGS), typically in amounts of 0-30%.

In still further particular embodiments, the animal feed composition of the present disclosure contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

The animal feed may comprise vegetable proteins. In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

In a particular embodiment, the vegetable protein source is material from one or more plants of the family Fabaceae, e.g., soybean, lupine, pea, or bean. In another particular embodiment, the vegetable protein source is material from one or more plants of the family Chenopodiaceae, e.g. beet, sugar beet, spinach or quinoa. Other examples of vegetable protein sources are rapeseed, and cabbage. In another particular embodiment, soybean is a preferred vegetable protein source. Other examples of vegetable protein sources are cereals such as barley, wheat, rye, oat, maize (corn), rice, and sorghum.

Animal diets can e.g. be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) lysozyme/enzyme preparation may also be added before or during the feed ingredient step. Typically a liquid lysozyme/enzyme preparation comprises the lysozyme of the present disclosure optionally with a polyol, such as glycerol, ethylene glycol or propylene glycol, and is added after the pelleting step, such as by spraying the liquid formulation onto the pellets. The enzyme may also be incorporated in a feed additive or premix.

Alternatively, the lysozyme can be prepared by freezing a mixture of liquid enzyme solution with a bulking agent such as ground soybean meal, and then lyophilizing the mixture.

The final enzyme concentration in the diet is within the range of 0.01-200 mg enzyme protein per kg diet, preferably between 0.05-100 mg/kg diet, more preferably 0.1-50 mg, even more preferably 0.2-20 mg enzyme protein per kg animal diet.

It is at present contemplated that the enzyme is administered in one or more of the following amounts (dosage ranges): 0.01-200; 0.05-100; 0.1-50; 0.2-20; 0.1-1; 0.2-2; 0.5-5; or 1-10;—all these ranges being in mg lysozyme protein per kg feed (ppm).

For determining mg lysozyme protein per kg feed, the lysozyme is purified from the feed composition, and the specific activity of the purified lysozyme is determined using a relevant assay (see under lysozyme activity). The lysozyme activity of the feed composition as such is also determined using the same assay, and on the basis of these two determinations, the dosage in mg lysozyme protein per kg feed is calculated.

In a particular embodiment, the animal feed additive of the present disclosure is intended for being included (or prescribed as having to be included) in animal diets or feed at levels of 0.01 to 10.0%; more particularly 0.05 to 5.0%; or 0.2 to 1.0% (% meaning g additive per 100 g feed). This is so in particular for premixes.

The same principles apply for determining mg lysozyme protein in feed additives. Of course, if a sample is available of the lysozyme used for preparing the feed additive or the feed, the specific activity is determined from this sample (no need to purify the lysozyme from the feed composition or the additive).

Thus in a further aspect, the present disclosure also relates to an animal feed comprising one or more lysozymes of the present disclosure and plant based material. In another aspect, the present disclosure also relates to an animal feed comprising the animal feed additive of the present disclosure (as described herein above) and plant based material.

Thus in one embodiment, the present disclosure relates to an animal feed additive comprising plant based material and one or more GH25 polypeptides having lysozyme activity, wherein the polypeptide is selected from the group consisting of:
(a) a polypeptide having at least 80%, such as at least 85%, at least 90% or at least 95% sequence identity to the polypeptide of SEQ ID NO: 3;
(b) a polypeptide having at least 84%, such as at least 85%, at least 90% or at least 95% sequence identity to the polypeptide of SEQ ID NO: 6;
(c) a polypeptide having at least 80%, such as at least 85%, at least 90% or at least 95% sequence identity to the polypeptide of SEQ ID NO: 9;
(d) a polypeptide having at least 80%, such as at least 85%, at least 90% or at least 95% sequence identity to the polypeptide of SEQ ID NO: 12;
(e) a polypeptide having at least 80%, such as at least 85%, at least 90% or at least 95% sequence identity to the polypeptide of SEQ ID NO: 15;
(f) a polypeptide having at least 80%, such as at least 85%, at least 90% or at least 95% sequence identity to the polypeptide of SEQ ID NO: 18;
(g) a polypeptide having at least 80%, such as at least 85%, at least 90% or at least 95% sequence identity to the polypeptide of SEQ ID NO: 21;
(h) a polypeptide having at least 80%, such as at least 85%, at least 90% or at least 95% sequence identity to the polypeptide of SEQ ID NO: 24;
(i) a polypeptide having at least 80%, such as at least 85%, at least 90% or at least 95% sequence identity to the polypeptide of SEQ ID NO: 27;
(j) a polypeptide having at least 84%, such as at least 85%, at least 90% or at least 95% sequence identity to the polypeptide of SEQ ID NO: 30;
(k) a polypeptide having at least 80%, such as at least 85%, at least 90% or at least 95% sequence identity to the polypeptide of SEQ ID NO: 33;
(l) a polypeptide having at least 80%, such as at least 85%, at least 90% or at least 95% sequence identity to the polypeptide of SEQ ID NO: 36;
(m) a polypeptide having at least 84%, such as at least 85%, at least 90% or at least 95% sequence identity to the polypeptide of SEQ ID NO: 39;
(n) a polypeptide having at least 80%, such as at least 85%, at least 90% or at least 95% sequence identity to the polypeptide of SEQ ID NO: 42;
(o) a polypeptide having at least 80%, such as at least 85%, at least 90% or at least 95% sequence identity to the polypeptide of SEQ ID NO: 45;
(p) a polypeptide having at least 80%, such as at least 85%, at least 90% or at least 95% sequence identity to the polypeptide of SEQ ID NO: 48;
(q) a polypeptide having at least 80%, such as at least 85%, at least 90% or at least 95% sequence identity to the polypeptide of SEQ ID NO: 51;
(r) a polypeptide having at least 80%, such as at least 85%, at least 90% or at least 95% sequence identity to the polypeptide of SEQ ID NO: 54;
(s) a polypeptide having at least 80%, such as at least 85%, at least 90% or at least 95% sequence identity to the polypeptide of SEQ ID NO: 57;
(t) a polypeptide having at least 80%, such as at least 85%, at least 90% or at least 95% sequence identity to the polypeptide of SEQ ID NO: 60;
(u) a polypeptide having at least 80%, such as at least 85%, at least 90% or at least 95% sequence identity to the polypeptide of SEQ ID NO: 63;
(v) a polypeptide having at least 80%, such as at least 85%, at least 90% or at least 95% sequence identity to the polypeptide of SEQ ID NO: 66;
(w) a polypeptide having at least 80%, such as at least 85%, at least 90% or at least 95% sequence identity to the polypeptide of SEQ ID NO: 69;
(x) a polypeptide having at least 84%, such as at least 85%, at least 90% or at least 95% sequence identity to the polypeptide of SEQ ID NO: 72;
(y) a polypeptide having at least 80%, such as at least 85%, at least 90% or at least 95% sequence identity to the polypeptide of SEQ ID NO: 75;
(z) a polypeptide having at least 80%, such as at least 85%, at least 90% or at least 95% sequence identity to the polypeptide of SEQ ID NO: 80;
(aa) a polypeptide having at least 80%, such as at least 85%, at least 90% or at least 95% sequence identity to the polypeptide of SEQ ID NO: 83;
(ab) a polypeptide having at least 80%, such as at least 85%, at least 90% or at least 95% sequence identity to the polypeptide of SEQ ID NO: 86;
(ac) a polypeptide having at least 86%, such as at least 90% or at least 95% sequence identity to the polypeptide of SEQ ID NO: 89;
(ad) a polypeptide having at least 82%, such as at least 85%, at least 90% or at least 95% sequence identity to the polypeptide of SEQ ID NO: 92;
(ae) a polypeptide having at least 82%, such as at least 85%, at least 90% or at least 95% sequence identity to the polypeptide of SEQ ID NO: 95;
(af) a polypeptide having at least 80%, such as at least 85%, at least 90% or at least 95% sequence identity to the polypeptide of SEQ ID NO: 98;
(ag) a polypeptide having at least 80%, such as at least 85%, at least 90% or at least 95% sequence identity to the polypeptide of SEQ ID NO: 101;
(ah) a polypeptide having at least 80%, such as at least 85%, at least 90% or at least 95% sequence identity to the polypeptide of SEQ ID NO: 104;
(ai) a polypeptide having at least 80%, such as at least 85%, at least 90% or at least 95% sequence identity to the polypeptide of SEQ ID NO: 107;
(aj) a polypeptide having at least 82%, such as at least 85%, at least 90% or at least 95% sequence identity to the polypeptide of SEQ ID NO: 110;
(ak) a polypeptide having at least 81%, such as at least 85%, at least 90% or at least 95% sequence identity to the polypeptide of SEQ ID NO: 113;
(al) a polypeptide having at least 80%, such as at least 85%, at least 90% or at least 95% sequence identity to the polypeptide of SEQ ID NO: 116;
(am) a polypeptide having at least 80%, such as at least 85%, at least 90% or at least 95% sequence identity to the polypeptide of SEQ ID NO: 119;
(an) a polypeptide having at least 80%, such as at least 85%, at least 90% or at least 95% sequence identity to the polypeptide of SEQ ID NO: 122;
(ao) a polypeptide having at least 80%, such as at least 85%, at least 90% or at least 95% sequence identity to the polypeptide of SEQ ID NO: 125;
(ap) a polypeptide having at least 80%, such as at least 85%, at least 90% or at least 95% sequence identity to the polypeptide of SEQ ID NO: 128;
(aq) a polypeptide having at least 80%, such as at least 85%, at least 90% or at least 95% sequence identity to the polypeptide of SEQ ID NO: 131;
(ar) a polypeptide having at least 80%, such as at least 85%, at least 90% or at least 95% sequence identity to the polypeptide of SEQ ID NO: 134;
(as) a polypeptide having at least 80%, such as at least 85%, at least 90% or at least 95% sequence identity to the polypeptide of SEQ ID NO: 137;
(at) a polypeptide having at least 80%, such as at least 85%, at least 90% or at least 95% sequence identity to the polypeptide of SEQ ID NO: 140;
(au) a polypeptide having at least 80%, such as at least 85%, at least 90% or at least 95% sequence identity to the polypeptide of SEQ ID NO: 143;
(av) a polypeptide having at least 80%, such as at least 85%, at least 90% or at least 95% sequence identity to the polypeptide of SEQ ID NO: 146;
(aw) a polypeptide having at least 80%, such as at least 85%, at least 90% or at least 95% sequence identity to the polypeptide of SEQ ID NO: 149;
(ax) a polypeptide having at least 80%, such as at least 85%, at least 90% or at least 95% sequence identity to the polypeptide of SEQ ID NO: 152;
(ay) a polypeptide having at least 80%, such as at least 85%, at least 90% or at least 95% sequence identity to the polypeptide of SEQ ID NO: 155;
(az) a polypeptide having at least 80%, such as at least 85%, at least 90% or at least 95% sequence identity to the polypeptide of SEQ ID NO: 158;
(ba) a polypeptide having at least 80%, such as at least 85%, at least 90% or at least 95% sequence identity to the polypeptide of SEQ ID NO: 221;
(bb) a polypeptide having at least 80%, such as at least 85%, at least 90% or at least 95% sequence identity to the polypeptide of SEQ ID NO: 224;
(bc) a polypeptide having at least 80%, such as at least 85%, at least 90% or at least 95% sequence identity to the polypeptide of SEQ ID NO: 227;
(bd) a polypeptide having at least 80%, such as at least 85%, at least 90% or at least 95% sequence identity to the polypeptide of SEQ ID NO: 230;
(be) a polypeptide having at least 80%, such as at least 85%, at least 90% or at least 95% sequence identity to the polypeptide of SEQ ID NO: 233;
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27 or 28 positions;
(bg) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal His-tag and/or HQ-tag;
(bh) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal extension of up to 10 amino acids, e.g. 1, 2, 3, 4, 5, 6, 7, 8, 9 or 10 amino acids; and
(bi) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) having lysozyme activity and having at least 90% of the length of the mature polypeptide.

In an embodiment, the one or more GH25 polypeptides comprises the motif YKNA (SEQ ID NO: 160) and/or the motif FGGW[S/T] (SEQ ID NO: 161), and preferably is selected from the group selected from SEQ ID NO: 12, SEQ ID NO: 27, SEQ ID NO: 42, SEQ ID NO: 57, SEQ ID NO: 63, SEQ ID NO: 66, SEQ ID NO: 80, SEQ ID NO: 149, SEQ ID NO: 152, SEQ ID NO: 155, SEQ ID NO: 224, SEQ ID NO: 227, SEQ ID NO: 230 and SEQ ID NO: 233.

In an embodiment, the polypeptide comprises or consists of amino acids 1 to 207 of SEQ ID NO: 3, amino acids 1 to 201 of SEQ ID NO: 6, amino acids 1 to 201 of SEQ ID NO: 9, amino acids 1 to 203 of SEQ ID NO: 12, amino acids 1 to 208 of SEQ ID NO: 15, amino acids 1 to 207 of SEQ ID NO: 18, amino acids 1 to 208 of SEQ ID NO: 21, amino acids 1 to 207 of SEQ ID NO: 24, amino acids 1 to 207 of SEQ ID NO: 27, amino acids 1 to 207 of SEQ ID NO: 30, amino acids 1 to 208 of SEQ ID NO: 33, amino acids 1 to 208 of SEQ ID NO: 36, amino acids 1 to 206 of SEQ ID NO: 39, amino acids 1 to 207 of SEQ ID NO: 42, amino acids 1 to 216 of SEQ ID NO: 45, amino acids 1 to 218 of SEQ ID NO: 48, amino acids 1 to 204 of SEQ ID NO: 51, amino acids 1 to 203 of SEQ ID NO: 54, amino acids 1 to 209 of SEQ ID NO: 57, amino acids 1 to 208 of SEQ ID NO: 60, amino acids 1 to 210 of SEQ ID NO: 63, amino acids 1 to 207 of SEQ ID NO: 66, amino acids 1 to 207 of SEQ ID NO: 69, amino acids 1 to 208 of SEQ ID NO: 72, amino acids 1 to 217 of SEQ ID NO: 75, amino acids 1 to 208 of SEQ ID NO: 80, amino acids 1 to 201 of SEQ ID NO: 83, amino acids 1 to 202 of SEQ ID NO: 86, amino acids 1 to 207 of SEQ ID NO: 89, amino acids 1 to 202 of SEQ ID NO: 92, amino acids 1 to 201 of SEQ ID NO: 95, amino acids 1 to 202 of SEQ ID NO: 98, amino acids 1 to 206 of SEQ ID NO: 101, amino acids 1 to 202 of SEQ ID NO: 104, amino acids 1 to 202 of SEQ ID NO: 107, amino acids 1 to 202 of SEQ ID NO: 110, amino acids 1 to 202 of SEQ ID NO: 113, amino acids 1 to 206 of SEQ ID NO: 116, amino acids 1 to 207 of SEQ ID NO: 119, amino acids 1 to 208 of SEQ ID NO: 122, amino acids 1 to 215 of SEQ ID NO: 125, amino acids 1 to 217 of SEQ ID NO: 128, amino acids 1 to 214 of SEQ ID NO: 131, amino acids 1 to 208 of SEQ ID NO: 134, amino acids 1 to 203 of SEQ ID NO: 137, amino acids 1 to 216 of SEQ ID NO: 140, amino acids 1 to 207 of SEQ ID NO: 143, amino acids 1 to 208 of SEQ ID NO: 146, amino acids 1 to 207 of SEQ ID NO: 149, amino acids 1 to 207 of SEQ ID NO: 152, amino acids 1 to 207 of SEQ ID NO: 155, amino acids 1 to 208 of SEQ ID NO: 158, amino acids 1 to 207 of SEQ ID NO: 221, amino acids 1 to 207 of SEQ ID NO: 224, amino acids 1 to 208 of SEQ ID NO: 227, amino acids 1 to 207 of SEQ ID NO: 230 or amino acids 1 to 207 of SEQ ID NO: 233.

In a preferred embodiment, the polypeptide has at least 150%, such as at least 175%, at least 200%, at least 225%, at least 250%, at least 275%, at least 300%, at least 350%, or at least 400% of the lysozyme activity of SEQ ID NO: 159. In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

In one aspect, the polypeptides of the present disclosure have at least 150% (i.e. 1.5 times higher), e.g., at least 175%, at least 200%, at least 225%, at least 250%, at least 275%, at least 300%, at least 350%, or at least 400% of the lysozyme activity of SEQ ID NO: 159 wherein lysozyme activity is determined as follows: lysozyme (50 μL of 0.7 μg/mL lysozyme in phosphate buffer (5 mM citrate, 5 mM K₂HPO₄, 0.01% TritonX-100, pH 5.0)) is mixed with Micrococcus lysodeikticus solution (450 μL of 1% lyophilized Micrococcus lysodeikticus ATCC No. 4698 in milli-Q water) and incubated at 40° C. with shaking (500 rpm) for 45 min; the sample is centrifuged (4000 g, 5 min); supernatant (100 μL) is mixed with HCl (50 μL 3.2M) and incubated at 95° C. for 80 min; NaOH (50 μL, 3.5 M) is added and 150 μL of the sample is added to 4-hydroxybenzhydrazide in K—Na tartrate/NaOH buffer (75 μL of 50 g/L K—Na tartrate+20 g/L NaOH); the mixture is incubated at 95° C. for 10 min; and the optical density is measured at 405 nm.

In an embodiment, the plant based material is selected from the group consisting of legumes, cereals, oats, rye, barley, wheat, maize, corn, sorghum, switchgrass, millet, pearl millet, foxtail millet, soybean, wild soybean, beans, lupin, tepary bean, scarlet runner bean, slimjim bean, lima bean, French bean, Broad bean (fava bean), chickpea, lentil, peanut, Spanish peanut, canola, rapeseed (oilseed rape), rice, beet, cabbage, sugar beet, spinach, quinoa, or pea, in a processed form thereof (such as soybean meal, rapeseed meal) or any combination thereof.

In a further embodiment, the animal feed has been pelleted.

In another embodiment, the compositions described herein optionally include one or more enzymes. Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, xylanase, galactanase, mannanase, dextranase, lysozyme and galactosidase is described in Henrissat et al, “The carbohydrate-active enzymes database (CAZy) in 2013”, Nucl. Acids Res. (1 Jan. 2014) 42 (D1): D490-D495; see also www.cazy.org.

Thus the composition of the present disclosure may also comprise at least one other enzyme selected from the group comprising of phytase (EC 3.1.3.8 or 3.1.3.26); xylanase (EC 3.2.1.8); galactanase (EC 3.2.1.89); alpha-galactosidase (EC 3.2.1.22); protease (EC 3.4); phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); arabinofuranosidase (EC 3.2.1.55); beta-xylosidase (EC 3.2.1.37); acetyl xylan esterase (EC 3.1.1.72); feruloyl esterase (EC 3.1.1.73); cellulase (EC 3.2.1.4); cellobiohydrolases (EC 3.2.1.91); beta-glucosidase (EC 3.2.1.21); pullulanase (EC 3.2.1.41), alpha-mannosidase (EC 3.2.1.24), mannanase (EC 3.2.1.25) and beta-glucanase (EC 3.2.1.4 or EC 3.2.1.6), or any mixture thereof.

In a particular embodiment, the composition of the present disclosure comprises a phytase (EC 3.1.3.8 or 3.1.3.26). Examples of commercially available phytases include Bio-Feed™ Phytase (Novozymes), Ronozyme® P, Ronozyme® NP and Ronozyme® HiPhos (DSM Nutritional Products), Natuphos™ (BASF), Natuphos™ E (BASF), Finase® and Quantum® Blue (AB Enzymes), OptiPhos® (Huvepharma), AveMix® Phytase (Aveve Biochem), Phyzyme® XP (Verenium/DuPont) and Axtra® PHY (DuPont). Other preferred phytases include those described in e.g. WO 98/28408, WO 00/43503, and WO 03/066847.

In a particular embodiment, the composition of the present disclosure comprises a xylanase (EC 3.2.1.8). Examples of commercially available xylanases include Ronozyme® WX (DSM Nutritional Products), Econase® XT and Barley (AB Vista), Xylathin® (Verenium), Hostazym® X (Huvepharma), Axtra® XB (Xylanase/beta-glucanase, DuPont) and Axtra® XAP (Xylanase/amylase/protease, DuPont), AveMix® XG 10 (xylanase/glucanase) and AveMix® 02 CS (xylanase/glucanase/pectinase, Aveve Biochem), and Naturgrain (BASF).

In a particular embodiment, the composition of the present disclosure comprises a protease (EC 3.4). Examples of commercially available proteases include Ronozyme® ProAct (DSM Nutritional Products).

In a particular embodiment, the composition of the present disclosure comprises an alpha-amylase (EC 3.2.1.1). Examples of commercially available alpha-amylases include Ronozyme® A and RONOZYME® RumiStar™ (DSM Nutritional Products).

In one embodiment, the composition of the present disclosure comprises a multicomponent enzyme product, such as FRA® Octazyme (Framelco), Ronozyme® G2, Ronozyme® VP and Ronozyme® MultiGrain (DSM Nutritional Products), Rovabio® Excel or Rovabio® Advance (Adisseo).

Eubiotics are compounds which are designed to give a healthy balance of the micro-flora in the gastrointestinal tract. Eubiotics cover a number of different feed additives, such as probiotics, prebiotics, phytogenics (essential oils) and organic acids which are described in more detail below.

In an embodiment, the animal feed composition further comprises one or more additional probiotic. In a particular embodiment, the animal feed composition further comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

In a preferred embodiment, animal feed composition further comprises a bacterium from one or more of the following strains: Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, Lactobacillus rhamnosus, Clostridium butyricum, Bifidobacterium animalis ssp. animalis, Lactobacillus reuteri, Lactobacillus salivarius ssp. salivarius, Megasphaera elsdenii, Propionibacteria sp.

In a more preferred embodiment, composition, animal feed additive or animal feed further comprises a bacterium from one or more of the following strains of Bacillus subtilis: 3A-P4 (PTA-6506), 15A-P4 (PTA-6507), 22C-P1 (PTA-6508), 2084 (NRRL B-500130), LSSA01 (NRRL-B-50104), BS27 (NRRL B-501 05), BS 18 (NRRL B-50633), BS 278 (NRRL B-50634), DSM 29870, DSM 29871, DSM 32315, NRRL B-50136, NRRL B-50605, NRRL B-50606, NRRL B-50622 and PTA-7547.

In a more preferred embodiment, composition, animal feed additive or animal feed further comprises a bacterium from one or more of the following strains of Bacillus pumilus: NRRL B-50016, ATCC 700385, NRRL B-50885 or NRRL B-50886.

In a more preferred embodiment, composition, animal feed additive or animal feed further comprises a bacterium from one or more of the following strains of Bacillus lichenformis: NRRL B 50015, NRRL B-50621 or NRRL B-50623.

In a more preferred embodiment, composition, animal feed additive or animal feed further comprises a bacterium from one or more of the following strains of Bacillus amyloliquefaciens: DSM 29869, DSM 29869, NRRL B 50607, PTA-7543, PTA-7549, NRRL B-50349, NRRL B-50606, NRRL B-50013, NRRL B-50151, NRRL B-50141, NRRL B-50147 or NRRL B-50888.

The bacterial count of each of the bacterial strains in the animal feed composition is between 1×10⁴ and 1×10¹⁴ CFU/kg of dry matter, preferably between 1×10⁶ and 1×10¹² CFU/kg of dry matter, and more preferably between 1×10⁷ and 1×10¹¹ CFU/kg of dry matter. In a more preferred embodiment the bacterial count of each of the bacterial strains in the animal feed composition is between 1×10⁸ and 1×10¹⁰ CFU/kg of dry matter.

The bacterial count of each of the bacterial strains in the animal feed composition is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day. In a more preferred embodiment the bacterial count of each of the bacterial strains in the animal feed composition is between 1×10⁹ and 1×10¹¹ CFU/animal/day.

In another embodiment, the one or more bacterial strains are present in the form of a stable spore.

Examples of commercial products are Cylactin® (DSM Nutritional Products), Alterion (Adisseo), Enviva PRO (DuPont Animal Nutrition), Syncra® (mix enzyme+probiotic, DuPont Animal Nutrition), Ecobiol® and Fecinor® (Norel/Evonik) and GutCare® PY1 (Evonik).

Prebiotics are substances that induce the growth or activity of microorganisms (e.g., bacteria and fungi) that contribute to the well-being of their host. Prebiotics are typically non-digestible fiber compounds that pass undigested through the upper part of the gastrointestinal tract and stimulate the growth or activity of advantageous bacteria that colonize the large bowel by acting as substrate for them. Normally, prebiotics increase the number or activity of bifidobacteria and lactic acid bacteria in the GI tract.

Yeast derivatives (inactivated whole yeasts or yeast cell walls) can also be considered as prebiotics. They often comprise mannan-oligosaccharids, yeast beta-glucans or protein contents and are normally derived from the cell wall of the yeast, Saccharomyces cerevisiae.

Examples of yeast products are Yang® and Agrimos (Lallemand Animal Nutrition).

Phytogenics are a group of natural growth promoters or non-antibiotic growth promoters used as feed additives, derived from herbs, spices or other plants. Phytogenics can be single substances prepared from essential oils/extracts, essential oils/extracts, single plants and mixture of plants (herbal products) or mixture of essential oils/extracts/plants (specialized products).

Examples of phytogenics are rosemary, sage, oregano, thyme, clove, and lemongrass. Examples of essential oils are thymol, eugenol, meta-cresol, vaniline, salicylate, resorcine, guajacol, gingerol, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric and curcuma extract.

Examples of commercial products are Crina® (DSM Nutritional Products); Cinergy™, Biacid™, ProHacid™ Classic and ProHacid™ Advance™ (all Promivi/Cargill) and Envivo EO (DuPont Animal Nutrition).

Organic acids (C1-C7) are widely distributed in nature as normal constituents of plants or animal tissues. They are also formed through microbial fermentation of carbohydrates mainly in the large intestine. They are often used in swine and poultry production as a replacement of antibiotic growth promoters since they have a preventive effect on the intestinal problems like necrotic enteritis in chickens and Escherichia coli infection in young pigs. Organic acids can be sold as mono component or mixtures of typically 2 or 3 different organic acids. Examples of organic acids are propionic acid, formic acid, citric acid, lactic acid, sorbic acid, malic acid, acetic acid, fumaric acid, benzoic acid, butyric acid and tartaric acid or their salt (typically sodium or potassium salt such as potassium diformate or sodium butyrate). Examples of commercial products are VevoVitall® (DSM Nutritional Products), Amasil®, Luprisil®, Lupro-Grain®, Lupro-Cid®, Lupro-Mix® (BASF), n-Butyric Acid AF (OXEA) and Adimix Precision (Nutriad).

The incorporation of the composition of feed additives as exemplified herein above to animal feeds, for example poultry feeds, is in practice carried out using a concentrate or a premix. A premix designates a preferably uniform mixture of one or more microingredients with diluent and/or carrier. Premixes are used to facilitate uniform dispersion of micro-ingredients in a larger mix. A premix according to the present disclosure can be added to feed ingredients or to the drinking water as solids (for example as water soluble powder) or liquids.

The composition of the present disclosure may further comprise one or more amino acids. Examples of amino acids which are used in animal feed are lysine, alanine, beta-alanine, threonine, methionine and tryptophan.

In another embodiment, the animal feed may include one or more vitamins, such as one or more fat-soluble vitamins and/or one or more water-soluble vitamins. In another embodiment, the animal feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin C, vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, iodine, selenium and zinc.

Non-limiting examples of macro minerals include calcium, magnesium, phosphorus, potassium and sodium.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the present disclosure comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the present disclosure in such an amount as to provide an in-feed-concentration within the range indicated in column four, or column five, or column six of Table A.

In a still further embodiment, the animal feed additive of the present disclosure comprises at least one of the below vitamins, preferably to provide an in-feed-concentration within the ranges specified in the below Table 1 (for piglet diets, and broiler diets, respectively).

TABLE 1
Typical vitamin recommendations

| Vitamin | Piglet diet | | Broiler diet | |
|---|---|---|---|---|
| Vitamin A | 10,000-15,000 | IU/kg feed | 8-12,500 | IU/kg feed |
| Vitamin D3 | 1800-2000 | IU/kg feed | 3000-5000 | IU/kg feed |
| Vitamin E | 60-100 | mg/kg feed | 150-240 | mg/kg feed |
| Vitamin K3 | 2-4 | mg/kg feed | 2-4 | mg/kg feed |
| Vitamin B1 | 2-4 | mg/kg feed | 2-3 | mg/kg feed |
| Vitamin B2 | 6-10 | mg/kg feed | 7-9 | mg/kg feed |
| Vitamin B6 | 4-8 | mg/kg feed | 3-6 | mg/kg feed |
| Vitamin B12 | 0.03-0.05 | mg/kg feed | 0.015-0.04 | mg/kg feed |
| Niacin (Vitamin B3) | 30-50 | mg/kg feed | 50-80 | mg/kg feed |
| Pantothenic acid | 20-40 | mg/kg feed | 10-18 | mg/kg feed |
| Folic acid | 1-2 | mg/kg feed | 1-2 | mg/kg feed |
| Biotin | 0.15-0.4 | mg/kg feed | 0.15-0.3 | mg/kg feed |
| Choline chloride | 200-400 | mg/kg feed | 300-600 | mg/kg feed |

The composition of the present disclosure may further comprise colouring agents, stabilisers, growth improving additives and aroma compounds/flavourings, polyunsaturated fatty acids (PUFAs); reactive oxygen generating species, antioxidants, anti-microbial peptides, anti-fungal polypeptides and mycotoxin management compounds.

Examples of colouring agents are carotenoids such as beta-carotene, astaxanthin, and lutein.

Examples of aroma compounds/flavourings are creosol, anethol, deca-, undeca- and/or dodeca-lactones, ionones, irone, gingerol, piperidine, propylidene phatalide, butylidene phatalide, capsaicin and tannin.

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Antioxidants can be used to limit the number of reactive oxygen species which can be generated such that the level of reactive oxygen species is in balance with antioxidants.

Mycotoxins, such as deoxynivalenol, aflatoxin, zearalenone and fumonisin can be found in animal feed and can result in negative animal performance or illness. Compounds which can manage the levels of mycotoxin, such as via deactivation of the mycotoxin or via binding of the mycotoxin, can be added to the feed to ameliorate these negative effects. Examples of mycotoxin management compounds are Vitafix®, Vitafix Ultra (Nuscience), Mycofix®, Mycofix® Secure, FUMzyme®, Biomin® BBSH, Biomin® MTV (Biomin), Mold-Nil®, Toxy-Nil® and Unike® Plus (Nutriad).

A lysozyme of the present disclosure may also be used in animal feed, wherein the term “animal” refers to all animals except humans. Examples of animals are mono-gastric animals, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns).

In the use according to the present disclosure the lysozymes can be fed to the animal before, after, or simultaneously with the diet. The latter is preferred.

In a particular embodiment, the lysozyme, in the form in which it is added to the feed, or when being included in a feed additive, is well-defined. Well-defined means that the lysozyme preparation is at least 50% pure as determined by Size-exclusion chromatography (see Example 12 of WO 01/58275). In other particular embodiments the lysozyme preparation is at least 60, 70, 80, 85, 88, 90, 92, 94, or at least 95% pure as determined by this method.

A well-defined lysozyme preparation is advantageous. For instance, it is much easier to dose correctly to the feed a lysozyme that is essentially free from interfering or contaminating other lysozymes. The term dose correctly refers in particular to the objective of obtaining consistent and constant results, and the capability of optimizing dosage based upon the desired effect.

For the use in animal feed, however, the lysozyme need not be pure; it may e.g. include other enzymes, in which case it could be termed a lysozyme preparation.

The lysozyme preparation can be (a) added directly to the feed, or (b) it can be used in the production of one or more intermediate compositions such as feed additives or premixes that is subsequently added to the feed (or used in a treatment process). The degree of purity described above refers to the purity of the original lysozyme preparation, whether used according to (a) or (b) above.

The lysozyme of the present disclosure could also be used in the treatment of necrotic enteritis and/or Clostridium perfringens.

In an embodiment, the present disclosure also relates to a method of improving the performance of an animal comprising administering to the animal the animal feed or the animal feed additive of the present disclosure.

In a preferred embodiment, the method of improving the performance of an animal comprises administering to the animal the animal feed or the animal feed additive comprising the lysozyme selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233.

In an embodiment, the present disclosure also relates to the use of the animal feed or an animal feed additive of the present disclosure for improving the performance of an animal. In another embodiment, the present disclosure relates to the use of one or more lysozymes of the present disclosure for improving the performance of an animal.

In one embodiment, ‘improving the performance of an animal’ means that there is an increase in body weight gain. In another embodiment, ‘improving the performance of an animal’ means that there is an improved feed conversion ratio. In a further embodiment, ‘improving the performance of an animal’ means that there is an increased feed efficiency. In a further embodiment, ‘improving the performance of an animal’ means that there is an increase in body weight gain and/or an improved feed conversion ratio and/or an increased feed efficiency.

In an embodiment, the animal feed comprises plant based material selected from the group consisting of legumes, cereals, oats, rye, barley, wheat, maize, corn, sorghum, switchgrass, millet, pearl millet, foxtail millet, soybean, wild soybean, beans, lupin, tepary bean, scarlet runner bean, slimjim bean, lima bean, French bean, Broad bean (fava bean), chickpea, lentil, peanut, Spanish peanut, canola, rapeseed (oilseed rape), rice, beet, cabbage, sugar beet, spinach, quinoa, or pea, in a processed form thereof (such as soybean meal, rapeseed meal) or any combination thereof.

In an embodiment, the present disclosure provides a method for preparing an animal feed comprising adding one or more lysozymes of the present disclosure to one or more animal feed ingredients. Animal feed ingredients include, but are not limited to concentrates (as defined herein), forage (as defined herein), enzymes, probiotic, vitamins, minerals and amino acids.

In a preferred embodiment, the method of preparing an animal feed comprises mixing plant based material with the lysozyme selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233.

In an embodiment, the plant based material is selected from the group consisting of legumes, cereals, oats, rye, barley, wheat, maize, corn, sorghum, switchgrass, millet, pearl millet, foxtail millet, soybean, wild soybean, beans, lupin, tepary bean, scarlet runner bean, slimjim bean, lima bean, French bean, Broad bean (fava bean), chickpea, lentil, peanut, Spanish peanut, canola, rapeseed (oilseed rape), rice, beet, cabbage, sugar beet, spinach, quinoa, or pea, in a processed form thereof (such as soybean meal, rapeseed meal) or any combination thereof.

Herein follows a list of embodiments of the present disclosure:

1. A method of hydrolysing peptidoglycan in bacterial cell walls comprising treating bacterial cells with one or more GH25 polypeptides having lysozyme activity, wherein the polypeptide is selected from the group consisting of:
(a) a polypeptide having at least 80% sequence identity to the polypeptide of SEQ ID NO: 3;
(b) a polypeptide having at least 84% sequence identity to the polypeptide of SEQ ID NO: 6;
(c) a polypeptide having at least 80% sequence identity to the polypeptide of SEQ ID NO: 9;
(d) a polypeptide having at least 80% sequence identity to the polypeptide of SEQ ID NO: 12;
(e) a polypeptide having at least 80% sequence identity to the polypeptide of SEQ ID NO: 15;
(f) a polypeptide having at least 80% sequence identity to the polypeptide of SEQ ID NO: 18;
(g) a polypeptide having at least 80% sequence identity to the polypeptide of SEQ ID NO: 21;
(h) a polypeptide having at least 80% sequence identity to the polypeptide of SEQ ID NO: 24;
(i) a polypeptide having at least 80% sequence identity to the polypeptide of SEQ ID NO: 27;
(j) a polypeptide having at least 84% sequence identity to the polypeptide of SEQ ID NO: 30;
(k) a polypeptide having at least 80% sequence identity to the polypeptide of SEQ ID NO: 33;
(l) a polypeptide having at least 80% sequence identity to the polypeptide of SEQ ID NO: 36;
(m) a polypeptide having at least 84% sequence identity to the polypeptide of SEQ ID NO: 39;
(n) a polypeptide having at least 80% sequence identity to the polypeptide of SEQ ID NO: 42;
(o) a polypeptide having at least 80% sequence identity to the polypeptide of SEQ ID NO: 45;
(p) a polypeptide having at least 80% sequence identity to the polypeptide of SEQ ID NO: 48;
(q) a polypeptide having at least 80% sequence identity to the polypeptide of SEQ ID NO: 51;
(r) a polypeptide having at least 80% sequence identity to the polypeptide of SEQ ID NO: 54;
(s) a polypeptide having at least 80% sequence identity to the polypeptide of SEQ ID NO: 57;
(t) a polypeptide having at least 80% sequence identity to the polypeptide of SEQ ID NO: 60;
(u) a polypeptide having at least 80% sequence identity to the polypeptide of SEQ ID NO: 63;
(v) a polypeptide having at least 80% sequence identity to the polypeptide of SEQ ID NO: 66;
(w) a polypeptide having at least 80% sequence identity to the polypeptide of SEQ ID NO: 69;
(x) a polypeptide having at least 84% sequence identity to the polypeptide of SEQ ID NO: 72;
(y) a polypeptide having at least 80% sequence identity to the polypeptide of SEQ ID NO: 75;
(z) a polypeptide having at least 80% sequence identity to the polypeptide of SEQ ID NO: 80;
(aa) a polypeptide having at least 80% sequence identity to the polypeptide of SEQ ID NO: 83;
(ab) a polypeptide having at least 80% sequence identity to the polypeptide of SEQ ID NO: 86;
(ac) a polypeptide having at least 86% sequence identity to the polypeptide of SEQ ID NO: 89;
(ad) a polypeptide having at least 82% sequence identity to the polypeptide of SEQ ID NO: 92;
(ae) a polypeptide having at least 82% sequence identity to the polypeptide of SEQ ID NO: 95;
(af) a polypeptide having at least 80% sequence identity to the polypeptide of SEQ ID NO: 98;
(ag) a polypeptide having at least 80% sequence identity to the polypeptide of SEQ ID NO: 101;
(ah) a polypeptide having at least 80% sequence identity to the polypeptide of SEQ ID NO: 104;
(ai) a polypeptide having at least 80% sequence identity to the polypeptide of SEQ ID NO: 107;
(aj) a polypeptide having at least 82% sequence identity to the polypeptide of SEQ ID NO: 110;
(ak) a polypeptide having at least 81% sequence identity to the polypeptide of SEQ ID NO: 113;
(al) a polypeptide having at least 80% sequence identity to the polypeptide of SEQ ID NO: 116;
(am) a polypeptide having at least 80% sequence identity to the polypeptide of SEQ ID NO: 119;
(an) a polypeptide having at least 80% sequence identity to the polypeptide of SEQ ID NO: 122;
(ao) a polypeptide having at least 80% sequence identity to the polypeptide of SEQ ID NO: 125;
(ap) a polypeptide having at least 80% sequence identity to the polypeptide of SEQ ID NO: 128;
(aq) a polypeptide having at least 80% sequence identity to the polypeptide of SEQ ID NO: 131;
(ar) a polypeptide having at least 80% sequence identity to the polypeptide of SEQ ID NO: 134;
(as) a polypeptide having at least 80% sequence identity to the polypeptide of SEQ ID NO: 137;
(at) a polypeptide having at least 80% sequence identity to the polypeptide of SEQ ID NO: 140;
(au) a polypeptide having at least 80% sequence identity to the polypeptide of SEQ ID NO: 143;
(av) a polypeptide having at least 80% sequence identity to the polypeptide of SEQ ID NO: 146;
(aw) a polypeptide having at least 80% sequence identity to the polypeptide of SEQ ID NO: 149;
(ax) a polypeptide having at least 80% sequence identity to the polypeptide of SEQ ID NO: 152;
(ay) a polypeptide having at least 80% sequence identity to the polypeptide of SEQ ID NO: 155;
(az) a polypeptide having at least 80% sequence identity to the polypeptide of SEQ ID NO: 158;
(ba) a polypeptide having at least 80% sequence identity to the polypeptide of SEQ ID NO: 221;
(bb) a polypeptide having at least 80% sequence identity to the polypeptide of SEQ ID NO: 224;
(bc) a polypeptide having at least 80% sequence identity to the polypeptide of SEQ ID NO: 227;
(bd) a polypeptide having at least 80% sequence identity to the polypeptide of SEQ ID NO: 230;
(be) a polypeptide having at least 80% sequence identity to the polypeptide of SEQ ID NO: 233;
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27 or 28 positions;
(bg) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal His-tag and/or HQ-tag;
(bh) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal extension of up to 10 amino acids, e.g. 1, 2, 3, 4, 5, 6, 7, 8, 9 or 10 amino acids; and
(bi) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) having lysozyme activity and having at least 90% of the length of the mature polypeptide.

2. The method of item 1, wherein the polypeptide is selected from the group consisting of:
(a) a polypeptide having at least 85% sequence identity to the polypeptide of SEQ ID NO: 3;
(b) a polypeptide having at least 85% sequence identity to the polypeptide of SEQ ID NO: 6;
(c) a polypeptide having at least 85% sequence identity to the polypeptide of SEQ ID NO: 9;
(d) a polypeptide having at least 85% sequence identity to the polypeptide of SEQ ID NO: 12;
(e) a polypeptide having at least 85% sequence identity to the polypeptide of SEQ ID NO: 15;
(f) a polypeptide having at least 85% sequence identity to the polypeptide of SEQ ID NO: 18;
(g) a polypeptide having at least 85% sequence identity to the polypeptide of SEQ ID NO: 21;
(h) a polypeptide having at least 85% sequence identity to the polypeptide of SEQ ID NO: 24;
(i) a polypeptide having at least 85% sequence identity to the polypeptide of SEQ ID NO: 27;
(j) a polypeptide having at least 85% sequence identity to the polypeptide of SEQ ID NO: 30;
(k) a polypeptide having at least 85% sequence identity to the polypeptide of SEQ ID NO: 33;
(l) a polypeptide having at least 85% sequence identity to the polypeptide of SEQ ID NO: 36;
(m) a polypeptide having at least 85% sequence identity to the polypeptide of SEQ ID NO: 39;
(n) a polypeptide having at least 85% sequence identity to the polypeptide of SEQ ID NO: 42;
(o) a polypeptide having at least 85% sequence identity to the polypeptide of SEQ ID NO: 45;
(p) a polypeptide having at least 85% sequence identity to the polypeptide of SEQ ID NO: 48;
(q) a polypeptide having at least 85% sequence identity to the polypeptide of SEQ ID NO: 51;
(r) a polypeptide having at least 85% sequence identity to the polypeptide of SEQ ID NO: 54;
(s) a polypeptide having at least 85% sequence identity to the polypeptide of SEQ ID NO: 57;
(t) a polypeptide having at least 85% sequence identity to the polypeptide of SEQ ID NO: 60;
(u) a polypeptide having at least 85% sequence identity to the polypeptide of SEQ ID NO: 63;
(v) a polypeptide having at least 85% sequence identity to the polypeptide of SEQ ID NO: 66;
(w) a polypeptide having at least 85% sequence identity to the polypeptide of SEQ ID NO: 69;
(x) a polypeptide having at least 85% sequence identity to the polypeptide of SEQ ID NO: 72;
(y) a polypeptide having at least 85% sequence identity to the polypeptide of SEQ ID NO: 75;
(z) a polypeptide having at least 85% sequence identity to the polypeptide of SEQ ID NO: 80;
(aa) a polypeptide having at least 85% sequence identity to the polypeptide of SEQ ID NO: 83;
(ab) a polypeptide having at least 85% sequence identity to the polypeptide of SEQ ID NO: 86;
(ac) a polypeptide having at least 86% sequence identity to the polypeptide of SEQ ID NO: 89;
(ad) a polypeptide having at least 85% sequence identity to the polypeptide of SEQ ID NO: 92;
(ae) a polypeptide having at least 85% sequence identity to the polypeptide of SEQ ID NO: 95;
(af) a polypeptide having at least 85% sequence identity to the polypeptide of SEQ ID NO: 98;
(ag) a polypeptide having at least 85% sequence identity to the polypeptide of SEQ ID NO: 101;
(ah) a polypeptide having at least 85% sequence identity to the polypeptide of SEQ ID NO: 104;
(ai) a polypeptide having at least 85% sequence identity to the polypeptide of SEQ ID NO: 107;
(aj) a polypeptide having at least 85% sequence identity to the polypeptide of SEQ ID NO: 110;
(ak) a polypeptide having at least 85% sequence identity to the polypeptide of SEQ ID NO: 113;
(al) a polypeptide having at least 85% sequence identity to the polypeptide of SEQ ID NO: 116;
(am) a polypeptide having at least 85% sequence identity to the polypeptide of SEQ ID NO: 119;
(an) a polypeptide having at least 85% sequence identity to the polypeptide of SEQ ID NO: 122;
(ao) a polypeptide having at least 85% sequence identity to the polypeptide of SEQ ID NO: 125;
(ap) a polypeptide having at least 85% sequence identity to the polypeptide of SEQ ID NO: 128;
(aq) a polypeptide having at least 85% sequence identity to the polypeptide of SEQ ID NO: 131;
(ar) a polypeptide having at least 85% sequence identity to the polypeptide of SEQ ID NO: 134;
(as) a polypeptide having at least 85% sequence identity to the polypeptide of SEQ ID NO: 137;
(at) a polypeptide having at least 85% sequence identity to the polypeptide of SEQ ID NO: 140;
(au) a polypeptide having at least 85% sequence identity to the polypeptide of SEQ ID NO: 143;
(av) a polypeptide having at least 85% sequence identity to the polypeptide of SEQ ID NO: 146;
(aw) a polypeptide having at least 85% sequence identity to the polypeptide of SEQ ID NO: 149;
(ax) a polypeptide having at least 85% sequence identity to the polypeptide of SEQ ID NO: 152;
(ay) a polypeptide having at least 85% sequence identity to the polypeptide of SEQ ID NO: 155;
(az) a polypeptide having at least 85% sequence identity to the polypeptide of SEQ ID NO: 158;
(ba) a polypeptide having at least 85% sequence identity to the polypeptide of SEQ ID NO: 221;
(bb) a polypeptide having at least 85% sequence identity to the polypeptide of SEQ ID NO: 224;
(bc) a polypeptide having at least 85% sequence identity to the polypeptide of SEQ ID NO: 227;
(bd) a polypeptide having at least 85% sequence identity to the polypeptide of SEQ ID NO: 230; and
(be) a polypeptide having at least 85% sequence identity to the polypeptide of SEQ ID NO: 233.

3. The method of item 1, wherein the polypeptide is selected from the group consisting of:
(a) a polypeptide having at least 90% sequence identity to the polypeptide of SEQ ID NO: 3;
(b) a polypeptide having at least 90% sequence identity to the polypeptide of SEQ ID NO: 6;
(c) a polypeptide having at least 90% sequence identity to the polypeptide of SEQ ID NO: 9;
(d) a polypeptide having at least 90% sequence identity to the polypeptide of SEQ ID NO: 12;
(e) a polypeptide having at least 90% sequence identity to the polypeptide of SEQ ID NO: 15;
(f) a polypeptide having at least 90% sequence identity to the polypeptide of SEQ ID NO: 18;
(g) a polypeptide having at least 90% sequence identity to the polypeptide of SEQ ID NO: 21;
(h) a polypeptide having at least 90% sequence identity to the polypeptide of SEQ ID NO: 24;
(i) a polypeptide having at least 90% sequence identity to the polypeptide of SEQ ID NO: 27;
(j) a polypeptide having at least 90% sequence identity to the polypeptide of SEQ ID NO: 30;
(k) a polypeptide having at least 90% sequence identity to the polypeptide of SEQ ID NO: 33;
(l) a polypeptide having at least 90% sequence identity to the polypeptide of SEQ ID NO: 36;
(m) a polypeptide having at least 90% sequence identity to the polypeptide of SEQ ID NO: 39;
(n) a polypeptide having at least 90% sequence identity to the polypeptide of SEQ ID NO: 42;
(o) a polypeptide having at least 90% sequence identity to the polypeptide of SEQ ID NO: 45;
(p) a polypeptide having at least 90% sequence identity to the polypeptide of SEQ ID NO: 48;
(q) a polypeptide having at least 90% sequence identity to the polypeptide of SEQ ID NO: 51;
(r) a polypeptide having at least 90% sequence identity to the polypeptide of SEQ ID NO: 54;
(s) a polypeptide having at least 90% sequence identity to the polypeptide of SEQ ID NO: 57;
(t) a polypeptide having at least 90% sequence identity to the polypeptide of SEQ ID NO: 60;
(u) a polypeptide having at least 90% sequence identity to the polypeptide of SEQ ID NO: 63;
(v) a polypeptide having at least 90% sequence identity to the polypeptide of SEQ ID NO: 66;
(w) a polypeptide having at least 90% sequence identity to the polypeptide of SEQ ID NO: 69;
(x) a polypeptide having at least 90% sequence identity to the polypeptide of SEQ ID NO: 72;
(y) a polypeptide having at least 90% sequence identity to the polypeptide of SEQ ID NO: 75;
(z) a polypeptide having at least 90% sequence identity to the polypeptide of SEQ ID NO: 80;
(aa) a polypeptide having at least 90% sequence identity to the polypeptide of SEQ ID NO: 83;
(ab) a polypeptide having at least 90% sequence identity to the polypeptide of SEQ ID NO: 86;
(ac) a polypeptide having at least 90% sequence identity to the polypeptide of SEQ ID NO: 89;
(ad) a polypeptide having at least 90% sequence identity to the polypeptide of SEQ ID NO: 92;
(ae) a polypeptide having at least 90% sequence identity to the polypeptide of SEQ ID NO: 95;
(af) a polypeptide having at least 90% sequence identity to the polypeptide of SEQ ID NO: 98;
(ag) a polypeptide having at least 90% sequence identity to the polypeptide of SEQ ID NO: 101;
(ah) a polypeptide having at least 90% sequence identity to the polypeptide of SEQ ID NO: 104;
(ai) a polypeptide having at least 90% sequence identity to the polypeptide of SEQ ID NO: 107;
(aj) a polypeptide having at least 90% sequence identity to the polypeptide of SEQ ID NO: 110;
(ak) a polypeptide having at least 90% sequence identity to the polypeptide of SEQ ID NO: 113;
(al) a polypeptide having at least 90% sequence identity to the polypeptide of SEQ ID NO: 116;
(am) a polypeptide having at least 90% sequence identity to the polypeptide of SEQ ID NO: 119;
(an) a polypeptide having at least 90% sequence identity to the polypeptide of SEQ ID NO: 122;
(ao) a polypeptide having at least 90% sequence identity to the polypeptide of SEQ ID NO: 125;
(ap) a polypeptide having at least 90% sequence identity to the polypeptide of SEQ ID NO: 128;
(aq) a polypeptide having at least 90% sequence identity to the polypeptide of SEQ ID NO: 131;
(ar) a polypeptide having at least 90% sequence identity to the polypeptide of SEQ ID NO: 134;
(as) a polypeptide having at least 90% sequence identity to the polypeptide of SEQ ID NO: 137;
(at) a polypeptide having at least 90% sequence identity to the polypeptide of SEQ ID NO: 140;
(au) a polypeptide having at least 90% sequence identity to the polypeptide of SEQ ID NO: 143;
(av) a polypeptide having at least 90% sequence identity to the polypeptide of SEQ ID NO: 146;
(aw) a polypeptide having at least 90% sequence identity to the polypeptide of SEQ ID NO: 149;
(ax) a polypeptide having at least 90% sequence identity to the polypeptide of SEQ ID NO: 152;
(ay) a polypeptide having at least 90% sequence identity to the polypeptide of SEQ ID NO: 155;
(az) a polypeptide having at least 90% sequence identity to the polypeptide of SEQ ID NO: 158;
(ba) a polypeptide having at least 90% sequence identity to the polypeptide of SEQ ID NO: 221;
(bb) a polypeptide having at least 90% sequence identity to the polypeptide of SEQ ID NO: 224;
(bc) a polypeptide having at least 90% sequence identity to the polypeptide of SEQ ID NO: 227;
(bd) a polypeptide having at least 90% sequence identity to the polypeptide of SEQ ID NO: 230;
(be) a polypeptide having at least 90% sequence identity to the polypeptide of SEQ ID NO: 233; and
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions.

4. The method of item 1, wherein the polypeptide is selected from the group consisting of:
(a) a polypeptide having at least 95% sequence identity to the polypeptide of SEQ ID NO: 3;
(b) a polypeptide having at least 95% sequence identity to the polypeptide of SEQ ID NO: 6;
(c) a polypeptide having at least 95% sequence identity to the polypeptide of SEQ ID NO: 9;
(d) a polypeptide having at least 95% sequence identity to the polypeptide of SEQ ID NO: 12;
(e) a polypeptide having at least 95% sequence identity to the polypeptide of SEQ ID NO: 15;
(f) a polypeptide having at least 95% sequence identity to the polypeptide of SEQ ID NO: 18;
(g) a polypeptide having at least 95% sequence identity to the polypeptide of SEQ ID NO: 21;
(h) a polypeptide having at least 95% sequence identity to the polypeptide of SEQ ID NO: 24;
(i) a polypeptide having at least 95% sequence identity to the polypeptide of SEQ ID NO: 27;
(j) a polypeptide having at least 95% sequence identity to the polypeptide of SEQ ID NO: 30;
(k) a polypeptide having at least 95% sequence identity to the polypeptide of SEQ ID NO: 33;
(l) a polypeptide having at least 95% sequence identity to the polypeptide of SEQ ID NO: 36;
(m) a polypeptide having at least 95% sequence identity to the polypeptide of SEQ ID NO: 39;
(n) a polypeptide having at least 95% sequence identity to the polypeptide of SEQ ID NO: 42;
(o) a polypeptide having at least 95% sequence identity to the polypeptide of SEQ ID NO: 45;
(p) a polypeptide having at least 95% sequence identity to the polypeptide of SEQ ID NO: 48;
(q) a polypeptide having at least 95% sequence identity to the polypeptide of SEQ ID NO: 51;
(r) a polypeptide having at least 95% sequence identity to the polypeptide of SEQ ID NO: 54;
(s) a polypeptide having at least 95% sequence identity to the polypeptide of SEQ ID NO: 57;
(t) a polypeptide having at least 95% sequence identity to the polypeptide of SEQ ID NO: 60;
(u) a polypeptide having at least 95% sequence identity to the polypeptide of SEQ ID NO: 63;
(v) a polypeptide having at least 95% sequence identity to the polypeptide of SEQ ID NO: 66;
(w) a polypeptide having at least 95% sequence identity to the polypeptide of SEQ ID NO: 69;
(x) a polypeptide having at least 95% sequence identity to the polypeptide of SEQ ID NO: 72;
(y) a polypeptide having at least 95% sequence identity to the polypeptide of SEQ ID NO: 75;
(z) a polypeptide having at least 95% sequence identity to the polypeptide of SEQ ID NO: 80;
(aa) a polypeptide having at least 95% sequence identity to the polypeptide of SEQ ID NO: 83;
(ab) a polypeptide having at least 95% sequence identity to the polypeptide of SEQ ID NO: 86;
(ac) a polypeptide having at least 95% sequence identity to the polypeptide of SEQ ID NO: 89;
(ad) a polypeptide having at least 95% sequence identity to the polypeptide of SEQ ID NO: 92;
(ae) a polypeptide having at least 95% sequence identity to the polypeptide of SEQ ID NO: 95;
(af) a polypeptide having at least 95% sequence identity to the polypeptide of SEQ ID NO: 98;
(ag) a polypeptide having at least 95% sequence identity to the polypeptide of SEQ ID NO: 101;
(ah) a polypeptide having at least 95% sequence identity to the polypeptide of SEQ ID NO: 104;
(ai) a polypeptide having at least 95% sequence identity to the polypeptide of SEQ ID NO: 107;
(aj) a polypeptide having at least 95% sequence identity to the polypeptide of SEQ ID NO: 110;
(ak) a polypeptide having at least 95% sequence identity to the polypeptide of SEQ ID NO: 113;
(al) a polypeptide having at least 95% sequence identity to the polypeptide of SEQ ID NO: 116;
(am) a polypeptide having at least 95% sequence identity to the polypeptide of SEQ ID NO: 119;
(an) a polypeptide having at least 95% sequence identity to the polypeptide of SEQ ID NO: 122;
(ao) a polypeptide having at least 95% sequence identity to the polypeptide of SEQ ID NO: 125;
(ap) a polypeptide having at least 95% sequence identity to the polypeptide of SEQ ID NO: 128;
(aq) a polypeptide having at least 95% sequence identity to the polypeptide of SEQ ID NO: 131;
(ar) a polypeptide having at least 95% sequence identity to the polypeptide of SEQ ID NO: 134;
(as) a polypeptide having at least 95% sequence identity to the polypeptide of SEQ ID NO: 137;
(at) a polypeptide having at least 95% sequence identity to the polypeptide of SEQ ID NO: 140;
(au) a polypeptide having at least 95% sequence identity to the polypeptide of SEQ ID NO: 143;
(av) a polypeptide having at least 95% sequence identity to the polypeptide of SEQ ID NO: 146;
(aw) a polypeptide having at least 95% sequence identity to the polypeptide of SEQ ID NO: 149;
(ax) a polypeptide having at least 95% sequence identity to the polypeptide of SEQ ID NO: 152;
(ay) a polypeptide having at least 95% sequence identity to the polypeptide of SEQ ID NO: 155;
(az) a polypeptide having at least 95% sequence identity to the polypeptide of SEQ ID NO: 158;
(ba) a polypeptide having at least 95% sequence identity to the polypeptide of SEQ ID NO: 221;
(bb) a polypeptide having at least 95% sequence identity to the polypeptide of SEQ ID NO: 224;
(bc) a polypeptide having at least 95% sequence identity to the polypeptide of SEQ ID NO: 227;
(bd) a polypeptide having at least 95% sequence identity to the polypeptide of SEQ ID NO: 230;
(be) a polypeptide having at least 95% sequence identity to the polypeptide of SEQ ID NO: 233; and
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9 or 10 positions.

5. The method of item 1, wherein the polypeptide comprises or consists of amino acids 1 to 207 of SEQ ID NO: 3, amino acids 1 to 201 of SEQ ID NO: 6, amino acids 1 to 201 of SEQ ID NO: 9, amino acids 1 to 203 of SEQ ID NO: 12, amino acids 1 to 208 of SEQ ID NO: 15, amino acids 1 to 207 of SEQ ID NO: 18, amino acids 1 to 208 of SEQ ID NO: 21, amino acids 1 to 207 of SEQ ID NO: 24, amino acids 1 to 207 of SEQ ID NO: 27, amino acids 1 to 207 of SEQ ID NO: 30, amino acids 1 to 208 of SEQ ID NO: 33, amino acids 1 to 208 of SEQ ID NO: 36, amino acids 1 to 206 of SEQ ID NO: 39, amino acids 1 to 207 of SEQ ID NO: 42, amino acids 1 to 216 of SEQ ID NO: 45, amino acids 1 to 218 of SEQ ID NO: 48, amino acids 1 to 204 of SEQ ID NO: 51, amino acids 1 to 203 of SEQ ID NO: 54, amino acids 1 to 209 of SEQ ID NO: 57, amino acids 1 to 208 of SEQ ID NO: 60, amino acids 1 to 210 of SEQ ID NO: 63, amino acids 1 to 207 of SEQ ID NO: 66, amino acids 1 to 207 of SEQ ID NO: 69, amino acids 1 to 208 of SEQ ID NO: 72, amino acids 1 to 217 of SEQ ID NO: 75, amino acids 1 to 208 of SEQ ID NO: 80, amino acids 1 to 201 of SEQ ID NO: 83, amino acids 1 to 202 of SEQ ID NO: 86, amino acids 1 to 207 of SEQ ID NO: 89, amino acids 1 to 202 of SEQ ID NO: 92, amino acids 1 to 201 of SEQ ID NO: 95, amino acids 1 to 202 of SEQ ID NO: 98, amino acids 1 to 206 of SEQ ID NO: 101, amino acids 1 to 202 of SEQ ID NO: 104, amino acids 1 to 202 of SEQ ID NO: 107, amino acids 1 to 202 of SEQ ID NO: 110, amino acids 1 to 202 of SEQ ID NO: 113, amino acids 1 to 206 of SEQ ID NO: 116, amino acids 1 to 207 of SEQ ID NO: 119, amino acids 1 to 208 of SEQ ID NO: 122, amino acids 1 to 215 of SEQ ID NO: 125, amino acids 1 to 217 of SEQ ID NO: 128, amino acids 1 to 214 of SEQ ID NO: 131, amino acids 1 to 208 of SEQ ID NO: 134, amino acids 1 to 203 of SEQ ID NO: 137, amino acids 1 to 216 of SEQ ID NO: 140, amino acids 1 to 207 of SEQ ID NO: 143, amino acids 1 to 208 of SEQ ID NO: 146, amino acids 1 to 207 of SEQ ID NO: 149, amino acids 1 to 207 of SEQ ID NO: 152, amino acids 1 to 207 of SEQ ID NO: 155, amino acids 1 to 208 of SEQ ID NO: 158, amino acids 1 to 207 of SEQ ID NO: 221, amino acids 1 to 207 of SEQ ID NO: 224, amino acids 1 to 208 of SEQ ID NO: 227, amino acids 1 to 207 of SEQ ID NO: 230 or amino acids 1 to 207 of SEQ ID NO: 233.

6. The method of any of items 1 to 5, wherein the polypeptide has at least 150%, at least 175%, at least 200%, at least 225%, at least 250%, at least 275%, at least 300%, at least 350%, or at least 400% of the lysozyme activity of SEQ ID NO: 159.

7. The method of item 6, wherein the polypeptide has at least 150%, at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

8. The method of item 6, wherein the polypeptide has at least 150%, at least 175%, at least 200%, at least 225%, at least 250%, at least 275%, at least 300%, at least 350%, or at least 400% of the lysozyme activity of SEQ ID NO: 159 wherein lysozyme activity is determined as follows: lysozyme (50 μL of 0.7 μg/mL lysozyme in phosphate buffer (5 mM citrate, 5 mM K₂HPO₄, 0.01% TritonX-100, pH 5.0)) is mixed with Micrococcus lysodeikticus solution (450 μL of 1% lyophilized Micrococcus lysodeikticus ATCC No. 4698 in milli-Q water) and incubated at 40° C. with shaking (500 rpm) for 45 min; the sample is centrifuged (4000 g, 5 min); supernatant (100 μL) is mixed with HCl (50 μL 3.2M) and incubated at 95° C. for 80 min; NaOH (50 μL, 3.5 M) is added and 150 μL of the sample is added to 4-hydroxybenzhydrazide in K—Na tartrate/NaOH buffer (75 μL of 50 g/L K—Na tartrate+20 g/L NaOH); the mixture is incubated at 95° C. for 10 min; and the optical density is measured at 405 nm.

9. A granule comprising one or more GH25 polypeptides having lysozyme activity, wherein the polypeptide is selected from the group consisting of:
(a) a polypeptide having at least 80% sequence identity to the polypeptide of SEQ ID NO: 3;
(b) a polypeptide having at least 84% sequence identity to the polypeptide of SEQ ID NO: 6;
(c) a polypeptide having at least 80% sequence identity to the polypeptide of SEQ ID NO: 9;
(d) a polypeptide having at least 80% sequence identity to the polypeptide of SEQ ID NO: 12;
(e) a polypeptide having at least 80% sequence identity to the polypeptide of SEQ ID NO: 15;
(f) a polypeptide having at least 80% sequence identity to the polypeptide of SEQ ID NO: 18;
(g) a polypeptide having at least 80% sequence identity to the polypeptide of SEQ ID NO: 21;
(h) a polypeptide having at least 80% sequence identity to the polypeptide of SEQ ID NO: 24;
(i) a polypeptide having at least 80% sequence identity to the polypeptide of SEQ ID NO: 27;
(j) a polypeptide having at least 84% sequence identity to the polypeptide of SEQ ID NO: 30;
(k) a polypeptide having at least 80% sequence identity to the polypeptide of SEQ ID NO: 33;
(l) a polypeptide having at least 80% sequence identity to the polypeptide of SEQ ID NO: 36;
(m) a polypeptide having at least 84% sequence identity to the polypeptide of SEQ ID NO: 39;
(n) a polypeptide having at least 80% sequence identity to the polypeptide of SEQ ID NO: 42;
(o) a polypeptide having at least 80% sequence identity to the polypeptide of SEQ ID NO: 45;
(p) a polypeptide having at least 80% sequence identity to the polypeptide of SEQ ID NO: 48;
(q) a polypeptide having at least 80% sequence identity to the polypeptide of SEQ ID NO: 51;
(r) a polypeptide having at least 80% sequence identity to the polypeptide of SEQ ID NO: 54;
(s) a polypeptide having at least 80% sequence identity to the polypeptide of SEQ ID NO: 57;
(t) a polypeptide having at least 80% sequence identity to the polypeptide of SEQ ID NO: 60;
(u) a polypeptide having at least 80% sequence identity to the polypeptide of SEQ ID NO: 63;
(v) a polypeptide having at least 80% sequence identity to the polypeptide of SEQ ID NO: 66;
(w) a polypeptide having at least 80% sequence identity to the polypeptide of SEQ ID NO: 69;
(x) a polypeptide having at least 84% sequence identity to the polypeptide of SEQ ID NO: 72;
(y) a polypeptide having at least 80% sequence identity to the polypeptide of SEQ ID NO: 75;
(z) a polypeptide having at least 80% sequence identity to the polypeptide of SEQ ID NO: 80;
(aa) a polypeptide having at least 80% sequence identity to the polypeptide of SEQ ID NO: 83;
(ab) a polypeptide having at least 80% sequence identity to the polypeptide of SEQ ID NO: 86;
(ac) a polypeptide having at least 86% sequence identity to the polypeptide of SEQ ID NO: 89;
(ad) a polypeptide having at least 82% sequence identity to the polypeptide of SEQ ID NO: 92;
(ae) a polypeptide having at least 82% sequence identity to the polypeptide of SEQ ID NO: 95;
(af) a polypeptide having at least 80% sequence identity to the polypeptide of SEQ ID NO: 98;
(ag) a polypeptide having at least 80% sequence identity to the polypeptide of SEQ ID NO: 101;
(ah) a polypeptide having at least 80% sequence identity to the polypeptide of SEQ ID NO: 104;
(ai) a polypeptide having at least 80% sequence identity to the polypeptide of SEQ ID NO: 107;
(aj) a polypeptide having at least 82% sequence identity to the polypeptide of SEQ ID NO: 110;
(ak) a polypeptide having at least 81% sequence identity to the polypeptide of SEQ ID NO: 113;
(al) a polypeptide having at least 80% sequence identity to the polypeptide of SEQ ID NO: 116;
(am) a polypeptide having at least 80% sequence identity to the polypeptide of SEQ ID NO: 119;
(an) a polypeptide having at least 80% sequence identity to the polypeptide of SEQ ID NO: 122;
(ao) a polypeptide having at least 80% sequence identity to the polypeptide of SEQ ID NO: 125;
(ap) a polypeptide having at least 80% sequence identity to the polypeptide of SEQ ID NO: 128;
(aq) a polypeptide having at least 80% sequence identity to the polypeptide of SEQ ID NO: 131;
(ar) a polypeptide having at least 80% sequence identity to the polypeptide of SEQ ID NO: 134;
(as) a polypeptide having at least 80% sequence identity to the polypeptide of SEQ ID NO: 137;
(at) a polypeptide having at least 80% sequence identity to the polypeptide of SEQ ID NO: 140;
(au) a polypeptide having at least 80% sequence identity to the polypeptide of SEQ ID NO: 143;
(av) a polypeptide having at least 80% sequence identity to the polypeptide of SEQ ID NO: 146;
(aw) a polypeptide having at least 80% sequence identity to the polypeptide of SEQ ID NO: 149;
(ax) a polypeptide having at least 80% sequence identity to the polypeptide of SEQ ID NO: 152;
(ay) a polypeptide having at least 80% sequence identity to the polypeptide of SEQ ID NO: 155;
(az) a polypeptide having at least 80% sequence identity to the polypeptide of SEQ ID NO: 158;
(ba) a polypeptide having at least 80% sequence identity to the polypeptide of SEQ ID NO: 221;
(bb) a polypeptide having at least 80% sequence identity to the polypeptide of SEQ ID NO: 224;
(bc) a polypeptide having at least 80% sequence identity to the polypeptide of SEQ ID NO: 227;
(bd) a polypeptide having at least 80% sequence identity to the polypeptide of SEQ ID NO: 230;
(be) a polypeptide having at least 80% sequence identity to the polypeptide of SEQ ID NO: 233;
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27 or 28 positions;
(bg) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal His-tag and/or HQ-tag;
(bh) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal extension of up to 10 amino acids, e.g. 1, 2, 3, 4, 5, 6, 7, 8, 9 or 10 amino acids; and
(bi) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) having lysozyme activity and having at least 90% of the length of the mature polypeptide.

10. The granule of item 9, wherein the polypeptide is selected from the group consisting of:
(a) a polypeptide having at least 85% sequence identity to the polypeptide of SEQ ID NO: 3;
(b) a polypeptide having at least 85% sequence identity to the polypeptide of SEQ ID NO: 6;
(c) a polypeptide having at least 85% sequence identity to the polypeptide of SEQ ID NO: 9;
(d) a polypeptide having at least 85% sequence identity to the polypeptide of SEQ ID NO: 12;
(e) a polypeptide having at least 85% sequence identity to the polypeptide of SEQ ID NO: 15;
(f) a polypeptide having at least 85% sequence identity to the polypeptide of SEQ ID NO: 18;
(g) a polypeptide having at least 85% sequence identity to the polypeptide of SEQ ID NO: 21;
(h) a polypeptide having at least 85% sequence identity to the polypeptide of SEQ ID NO: 24;
(i) a polypeptide having at least 85% sequence identity to the polypeptide of SEQ ID NO: 27;
(j) a polypeptide having at least 85% sequence identity to the polypeptide of SEQ ID NO: 30;
(k) a polypeptide having at least 85% sequence identity to the polypeptide of SEQ ID NO: 33;
(l) a polypeptide having at least 85% sequence identity to the polypeptide of SEQ ID NO: 36;
(m) a polypeptide having at least 85% sequence identity to the polypeptide of SEQ ID NO: 39;
(n) a polypeptide having at least 85% sequence identity to the polypeptide of SEQ ID NO: 42;
(o) a polypeptide having at least 85% sequence identity to the polypeptide of SEQ ID NO: 45;
(p) a polypeptide having at least 85% sequence identity to the polypeptide of SEQ ID NO: 48;
(q) a polypeptide having at least 85% sequence identity to the polypeptide of SEQ ID NO: 51;
(r) a polypeptide having at least 85% sequence identity to the polypeptide of SEQ ID NO: 54;
(s) a polypeptide having at least 85% sequence identity to the polypeptide of SEQ ID NO: 57;
(t) a polypeptide having at least 85% sequence identity to the polypeptide of SEQ ID NO: 60;
(u) a polypeptide having at least 85% sequence identity to the polypeptide of SEQ ID NO: 63;
(v) a polypeptide having at least 85% sequence identity to the polypeptide of SEQ ID NO: 66;
(w) a polypeptide having at least 85% sequence identity to the polypeptide of SEQ ID NO: 69;
(x) a polypeptide having at least 85% sequence identity to the polypeptide of SEQ ID NO: 72;
(y) a polypeptide having at least 85% sequence identity to the polypeptide of SEQ ID NO: 75;
(z) a polypeptide having at least 85% sequence identity to the polypeptide of SEQ ID NO: 80;
(aa) a polypeptide having at least 85% sequence identity to the polypeptide of SEQ ID NO: 83;
(ab) a polypeptide having at least 85% sequence identity to the polypeptide of SEQ ID NO: 86;
(ac) a polypeptide having at least 86% sequence identity to the polypeptide of SEQ ID NO: 89;
(ad) a polypeptide having at least 85% sequence identity to the polypeptide of SEQ ID NO: 92;
(ae) a polypeptide having at least 85% sequence identity to the polypeptide of SEQ ID NO: 95;
(af) a polypeptide having at least 85% sequence identity to the polypeptide of SEQ ID NO: 98;
(ag) a polypeptide having at least 85% sequence identity to the polypeptide of SEQ ID NO: 101;
(ah) a polypeptide having at least 85% sequence identity to the polypeptide of SEQ ID NO: 104;
(ai) a polypeptide having at least 85% sequence identity to the polypeptide of SEQ ID NO: 107;
(aj) a polypeptide having at least 85% sequence identity to the polypeptide of SEQ ID NO: 110;
(ak) a polypeptide having at least 85% sequence identity to the polypeptide of SEQ ID NO: 113;
(al) a polypeptide having at least 85% sequence identity to the polypeptide of SEQ ID NO: 116;
(am) a polypeptide having at least 85% sequence identity to the polypeptide of SEQ ID NO: 119;
(an) a polypeptide having at least 85% sequence identity to the polypeptide of SEQ ID NO: 122;
(ao) a polypeptide having at least 85% sequence identity to the polypeptide of SEQ ID NO: 125;
(ap) a polypeptide having at least 85% sequence identity to the polypeptide of SEQ ID NO: 128;
(aq) a polypeptide having at least 85% sequence identity to the polypeptide of SEQ ID NO: 131;
(ar) a polypeptide having at least 85% sequence identity to the polypeptide of SEQ ID NO: 134;
(as) a polypeptide having at least 85% sequence identity to the polypeptide of SEQ ID NO: 137;
(at) a polypeptide having at least 85% sequence identity to the polypeptide of SEQ ID NO: 140;
(au) a polypeptide having at least 85% sequence identity to the polypeptide of SEQ ID NO: 143;
(av) a polypeptide having at least 85% sequence identity to the polypeptide of SEQ ID NO: 146;
(aw) a polypeptide having at least 85% sequence identity to the polypeptide of SEQ ID NO: 149;
(ax) a polypeptide having at least 85% sequence identity to the polypeptide of SEQ ID NO: 152;
(ay) a polypeptide having at least 85% sequence identity to the polypeptide of SEQ ID NO: 155;
(az) a polypeptide having at least 85% sequence identity to the polypeptide of SEQ ID NO: 158;
(ba) a polypeptide having at least 85% sequence identity to the polypeptide of SEQ ID NO: 221;
(bb) a polypeptide having at least 85% sequence identity to the polypeptide of SEQ ID NO: 224;
(bc) a polypeptide having at least 85% sequence identity to the polypeptide of SEQ ID NO: 227;
(bd) a polypeptide having at least 85% sequence identity to the polypeptide of SEQ ID NO: 230; and
(be) a polypeptide having at least 85% sequence identity to the polypeptide of SEQ ID NO: 233.

11. The granule of item 9, wherein the polypeptide is selected from the group consisting of:
(a) a polypeptide having at least 90% sequence identity to the polypeptide of SEQ ID NO: 3;
(b) a polypeptide having at least 90% sequence identity to the polypeptide of SEQ ID NO: 6;
(c) a polypeptide having at least 90% sequence identity to the polypeptide of SEQ ID NO: 9;
(d) a polypeptide having at least 90% sequence identity to the polypeptide of SEQ ID NO: 12;
(e) a polypeptide having at least 90% sequence identity to the polypeptide of SEQ ID NO: 15;
(f) a polypeptide having at least 90% sequence identity to the polypeptide of SEQ ID NO: 18;
(g) a polypeptide having at least 90% sequence identity to the polypeptide of SEQ ID NO: 21;
(h) a polypeptide having at least 90% sequence identity to the polypeptide of SEQ ID NO: 24;
(i) a polypeptide having at least 90% sequence identity to the polypeptide of SEQ ID NO: 27;
(j) a polypeptide having at least 90% sequence identity to the polypeptide of SEQ ID NO: 30;
(k) a polypeptide having at least 90% sequence identity to the polypeptide of SEQ ID NO: 33;
(l) a polypeptide having at least 90% sequence identity to the polypeptide of SEQ ID NO: 36;
(m) a polypeptide having at least 90% sequence identity to the polypeptide of SEQ ID NO: 39;
(n) a polypeptide having at least 90% sequence identity to the polypeptide of SEQ ID NO: 42;
(o) a polypeptide having at least 90% sequence identity to the polypeptide of SEQ ID NO: 45;
(p) a polypeptide having at least 90% sequence identity to the polypeptide of SEQ ID NO: 48;
(q) a polypeptide having at least 90% sequence identity to the polypeptide of SEQ ID NO: 51;
(r) a polypeptide having at least 90% sequence identity to the polypeptide of SEQ ID NO: 54;
(s) a polypeptide having at least 90% sequence identity to the polypeptide of SEQ ID NO: 57;
(t) a polypeptide having at least 90% sequence identity to the polypeptide of SEQ ID NO: 60;
(u) a polypeptide having at least 90% sequence identity to the polypeptide of SEQ ID NO: 63;
(v) a polypeptide having at least 90% sequence identity to the polypeptide of SEQ ID NO: 66;
(w) a polypeptide having at least 90% sequence identity to the polypeptide of SEQ ID NO: 69;
(x) a polypeptide having at least 90% sequence identity to the polypeptide of SEQ ID NO: 72;
(y) a polypeptide having at least 90% sequence identity to the polypeptide of SEQ ID NO: 75;
(z) a polypeptide having at least 90% sequence identity to the polypeptide of SEQ ID NO: 80;
(aa) a polypeptide having at least 90% sequence identity to the polypeptide of SEQ ID NO: 83;
(ab) a polypeptide having at least 90% sequence identity to the polypeptide of SEQ ID NO: 86;
(ac) a polypeptide having at least 90% sequence identity to the polypeptide of SEQ ID NO: 89;
(ad) a polypeptide having at least 90% sequence identity to the polypeptide of SEQ ID NO: 92;
(ae) a polypeptide having at least 90% sequence identity to the polypeptide of SEQ ID NO: 95;
(af) a polypeptide having at least 90% sequence identity to the polypeptide of SEQ ID NO: 98;
(ag) a polypeptide having at least 90% sequence identity to the polypeptide of SEQ ID NO: 101;
(ah) a polypeptide having at least 90% sequence identity to the polypeptide of SEQ ID NO: 104;
(ai) a polypeptide having at least 90% sequence identity to the polypeptide of SEQ ID NO: 107;
(aj) a polypeptide having at least 90% sequence identity to the polypeptide of SEQ ID NO: 110;
(ak) a polypeptide having at least 90% sequence identity to the polypeptide of SEQ ID NO: 113;
(al) a polypeptide having at least 90% sequence identity to the polypeptide of SEQ ID NO: 116;
(am) a polypeptide having at least 90% sequence identity to the polypeptide of SEQ ID NO: 119;
(an) a polypeptide having at least 90% sequence identity to the polypeptide of SEQ ID NO: 122;
(ao) a polypeptide having at least 90% sequence identity to the polypeptide of SEQ ID NO: 125;
(ap) a polypeptide having at least 90% sequence identity to the polypeptide of SEQ ID NO: 128;
(aq) a polypeptide having at least 90% sequence identity to the polypeptide of SEQ ID NO: 131;
(ar) a polypeptide having at least 90% sequence identity to the polypeptide of SEQ ID NO: 134;
(as) a polypeptide having at least 90% sequence identity to the polypeptide of SEQ ID NO: 137;
(at) a polypeptide having at least 90% sequence identity to the polypeptide of SEQ ID NO: 140;
(au) a polypeptide having at least 90% sequence identity to the polypeptide of SEQ ID NO: 143;
(av) a polypeptide having at least 90% sequence identity to the polypeptide of SEQ ID NO: 146;
(aw) a polypeptide having at least 90% sequence identity to the polypeptide of SEQ ID NO: 149;
(ax) a polypeptide having at least 90% sequence identity to the polypeptide of SEQ ID NO: 152;
(ay) a polypeptide having at least 90% sequence identity to the polypeptide of SEQ ID NO: 155;
(az) a polypeptide having at least 90% sequence identity to the polypeptide of SEQ ID NO: 158;
(ba) a polypeptide having at least 90% sequence identity to the polypeptide of SEQ ID NO: 221;
(bb) a polypeptide having at least 90% sequence identity to the polypeptide of SEQ ID NO: 224;
(bc) a polypeptide having at least 90% sequence identity to the polypeptide of SEQ ID NO: 227;
(bd) a polypeptide having at least 90% sequence identity to the polypeptide of SEQ ID NO: 230;
(be) a polypeptide having at least 90% sequence identity to the polypeptide of SEQ ID NO: 233; and
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions.

12. The granule of item 9, wherein the polypeptide is selected from the group consisting of:
(a) a polypeptide having at least 95% sequence identity to the polypeptide of SEQ ID NO: 3;
(b) a polypeptide having at least 95% sequence identity to the polypeptide of SEQ ID NO: 6;
(c) a polypeptide having at least 95% sequence identity to the polypeptide of SEQ ID NO: 9;
(d) a polypeptide having at least 95% sequence identity to the polypeptide of SEQ ID NO: 12;
(e) a polypeptide having at least 95% sequence identity to the polypeptide of SEQ ID NO: 15;
(f) a polypeptide having at least 95% sequence identity to the polypeptide of SEQ ID NO: 18;
(g) a polypeptide having at least 95% sequence identity to the polypeptide of SEQ ID NO: 21;
(h) a polypeptide having at least 95% sequence identity to the polypeptide of SEQ ID NO: 24;
(i) a polypeptide having at least 95% sequence identity to the polypeptide of SEQ ID NO: 27;
(j) a polypeptide having at least 95% sequence identity to the polypeptide of SEQ ID NO: 30;
(k) a polypeptide having at least 95% sequence identity to the polypeptide of SEQ ID NO: 33;
(l) a polypeptide having at least 95% sequence identity to the polypeptide of SEQ ID NO: 36;
(m) a polypeptide having at least 95% sequence identity to the polypeptide of SEQ ID NO: 39;
(n) a polypeptide having at least 95% sequence identity to the polypeptide of SEQ ID NO: 42;
(o) a polypeptide having at least 95% sequence identity to the polypeptide of SEQ ID NO: 45;
(p) a polypeptide having at least 95% sequence identity to the polypeptide of SEQ ID NO: 48;
(q) a polypeptide having at least 95% sequence identity to the polypeptide of SEQ ID NO: 51;
(r) a polypeptide having at least 95% sequence identity to the polypeptide of SEQ ID NO: 54;
(s) a polypeptide having at least 95% sequence identity to the polypeptide of SEQ ID NO: 57;
(t) a polypeptide having at least 95% sequence identity to the polypeptide of SEQ ID NO: 60;
(u) a polypeptide having at least 95% sequence identity to the polypeptide of SEQ ID NO: 63;
(v) a polypeptide having at least 95% sequence identity to the polypeptide of SEQ ID NO: 66;
(w) a polypeptide having at least 95% sequence identity to the polypeptide of SEQ ID NO: 69;
(x) a polypeptide having at least 95% sequence identity to the polypeptide of SEQ ID NO: 72;
(y) a polypeptide having at least 95% sequence identity to the polypeptide of SEQ ID NO: 75;
(z) a polypeptide having at least 95% sequence identity to the polypeptide of SEQ ID NO: 80;
(aa) a polypeptide having at least 95% sequence identity to the polypeptide of SEQ ID NO: 83;
(ab) a polypeptide having at least 95% sequence identity to the polypeptide of SEQ ID NO: 86;
(ac) a polypeptide having at least 95% sequence identity to the polypeptide of SEQ ID NO: 89;
(ad) a polypeptide having at least 95% sequence identity to the polypeptide of SEQ ID NO: 92;
(ae) a polypeptide having at least 95% sequence identity to the polypeptide of SEQ ID NO: 95;
(af) a polypeptide having at least 95% sequence identity to the polypeptide of SEQ ID NO: 98;
(ag) a polypeptide having at least 95% sequence identity to the polypeptide of SEQ ID NO: 101;
(ah) a polypeptide having at least 95% sequence identity to the polypeptide of SEQ ID NO: 104;
(ai) a polypeptide having at least 95% sequence identity to the polypeptide of SEQ ID NO: 107;
(aj) a polypeptide having at least 95% sequence identity to the polypeptide of SEQ ID NO: 110;
(ak) a polypeptide having at least 95% sequence identity to the polypeptide of SEQ ID NO: 113;
(al) a polypeptide having at least 95% sequence identity to the polypeptide of SEQ ID NO: 116;
(am) a polypeptide having at least 95% sequence identity to the polypeptide of SEQ ID NO: 119;
(an) a polypeptide having at least 95% sequence identity to the polypeptide of SEQ ID NO: 122;
(ao) a polypeptide having at least 95% sequence identity to the polypeptide of SEQ ID NO: 125;
(ap) a polypeptide having at least 95% sequence identity to the polypeptide of SEQ ID NO: 128;
(aq) a polypeptide having at least 95% sequence identity to the polypeptide of SEQ ID NO: 131;
(ar) a polypeptide having at least 95% sequence identity to the polypeptide of SEQ ID NO: 134;
(as) a polypeptide having at least 95% sequence identity to the polypeptide of SEQ ID NO: 137;
(at) a polypeptide having at least 95% sequence identity to the polypeptide of SEQ ID NO: 140;
(au) a polypeptide having at least 95% sequence identity to the polypeptide of SEQ ID NO: 143;
(av) a polypeptide having at least 95% sequence identity to the polypeptide of SEQ ID NO: 146;
(aw) a polypeptide having at least 95% sequence identity to the polypeptide of SEQ ID NO: 149;
(ax) a polypeptide having at least 95% sequence identity to the polypeptide of SEQ ID NO: 152;
(ay) a polypeptide having at least 95% sequence identity to the polypeptide of SEQ ID NO: 155;
(az) a polypeptide having at least 95% sequence identity to the polypeptide of SEQ ID NO: 158;
(ba) a polypeptide having at least 95% sequence identity to the polypeptide of SEQ ID NO: 221;
(bb) a polypeptide having at least 95% sequence identity to the polypeptide of SEQ ID NO: 224;
(bc) a polypeptide having at least 95% sequence identity to the polypeptide of SEQ ID NO: 227;
(bd) a polypeptide having at least 95% sequence identity to the polypeptide of SEQ ID NO: 230;
(be) a polypeptide having at least 95% sequence identity to the polypeptide of SEQ ID NO: 233; and
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9 or 10 positions.

13. The granule of item 9, wherein the polypeptide comprises or consists of amino acids 1 to 207 of SEQ ID NO: 3, amino acids 1 to 201 of SEQ ID NO: 6, amino acids 1 to 201 of SEQ ID NO: 9, amino acids 1 to 203 of SEQ ID NO: 12, amino acids 1 to 208 of SEQ ID NO: 15, amino acids 1 to 207 of SEQ ID NO: 18, amino acids 1 to 208 of SEQ ID NO: 21, amino acids 1 to 207 of SEQ ID NO: 24, amino acids 1 to 207 of SEQ ID NO: 27, amino acids 1 to 207 of SEQ ID NO: 30, amino acids 1 to 208 of SEQ ID NO: 33, amino acids 1 to 208 of SEQ ID NO: 36, amino acids 1 to 206 of SEQ ID NO: 39, amino acids 1 to 207 of SEQ ID NO: 42, amino acids 1 to 216 of SEQ ID NO: 45, amino acids 1 to 218 of SEQ ID NO: 48, amino acids 1 to 204 of SEQ ID NO: 51, amino acids 1 to 203 of SEQ ID NO: 54, amino acids 1 to 209 of SEQ ID NO: 57, amino acids 1 to 208 of SEQ ID NO: 60, amino acids 1 to 210 of SEQ ID NO: 63, amino acids 1 to 207 of SEQ ID NO: 66, amino acids 1 to 207 of SEQ ID NO: 69, amino acids 1 to 208 of SEQ ID NO: 72, amino acids 1 to 217 of SEQ ID NO: 75, amino acids 1 to 208 of SEQ ID NO: 80, amino acids 1 to 201 of SEQ ID NO: 83, amino acids 1 to 202 of SEQ ID NO: 86, amino acids 1 to 207 of SEQ ID NO: 89, amino acids 1 to 202 of SEQ ID NO: 92, amino acids 1 to 201 of SEQ ID NO: 95, amino acids 1 to 202 of SEQ ID NO: 98, amino acids 1 to 206 of SEQ ID NO: 101, amino acids 1 to 202 of SEQ ID NO: 104, amino acids 1 to 202 of SEQ ID NO: 107, amino acids 1 to 202 of SEQ ID NO: 110, amino acids 1 to 202 of SEQ ID NO: 113, amino acids 1 to 206 of SEQ ID NO: 116, amino acids 1 to 207 of SEQ ID NO: 119, amino acids 1 to 208 of SEQ ID NO: 122, amino acids 1 to 215 of SEQ ID NO: 125, amino acids 1 to 217 of SEQ ID NO: 128, amino acids 1 to 214 of SEQ ID NO: 131, amino acids 1 to 208 of SEQ ID NO: 134, amino acids 1 to 203 of SEQ ID NO: 137, amino acids 1 to 216 of SEQ ID NO: 140, amino acids 1 to 207 of SEQ ID NO: 143, amino acids 1 to 208 of SEQ ID NO: 146, amino acids 1 to 207 of SEQ ID NO: 149, amino acids 1 to 207 of SEQ ID NO: 152, amino acids 1 to 207 of SEQ ID NO: 155, amino acids 1 to 208 of SEQ ID NO: 158, amino acids 1 to 207 of SEQ ID NO: 221, amino acids 1 to 207 of SEQ ID NO: 224, amino acids 1 to 208 of SEQ ID NO: 227, amino acids 1 to 207 of SEQ ID NO: 230 or amino acids 1 to 207 of SEQ ID NO: 233.

14. The granule of any of items 9 to 13, wherein the polypeptide has at least 150%, at least 175%, at least 200%, at least 225%, at least 250%, at least 275%, at least 300%, at least 350%, or at least 400% of the lysozyme activity of SEQ ID NO: 159.

15. The granule of item 14, wherein the polypeptide has at least 150%, at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

16. The granule of item 14, wherein the polypeptide has at least 150%, at least 175%, at least 200%, at least 225%, at least 250%, at least 275%, at least 300%, at least 350%, or at least 400% of the lysozyme activity of SEQ ID NO: 159 wherein lysozyme activity is determined as follows: lysozyme (50 μL of 0.7 μg/mL lysozyme in phosphate buffer (5 mM citrate, 5 mM K₂HPO₄, 0.01% TritonX-100, pH 5.0)) is mixed with Micrococcus lysodeikticus solution (450 μL of 1% lyophilized Micrococcus lysodeikticus ATCC No. 4698 in milli-Q water) and incubated at 40° C. with shaking (500 rpm) for 45 min; the sample is centrifuged (4000 g, 5 min); supernatant (100 μL) is mixed with HCl (50 μL 3.2M) and incubated at 95° C. for 80 min; NaOH (50 μL, 3.5 M) is added and 150 μL of the sample is added to 4-hydroxybenzhydrazide in K—Na tartrate/NaOH buffer (75 L of 50 g/L K—Na tartrate+20 g/L NaOH); the mixture is incubated at 95° C. for 10 min; and the optical density is measured at 405 nm.

17. The granule of any of items 9 to 16, wherein the granule comprises one or more formulating agents.

18. The granule of item 17, wherein the formulating agent is selected from the list consisting of glycerol, ethylene glycol, 1,2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose, preferably selected from the list consisting of 1, 2-propylene glycol, 1,3-propylene glycol, sodium sulfate, dextrin, cellulose, sodium thiosulfate, kaolin and calcium carbonate.

19. The granule of any of items 9 to 18, wherein the granule comprises a core particle and one or more coatings.

20. The granule of item 19, wherein the coating comprises salt and/or wax and/or flour.

21. The granule of any of items 9 to 20 further comprising one or more additional enzymes.

22. The granule of item 21, wherein the one or more additional enzymes is selected from the group consisting of phytase, xylanase, galactanase, alpha-galactosidase, beta-galactosidase, protease, phospholipase A1, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.

23. The granule of any of items 9 to 22 further comprising one or more probiotics.

24. The granule of item 23, wherein the one or more probiotics is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococsus acidilactici, Pediococcus sp., Propionibacterium thoenii, Propionibacterium sp. and Streptococcus sp. or any combination thereof.

25. An isolated polypeptide having lysozyme activity, selected from the group consisting of:
(a) a polypeptide having at least 80% sequence identity to the polypeptide of SEQ ID NO: 3;
(b) a polypeptide having at least 89% sequence identity to the polypeptide of SEQ ID NO: 6;
(c) a polypeptide having at least 81% sequence identity to the polypeptide of SEQ ID NO: 9;
(d) a polypeptide having at least 80% sequence identity to the polypeptide of SEQ ID NO: 12;
(e) a polypeptide having at least 97% sequence identity to the polypeptide of SEQ ID NO: 15;
(f) a polypeptide having at least 80% sequence identity to the polypeptide of SEQ ID NO: 18;
(g) a polypeptide having at least 80% sequence identity to the polypeptide of SEQ ID NO: 24;
(h) a polypeptide having at least 80% sequence identity to the polypeptide of SEQ ID NO: 27;
(i) a polypeptide having at least 83% sequence identity to the polypeptide of SEQ ID NO: 30;
(j) a polypeptide having at least 80% sequence identity to the polypeptide of SEQ ID NO: 33;
(k) a polypeptide having at least 80% sequence identity to the polypeptide of SEQ ID NO: 36;
(l) a polypeptide having at least 83% sequence identity to the polypeptide of SEQ ID NO: 39;
(m) a polypeptide having at least 80% sequence identity to the polypeptide of SEQ ID NO: 42;
(n) a polypeptide having at least 86% sequence identity to the polypeptide of SEQ ID NO: 45;
(0) a polypeptide having at least 80% sequence identity to the polypeptide of SEQ ID NO: 48;
(p) a polypeptide having at least 92% sequence identity to the polypeptide of SEQ ID NO: 51;
(q) a polypeptide having at least 81% sequence identity to the polypeptide of SEQ ID NO: 54;
(r) a polypeptide having at least 80% sequence identity to the polypeptide of SEQ ID NO: 57;
(s) a polypeptide having at least 80% sequence identity to the polypeptide of SEQ ID NO: 60;
(t) a polypeptide having at least 80% sequence identity to the polypeptide of SEQ ID NO: 63;
(u) a polypeptide having at least 80% sequence identity to the polypeptide of SEQ ID NO: 66;
(v) a polypeptide having at least 80% sequence identity to the polypeptide of SEQ ID NO: 69;
(w) a polypeptide having at least 84% sequence identity to the polypeptide of SEQ ID NO: 72;
(x) a polypeptide having at least 80% sequence identity to the polypeptide of SEQ ID NO: 75;
(y) a polypeptide having at least 84% sequence identity to the polypeptide of SEQ ID NO: 80;
(z) a polypeptide having at least 82% sequence identity to the polypeptide of SEQ ID NO: 83;
(aa) a polypeptide having at least 80% sequence identity to the polypeptide of SEQ ID NO: 86;
(ab) a polypeptide having at least 86% sequence identity to the polypeptide of SEQ ID NO: 89;
(ac) a polypeptide having at least 82% sequence identity to the polypeptide of SEQ ID NO: 92;
(ad) a polypeptide having at least 94% sequence identity to the polypeptide of SEQ ID NO: 95;
(ae) a polypeptide having at least 94% sequence identity to the polypeptide of SEQ ID NO: 98;
(af) a polypeptide having at least 80% sequence identity to the polypeptide of SEQ ID NO: 101;
(ag) a polypeptide having at least 81% sequence identity to the polypeptide of SEQ ID NO: 104;
(ah) a polypeptide having at least 80% sequence identity to the polypeptide of SEQ ID NO: 107;
(ai) a polypeptide having at least 88% sequence identity to the polypeptide of SEQ ID NO: 110;
(aj) a polypeptide having at least 85% sequence identity to the polypeptide of SEQ ID NO: 113;
(ak) a polypeptide having at least 80% sequence identity to the polypeptide of SEQ ID NO: 116;
(al) a polypeptide having at least 80% sequence identity to the polypeptide of SEQ ID NO: 119;
(am) a polypeptide having at least 96.5% sequence identity to the polypeptide of SEQ ID NO: 122;
(an) a polypeptide having at least 81% sequence identity to the polypeptide of SEQ ID NO: 125;
(ao) a polypeptide having at least 89% sequence identity to the polypeptide of SEQ ID NO: 128;
(ap) a polypeptide having at least 80% sequence identity to the polypeptide of SEQ ID NO: 131;
(aq) a polypeptide having at least 80% sequence identity to the polypeptide of SEQ ID NO: 134;
(ar) a polypeptide having at least 90% sequence identity to the polypeptide of SEQ ID NO: 137;
(as) a polypeptide having at least 80% sequence identity to the polypeptide of SEQ ID NO: 140;
(at) a polypeptide having at least 80% sequence identity to the polypeptide of SEQ ID NO: 143;
(au) a polypeptide having at least 80% sequence identity to the polypeptide of SEQ ID NO: 146;
(av) a polypeptide having at least 85% sequence identity to the polypeptide of SEQ ID NO: 149;
(aw) a polypeptide having at least 85% sequence identity to the polypeptide of SEQ ID NO: 152;
(ax) a polypeptide having at least 84% sequence identity to the polypeptide of SEQ ID NO: 155;
(ay) a polypeptide having at least 95% sequence identity to the polypeptide of SEQ ID NO: 158;
(az) a polypeptide having at least 80% sequence identity to the polypeptide of SEQ ID NO: 221;
(ba) a polypeptide having at least 80% sequence identity to the polypeptide of SEQ ID NO: 224;
(bb) a polypeptide having at least 80% sequence identity to the polypeptide of SEQ ID NO: 227;
(bc) a polypeptide having at least 85% sequence identity to the polypeptide of SEQ ID NO: 230;
(bd) a polypeptide having at least 85% sequence identity to the polypeptide of SEQ ID NO: 233;
(be) a variant of the polypeptide selected from the group consisting of SEQ ID NO: 3, SEQ ID NO: 12, SEQ ID NO: 18, SEQ ID NO: 24 SEQ ID NO: 27, SEQ ID NO: 33, SEQ ID NO: 36, SEQ ID NO: 42, SEQ ID NO: 48, SEQ ID NO: 57, SEQ ID NO: 60, SEQ ID NO: 63, SEQ ID NO: 66, SEQ ID NO: 69, SEQ ID NO: 75, SEQ ID NO: 86, SEQ ID NO:101, SEQ ID NO: 107, SEQ ID NO: 116, SEQ ID NO: 119, SEQ ID NO: 125, SEQ ID NO: 131, SEQ ID NO: 134, SEQ ID NO: 140, SEQ ID NO: 143, SEQ ID NO: 146, SEQ ID NO: 221, SEQ ID NO: 224 and SEQ ID NO: 227, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 positions;
(bf) a variant of the polypeptide selected from the group consisting of SEQ ID NO: 9, SEQ ID NO: 54 and SEQ ID NO: 104, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 positions;
(bg) a variant of the polypeptide selected from the group consisting of SEQ ID NO: 83 and SEQ ID NO: 92, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 positions;
(bh) a variant of the polypeptide selected from the group consisting of SEQ ID NO: 30 and SEQ ID NO: 39, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 positions;
(bi) a variant of the polypeptide selected from the group consisting of SEQ ID NO: 72, SEQ ID NO: 80 and SEQ ID NO: 155, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 positions;
(bj) a variant of the polypeptide selected from the group consisting of SEQ ID NO: 149, SEQ ID NO: 152, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 positions;
(bk) a variant of the polypeptide selected from the group consisting of SEQ ID NO: 45 and SEQ ID NO: 113, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 positions;
(bl) a variant of SEQ ID NO: 89, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27 or 28 positions;
(bm) a variant of SEQ ID NO: 110, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23 or 24 positions;
(bn) a variant of SEQ ID NO: 128, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22 or 23 positions;
(bo) a variant of SEQ ID NO: 6, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21 or 22 positions;
(bp) a variant of SEQ ID NO: 137, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions;
(bq) a variant of SEQ ID NO: 51, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15 or 16 positions;
(br) a variant of the polypeptide selected from the group consisting of SEQ ID NO: 95 and SEQ ID NO: 98, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11 or 12 positions;
(bs) a variant of SEQ ID NO: 158, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9 or 10 positions;
(bt) a variant of SEQ ID NO: 122, wherein the variant has lysozyme activity and comprises one or more amino acid substitutions, and/or one or more amino acid deletions, and/or one or more amino acid insertions or any combination thereof in 1, 2, 3, 4, 5, 6 or 7 positions;
(bu) a variant of SEQ ID NO: 15, wherein the variant has lysozyme activity and comprises one or more amino acid substitutions, and/or one or more amino acid deletions, and/or one or more amino acid insertions or any combination thereof in 1, 2, 3, 4, 5 or 6 positions;
(bv) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs), (bt) or (bu) and a N-terminal and/or C-terminal His-tag and/or HQ-tag;
(bw) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs), (bt) or (bu) and a N-terminal and/or C-terminal extension of up to 10 amino acids, e.g. 1, 2, 3, 4, 5, 6, 7, 8, 9 or 10 amino acids; and
(bx) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs), (bt) or (bu) having lysozyme activity and having at least 90% of the length of the mature polypeptide.

26. The polypeptide of item 25, wherein the polypeptide comprises or consists of amino acids 1 to 207 of SEQ ID NO: 3, amino acids 1 to 201 of SEQ ID NO: 6, amino acids 1 to 201 of SEQ ID NO: 9, amino acids 1 to 203 of SEQ ID NO: 12, amino acids 1 to 208 of SEQ ID NO: 15, amino acids 1 to 207 of SEQ ID NO: 18, amino acids 1 to 208 of SEQ ID NO: 21, amino acids 1 to 207 of SEQ ID NO: 27, amino acids 1 to 207 of SEQ ID NO: 30, amino acids 1 to 208 of SEQ ID NO: 33, amino acids 1 to 208 of SEQ ID NO: 36, amino acids 1 to 206 of SEQ ID NO: 39, amino acids 1 to 207 of SEQ ID NO: 42, amino acids 1 to 216 of SEQ ID NO: 45, amino acids 1 to 218 of SEQ ID NO: 48, amino acids 1 to 204 of SEQ ID NO: 51, amino acids 1 to 203 of SEQ ID NO: 54, amino acids 1 to 209 of SEQ ID NO: 57, amino acids 1 to 208 of SEQ ID NO: 60, amino acids 1 to 210 of SEQ ID NO: 63, amino acids 1 to 207 of SEQ ID NO: 66, amino acids 1 to 207 of SEQ ID NO: 69, amino acids 1 to 208 of SEQ ID NO: 72, amino acids 1 to 217 of SEQ ID NO: 75, amino acids 1 to 208 of SEQ ID NO: 80, amino acids 1 to 201 of SEQ ID NO: 83, amino acids 1 to 202 of SEQ ID NO: 86, amino acids 1 to 207 of SEQ ID NO: 89, amino acids 1 to 202 of SEQ ID NO: 92, amino acids 1 to 201 of SEQ ID NO: 95, amino acids 1 to 202 of SEQ ID NO: 98, amino acids 1 to 206 of SEQ ID NO: 101, amino acids 1 to 202 of SEQ ID NO: 104, amino acids 1 to 202 of SEQ ID NO: 107, amino acids 1 to 202 of SEQ ID NO: 110, amino acids 1 to 202 of SEQ ID NO: 113, amino acids 1 to 206 of SEQ ID NO: 116, amino acids 1 to 207 of SEQ ID NO: 119, amino acids 1 to 208 of SEQ ID NO: 122, amino acids 1 to 215 of SEQ ID NO: 125, amino acids 1 to 217 of SEQ ID NO: 128, amino acids 1 to 214 of SEQ ID NO: 131, amino acids 1 to 208 of SEQ ID NO: 134, amino acids 1 to 203 of SEQ ID NO: 137, amino acids 1 to 216 of SEQ ID NO: 140, amino acids 1 to 207 of SEQ ID NO: 143, amino acids 1 to 208 of SEQ ID NO: 146, amino acids 1 to 207 of SEQ ID NO: 149, amino acids 1 to 207 of SEQ ID NO: 152, amino acids 1 to 207 of SEQ ID NO: 155, amino acids 1 to 208 of SEQ ID NO: 158, amino acids 1 to 207 of SEQ ID NO: 221, amino acids 1 to 207 of SEQ ID NO: 224, amino acids 1 to 208 of SEQ ID NO: 227, amino acids 1 to 207 of SEQ ID NO: 230 or amino acids 1 to 207 of SEQ ID NO: 233.

27. The polypeptide of any of items 25 to 26, wherein the polypeptide has at least 150%, at least 175%, at least 200%, at least 225%, at least 250%, at least 275%, at least 300%, at least 350%, or at least 400% of the lysozyme activity of SEQ ID NO: 159.

28. The polypeptide of any of items 25 to 27, wherein the polypeptide has at least 150%, at least 175%, at least 200%, at least 225%, at least 250%, at least 275%, at least 300%, at least 350%, or at least 400% of the lysozyme activity of SEQ ID NO: 159 wherein lysozyme activity is determined as described in example 1.

29. The polypeptide of item 28, wherein the polypeptide has at least 150%, at least 175%, at least 200%, at least 225%, at least 250%, at least 275%, at least 300%, at least 350%, or at least 400% of the lysozyme activity of SEQ ID NO: 159 wherein lysozyme activity is determined as follows: lysozyme (50 μL of 0.7 μg/mL lysozyme in phosphate buffer (5 mM citrate, 5 mM K₂HPO₄, 0.01% TritonX-100, pH 5.0)) is mixed with Micrococcus lysodeikticus solution (450 μL of 1% lyophilized Micrococcus lysodeikticus ATCC No. 4698 in milli-Q water) and incubated at 40° C. with shaking (500 rpm) for 45 min; the sample is centrifuged (4000 g, 5 min); supernatant (100 μL) is mixed with HCl (50 μL 3.2M) and incubated at 95° C. for 80 min; NaOH (50 μL, 3.5 M) is added and 150 μL of the sample is added to 4-hydroxybenzhydrazide in K—Na tartrate/NaOH buffer (75 μL of 50 g/L K—Na tartrate+20 g/L NaOH); the mixture is incubated at 95° C. for 10 min; and the optical density is measured at 405 nm.

30. A composition comprising the polypeptide of any of items 25 to 29.

31. The composition of item 30 further comprising one or more formulating agents.

32. The composition of item 31 wherein the formulating agent comprises one or more of the following compounds: glycerol, ethylene glycol, 1,2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose.

33. An animal feed additive comprising the lysozyme as disclosed in any of items 1 to 8, the granule of any of items 9 to 24, the polypeptide of any of items 25 to 29 or the composition of any of items 30 to 32.

34. The animal feed additive of item 33 further comprising one or more components selected from the list consisting of:
one or more vitamins;
one or more minerals;
one or more amino acids;
one or more prebiotics;
one or more organic acids; and
one or more other feed ingredients.

35. The animal feed additive of any of items 33 to 34 further comprising one or more additional enzymes.

36. The animal feed additive of item 35, wherein the one or more additional enzymes is selected from the group consisting of phytase, lysozyme, galactanase, alpha-galactosidase, beta-galactosidase, protease, phospholipase A1, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.

37. The animal feed additive of any of items 33 to 36 further comprising one or more probiotics.

38. The animal feed additive of item 37, wherein the one or more probiotics is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococsus acidilactici, Pediococcus sp., Propionibacterium thoenii, Propionibacterium sp. and Streptococcus sp. or any combination thereof.

39. The animal feed additive of any of items 33 to 38 further comprising one or more phytogenics.

40. The animal feed additive of item 39, wherein the phytogenic is selected from the group consisting of rosemary, sage, oregano, thyme, clove, lemongrass, essential oils, thymol, eugenol, meta-cresol, vaniline, salicylate, resorcine, guajacol, gingerol, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric and curcuma extract or any combination thereof.

41. An animal feed comprising the lysozyme as disclosed in any of items 1 to 8, the granule of any of items 9 to 24, the polypeptide of any of items 25 to 29, the composition of any of items 30 to 32 or the animal feed additive of any of items 33 to 40 and plant based material.

42. The animal feed of item 41, wherein the plant based material is selected from the group consisting of legumes, cereals, oats, rye, barley, wheat, maize, corn, sorghum, switchgrass, millet, pearl millet, foxtail millet, soybean, wild soybean, beans, lupin, tepary bean, scarlet runner bean, slimjim bean, lima bean, French bean, Broad bean (fava bean), chickpea, lentil, peanut, Spanish peanut, canola, rapeseed (oilseed rape), rice, beet, cabbage, sugar beet, spinach, quinoa, or pea, in a processed form thereof (such as soybean meal, rapeseed meal) or any combination thereof.

43. A pelleted animal feed comprising the lysozyme as disclosed in any of items 1 to 8, the granule of any of items 9 to 24, the polypeptide of any of items 25 to 29, the composition of any of items 30 to 32 or the animal feed additive of any of items 33 to 40 and plant based material.

44. The pelleted animal feed of item 43, wherein the plant based material is selected from the group consisting of legumes, cereals, oats, rye, barley, wheat, maize, corn, sorghum, switchgrass, millet, pearl millet, foxtail millet, soybean, wild soybean, beans, lupin, tepary bean, scarlet runner bean, slimjim bean, lima bean, French bean, Broad bean (fava bean), chickpea, lentil, peanut, Spanish peanut, canola, rapeseed (oilseed rape), rice, beet, cabbage, sugar beet, spinach, quinoa, or pea, in a processed form thereof (such as soybean meal, rapeseed meal) or any combination thereof.

45. A method of improving one or more performance parameters of an animal comprising administering to one or more animals the lysozyme as disclosed in any of items 1 to 8, the granule of any of items 9 to 24, the polypeptide of any of items 25 to 29, the composition of any of items 30 to 32, the animal feed additive of any of items 33 to 40, the animal feed of any of items 41 to 42 or the pelleted animal feed of any of items 43 to 44.

46. The method of item 45, wherein improving the performance of an animal means improved body weight gain, improved European Production Efficiency Factor (EPEF) and/or improved FCR.

47. A method of preparing an animal feed comprising mixing the lysozyme as disclosed in any of items 1 to 8, the granule of any of items 9 to 24, the polypeptide of any of items 25 to 29, the composition of any of items 30 to 32 or the animal feed additive of any of items 33 to 40 with plant based material.

48. The method of item 47, wherein the plant based material is selected from the group consisting of legumes, cereals, oats, rye, barley, wheat, maize, corn, sorghum, switchgrass, millet, pearl millet, foxtail millet, soybean, wild soybean, beans, lupin, tepary bean, scarlet runner bean, slimjim bean, lima bean, French bean, Broad bean (fava bean), chickpea, lentil, peanut, Spanish peanut, canola, rapeseed (oilseed rape), rice, beet, cabbage, sugar beet, spinach, quinoa, or pea, in a processed form thereof (such as soybean meal, rapeseed meal) or any combination thereof.

49. A method for improving the nutritional value of an animal feed, comprising adding to the feed the lysozyme as disclosed in any of items 1 to 8, the granule of any of items 9 to 24, the polypeptide of any of items 25 to 29, the composition of any of items 30 to 32 or the animal feed additive of any of items 33 to 40.

50. The method of item 49, wherein the feed is selected from the group consisting of legumes, cereals, oats, rye, barley, wheat, maize, corn, sorghum, switchgrass, millet, pearl millet, foxtail millet, soybean, wild soybean, beans, lupin, tepary bean, scarlet runner bean, slimjim bean, lima bean, French bean, Broad bean (fava bean), chickpea, lentil, peanut, Spanish peanut, canola, rapeseed (oilseed rape), rice, beet, cabbage, sugar beet, spinach, quinoa, or pea, in a processed form thereof (such as soybean meal, rapeseed meal) or any combination thereof.

51. A polynucleotide encoding the polypeptide of any of items 25 to 29.

52. A nucleic acid construct or expression vector comprising the polynucleotide of item 51 operably linked to one or more control sequences that direct the production of the polypeptide in an expression host.

53. A recombinant host cell comprising the polynucleotide of item 51 operably linked to one or more control sequences that direct the production of the polypeptide.

54. A method of producing the polypeptide of any of items 25 to 29, comprising:
(a) cultivating a cell, which in its wild-type form produces the polypeptide, under conditions conductive for production of the polypeptide; and
(b) recovering the polypeptide.

55. A method of producing the polypeptide of any of items 25 to 29, comprising:
(a) cultivating a host cell of item 53 under conditions conducive for production of the polypeptide; and
(b) recovering the polypeptide.

56. A transgenic plant, plant part or plant cell transformed with a polynucleotide encoding the polypeptide of any of items 25 to 29.

57. A whole broth formulation or cell culture composition comprising a polypeptide of any of items 25 to 29.

58. Use of the lysozyme as disclosed in any of any of items 1 to 8, the granule of any of items 9 to 24, the polypeptide of any of items 25 to 29, the composition of any of items 30 to 32 or the animal feed additive of any of items 33 to 40:
in animal feed;
in animal feed additives;
in the preparation of a composition for use in animal feed;
for improving the nutritional value of an animal feed; and/or for improving one or more performance parameters in an animal.

59. An isolated GH25 polypeptide having lysozyme activity selected from the group consisting of:
(a) a polypeptide having at least 80% sequence identity to the polypeptide of SEQ ID NO: 3;
(b) a polypeptide having at least 84% sequence identity to the polypeptide of SEQ ID NO: 6;
(c) a polypeptide having at least 80% sequence identity to the polypeptide of SEQ ID NO: 9;
(d) a polypeptide having at least 80% sequence identity to the polypeptide of SEQ ID NO: 12;
(e) a polypeptide having at least 80% sequence identity to the polypeptide of SEQ ID NO: 15;
(f) a polypeptide having at least 80% sequence identity to the polypeptide of SEQ ID NO: 18;
(g) a polypeptide having at least 80% sequence identity to the polypeptide of SEQ ID NO: 21;
(h) a polypeptide having at least 80% sequence identity to the polypeptide of SEQ ID NO: 24;
(i) a polypeptide having at least 80% sequence identity to the polypeptide of SEQ ID NO: 27;
(j) a polypeptide having at least 84% sequence identity to the polypeptide of SEQ ID NO: 30;
(k) a polypeptide having at least 80% sequence identity to the polypeptide of SEQ ID NO: 33;
(l) a polypeptide having at least 80% sequence identity to the polypeptide of SEQ ID NO: 36;
(m) a polypeptide having at least 84% sequence identity to the polypeptide of SEQ ID NO: 39;
(n) a polypeptide having at least 80% sequence identity to the polypeptide of SEQ ID NO: 42;
(o) a polypeptide having at least 80% sequence identity to the polypeptide of SEQ ID NO: 45;
(p) a polypeptide having at least 80% sequence identity to the polypeptide of SEQ ID NO: 48;
(q) a polypeptide having at least 80% sequence identity to the polypeptide of SEQ ID NO: 51;
(r) a polypeptide having at least 80% sequence identity to the polypeptide of SEQ ID NO: 54;
(s) a polypeptide having at least 80% sequence identity to the polypeptide of SEQ ID NO: 57;
(t) a polypeptide having at least 80% sequence identity to the polypeptide of SEQ ID NO: 60;
(u) a polypeptide having at least 80% sequence identity to the polypeptide of SEQ ID NO: 63;
(v) a polypeptide having at least 80% sequence identity to the polypeptide of SEQ ID NO: 66;
(w) a polypeptide having at least 80% sequence identity to the polypeptide of SEQ ID NO: 69;
(x) a polypeptide having at least 84% sequence identity to the polypeptide of SEQ ID NO: 72;
(y) a polypeptide having at least 80% sequence identity to the polypeptide of SEQ ID NO: 75;
(z) a polypeptide having at least 80% sequence identity to the polypeptide of SEQ ID NO: 80;
(aa) a polypeptide having at least 80% sequence identity to the polypeptide of SEQ ID NO: 83;
(ab) a polypeptide having at least 80% sequence identity to the polypeptide of SEQ ID NO: 86;
(ac) a polypeptide having at least 86% sequence identity to the polypeptide of SEQ ID NO: 89;
(ad) a polypeptide having at least 82% sequence identity to the polypeptide of SEQ ID NO: 92;
(ae) a polypeptide having at least 82% sequence identity to the polypeptide of SEQ ID NO: 95;
(af) a polypeptide having at least 80% sequence identity to the polypeptide of SEQ ID NO: 98;
(ag) a polypeptide having at least 80% sequence identity to the polypeptide of SEQ ID NO: 101;
(ah) a polypeptide having at least 80% sequence identity to the polypeptide of SEQ ID NO: 104;
(ai) a polypeptide having at least 80% sequence identity to the polypeptide of SEQ ID NO: 107;
(aj) a polypeptide having at least 82% sequence identity to the polypeptide of SEQ ID NO: 110;
(ak) a polypeptide having at least 81% sequence identity to the polypeptide of SEQ ID NO: 113;
(al) a polypeptide having at least 80% sequence identity to the polypeptide of SEQ ID NO: 116;
(am) a polypeptide having at least 80% sequence identity to the polypeptide of SEQ ID NO: 119;
(an) a polypeptide having at least 80% sequence identity to the polypeptide of SEQ ID NO: 122;
(ao) a polypeptide having at least 80% sequence identity to the polypeptide of SEQ ID NO: 125;
(ap) a polypeptide having at least 80% sequence identity to the polypeptide of SEQ ID NO: 128;
(aq) a polypeptide having at least 80% sequence identity to the polypeptide of SEQ ID NO: 131;
(ar) a polypeptide having at least 80% sequence identity to the polypeptide of SEQ ID NO: 134;
(as) a polypeptide having at least 80% sequence identity to the polypeptide of SEQ ID NO: 137;
(at) a polypeptide having at least 80% sequence identity to the polypeptide of SEQ ID NO: 140;
(au) a polypeptide having at least 80% sequence identity to the polypeptide of SEQ ID NO: 143;
(av) a polypeptide having at least 80% sequence identity to the polypeptide of SEQ ID NO: 146;
(aw) a polypeptide having at least 80% sequence identity to the polypeptide of SEQ ID NO: 149;
(ax) a polypeptide having at least 80% sequence identity to the polypeptide of SEQ ID NO: 152;
(ay) a polypeptide having at least 80% sequence identity to the polypeptide of SEQ ID NO: 155;
(az) a polypeptide having at least 80% sequence identity to the polypeptide of SEQ ID NO: 158;
(ba) a polypeptide having at least 80% sequence identity to the polypeptide of SEQ ID NO: 221;
(bb) a polypeptide having at least 80% sequence identity to the polypeptide of SEQ ID NO: 224;
(bc) a polypeptide having at least 80% sequence identity to the polypeptide of SEQ ID NO: 227;
(bd) a polypeptide having at least 80% sequence identity to the polypeptide of SEQ ID NO: 230;
(be) a polypeptide having at least 80% sequence identity to the polypeptide of SEQ ID NO: 233;
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27 or 28 positions;
(bg) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal His-tag and/or HQ-tag;
(bh) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) and a N-terminal and/or C-terminal extension of up to 10 amino acids, e.g. 1, 2, 3, 4, 5, 6, 7, 8, 9 or 10 amino acids; and
(bi) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be) or (bf) having lysozyme activity and having at least 90% of the length of the mature polypeptide.

60. The polypeptide according to embodiment 59, wherein the polypeptide is selected from the group consisting of:
(a) a polypeptide having at least 90% sequence identity to the polypeptide of SEQ ID NO: 3;
(b) a polypeptide having at least 90% sequence identity to the polypeptide of SEQ ID NO: 6;
(c) a polypeptide having at least 90% sequence identity to the polypeptide of SEQ ID NO: 9;
(d) a polypeptide having at least 90% sequence identity to the polypeptide of SEQ ID NO: 12;
(e) a polypeptide having at least 90% sequence identity to the polypeptide of SEQ ID NO: 15;
(f) a polypeptide having at least 90% sequence identity to the polypeptide of SEQ ID NO: 18;
(g) a polypeptide having at least 90% sequence identity to the polypeptide of SEQ ID NO: 21;
(h) a polypeptide having at least 90% sequence identity to the polypeptide of SEQ ID NO: 24;
(i) a polypeptide having at least 90% sequence identity to the polypeptide of SEQ ID NO: 27;
(j) a polypeptide having at least 90% sequence identity to the polypeptide of SEQ ID NO: 30;
(k) a polypeptide having at least 90% sequence identity to the polypeptide of SEQ ID NO: 33;
(l) a polypeptide having at least 90% sequence identity to the polypeptide of SEQ ID NO: 36;
(m) a polypeptide having at least 90% sequence identity to the polypeptide of SEQ ID NO: 39;
(n) a polypeptide having at least 90% sequence identity to the polypeptide of SEQ ID NO: 42;
(o) a polypeptide having at least 90% sequence identity to the polypeptide of SEQ ID NO: 45;
(p) a polypeptide having at least 90% sequence identity to the polypeptide of SEQ ID NO: 48;
(q) a polypeptide having at least 90% sequence identity to the polypeptide of SEQ ID NO: 51;
(r) a polypeptide having at least 90% sequence identity to the polypeptide of SEQ ID NO: 54;
(s) a polypeptide having at least 90% sequence identity to the polypeptide of SEQ ID NO: 57;
(t) a polypeptide having at least 90% sequence identity to the polypeptide of SEQ ID NO: 60;
(u) a polypeptide having at least 90% sequence identity to the polypeptide of SEQ ID NO: 63;
(v) a polypeptide having at least 90% sequence identity to the polypeptide of SEQ ID NO: 66;
(w) a polypeptide having at least 90% sequence identity to the polypeptide of SEQ ID NO: 69;
(x) a polypeptide having at least 90% sequence identity to the polypeptide of SEQ ID NO: 72;
(y) a polypeptide having at least 90% sequence identity to the polypeptide of SEQ ID NO: 75;
(z) a polypeptide having at least 90% sequence identity to the polypeptide of SEQ ID NO: 80;
(aa) a polypeptide having at least 90% sequence identity to the polypeptide of SEQ ID NO: 83;
(ab) a polypeptide having at least 90% sequence identity to the polypeptide of SEQ ID NO: 86;
(ac) a polypeptide having at least 90% sequence identity to the polypeptide of SEQ ID NO: 89;
(ad) a polypeptide having at least 90% sequence identity to the polypeptide of SEQ ID NO: 92;
(ae) a polypeptide having at least 90% sequence identity to the polypeptide of SEQ ID NO: 95;
(af) a polypeptide having at least 90% sequence identity to the polypeptide of SEQ ID NO: 98;
(ag) a polypeptide having at least 90% sequence identity to the polypeptide of SEQ ID NO: 101;
(ah) a polypeptide having at least 90% sequence identity to the polypeptide of SEQ ID NO: 104;
(ai) a polypeptide having at least 90% sequence identity to the polypeptide of SEQ ID NO: 107;
(aj) a polypeptide having at least 90% sequence identity to the polypeptide of SEQ ID NO: 110;
(ak) a polypeptide having at least 90% sequence identity to the polypeptide of SEQ ID NO: 113;
(al) a polypeptide having at least 90% sequence identity to the polypeptide of SEQ ID NO: 116;
(am) a polypeptide having at least 90% sequence identity to the polypeptide of SEQ ID NO: 119;
(an) a polypeptide having at least 90% sequence identity to the polypeptide of SEQ ID NO: 122;
(ao) a polypeptide having at least 90% sequence identity to the polypeptide of SEQ ID NO: 125;
(ap) a polypeptide having at least 90% sequence identity to the polypeptide of SEQ ID NO: 128;
(aq) a polypeptide having at least 90% sequence identity to the polypeptide of SEQ ID NO: 131;
(ar) a polypeptide having at least 90% sequence identity to the polypeptide of SEQ ID NO: 134;
(as) a polypeptide having at least 90% sequence identity to the polypeptide of SEQ ID NO: 137;
(at) a polypeptide having at least 90% sequence identity to the polypeptide of SEQ ID NO: 140;
(au) a polypeptide having at least 90% sequence identity to the polypeptide of SEQ ID NO: 143;
(av) a polypeptide having at least 90% sequence identity to the polypeptide of SEQ ID NO: 146;
(aw) a polypeptide having at least 90% sequence identity to the polypeptide of SEQ ID NO: 149;
(ax) a polypeptide having at least 90% sequence identity to the polypeptide of SEQ ID NO: 152;
(ay) a polypeptide having at least 90% sequence identity to the polypeptide of SEQ ID NO: 155;
(az) a polypeptide having at least 90% sequence identity to the polypeptide of SEQ ID NO: 158;
(ba) a polypeptide having at least 90% sequence identity to the polypeptide of SEQ ID NO: 221;
(bb) a polypeptide having at least 90% sequence identity to the polypeptide of SEQ ID NO: 224;
(bc) a polypeptide having at least 90% sequence identity to the polypeptide of SEQ ID NO: 227;
(bd) a polypeptide having at least 90% sequence identity to the polypeptide of SEQ ID NO: 230;
(be) a polypeptide having at least 90% sequence identity to the polypeptide of SEQ ID NO: 233; and
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions.

61. An isolated GH25 polypeptide having lysozyme activity selected from the group consisting of:
(a) a polypeptide having at least 90% sequence identity to the polypeptide of SEQ ID NO: 3;
(b) a polypeptide having at least 90% sequence identity to the polypeptide of SEQ ID NO: 6;
(e) a polypeptide having at least 90% sequence identity to the polypeptide of SEQ ID NO: 15;
(f) a polypeptide having at least 90% sequence identity to the polypeptide of SEQ ID NO: 18;
(g) a polypeptide having at least 90% sequence identity to the polypeptide of SEQ ID NO: 21;
(h) a polypeptide having at least 90% sequence identity to the polypeptide of SEQ ID NO: 24;
(k) a polypeptide having at least 90% sequence identity to the polypeptide of SEQ ID NO: 33;
(l) a polypeptide having at least 90% sequence identity to the polypeptide of SEQ ID NO: 36;
(m) a polypeptide having at least 90% sequence identity to the polypeptide of SEQ ID NO: 39;
(0) a polypeptide having at least 90% sequence identity to the polypeptide of SEQ ID NO: 45;
(p) a polypeptide having at least 90% sequence identity to the polypeptide of SEQ ID NO: 48;
(q) a polypeptide having at least 90% sequence identity to the polypeptide of SEQ ID NO: 51;
(r) a polypeptide having at least 90% sequence identity to the polypeptide of SEQ ID NO: 54;
(s) a polypeptide having at least 90% sequence identity to the polypeptide of SEQ ID NO: 57;
(t) a polypeptide having at least 90% sequence identity to the polypeptide of SEQ ID NO: 60;
(x) a polypeptide having at least 90% sequence identity to the polypeptide of SEQ ID NO: 72;
(y) a polypeptide having at least 90% sequence identity to the polypeptide of SEQ ID NO: 75;
(z) a polypeptide having at least 90% sequence identity to the polypeptide of SEQ ID NO: 80;
(ac) a polypeptide having at least 90% sequence identity to the polypeptide of SEQ ID NO: 89;
(ae) a polypeptide having at least 90% sequence identity to the polypeptide of SEQ ID NO: 95;
(af) a polypeptide having at least 90% sequence identity to the polypeptide of SEQ ID NO: 98;
(ag) a polypeptide having at least 90% sequence identity to the polypeptide of SEQ ID NO: 101;
(ak) a polypeptide having at least 90% sequence identity to the polypeptide of SEQ ID NO: 113;
(al) a polypeptide having at least 90% sequence identity to the polypeptide of SEQ ID NO: 116;
(am) a polypeptide having at least 90% sequence identity to the polypeptide of SEQ ID NO: 119;
(an) a polypeptide having at least 90% sequence identity to the polypeptide of SEQ ID NO: 122;
(aq) a polypeptide having at least 90% sequence identity to the polypeptide of SEQ ID NO: 131;
(as) a polypeptide having at least 90% sequence identity to the polypeptide of SEQ ID NO: 137;
(au) a polypeptide having at least 90% sequence identity to the polypeptide of SEQ ID NO: 143;
(av) a polypeptide having at least 90% sequence identity to the polypeptide of SEQ ID NO: 146;
(aw) a polypeptide having at least 90% sequence identity to the polypeptide of SEQ ID NO: 149;
(ax) a polypeptide having at least 90% sequence identity to the polypeptide of SEQ ID NO: 152;
(ay) a polypeptide having at least 90% sequence identity to the polypeptide of SEQ ID NO: 155;
(ba) a polypeptide having at least 90% sequence identity to the polypeptide of SEQ ID NO: 221;
(bd) a polypeptide having at least 90% sequence identity to the polypeptide of SEQ ID NO: 230;
(be) a polypeptide having at least 90% sequence identity to the polypeptide of SEQ ID NO: 233; and
(bf) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 80, SEQ ID NO: 83, SEQ ID NO: 86, SEQ ID NO: 89, SEQ ID NO: 92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 128, SEQ ID NO: 131, SEQ ID NO: 134, SEQ ID NO: 137, SEQ ID NO: 140, SEQ ID NO: 143, SEQ ID NO: 146, SEQ ID NO: 149, SEQ ID NO: 152, SEQ ID NO: 155, SEQ ID NO: 158, SEQ ID NO: 221, SEQ ID NO: 224, SEQ ID NO: 227, SEQ ID NO: 230 and SEQ ID NO: 233, wherein the variant has lysozyme activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions.

62. The polypeptide according to embodiments 59 to 61 having improved lysozyme activity a) compared to the lysozyme activity of hen eggwhite lysozyme (HEWL) or b) compared to the lysozyme activity of SEQ ID NO: 39 as determined by the Method for the Determination of Lysozyme Activity Against Micrococcus lysodeikticus.

63. The polypeptide according to embodiment 59 to 61 having improved lysozyme activity a) compared to the lysozyme activity of hen eggwhite lysozyme (HEWL) and/or b) compared to the lysozyme activity of SEQ ID NO: 39 as determined by any one of i) Method for the Determination of Lysozyme Activity Against Micrococcus lysodeikticus and ii) Method for the Determination of Lysozyme Activity Against Lactobacillus johnsonii.

64. The peptide according any of embodiment 59 to 63, wherein the polypeptide comprises or consists of amino acids 1 to 207 of SEQ ID NO: 3, amino acids 1 to 201 of SEQ ID NO: 6, amino acids 1 to 208 of SEQ ID NO: 15, amino acids 1 to 207 of SEQ ID NO: 18, amino acids 1 to 208 of SEQ ID NO: 21, amino acids 1 to 207 of SEQ ID NO: 24, amino acids 1 to 208 of SEQ ID NO: 33, amino acids 1 to 208 of SEQ ID NO: 36, amino acids 1 to 206 of SEQ ID NO: 39, amino acids 1 to 216 of SEQ ID NO: 45, amino acids 1 to 218 of SEQ ID NO: 48, amino acids 1 to 204 of SEQ ID NO: 51, amino acids 1 to 203 of SEQ ID NO: 54, amino acids 1 to 208 of SEQ ID NO: 60, amino acids 1 to 208 of SEQ ID NO: 72, amino acids 1 to 217 of SEQ ID NO: 75, amino acids 1 to 208 of SEQ ID NO: 80, amino acids 1 to 207 of SEQ ID NO: 89, amino acids 1 to 201 of SEQ ID NO: 95, amino acids 1 to 202 of SEQ ID NO: 98, amino acids 1 to 206 of SEQ ID NO: 101, amino acids 1 to 202 of SEQ ID NO: 113, amino acids 1 to 206 of SEQ ID NO: 116, amino acids 1 to 207 of SEQ ID NO: 119, amino acids 1 to 208 of SEQ ID NO: 122, amino acids 1 to 215 of SEQ ID NO: 125, amino acids 1 to 217 of SEQ ID NO: 128, amino acids 1 to 214 of SEQ ID NO: 131, amino acids 1 to 203 of SEQ ID NO: 137, amino acids 1 to 207 of SEQ ID NO: 143, amino acids 1 to 208 of SEQ ID NO: 146, amino acids 1 to 207 of SEQ ID NO: 155, amino acids 1 to 207 of SEQ ID NO: 221, amino acids 1 to 207 of SEQ ID NO: 230 or amino acids 1 to 207 of SEQ ID NO: 233.

65. The polypeptide according to embodiment 59 to 63, wherein the polypeptide is selected from the group consisting of a polpypeptide having at least 90% sequence identity to the polypeptide of SEQ ID NO: 15, a polpypeptide having at least 90% sequence identity to the polypeptide of SEQ ID NO: 36, a polpypeptide having at least 90% sequence identity to the polypeptide of SEQ ID NO: 75, a polpypeptide having at least 90% sequence identity to the polypeptide of SEQ ID NO: 80, and a polpypeptide having at least 90% sequence identity to the polypeptide of SEQ ID NO: 119.

66. A method of hydrolysing peptidoglycan in bacterial cell walls comprising treating bacterial cells with one or more GH25 polypeptides having lysozyme activity, wherein the polypeptide is as defined in any one of embodiment 59 to 63.

67. A method of increasing the digestibility of peptidoglycans in animal feed comprising the use of a peptide as defined in any of embodiment 59 to 63.

68. An animal feed additive comprising the polypeptide as defined in any of embodiment 59 to 63.

68. An animal feed additive according to embodiment 67 further comprising one or more components selected from the list consisting of:
one or more vitamins;
one or more minerals;
one or more amino acids;
one or more prebiotics;
one or more organic acids;
one or more other feed ingredients;
one or more additional enzymes;
one or more probiotics; and
one or more phytogenics

69. An animal feed comprising the polypeptide as defined in any of embodiment 59 to 63.

70. An animal feed according to embodiment 69 further comprising plant based material.

71. A composition comprising the polypeptide of any of embodiment 59 to 63.

72. A polynucleotide encoding the polypeptide of any of embodiment 59 to 63.

73. A recombinant host cell comprising the polynucleotide of embodiment 72 operably linked to one or more control sequences that direct the production of the polypeptide.

74. A method of producing the polypeptide of any of embodiment 59 to 63 comprising:
(a) cultivating a host cell of claim 16 under conditions conducive for production of the polypeptide; and
(b) recovering the polypeptide.

75. Use of the polypeptide as disclosed in any of embodiment 59 to 63:
in animal feed;
in animal feed additives;
in the preparation of a composition for use in animal feed; and/or for improving the intestinal health in an animal.

76. A zootechnical additive for use in feed for poultry or swine, said additive comprising the polypeptide as defined in any of embodiment 59 to 63.

77. A method of improving the intestinal health in an animal comprising reducing the amount of dead Lactobacillus johnsonii cells in the digestive tract of said animal, comprising feeding the animal with a feed or feed additive comprising a polypeptide as defined in any of embodiments 59 to 63.

78. A method of improving the intestinal health in an animal comprising reducing the amount of dead Lactobacillus johnsonii cells, or cell wall debris therefrom, in the digestive tract of said animal, comprising feeding the animal with a feed or feed additive comprising a polypeptide selected from the group consisting of a polpypeptide having at least 90% sequence identity to the polypeptide of SEQ ID NO: 15, a polpypeptide having at least 90% sequence identity to the polypeptide of SEQ ID NO: 36, a polpypeptide having at least 90% sequence identity to the polypeptide of SEQ ID NO: 75, a polpypeptide having at least 90% sequence identity to the polypeptide of SEQ ID NO: 80, and a polpypeptide having at least 90% sequence identity to the polypeptide of SEQ ID NO: 119, preferably selected from the group consisting of a polpypeptide having at least 90% sequence identity to the polypeptide of SEQ ID NO: 15, a polpypeptide having at least 90% sequence identity to the polypeptide of SEQ ID NO: 36, a polpypeptide having at least 90% sequence identity to the polypeptide of SEQ ID NO: 80, and a polpypeptide having at least 90% sequence identity to the polypeptide of SEQ ID NO: 119, more preferably a polpypeptide having at least 90% sequence identity to the polypeptide of SEQ ID NO: 36 and a polpypeptide having at least 90% sequence identity to the polypeptide of SEQ ID NO: 80.

79. A method of improving the intestinal health in an animal comprising reducing the amount of dead Lactobacillus johnsonii cells, or cell wall debris therefrom, in the digestive tract of said animal, comprising feeding the animal with a feed or feed additive comprising a polypeptide selected from the group consisting of a polypeptide having at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO 159.

80. A method of promoting the elimination of dead Lactobacillus johnsonii cells, or cell wall debris therefrom, from the digestive tract of an animal comprising feeding said animal a source of a polypeptide as defined in any of embodiment 61 to 63.

81. A method of promoting the elimination of dead Lactobacillus johnsonii cells, or cell wall debris therefrom, from the digestive tract of an animal comprising feeding said animal a source of a polypeptide selected from the group consisting of a polpypeptide having at least 90% sequence identity to the polypeptide of SEQ ID NO: 15, a polpypeptide having at least 90% sequence identity to the polypeptide of SEQ ID NO: 36, a polpypeptide having at least 90% sequence identity to the polypeptide of SEQ ID NO: 75, a polpypeptide having at least 90% sequence identity to the polypeptide of SEQ ID NO: 80, and a polpypeptide having at least 90% sequence identity to the polypeptide of SEQ ID NO: 119, preferably selected from the group consisting of a polpypeptide having at least 90% sequence identity to the polypeptide of SEQ ID NO: 15, a polpypeptide having at least 90% sequence identity to the polypeptide of SEQ ID NO: 36, a polpypeptide having at least 90% sequence identity to the polypeptide of SEQ ID NO: 80, and a polpypeptide having at least 90% sequence identity to the polypeptide of SEQ ID NO: 119, more preferably a polpypeptide having at least 90% sequence identity to the polypeptide of SEQ ID NO: 36 and a polpypeptide having at least 90% sequence identity to the polypeptide of SEQ ID NO: 80.

82. A method of promoting the elimination of dead Lactobacillus johnsonii cells, or cell wall debris therefrom, from the digestive tract of an animal comprising feeding said animal a source of a polypeptide selected from the group consisting of a polypeptide having at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO 159.

83. The method according to any one of embodiments 77 to 82 wherein the polypeptide has a lysozyme activity against Lactobacillus johnsonii at 5 ppm that increases optical density (OD) measurement at 405 nm of at least 0.20 as determined by Method for the Determination of Lysozyme Activity Against Lactobacillus johnsonii.

The present disclosure is further described by the following examples that should not be construed as limiting the scope of the present disclosure.

### EXAMPLES

### Strains

Escherichia coli Top-10 strain was purchased from Invitrogen (Life Technologies, Carlsbad, CA, USA) and was used to propagate the expression vectors encoding for lysozyme polypeptides.

Aspergillus oryzae strain MT3568 was used for heterologous expression of the lysozyme polypeptide encoding sequences. A. oryzae MT3568 is an amdS (acetamidase) disrupted gene derivative of Aspergillus oryzae JaL355 (WO 2002/40694) in which pyrG auxotrophy was restored by disrupting the A. oryzae acetamidase (amdS) gene with the pyrG gene.

Aspergillus niger MBin118 is disclosed in WO 2004/090155.

The fungal strain NN047801 was isolated from litter samples collected from China, in 2003 by the dilution plate method with PDA medium, 25 C. It was then purified by transferring a single conidium onto a PDA agar plate. The strain NN047801 was identified as Sporormia fimetaria, based on both morphological characteristics and ITS rDNA sequence.

Strain Poronia punctata NN009607 was isolated from Sweden. The strain was inoculated onto a PDA plate and incubated for 7 days at 26° C. in the darkness. Mycelia and spores from the plate were inoculated into 500 ml shake flasks containing 100 mls of YPG medium. The flasks were incubated for 5 days at 26° C. with shaking at 100 rpm.

The fungal strain NN054002 was isolated from soil samples collected from Tibet, China, in 2011 by the dilution plate method with PDA medium, 10 C. It was then purified by transferring a single conidium onto a PDA agar plate. The strain NN054002 was identified as Lecanicillium sp., based on both morphological characteristics and ITS rDNA sequence.

Strain Onygena equina NN056731 was isolated on Gotland, Sweden. The strain was inoculated onto a PDA plate and incubated for 18 days at 20° C. in the darkness. Mycelia and spores from the plate were inoculated into 500 ml shake flasks containing 100 mls of YPG medium. The flasks were incubated for 18 days at 20° C. with shaking at 100 rpm.

Strain Purpureocillium lilacinum NN070261 was isolated from a soil sample from the United States, and inoculated onto a PDA plate and incubated for 8 days at 26° C. in the darkness. Mycelia and spores from the plate were inoculated into 500 ml shake flasks containing 100 mls of YPG medium. The flasks were incubated for 3 days at 26° C. with shaking at 100 rpm.

Strain Trichobolus zukalii CBS720.69 was purchased CBS-KNAW Fungal Biodiversity Centre, and inoculated onto a PDA plate and incubated for 7 days at 26° C. in the darkness. Mycelia and spores from the plate were inoculated into 500 ml shake flasks containing 100 mls of YPG medium. The flasks were incubated for 3 days at 26° C. with shaking at 100 rpm. Strain Penicillium citrinum NN070248 was isolated from Antarctica. The strain was inoculated onto a PDA plate and incubated for 7 days at 26° C. in the darkness. Mycelia and spores from the plate were inoculated into 500 ml shake flasks containing 100 mls of YPG medium. The flasks were incubated for 6 days at 26° C. with shaking at 100 rpm.

The fungal strain NN057876 was from Professor Cai Lei in Institude of Microbiology, CAS. The strain was collected from a compost sample from China, in 2014 by the dilution plate method with PDA medium, 37 C. It was then purified by transferring a single conidium onto a PDA agar plate. The strain NN047801 was identified as Cladorrhinum bulbillosum, based on both morphological characteristics and ITS rDNA sequence.

Strain Umbelopsis westeae CBS871.85 was purchased CBS-KNAW Fungal Biodiversity Centre, and inoculated onto a PDA plate and incubated for 7 days at 26° C. in the darkness. Mycelia and spores from the plate were inoculated into 500 ml shake flasks containing 100 mls of YPG medium. The flasks were incubated for 3 days at 26° C. with shaking at 100 rpm.

The fungal strain NN058098 was isolated from soil samples collected from Guizhou Province, China, in 2014 by the dilution plate method with PDA medium pH3, 25 C. It was then purified by transferring a single conidium onto a PDA agar plate. The strain NN058098 was identified as Zygomycetes sp., based on both morphological characteristics and ITS rDNA sequence.

The fungal strain NN058297 was isolated from litter samples collected from Guizhou Province, China, in 2014 by the dilution plate method with PDA medium, 25 C. It was then purified by transferring a single conidium onto a PDA agar plate. The strain NN058297 was identified as Chaetomium cupreum, based on both morphological characteristics and ITS rDNA sequence.

Cordyceps cardinalis CBS113411 was purchased from CBS-KNAW Fungal Biodiversity Centre, Uppsalalaan 8, 3584 CT, Utrecht, The Netherlands and inoculated onto a PDA plate and incubated for 8 days at 26° C. in the darkness. Several mycelia-PDA plugs were inoculated into 500 ml shake flasks containing 100 mls of YPG medium. The flasks were incubated for 5 days at 26° C. with shaking at 100 rpm.

The fungal strain NN044232 was isolated from soil samples collected from China, in 1998 by the dilution plate method with PDA medium, pH7, 25 C. It was then purified by transferring a single conidium onto a PDA agar plate. The strain NN044232 was identified as Penicillium sp. ‘qii’, based on both morphological characteristics and ITS rDNA sequence. The fungal strain NN058292 was isolated from soil samples collected from Guizhou Province, China, in 2014 by the dilution plate method with PDA medium pH3. It was then purified by transferring a single conidium onto a PDA agar plate. The strain NN058292 was identified as Aspergillus sp. nov. XZ2609, based on both morphological characteristics and ITS rDNA sequence.

The fungal strain NN058101 was isolated from soil samples collected from Guizhou Province, China, in 2014 by the dilution plate method with PDA medium pH3, 25 C. It was then purified by transferring a single conidium onto a PDA agar plate. The strain NN058101 was identified as Paecilomyces sp. XZ2658, based on both morphological characteristics and ITS rDNA sequence.

The fungal strain NN046782 was isolated from soil samples collected from China, in 1998 by the dilution plate method with YG medium, pH7, 45 C. It was then purified by transferring a single conidium onto another YG plate. The strain NN046782 was identified as Rhizomucor pusillus T185-2, based on both morphological characteristics and ITS rDNA sequence.

The fungal strain NN051201 was isolated from litter samples collected from China, in 2008 by the dilution plate method with PDA medium, 25 C. It was then purified by transferring a single conidium onto a PDA agar plate. The strain NN051201 was identified as Pycnidiophora cf. dispera, based on both morphological characteristics and ITS rDNA sequence.

The fungal strain NN000400 was from CBS with access number as CBS104.75. The strain NN000400 was identified as Thermomucor indicae-seudaticae, based on both morphological characteristics and ITS rDNA sequence.

The fungal strain NN054449 was isolated from dead insect samples collected from Tibet, China, in 2012 by the dilution plate method with Horikoshi medium, pH10, 15 C. It was then purified by transferring a single conidium onto a PDA agar plate. The strain NN054449 was identified as Isaria farinosa, based on both morphological characteristics and ITS rDNA sequence.

The fungal strain NN046871 was isolated from soil samples collected from China, in 2000 by the dilution plate method with YG medium, 37 C. It was then purified by transferring a single conidium onto a PDA agar plate. The strain NN046871 was identified as Zopfiella sp., based on both morphological characteristics and ITS rDNA sequence.

Strain Malbranchea flava CBS132.77 was purchased CBS-KNAW Fungal Biodiversity Centre, and inoculated onto a PDA plate and incubated for 14 days at 26° C. in the darkness. Mycelia and spores from the plate were inoculated into 500 ml shake flasks containing 100 mls of YPG medium. The flasks were incubated for 20 days at 26° C. with shaking at 100 rpm.

Strain Hypholoma polytrichi NN057040 was isolated from the Utsjoki region at the northernmost tip of Finland (69°45′ N, 27° 01′ E). The strain was inoculated onto a PDA plate and incubated for 10 days at 26° C. in the darkness. Mycelia and spores from the plate were inoculated into 500 ml shake flasks containing 100 mls of YPG medium. The flasks were incubated for 15 days at 26° C. with shaking at 100 rpm.

Strain Aspergillus deflectus NN051662 was isolated from Jilin province in China. Please consult NZ China for propagation and DNA isolation conditions otherwise, the following would work: The strain was inoculated onto a PDA plate and incubated for 7 days at 26° C. in the darkness. Mycelia and spores from the plate were inoculated into 500 ml shake flasks containing 100 mls of YPG medium. The flasks were incubated for 5 days at 26° C. with shaking at 100 rpm.

Strain Ascobolus stictoideus NN007614 (original code A4-1.1) an isolate from Denmark was purchased from the Institut for Sporeplanter, University of Copenhagen. The strain was inoculated onto a PDA plate and incubated for 7 days at 26° C. in the darkness. Mycelia and spores from the plate were inoculated into 500 ml shake flasks containing 100 mls of YPG medium. The flasks were incubated for 5 days at 26° C. with shaking at 100 rpm.

The fungal strain NN053834 was isolated from soil samples collected from China with a collaboration with the Institute of Microbiology, CAS, in 2011, by the dilution plate method with PDA medium, pH7, 10 C. It was then purified by transferring a single conidium onto a PDA agar plate. The strain NN053834 was identified as Coniochaeta sp., based on both morphological characteristics and ITS rDNA sequence.

The fungal strain NN054033 was isolated from soil samples collected from China with a collaboration with the Institute of Microbiology, CAS, in 2011, by the dilution plate method with PDA medium, pH7, 10 C. It was then purified by transferring a single conidium onto a PDA agar plate. The strain NN054033 was identified as Daldinia fissa, based on both morphological characteristics and ITS rDNA sequence.

The fungal strain NN051542 was isolated from rotten corn stover samples collected from China, in 2007 by the dilution plate method with PDA medium, pH7, 25 C. It was then purified by transferring a single conidium onto a PDA agar plate. The strain NN051542 was identified as Rosellinia sp., based on both morphological characteristics and ITS rDNA sequence.

The fungal strain NN057909 was obtained through a collaboration with Professor Cai Lei in Institude of Microbiology, CAS, in 2014. The strain was collected from China. It was identified as Ascobolus sp., based on both morphological characteristics and ITS rDNA sequence.

The fungal strain NN058306 was isolated from soil samples collected from Guizhou Province, China, in 2014 by the dilution plate method with PDA medium pH3. It was then purified by transferring a single conidium onto a PDA agar plate. The strain NN058306 was identified as Curreya sp., based on both morphological characteristics and ITS rDNA sequence.

The fungal strain NN043614 was isolated from soil samples collected from, China, in 1998 by the dilution plate method with PDA medium, pH7, 25 C. It was then purified by transferring a single conidium onto a PDA agar plate. The strain NN043614 was identified as Coniothyrium sp., based on both morphological characteristics and ITS rDNA sequence.

The fungal strain NN047189 was isolated from soil samples collected from, China, in 1998 by the dilution plate method with PDA medium, pH7, 25 C. It was then purified by transferring a single conidium onto a PDA agar plate. The strain NN047189 was identified as Hypoxylon sp., based on both morphological characteristics and ITS rDNA sequence.

The fungal strain NN043914 was isolated from soil samples collected from, China, in 1998 by the dilution plate method with PDA medium, pH7, 25 C. It was then purified by transferring a single conidium onto a PDA agar plate. The strain NN043914 was identified as Xylariaceae sp., based on both morphological characteristics and ITS rDNA sequence.

The fungal strain NN046688 was isolated from soil samples collected from China, in 1998 by the dilution plate method with YG medium, pH7, 45 C. It was then purified by transferring a single conidium onto another YG plate. The strain NN046688 was identified as Hypoxylon sp., based on both morphological characteristics and ITS rDNA sequence.

The fungal strain NN046158 was isolated from soil samples collected from China, in 1998 by the dilution plate method with PDA medium, pH10, 25 C. It was then purified by transferring a single conidium onto a PDA agar plate. The strain NN046158 was identified as Yunnania penicillata, based on both morphological characteristics and ITS rDNA sequence.

Strain Engyodontium album NN042720 was isolated in Denmark. The strain was inoculated onto a PDA plate and incubated for 5 days at 26° C. in the darkness. Mycelia and spores from the plate were inoculated into 500 ml shake flasks containing 100 mls of YPG medium. The flasks were incubated for 5 days at 26° C. with shaking at 100 rpm.

Strain Metapochonia bulbillosa XZ2653 NN058096 was isolated from a soil sample from Guizhou China. The strain was inoculated onto a PDA plate and incubated for 5 days at 26° C. in the darkness. Mycelia and spores from the plate were inoculated into 500 ml shake flasks containing 100 mls of YPG medium. The flasks were incubated for 5 days at 26° C. with shaking at 100 rpm.

Strain Hamigera paravellanea A3661 NN102174 was isolated in Japan. The strain was inoculated onto a PDA plate and incubated for 5 days at 26° C. in the darkness. Mycelia and spores from the plate were inoculated into 500 ml shake flasks containing 100 mls of YPG medium. The flasks were incubated for 5 days at 26° C. with shaking at 100 rpm.

The fungal strain NN047633 was isolated from dead insect samples collected from China, in 2002 by the dilution plate method with PDA medium, pH7, 25 C. It was then purified by transferring a single conidium onto a PDA agar plate. The strain NN047633 was identified as Metarhizium iadini, based on both morphological characteristics and ITS rDNA sequence.

The fungal strain NN044936 was isolated from soil samples collected from China, in 1998 by the dilution plate method with YG medium, pH7, 45 C. It was then purified by transferring a single conidium onto another YG agar plate. The strain NN044936 was identified as Thermoascus aurantiacus, based on both morphological characteristics and ITS rDNA sequence.

The fungal strain NN071248 was isolated from soil samples collected from Shandong Province, China, in 2015 by the dilution plate method with Horikoshi medium, pH10, 25 C. It was then purified by transferring a single conidium onto another Horikoshi plate. The strain NN071248 was identified as Clonostachys rossmaniae, based on both morphological characteristics and ITS rDNA sequence.

The fungal strain NN046572 was isolated from soil samples collected from China, in 1998 by the dilution plate method with PDA medium, pH7, 25 C. It was then purified by transferring a single conidium onto a PDA agar plate. The strain NN046572 was identified as Simplicillium obclavatum, based on both morphological characteristics and ITS rDNA sequence.

The fungal strain NN058092 was isolated from soil samples collected from Guizhou Province, China, in 2014 by the dilution plate method with PDA medium pH3, 25 C. It was then purified by transferring a single conidium onto a PDA agar plate. The strain NN058092 was identified as Aspergillus inflatus, based on both morphological characteristics and ITS rDNA sequence.

The fungal strain NN071247 was isolated from soil samples collected from Shandong Province, China, in 2015 by the dilution plate method with Horikoshi medium, pH10, 25 C. It was then purified by transferring a single conidium onto another Horikoshi agar plate. The strain NN071247 was identified as Paracremonium inflatum, based on both morphological characteristics and ITS rDNA sequence.

The fungal strain NN046156 was isolated from soil samples collected from China, in 1998 by the dilution plate method with Horikoshi medium, pH10, 25 C. It was then purified by transferring a single conidium onto another Horikoshi agar plate. The strain NN046156 was identified as Westerdykella sp., based on both morphological characteristics and ITS rDNA sequence.

Strain Stropharia semiglobata NN070885 was isolated in Kongstrup Bakker in Denmark. The strain was inoculated onto a PDA plate and incubated for 32 days at 26° C. in the darkness. Mycelia and spores from the plate were inoculated into 500 ml shake flasks containing 100 mls of YPG medium. The flasks were incubated for 14 days at 26° C. with shaking at 100 rpm. Strain Gelasinospora cratophora NN070952 was isolated from a soil sample in Denmark. The strain was inoculated onto a PDA plate and incubated for 2 days at 26° C. in the darkness. Mycelia and spores from the plate were inoculated into 500 ml shake flasks containing 100 mls of YPG medium. The flasks were incubated for 4 days at 26° C. with shaking at 100 rpm.

Strain Deconica coprophila MS02928 was isolated from the Denmark. The strain was inoculated onto a half strength PDA plate and incubated for 10 days at 20° C. in the darkness. Mycelia and spores from the plate were inoculated into 500 ml shake flasks containing 100 mls of YPG medium. The flasks were incubated for 15 days at 26° C. with shaking at 100 rpm.

### Media and Solutions

DAP4C-1 medium was composed of 0.5 g yeast extract, 10 g maltose, 20 g dextrose, 11 g magnesium sulphate heptahydrate, 1 g dipotassium phosphate, 2 g citric acid monohydrate, 5.2 g potassium phosphate tribasic monohydrate, 1 mL Dowfax 63N10 (antifoaming agent), 2.5 g calcium carbonate, supplemented with 1 mL KU6 metal solution, and deionised water to 1000 mL.

KU6 metal solution was composed of 6.8 g ZnCl₂, 2.5 g CuSO₄·5H₂O, 0.13 g NiCl₂, 13.9 g FeSO₄·7H₂O, 8.45 g MnSO₄·H₂O, 3 g C₆H₈O₇·H₂O, and deionised water to 1000 mL.

YP 2% glucose medium was composed of 10 g yeast extract, 20 g Bacto-peptone, 20 g glucose, and deionised water to 1000 mL.

LB plates were composed of 10 g of Bacto-tryptone, 5 g of yeast extract, 10 g of sodium chloride, 15 g of Bacto-agar, and deionised water to 1000 mL.

LB medium was composed of 10 g of Bacto-tryptone, 5 g of yeast extract, and 10 g of sodium chloride, and deionised water to 1000 mL.

COVE-Sucrose-T plates were composed of 342 g of sucrose, 20 g of agar powder, 20 mL of COVE salt solution, and deionised water to 1000 mL. The medium was sterilized by autoclaving at 15 psi for 15 minutes (Bacteriological Analytical Manual, 8th Edition, Revision A, 1998). The medium was cooled to 60° C. and 10 mM acetamide, Triton X-100 (50 μL/500 mL) were added.

COVE-N-Agar tubes were composed of 218 g Sorbitol, 10 g Dextrose, 2.02 g KNO3, 25 g agar, 50 mL Cove salt solution, and deionised water up to 1000 mL.

COVE salt solution was composed of 26 g of MgSO₄·7H₂O, 26 g of KCL, 26 g of KH₂PO₄, 50 mL of COVE trace metal solution, and deionised water to 1000 mL.

COVE trace metal solution was composed of 0.04 g of Na₂B₄O₇·10H₂O, 0.4 g of CuSO₄·5H₂O, 1.2 g of FeSO₄·7H₂O, 0.7 g of MnSO₄·H₂O, 0.8 g of Na₂MoO₄·2H₂O, 10 g of ZnSO₄·7H₂O, and deionised water to 1000 mL.

YPM medium contained 1% of Yeast extract, 2% of Peptone and 2% of Maltose.

Hen eggwhite lysozyme (HEWL) was obtained from Sigma-Aldrich #62972

### Example 1: Method for the Determination of Lysozyme Activity Against Micrococcus lysodeikticus (Reducing End Method)

The lysozyme was diluted in phosphate buffer (5 mM citrate, 5 mM K₂HPO₄, 0.01% TritonX-100, pH 5.0) to 50 μg/mL in polypropylene tubes. The diluted lysozyme was further diluted in a 96-well polypropylene microtiter plate to a concentration of 0.7 μg/mL in phosphate buffer (5 mM citrate, 5 mM K₂HPO₄, 0.01% TritonX-100, pH 5.0). In a polypropylene deepwell plate 50 μL of the lysozyme dilution was mixed with 450 μL 1% Micrococcus lysodeikticus solution (lyophilized Micrococcus lysodeikticus ATCC No. 4698 (Sigma M3770) in milli-Q water) and incubated at 40° C. with shaking (500 rpm) for 45 min. After incubation the deepwell plate was centrifuged (4000 g, 5 min) to pellet insoluble material and 100 μL of the supernatant was mixed with 50 μL 3.2M HCl in a 96-well PCR plate and incubated at 95° C. for 80 min. 50 μL of 3.5 M NaOH was added to each well of the PCR plate, and 150 μL of each sample was transferred to a new PCR plate containing 75 μL/well 4-hydroxybenzhydrazide solution in K—Na tartrate/NaOH buffer (50 g/L K—Na tartrate+20 g/L NaOH). The plate was incubated at 95° C. for 10 min before 100 L/sample was transferred to a clear flat-bottomed microtiter plate for optical density (OD) measurement at 405 nm. OD measurements were performed on three times diluted samples (50 μL sample diluted in 100 μL in Milli-Q water). The OD measurement values represent the difference after the original (background) reading was subtracted. The average OD measurement for hen eggwhite lysozyme by the present method is 0.55 at 0.9 ppm and the average OD measurement for SEQ ID NO 159 is 0.34 at 0.9 ppm (data not shown).

### Example 2: Genomic DNA Extraction from Strains of Sporormia fimetaria (SEQ ID NO: 1), Poronia punctata (SEQ ID NO: 4 and 7), Lecanicillium sp. (SEQ ID NO: 10, 13 and 67), Onygena equina (SEQ ID NO: 16), Purpureocillium lilacinum (SEQ ID NO: 19), Trichobolus zukalii (SEQ ID NO: 22), Penicillium citrinum (SEQ ID NO: 25), Cladorrhinum bulbillosum (SEQ ID NO: 28), Umbelopsis westeae (SEQ ID NO: 31), Zygomycetes sp. (SEQ ID NO: 34), Chaetomium cupreum (SEQ ID NO: 37), Cordyceps cardinalis (SEQ ID NO: 40), Penicillium sp. ‘Qii’ (SEQ ID NO: 43), Aspergillus sp. Nov. XZ2609 (SEQ ID NO: 46), Paecilomyces sp. XZ2658 (SEQ ID NO: 49 and 52), Pycnidiophora cf. Dispera (SEQ ID NO: 58), Isaria farinosa (SEQ ID NO: 64), Zopfiella sp. (SEQ ID NO: 70), Malbranchea flava (SEQ ID NO: 73), Hypholoma polytrichi (SEQ ID NO: 76), Aspergillus deflectus (SEQ ID NO: 81), Ascobolus stictoideus (SEQ ID NO: 84), Coniochaeta sp. (SEQ ID NO: 87), Ascobolus sp. ZY179 (SEQ ID NO: 96), Curreya sp. XZ2623 (SEQ ID NO: 99), Coniothyrium sp. (SEQ ID NO: 102), Hypoxylon sp. (105 and 111), Xylariaceae sp. 1653h (SEQ ID NO: 108), Engyodontium album (SEQ ID NO: 117), Metapochonia bulbillosa XZ2653 (SEQ ID NO: 120), Hamigera paravellanea A3661 (SEQ ID NO: 123), Metarhizium iadini (SEQ ID NO: 126), Clonostachys rossmaniae (SEQ ID NO: 132), Aspergillus inflatus (SEQ ID NO: 138), Paracremonium inflatum (SEQ ID NO: 141), Westerdykella sp. (SEQ ID NO: 144), Stropharia semiglobata (SEQ ID NO: 228, 231 and 153), and Gelasinospora cratophora (SEQ ID NO: 156)

Sporormia fimetaria strain NN047801 was inoculated onto a PDA plate and incubated for 7 days at 28° C. in the darkness. Several mycelia-PDA plugs were inoculated into 500 ml shake flasks containing 100 ml of YPG medium. The flasks were incubated for 3 days at 28° C. with shaking at 160 rpm.

Strain Poronia punctata NN009607 was inoculated onto a PDA plate and incubated for 7 days at 26° C. in the darkness. Mycelia and spores from the plate were inoculated into 500 ml shake flasks containing 100 mls of YPG medium. The flasks were incubated for 5 days at 26° C. with shaking at 100 rpm.

Lecanicillium sp. strain NN054002 was inoculated onto a PDA plate and incubated for 7 days at 15° C. in the darkness. Several mycelia-PDA plugs were inoculated into 500 ml shake flasks containing 100 ml of YPG medium. The flasks were incubated for 5 days at 20° C. with shaking at 160 rpm.

Strain Onygena equina NN056731 was inoculated onto a PDA plate and incubated for 18 days at 20° C. in the darkness. Mycelia and spores from the plate were inoculated into 500 ml shake flasks containing 100 mls of YPG medium. The flasks were incubated for 18 days at 20° C. with shaking at 100 rpm.

Strain Purpureocillium lilacinum NN070261 was isolated from a soil sample from the United States, and inoculated onto a PDA plate and incubated for 8 days at 26° C. in the darkness. Mycelia and spores from the plate were inoculated into 500 ml shake flasks containing 100 mls of YPG medium. The flasks were incubated for 3 days at 26° C. with shaking at 100 rpm.

Strain Trichobolus zukalii CBS720.69 was purchased CBS-KNAW Fungal Biodiversity Centre, and inoculated onto a PDA plate and incubated for 7 days at 26° C. in the darkness. Mycelia and spores from the plate were inoculated into 500 ml shake flasks containing 100 mls of YPG medium. The flasks were incubated for 3 days at 26° C. with shaking at 100 rpm.

Strain Penicillium citrinum NN070248 was inoculated onto a PDA plate and incubated for 7 days at 26° C. in the darkness. Mycelia and spores from the plate were inoculated into 500 ml shake flasks containing 100 mls of YPG medium. The flasks were incubated for 6 days at 26° C. with shaking at 100 rpm.

Cladorrhinum bulbillosum strain NN057876 was inoculated onto a PDA plate and incubated for 7 days at 37° C. in the darkness. Several mycelia-PDA plugs were inoculated into 500 ml shake flasks containing 100 ml of YPG medium. The flasks were incubated for 3 days at 37° C. with shaking at 160 rpm.

Strain Umbelopsis westeae NN070463 was inoculated onto a PDA plate and incubated for 7 days at 26° C. in the darkness. Mycelia and spores from the plate were inoculated into 500 ml shake flasks containing 100 mls of YPG medium. The flasks were incubated for 3 days at 26° C. with shaking at 100 rpm.

Zygomycetes sp. Strain NN058098, Chaetomium cupreum strain NN058297 and Pycnidiophora cf. dispera strain NN051201 were inoculated onto a PDA plate and incubated for 7 days at 25° C. in the darkness. Several mycelia-PDA plugs were inoculated into 500 ml shake flasks containing 100 ml of YPG medium. The flasks were incubated for 3 days at 25° C. with shaking at 160 rpm.

Cordyceps cardinalis NN070475 was inoculated onto a PDA plate and incubated for 8 days at 26° C. in the darkness. Several mycelia-PDA plugs were inoculated into 500 ml shake flasks containing 100 mls of YPG medium. The flasks were incubated for 5 days at 26° C. with shaking at 100 rpm.

Strain Penicillium sp. ‘qii’ NN044232 was inoculated onto a PDA plate and incubated for 5 days at 25° C. in the darkness. Mycelia and spores from the plate were inoculated into 500 ml shake flasks containing 100 ml of YPG medium. The flasks were incubated for 4 days at 25° C. with shaking at 100 rpm.

Strain Aspergillus sp. nov. XZ2609 NN058292 was inoculated onto a PDA plate and incubated for 5 days at 25° C. in the darkness. Mycelia and spores from the plate were inoculated into 500 ml shake flasks containing 100 ml of YPG medium. The flasks were incubated for 7 days at 25° C. with shaking at 100 rpm.

Strain Paecilomyces sp. XZ2658 NN058101 was inoculated onto a PDA plate and incubated for 5 days at 25° C. in the darkness. Mycelia and spores from the plate were inoculated into 500 ml shake flasks containing 100 mls of YPG medium. The flasks were incubated for 5 days at 25° C. with shaking at 100 rpm.

Isaria farinosa strain NN054449 was inoculated onto a PDA plate and incubated for 7 days at 15° C. in the darkness. Several mycelia-PDA plugs were inoculated into 500 ml shake flasks containing 100 ml of YPG medium. The flasks were incubated for 4 days at 25° C. with shaking at 160 rpm.

Zopfiella sp. strain NN046871 was inoculated onto a PDA plate and incubated for 7 days at 37° C. in the darkness. Several mycelia-PDA plugs were inoculated into 500 ml shake flasks containing 100 ml of YPG medium. The flasks were incubated for 2 days at 37° C. with shaking at 160 rpm.

Strain Malbranchea flava NN070411 was inoculated onto a PDA plate and incubated for 14 days at 26° C. in the darkness. Mycelia and spores from the plate were inoculated into 500 ml shake flasks containing 100 mls of YPG medium. The flasks were incubated for 20 days at 26° C. with shaking at 100 rpm.

Strain Hypholoma polytrichi NN057040 was inoculated onto a PDA plate and incubated for 10 days at 26° C. in the darkness. Mycelia and spores from the plate were inoculated into 500 ml shake flasks containing 100 mls of YPG medium. The flasks were incubated for 15 days at 26° C. with shaking at 100 rpm.

Strain Aspergillus deflectus NN051662 was inoculated onto a PDA plate and incubated for 7 days at 26° C. in the darkness. Mycelia and spores from the plate were inoculated into 500 ml shake flasks containing 100 mls of YPG medium. The flasks were incubated for 5 days at 26° C. with shaking at 100 rpm.

Strain Ascobolus stictoideus NN007614 was inoculated onto a PDA plate and incubated for 7 days at 26° C. in the darkness. Mycelia and spores from the plate were inoculated into 500 ml shake flasks containing 100 mls of YPG medium. The flasks were incubated for 5 days at 26° C. with shaking at 100 rpm.

Strain Coniochaeta sp. NN053834 were inoculated onto a PDA plate and incubated for 7 days at 20° C. in the darkness. Several mycelia-PDA plugs were inoculated into 500 ml shake flasks containing 100 ml of YPG medium. The flasks were incubated for 4 days at 20° C. with shaking at 160 rpm.

Strain Ascobolus sp. ZY179 NN057909 were inoculated onto a PDA plate and incubated for 7 days at 37° C. in the darkness. Several mycelia-PDA plugs were inoculated into 500 ml shake flasks containing 100 ml of YPG medium. The flasks were incubated for 5 days at 37° C. with shaking at 160 rpm.

Strain Curreya sp. XZ2623 NN058306 were inoculated onto a PDA plate and incubated for 7 days at 25° C. in the darkness. Several mycelia-PDA plugs were inoculated into 500 ml shake flasks containing 100 ml of YPG medium. The flasks were incubated for 7 days at 25° C. with shaking at 160 rpm.

Strain Coniothyrium sp. NN043614 were inoculated onto a PDA plate and incubated for 7 days at 25° C. in the darkness. Several mycelia-PDA plugs were inoculated into 500 ml shake flasks containing 100 ml of YPG medium. The flasks were incubated for 7 days at 25° C. with shaking at 160 rpm.

Strain Hypoxylon sp. NN047189 were inoculated onto a PDA plate and incubated for 7 days at 25° C. in the darkness. Several mycelia-PDA plugs were inoculated into 500 ml shake flasks containing 100 ml of YPG medium. The flasks were incubated for 7 days at 25° C. with shaking at 160 rpm.

Strain Xylariaceae sp. 1653h NN043914 were inoculated onto a PDA plate and incubated for 7 days at 37° C. in the darkness. Several mycelia-PDA plugs were inoculated into 500 ml shake flasks containing 100 ml of YPG medium. The flasks were incubated for 5 days at 37° C. with shaking at 160 rpm.

Strain Hypoxylon sp. NN046688 were inoculated onto a PDA plate and incubated for 7 days at 37° C. in the darkness. Several mycelia-PDA plugs were inoculated into 500 ml shake flasks containing 100 ml of YPG medium. The flasks were incubated for 4 days at 37° C. with shaking at 160 rpm.

Strain Engyodontium album NN042720 was inoculated onto a PDA plate and incubated for 5 days at 26° C. in the darkness. Mycelia and spores from the plate were inoculated into 500 ml shake flasks containing 100 mls of YPG medium. The flasks were incubated for 5 days at 26° C. with shaking at 100 rpm.

Strain Metapochonia bulbillosa XZ2653 NN058096 was inoculated onto a PDA plate and incubated for 5 days at 26° C. in the darkness. Mycelia and spores from the plate were inoculated into 500 ml shake flasks containing 100 mls of YPG medium. The flasks were incubated for 5 days at 26° C. with shaking at 100 rpm.

Strain Hamigera paravellanea A3661 NN102174 was inoculated onto a PDA plate and incubated for 5 days at 26° C. in the darkness. Mycelia and spores from the plate were inoculated into 500 ml shake flasks containing 100 mls of YPG medium. The flasks were incubated for 5 days at 26° C. with shaking at 100 rpm.

Strain Metarhizium iadini NN047633 were inoculated onto a PDA plate and incubated for 7 days at 25° C. in the darkness. Several mycelia-PDA plugs were inoculated into 500 ml shake flasks containing 100 ml of YPG medium. The flasks were incubated for 5 days at 25° C. with shaking at 160 rpm.

Strain Clonostachys rossmaniae NN071248 were inoculated onto a PDA plate and incubated for 7 days at 25° C. in the darkness. Several mycelia-PDA plugs were inoculated into 500 ml shake flasks containing 100 ml of YPG medium. The flasks were incubated for 4 days at 25° C. with shaking at 160 rpm.

Strain Aspergillus inflatus NN058092 were inoculated onto a PDA plate and incubated for 7 days at 25° C. in the darkness. Several mycelia-PDA plugs were inoculated into 500 ml shake flasks containing 100 ml of YPG medium. The flasks were incubated for 5 days at 25° C. with shaking at 160 rpm.

Strain Paracremonium inflatum NN071247 were inoculated onto a PDA plate and incubated for 7 days at 25° C. in the darkness. Several mycelia-PDA plugs were inoculated into 500 ml shake flasks containing 100 ml of YPG medium. The flasks were incubated for 4 days at 25° C. with shaking at 160 rpm.

Strain Westerdykella sp. NN046156 were inoculated onto a PDA plate and incubated for 7 days at 25° C. in the darkness. Several mycelia-PDA plugs were inoculated into 500 ml shake flasks containing 100 ml of YPG medium. The flasks were incubated for 7 days at 25° C. with shaking at 160 rpm.

Strain Stropharia semiglobata NN070885 was inoculated onto a PDA plate and incubated for 32 days at 26° C. in the darkness. Mycelia and spores from the plate were inoculated into 500 ml shake flasks containing 100 mls of YPG medium. The flasks were incubated for 14 days at 26° C. with shaking at 100 rpm.

Strain Gelasinospora cratophora NN070952 was inoculated onto a PDA plate and incubated for 2 days at 26° C. in the darkness. Mycelia and spores from the plate were inoculated into 500 ml shake flasks containing 100 mls of YPG medium. The flasks were incubated for 4 days at 26° C. with shaking at 100 rpm.

Strain Deconica coprophila MS02928 was inoculated onto a half strength PDA plate and incubated for 10 days at 20° C. in the darkness. Mycelia and spores from the plate were inoculated into 500 ml shake flasks containing 100 mls of YPG medium. The flasks were incubated for 15 days at 26° C. with shaking at 100 rpm.

In all cases, the mycelia were collected by filtration through MIRACLOTH® (Calbiochem, La Jolla, CA, USA) and frozen under liquid nitrogen. Frozen mycelia were ground, by a mortar and a pestle, to a fine powder, and genomic DNA was isolated using DNeasy Plant Maxi Kit (24) (QIAGEN GmbH, Hilden, Germany) following the manufacturer's instructions.

### Example 3: Genomic DNA Extraction from Rhizomucor pusillus T185-2 (SEQ ID NO: 225) and Thermomucor indicae-Seudaticae (SEQ ID NO: 61)

Rhizomucor pusillus T185-2 NN046782 was inoculated onto a PDA plate and incubated for 3 days at 45° C. in the darkness. Mycelia and spores from the plate were inoculated into 500 ml shake flasks containing 100 mls of FG4 medium. The flasks were incubated for 3 days at 45° C. with shaking at 100 rpm.

Thermomucor indicae-seudaticae strain NN000400 was inoculated onto a PDA plate and incubated for 7 days at 45° C. in the darkness. Several mycelia-PDA plugs were inoculated into 500 ml shake flasks containing 100 ml of YPG medium. The flasks were incubated for 3 days at 45° C. with shaking at 160 rpm.

In all cases, the mycelia were collected by filtration through MIRACLOTH® (Calbiochem, La Jolla, CA, USA) and frozen under liquid nitrogen. Frozen mycelia were ground, by a mortar and a pestle, to a fine powder, and genomic DNA was isolated using a method developed by Scott O. Rogers & Arnold J. Bendich (Plant Molecular Biology 5:69-76, 1985).

### Example 4: Genomic DNA Extraction from Daldinia fissa (SEQ ID NO: 90)

Strain Daldinia fissa NN054033 were inoculated onto a PDA plate and incubated for 15 days at 15° C. in the darkness.

The mycelia were collected by scraping from agar plate with the sterilized scalpel and frozen under liquid nitrogen. Frozen mycelia were ground, by a mortar and a pestle, to a fine powder, and genomic DNA was isolated using FastDNA spin kit for soil (MP Biomedicals, Santa Ana, California, USA) following the manufacturer's instruction.

### Example 5: Genomic DNA Extraction from Rosellinia sp-51542 (SEQ ID NO: 93), Yunnania penicillata (SEQ ID NO: 114) and Simplicillium obclavatum (SEQ ID NO: 135)

Strain Rosellinia sp-51542 NN051542 were inoculated onto a PDA plate and incubated for 7 days at 37° C. in the darkness. Several mycelia-PDA plugs were inoculated into 500 ml shake flasks containing 100 ml of YPG medium. The flasks were incubated for 4 days at 37° C. with shaking at 160 rpm.

Strain Yunnania penicillata NN046158 were inoculated onto a PDA plate and incubated for 7 days at 25° C. in the darkness. Several mycelia-PDA plugs were inoculated into 500 ml shake flasks containing 100 ml of YPG medium. The flasks were incubated for 4 days at 25° C. with shaking at 160 rpm.

Strain Simplicillium obclavatum NN046572 were inoculated onto a PDA plate and incubated for 7 days at 25° C. in the darkness. Several mycelia-PDA plugs were inoculated into 500 ml shake flasks containing 100 ml of YPG medium. The flasks were incubated for 4 days at 25° C. with shaking at 160 rpm.

In all cases, the mycelia were collected by filtration through MIRACLOTH® (Calbiochem, La Jolla, CA, USA) and frozen under liquid nitrogen. Frozen mycelia were ground, by a mortar and a pestle, to a fine powder, and genomic DNA was isolated using MP Faster DNA spin Kit (MP Biomedicals, Santa Ana, California, USA) following the manufacturer's instruction.

### Example 6: Genomic DNA Extraction from Thermoascus aurantiacus (SEQ ID NO: 129)

Strain Thermoascus aurantiacus NN044936 were inoculated onto a PDA plate and incubated for 7 days at 45° C. in the darkness. Several mycelia-PDA plugs were inoculated into 500 ml shake flasks containing 100 ml of YPG medium. The flasks were incubated for 3 days at 45° C. with shaking at 160 rpm.

The mycelia were collected by filtration through MIRACLOTH® (Calbiochem, La Jolla, CA, USA) and frozen under liquid nitrogen. Frozen mycelia were ground, by a mortar and a pestle, to a fine powder, and genomic DNA was isolated using Large-Scale Column Fungal DNAout (Win Honor Bioscience Limited, Beijing, China) following the manufacturer's instruction.

### Example 7: Genome Sequencing, Assembly and Annotation of Sporormia fimetaria (SEQ ID NO: 1), Thermomucor indicae-Seudaticae (SEQ ID NO: 61), Thermoascus aurantiacus (SEQ ID NO: 129)

The extracted genomic DNA samples of Thermoascus aurantiacus, Sporormia fimetaria and Thermomucor indicae-seudaticae were delivered to Beijing Genome Institute (BGI, Shenzhen, China) for genome sequencing using an ILLUMINA® GA2 System (Illumina, Inc., San Diego, CA, USA). The raw reads were assembled at BGI using program SOAPdenovo (Li et al., 2010, Genome Research, 20:265-72). The assembled sequences were analyzed using standard bioinformatics methods for gene identification and functional prediction. GeneMark-ES fungal version (Ter-Hovhannisyan V et al., 2008, Genome Research, 18 (12): 1979-1990) was used for gene prediction. Blastall version 2.2.10 (Altschul et al., 1990, Journal of Molecular Biology, 215 (3): 403-410, ftp://ftp.ncbi.nlm.nih.gov/blast/executables/release/2.2.10/) and HMMER version 2.1.1 (National Center for Biotechnology Information (NCBI), Bethesda, MD, USA) were used to predict function based on structural homology. The GH25 family lysozymes were identified directly by analysis of the Blast results. The Agene program (Munch and Krogh, 2006, BMC Bioinformatics, 7:263) and SignalP program (Nielsen et al., 1997, Protein Engineering, 10:1-6) were used to identify start codons. SignalP program was further used to predict signal peptides. Pepstats (Rice et al., 2000, Trends Genet, 16 (6): 276-277) was used to predict isoelectric points and molecular weights.

### Example 8: Genome Sequencing, Assembly and Annotation of Cladorrhinum bulbillosum (SEQ ID NO: 28), Zygomycetes sp. (SEQ ID NO: 34), Chaetomium cupreum (SEQ ID NO: 37), Ascobolus sp. ZY179 (SEQ ID NO: 96), Curreya sp. XZ2623 (SEQ ID NO: 99), Hypoxylon sp. (105 and 111), Xylariaceae sp. 1653h (SEQ ID NO: 108), Metarhizium iadini (SEQ ID NO: 126), Clonostachys rossmaniae (SEQ ID NO: 132), Simplicillium obclavatum (SEQ ID NO: 135), Paracremonium inflatum (SEQ ID NO: 141) and Westerdykella sp. (SEQ ID NO: 144)

The extracted genomic DNA samples of Cladorrhinum bulbillosum, Zygomycetes sp. Ascobolus sp. ZY179, Curreya sp. XZ2623, Coniothyrium sp., Hypoxylon sp., Xylariaceae sp. 1653h, Metarhizium iadini, Clonostachys rossmaniae, Simplicillium obclavatum, Paracremonium inflatum, Westerdykella sp. and Chaetomium cupreum were delivered to Novozymes Davis (USA) for genome sequencing using an ILLUMINA® MiSeq System. The raw reads were assembled at Novozymes Denmark using program Spades (Anton Bankevich et al., 2012, Journal of Computational Biology, 19 (5): 455-477). The assembled sequences were analyzed using standard bioinformatics methods for gene identification and function prediction. GeneMark-ES fungal version (Ter-Hovhannisyan V et al., 2008, Genome Research 18 (12): 1979-1990) was used for gene prediction. Blastall version 2.2.10 (Altschul et al., 1990, Journal of Molecular Biology, 215 (3): 403-410, ftp://ftp.ncbi.nlm.nih.gov/blast/executables/release/2.2.10/) and HMMER version 2.1.1 (National Center for Biotechnology Information (NCBI), Bethesda, MD, USA) were used to predict function based on structural homology. The GH25 family lysozymes were identified directly by analysis of the Blast results. The Agene program (Munch and Krogh, 2006, BMC Bioinformatics, 7:263) and SignalP program (Nielsen et al., 1997, Protein Engineering, 10:1-6) were used to identify start codons. SignalP program was further used to predict signal peptides. Pepstats (Rice et al., 2000, Trends Genet, 16 (6): 276-277) was used to predict isoelectric points and molecular weights.

### Example 9: Genome Sequencing, Assembly and Annotation of Lecanicillium sp. (SEQ ID NO: 10, 13 and 67), Pycnidiophora cf. Dispera (SEQ ID NO: 58), Isaria farinosa (SEQ ID NO: 64), Zopfiella sp. (SEQ ID NO: 70), Coniochaeta sp. (SEQ ID NO: 87), Daldinia fissa (SEQ ID NO: 90), Rosellinia sp-51542 (SEQ ID NO: 93), Coniothyrium sp. (SEQ ID NO: 102) and Yunnania penicillata (SEQ ID NO: 114)

The extracted genomic DNA samples of Coniochaeta sp., Daldinia fissa, Rosellinia sp., Yunnania penicillata, Lecanicillium sp., Isaria farinosa, Pycnidiophora cf. dispera and Zopfiella sp. were delivered to Fasteris (Switzerland) for genome sequencing using an ILLUMINA® HiSeq 2000 System (Illumina, Inc., San Diego, CA, USA). The raw reads were assembled at Novozymes Denmark using program Idba (Peng, Yu et al., 2010, Research in Computational Molecular Biology, 6044:426-440. Springer Berlin Heidelberg.). The assembled sequences were analyzed using standard bioinformatics methods for gene identification and function prediction. GeneMark-ES fungal version (Ter-Hovhannisyan V et al., 2008, Genome Research 18 (12): 1979-1990) was used for gene prediction. Blastall version 2.2.10 (Altschul et al., 1990, Journal of Molecular Biology, 215 (3): 403-410, ftp://ftp.ncbi.nlm.nih.gov/blast/executables/release/2.2.10/) and HMMER version 2.1.1 (National Center for Biotechnology Information (NCBI), Bethesda, MD, USA) were used to predict function based on structural homology. The GH25 family lysozymes were identified directly by analysis of the Blast results. The Agene program (Munch and Krogh, 2006, BMC Bioinformatics, 7:263) and SignalP program (Nielsen et al., 1997, Protein Engineering, 10:1-6) were used to identify start codons. SignalP program was further used to predict signal peptides. Pepstats (Rice et al., 2000, Trends Genet, 16 (6): 276-277) was used to predict isoelectric points and molecular weights.

### Example 10: Genome Sequencing, Assembly and Annotation of Aspergillus inflatus (SEQ ID NO: 138)

The extracted genomic DNA samples of Aspergillus inflatus were delivered to Exiqon A/S (Denmark) for genome sequencing using an ILLUMINA® MiSeq System (Illumina, Inc., San Diego, CA, USA). The raw reads were assembled at Novozymes Denmark using program Spades (Anton Bankevich et al., 2012, Journal of Computational Biology, 19 (5): 455-477). The assembled sequences were analyzed using standard bioinformatics methods for gene identification and function prediction. GeneMark-ES fungal version (Ter-Hovhannisyan V et al., 2008, Genome Research 18 (12): 1979-1990) was used for gene prediction. Blastall version 2.2.10 (Altschul et al., 1990, Journal of Molecular Biology, 215 (3): 403-410, ftp://ftp.ncbi.nlm.nih.gov/blast/executables/release/2.2.10/) and HMMER version 2.1.1 (National Center for Biotechnology Information (NCBI), Bethesda, MD, USA) were used to predict function based on structural homology. The GH25 family lysozymes were identified directly by analysis of the Blast results. The Agene program (Munch and Krogh, 2006, BMC Bioinformatics, 7:263) and SignalP program (Nielsen et al., 1997, Protein Engineering, 10:1-6) were used to identify start codons. SignalP program was further used to predict signal peptides. Pepstats (Rice et al., 2000, Trends Genet, 16 (6): 276-277) was used to predict isoelectric points and molecular weights.

### Example 11: Genome Sequencing, Assembly and Annotation of Poronia punctata (SEQ ID NO: 4 and 7), Onygena equina (SEQ ID NO: 16), Purpureocillium lilacinum (SEQ ID NO: 19), Trichobolus zukalii (SEQ ID NO: 22), Penicillium citrinum (SEQ ID NO: 25), Umbelopsis westeae (SEQ ID NO: 31), Cordyceps cardinalis (SEQ ID NO: 40), Penicillium Sp. ‘Qii’ (SEQ ID NO: 43), Aspergillus sp. Nov. XZ2609 (SEQ ID NO: 46), Paecilomyces sp. XZ2658 (SEQ ID NO: 49 and 52), Rhizomucor pusillus T185-2 (SEQ ID NO: 225), Malbranchea flava (SEQ ID NO: 73), Hypholoma polytrichi (SEQ ID NO: 76), Aspergillus deflectus (SEQ ID NO: 81), Ascobolus stictoideus (SEQ ID NO: 84), Engyodontium album (SEQ ID NO: 117), Metapochonia bulbillosa XZ2653 (SEQ ID NO: 120), Hamigera paravellanea A3661 (SEQ ID NO: 123), Stropharia semiglobata (SEQ ID NO: 228, 231 and 153) and Gelasinospora cratophora (SEQ ID NO: 156)

The extracted genomic DNA samples of Cordyceps cardinalis, Malbranchea flava, Poronia punctata, Gelasinospora cratophora, Penicillium sp. ‘qii’, Paecilomyces sp. XZ2658 Aspergillus sp. nov. XZ2609, Metapochonia bulbillosa XZ2653, Stropharia semiglobata, Hamigera paravellanea, Rhizomucor pusillus T185-2, Umbelopsis westeae, Hypholoma polytrichi, Trichobolus zukalii, Purpureocillium lilacinum, Penicillium citrinum, Ascobolus stictoideus, Engyodontium album, Onygena equina and Aspergillus deflectus were genome sequenced using an ILLUMINA® HiSeq 2000 System (Illumina, Inc., San Diego, CA, USA).

The raw reads of Cordyceps cardinalis, Malbranchea flava, Poronia punctata, Gelasinospora cratophora, Penicillium sp. ‘qii’, Paecilomyces sp. XZ2658 Aspergillus sp. nov. XZ2609, Metapochonia bulbillosa XZ2653, Stropharia semiglobata and Umbelopsis westeae were assembled using program Spades (Anton Bankevich et al., 2012, Journal of Computational Biology, 19 (5): 455-477). Hypholoma polytrichi, Trichobolus zukalii, Purpureocillium lilacinum, Penicillium citrinum, Ascobolus stictoideus, Engyodontium album, Onygena equine, Hamigera paravellanea, Rhizomucor pusillus T185-2 and Aspergillus deflectus were assembled using program Idba (Peng Yu et al., 2010, Research in Computational Molecular Biology. 6044:426-440. Springer Berlin Heidelberg.) The assembled sequences were analyzed using standard bioinformatics methods for gene identification and function prediction. GeneMark-ES fungal version (Ter-Hovhannisyan V et al., 2008, Genome Research 18 (12): 1979-1990) was used for gene prediction. Blastall version 2.2.10 (Altschul et al., 1990, Journal of Molecular Biology. 215 (3): 403-410, ftp://ftp.ncbi.nlm.nih.gov/blast/executables/release/2.2.10/) and HMMER version 2.1.1 (National Center for Biotechnology Information (NCBI), Bethesda, MD, USA) were used to predict function based on structural homology. The GH25 family lysozyme polypeptides were identified directly by analysis of the Blast results. The Agene program (Munch and Krogh, 2006, BMC Bioinformatics 7:263) and SignalP program (Nielsen et al., 1997, Protein Engineering 10:1-6) were used to identify start codons. SignalP program was further used to predict signal peptides. Pepstats (Rice et al., 2000, Trends in Genetics. 16 (6): 276-277) was used to predict isoelectric points and molecular weights.

### Example 12: Cloning and Expression of GH25 Lysozymes (SEQ ID NO: 1, 10, 13, 28, 34, 37, 58, 61, 64, 67 and 70)

Eleven fungal GH25 lysozyme wild type sequences were cloned from Sporormia fimetaria (SEQ ID NO: 1), Lecanicillium sp. WMM742 (SEQ ID NO: 10, 13, 67), Cladorrhinum bulbillosum (SEQ ID NO: 28), Zygomycetes sp. (SEQ ID NO: 34), Chaetomium cupreum (SEQ ID NO: 37), Pycnidiophora cf. dispera (SEQ ID NO: 58), Thermomucor indicae-seudaticae (SEQ ID NO:61), Isaria farinosa (SEQ ID NO: 64) and Zopfiella sp. (SEQ ID NO: 70).

The fungal GH25 lysozymes were cloned into an Aspergillus oryzae expression vector pCaHj505 as described in WO2013029496. The transcription of the GH25 lysozyme coding sequence with the native secretion signal was under the control of an Aspergillus oryzae alpha-amylase gene promoter.

The final expression plasmids were individually transformed into an Aspergillus oryzae expression host. The GH25 lysozyme genes were integrated by homologous recombination into the Aspergillus oryzae host genome upon transformation. Four transformants of each transformation were selected from the selective media agar plate and inoculated to 3 ml of YPM medium in 24-well plate and incubated at 30° C., 150 rpm. After 3 days incubation, 20 μl of supernatant from each transformant were analyzed on NuPAGE Novex 4-12% Bis-Tris Gel w/MES according to the manufacturer's instructions. The resulting gel was stained with Instant Blue. SDS-PAGE profiles of the cultures showed that all 11 genes were expressed with 1 or 2 protein bands each detected at 20 KD, 25 KD, 25 KD, 20 KD, 20 KD, 20 KD, 20 KD, 18 & 22 KD, 25 KD, 25 KD and 20 KD respectively. The recombinant Aspergillus oryzae strain with the strongest protein band were selected for shaking flask culturing and were inoculated on slant made of slant medium and incubated at 37 C for 6-7 days. When strains were well grown to fully sporulated, they were inoculated to 2 L shaking flasks each containing 400 ml of YPM and 4-8 flasks for each strain. Flasks were shaking at 80 rpm, 30 C. Cultures were harvested on day 3 and filtered using a 0.45 μm DURAPORE Membrane and were purified as described in Examples 20 to 30 respectively.

### Example 13: Cloning and Expression of GH25 Lysozymes (SEQ ID NO: 87, 90, 93, 96, 99, 102, 105, 108, 111, 114, 126, 129, 132, 135, 138, 141 and 144)

Seventeen fungal GH25 lysozyme wild type sequences were cloned from Coniochaeta sp. (SEQ ID NO: 87), Daldinia fissa (SEQ ID NO: 90), Rosellinia sp. (SEQ ID NO: 93), Ascobolus sp. (SEQ ID NO: 96), Curreya sp. (SEQ ID NO: 99), Coniothyrium sp. (SEQ ID NO: 102), Hypoxylon sp. (SEQ ID NO:105), Xylariaceae sp. (SEQ ID NO: 108), Hypoxylon sp. (SEQ ID NO: 111), Yunnania penicillata (SEQ ID NO: 114), Metarhizium iadini (SEQ ID NO: 126), Thermoascus aurantiacus (SEQ ID NO: 129), Clonostachys rossmaniae (SEQ ID NO: 132), Simplicillium obclavatum (SEQ ID NO: 135), Aspergillus inflatus (SEQ ID NO: 138), Paracremonium inflatum (SEQ ID NO: 141), and Westerdykella sp. (SEQ ID NO: 144). The fungal GH25 lysozymes were cloned into an Aspergillus oryzae expression vector pCaHj505 as described in WO2013029496. The transcription of the GH25 lysozyme coding sequence with the native secretion signal was under the control of an Aspergillus oryzae alpha-amylase gene promoter.

The final expression plasmids were individually transformed into an Aspergillus oryzae expression host. The GH25 lysozyme genes were integrated by homologous recombination into the Aspergillus oryzae host genome upon transformation. Four transformants of each transformation were selected from the selective media agar plate and inoculated to 3 ml of YPM or Dap4C medium in 24-well plate and incubated at 30° C., 150 rpm. After 3 days incubation, 20 μl of supernatant from each transformant were analyzed on NuPAGE Novex 4-12% Bis-Tris Gel w/MES according to the manufacturer's instructions. The resulting gel was stained with Instant Blue. SDS-PAGE profiles of the cultures showed that all 17 genes were expressed with 1 or 2 protein bands each detected at 20 KD, 20 KD, 20 KD, 20 KD, 20 KD, 20 KD, 20 KD, 20 KD, 20 KD, 20 KD, 25 & 28 KD, 25 KD, 26 KD, 24 KD, 26 KD, 26 KD and 20 KD respectively. The recombinant Aspergillus oryzae strain with the strongest protein band were selected for shaking flask culturing and were inoculated on slant made of slant medium and incubated at 37 C for 6-7 days. When strains were well grown to fully sporulated, they were inoculated to 2 L shaking flasks each containing 400 ml of Dap4C and 2-6 flasks for each strain. Flasks were shaking at 80 rpm, 30 C. Cultures were harvested on day 3 and filtered using a 0.45 μm DURAPORE Membrane and were purified as described in Examples 31 to 47 respectively.

### Example 13: Construction of the Improved Split-Marker Aspergillus oryzae Host (DAU)

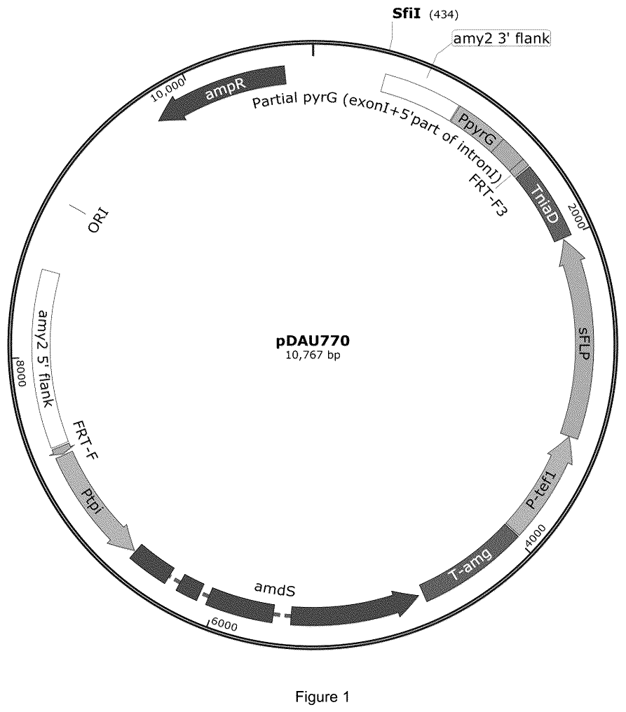

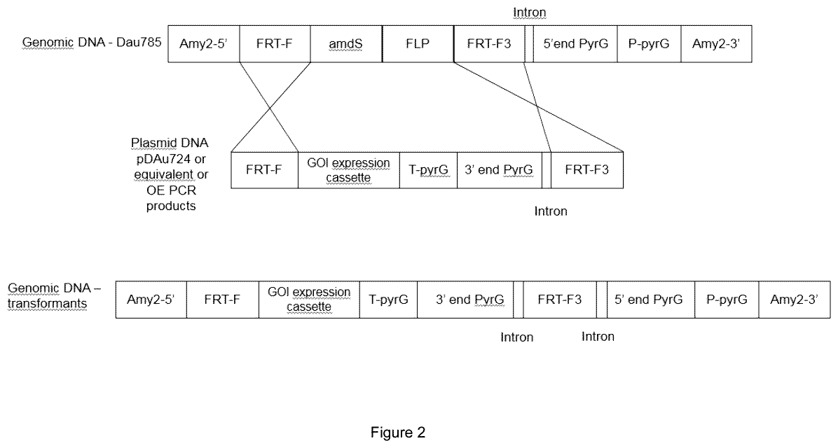

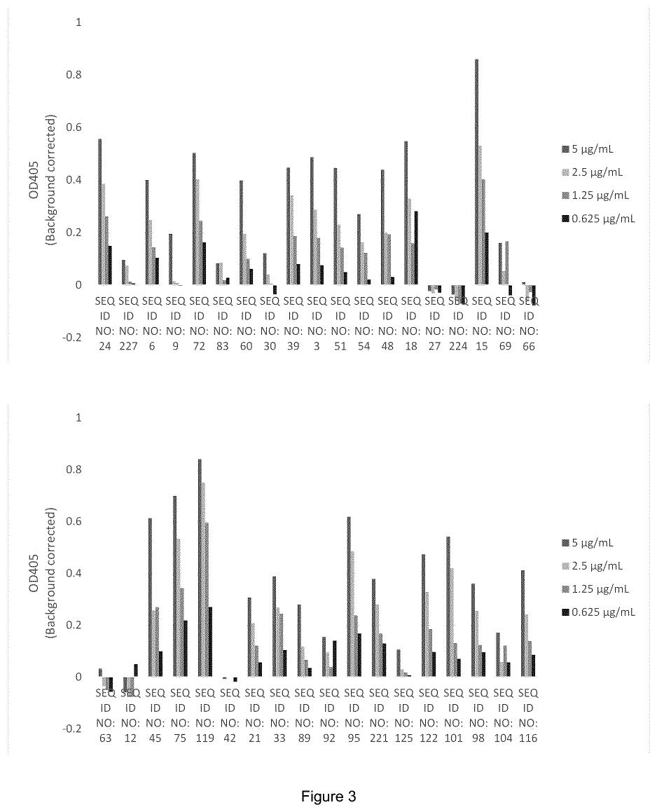

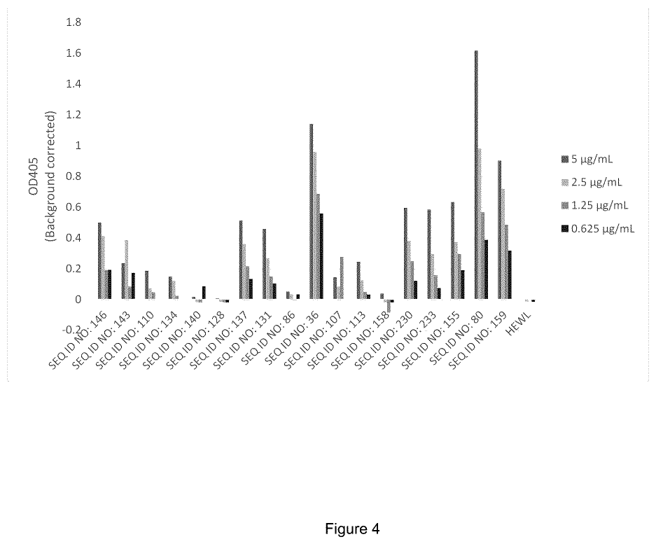

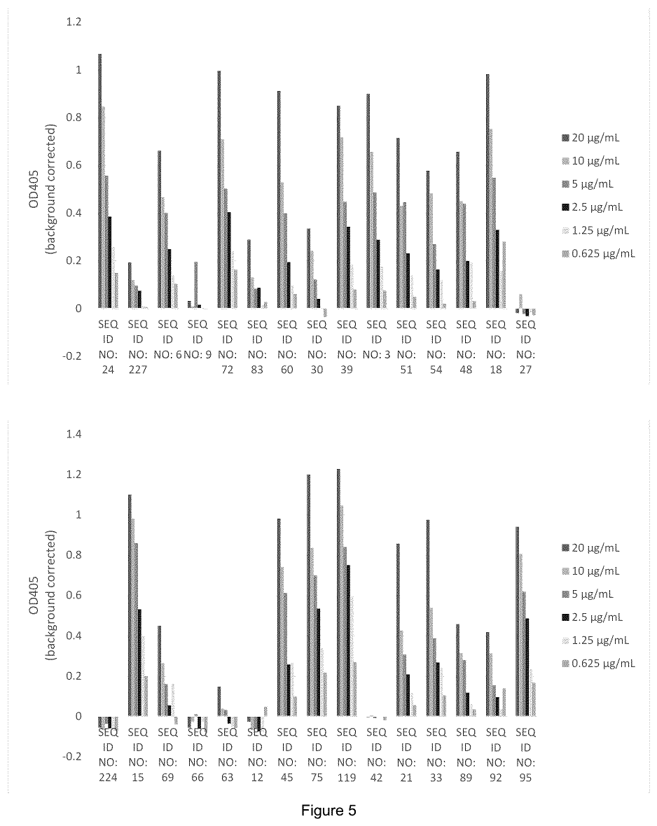

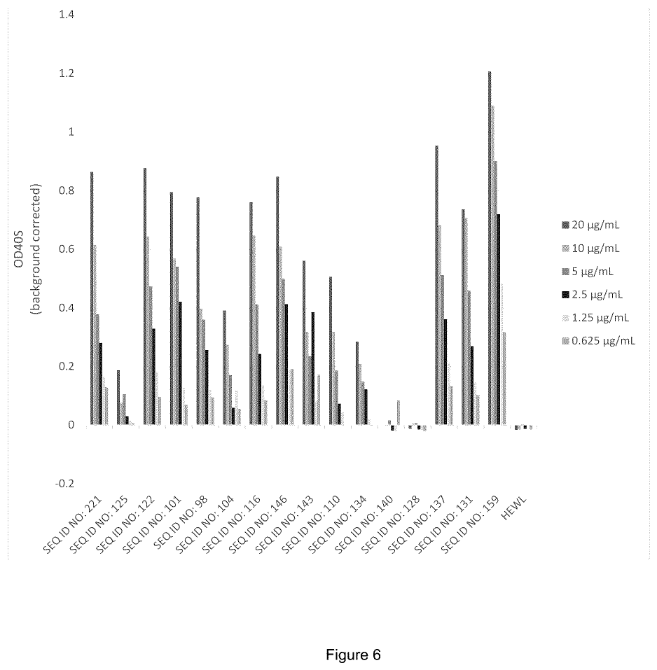

An improved Aspergillus oryzae host/vector system comparable to the one described in example 5 disclosed in WO 2016026938A1 was constructed. The improvement was made to reduce the size of the transforming DNA by moving the FLPase expression cassette located on PART-II of the plasmid pDAu724 (see page 34 in WO 2016026938A1, FIG. 7 and SEQ ID NO: 30) to the integration locus amy2 in the genome of the host strain. The cloning of the FLPase expression cassette into pDAu703 (WO 2016026938A1 page 32 and FIG. 6 and SEQ ID NO: 29) was done by amplification of the FLPase expression cassette from pDAu724 and cloning in between FRT-F3 and the amdS selection marker of pDAu703 to give the plasmid pDAu770 (FIG. 1, SEQ ID NO: 216). The same protocol as described in WO 2016026938A1 page 33 was used to transform the linearized plasmid pDAu770 into protoplasts of A. oryzae strain Jal1338 (disclosed in WO2012/160097). Transformants were selected on AmdS selection plates to obtain strain DAu785. The resulting recombinant host strain DAu785 has a modified amy2 locus comparable to the one in DAU716 (WO2016/026938) with the addition of the FLPase expression cassette (FIG. 2, top panel). The host strain DAu785 is now constitutively expressing the FLPase site specific recombinase allowing the integration at the FRT sites of the transforming DNA in this case the PCR fragments obtained by Overlap Extension PCR reaction (FIG. 2, middle and bottom panels) and described in Example 17 below.

### Example 14: PCR Amplification and InFusion Cloning of Lysozyme Encoding Sequences SEQ ID NO: 16, 19, 25, 31, 40, 73 and 117

Based on the lysozyme gene sequences identified by genome mining in Onygena equina, Purpureocillium lilacinum, Malbranchea flava, Engyodontium album, Penicillium citrinum, Umbelopsis westeae, and Cordyceps cardinalis, InFusion cloning primers were designed and ordered (Sigma Aldrich, Darmstadt, Germany) (see list in table 2 below).

TABLE 2
InFusion cloning primers

| | | Primer to | |
|---|---|---|---|
| | Primer | lysozyme | |
| | SEQ | SEQ | |
| Primer* | ID NO: | ID NO: | Sequence |
| C8VRJ-F | 162 | 16 | ACACAACTGGGGATCCACCAT |
| | | | GTTGAAAACAATTATCTATAC |
| | | | CACCCTTGCC |
| C8VRJ-R | 163 | 16 | AGATCTCGAGAAGCTTAGCCC |
| | | | TTTGCAAATCGTTGCAATCC |
| C8VRQ-F | 164 | 19 | ACACAACTGGGGATCCACCAT |
| | | | GAAGTTCGCATCCGTCGCC |
| C8VRQ-R | 165 | 19 | AGATCTCGAGAAGCTTAACCG |
| | | | GCGTTGGCAATCTTCTT |
| C8VS9-F | 166 | 25 | ACACAACTGGGGATCCACCAT |
| | | | GCGCCCCTCCGTCATATTGC |
| C8VS9-R | 167 | 25 | AGATCTCGAGAAGCTTAAGCA |
| | | | GAAAACACGTTCAAATCAAAC |
| | | | TTCTTACT |
| C8VSA-F | 168 | 31 | ACACAACTGGGGATCCACCAT |
| | | | GAAGCTCACCTTTGCCTCTCT |
| | | | AACT |
| C8VSA-R | 169 | 31 | AGATCTCGAGAAGCTTAGGCT |
| | | | CCTTTGGCCATCCTAGACA |
| C8VSC-F | 170 | 40 | ACACAACTGGGGATCCACCAT |
| | | | GCGCGCCTTTATTCCAGTCTT |
| C8VSC-R | 171 | 40 | AGATCTCGAGAAGCTTAGGCA |
| | | | GAGAAAACGTTGAGATCAAAT |
| | | | TTCTTG |
| C8VRT-F | 172 | 73 | ACACAACTGGGGATCCACCAT |
| | | | GAAGCTGTCTCTCCTCCTTAT |
| | | | TGTTGC |
| C8VRT-R | 173 | 73 | AGATCTCGAGAAGCTTAACCT |
| | | | AGGGCCATTCTCTTCAACCC |
| C8VS8-F | 174 | 117 | ACACAACTGGGGATCCACCAT |
| | | | GAAGTCTTTTGGTGTTATTGC |
| | | | TACCGG |
| C8VS8-R | 175 | 117 | AGATCTCGAGAAGCTTAGCCT |
| | | | CTGGCGATTCTCTGAAGC |

*-F—forward primer;
-R—reverse primer

PCR amplifications of SEQ ID NO: 16, 19, 25, 31, 40, 73 and 117 encoding for lysozyme polypeptides were carried out using Phusion High-Fidelity DNA polymerase (New 5 England Biolabs, BioNordika Denmark A/S, Herlev, Denmark) in a 50 μL volume reaction. The PCR reaction mixes were consisting of 10 μL Phusion reaction buffer HF (5×); 1 μL of PCR nucleotide Mix (10 mM); 2 μL forward cloning primers (2.5 mM); 2 μL reverse cloning primers (2.5 mM); 1 μL Phusion High-Fidelity DNA Polymerase #M0530L (2000 U/mL); and PCR grade water up to 50 μL. PCR reactions were incubated on a thermocycler T100 (Biorad, Hercules, California, USA) using the following program: initial denaturation of 2 min at 98° C. followed by 30 cycles of 10 sec at 98° C., 2 min at 72° C. and ending up by a final elongation of 10 min at 72° C. PCR amplicons were purified using AMPure XP beads system kit (Agencourt, Beverly, Massachusetts, USA) adapted on a Biomek FXp Liquid handler (Beckman Coulter, Brea, California, USA).

InFusion cloning was made using InFusion HD Plus EcoDry Cloning system kit (Takara, Kusatsu, Japan) in expression vector pDAu109 (WO 2005042735) previously digested with BamHI and HindIII restriction enzymes and following manufacturer's instructions.

A 2.5 μL volume of the five time diluted ligation mixtures was used to transform E. coli TOP10 (see strain chapter) chemically competent cells (Life Technologies, Carlsbad, CA, USA). Three colonies were selected from LB agar plates containing 100 μg of ampicillin per ml and cultivated overnight in 3 mL of LB medium supplemented with 100 μg of ampicillin per ml. Plasmids DNA were purified using a Qiagen Spin Miniprep kit (Cat. 27106) (QIAGEN GmbH, Hilden, Germany) according to the manufacturer's instructions.

Lysozyme sequences cloned by InFusion were scrutinized for errors by Sanger DNA sequencing.

Forward and reverse oligonucleotide primers shown below were designed to PCR amplify the GH25 open reading frame from the genomic DNA samples. An IN-FUSION™ Cloning Kit (Clontech, Mountain View, CA, USA) was used to clone the fragment directly into the expression vector pDau109 (WO 2005/042735).

### Example 15: PCR Amplification and InFusion Cloning of Lysozyme Encoding Sequences SEQ ID NO: 43, 46, 49 and 52

BamHI-XhoI based cloning from Penicillium sp. ‘qii’ (SEQ ID NO: 43), Paecilomyces sp. XZ2658 (SEQ ID NO: 46 and 49) and Aspergillus sp. nov. XZ2609 (SEQ ID NO: 52).

The forward and reverse PCR primers shown in table 3 were used to generate an EcoRI-XhoI flanked cloning cassette from the genomic DNA prepared above for the following samples:

TABLE 3
PCR primers

| | | Primer to | |
|---|---|---|---|
| | Primer | lysozyme | |
| | SEQ | SEQ | |
| Primer* | ID NO: | ID NO: | Sequence |
| WIN1054-F | 188 | 43 | 5'-ACACAACTGGGGATCCA |
| | | | CCATGAAGACTACGGGTGTC |
| WIN1054-R | 189 | 43 | 5'-CCCTCTAGATCTCGAGT |
| | | | TAAGAACCCTTGGCAAAG |
| WIN1065-F | 190 | 46 | 5'-ACACAACTGGGGATCCA |
| | | | CCATGAAGTTCACTACCATT |
| | | | GC |
| WIN1065-R | 191 | 46 | 5'-CCCTCTAGATCTCGAGC |
| | | | TACCCCTCGACAATCTT |
| WIN1057-F | 192 | 49 | 5'-ACACAACTGGGGATCCA |
| | | | CCATGAAGTCTGTTGCTGTC |
| | | | T |
| WIN1057-R | 193 | 49 | 5'-CCCTCTAGATCTCGAGC |
| | | | TAAGAAGCATTCGCAATGC |
| WIN1058-F | 194 | 52 | 5'-ACACAACTGGGGATCCA |
| | | | CCATGAAGCTCACGAGTGTG |
| WIN1058-R | 195 | 52 | 5'-CCCTCTAGATCTCGAGT |
| | | | TACGAACCTCTAGCAAGC |

*-F—forward primer;
-R—reverse primer

Bold letters represent coding sequence. The underlined sequence is homologous to the insertion sites of pDau109.

The PCR reaction (25 μl) was composed of 12.5 μl of 2× IPROOF™ HF Master Mix, 0.5 μl of appropriate forward primer (100 μM), 0.5 μl of the appropriate reverse primer (100 μM), 0.5 μl of genomic (100 ng/μl), and 11 μl of deionized water. The PCR reaction was incubated in a DYAD® Dual-Block Thermal Cycler (MJ Research Inc., Waltham, MA, USA) programmed for 1 cycle at 98° C. for 30 seconds; 30 cycles each at 98° C. for 10 seconds, 55° C. for 10 seconds, and 72° C. for 60 seconds; and 1 cycle at 72° C. for 10 minutes. Samples were cooled to 10° C. before removal and further processing.

Five μl of the PCR reaction were analyzed by 1% agarose gel electrophoresis using TAE buffer where an DNA band of the appropriate size was observed. In the case of WIN1054 for example, an approximately 700 base pair band was observed. The remaining PCR reactions were purified using an ILLUSTRA™ GFX™ PCR DNA and Gel Band Purification Kit according to the manufacturer's instructions.

The fragments were then cloned into BamHI and XhoI digested pDau109 using an IN-FUSION™ Cloning Kit resulting in plasmids containing the inserts. Cloning of the GH25 lysozyme PCR inserts into Bam HI-XhoI digested pDau109 resulted in the transcription of the cloned genes under the control of a NA2-tpi double promoter. NA2-tpi is a modified promoter from the gene encoding the Aspergillus niger neutral alpha-amylase in which the untranslated leader has been replaced by an untranslated leader from the gene encoding the Aspergillus nidulans triose phosphate isomerase. Isolated plasmids were sequenced with vector primers in order to determine a representative plasmid expression clone that was free of PCR errors.

### Example 16: PCR Amplification and InFusion Cloning of Lysozyme Encoding Sequences SEQ ID NO: 4, 7, 22, 225, 76, 81 and 84

BamHI-HindIII based cloning of Hypholoma polytrichi (SEQ ID NO: 76), Aspergillus deflectus (SEQ ID NO: 81), Trichobolus zukalii (SEQ ID NO: 22), Rhizomucor pusillus (SEQ ID NO: 225), Poronia punctata (SEQ ID NO: 4 and 7) and Ascobolus stictoideus (SEQ ID NO: 84).

The forward and reverse PCR primers shown in table 4 were used to generate an BamHI-HindIII flanked cloning cassette from the genomic DNA prepared above for the following samples:

TABLE 4
PCR primers

| | | Primer | |
|---|---|---|---|
| | | to | |
| | Primer | lyso- | |
| | SEQ | zyme | |
| | ID | SEQ | |
| Primer* | NO: | ID NO: | Sequence |
| KKSC0132-F | 196 | 4 | 5'-ACACAACTGGGGATCC |
| | | | ACCATGAAGTCTTACATCG |
| | | | CCCCC |
| KKSC0132-R | 197 | 4 | 5'-GTGCGGCCGCAAGCTT |
| | | | AATCAGAGGCTTCCTCCCA |
| | | | TAGG |
| KKSC0133-F | 198 | 7 | 5'-ACACAACTGGGGATCC |
| | | | ACCATGAAGTATCTCGTTC |
| | | | CCCTTTTG |
| KKSC0133-R | 199 | 7 | 5'-AGATCTCGAGAAGCTT |
| | | | ATTAGCCCTTAGCCAGTCT |
| | | | CTT |
| KKSC0311-F | 200 | 22 | 5'-ACACAACTGGGGATCC |
| | | | ACCATGAAGCTCACCACTT |
| | | | TTATCACG |
| KKSC0311-R | 201 | 22 | 5'-CTAGATCTCGAGAAGC |
| | | | TTTTAAGTCCCCTTGGCAA |
| | | | GCG |
| KKSC0314-F | 202 | 225 | 5'-ACACAACTGGGGATCC |
| | | | ACCATGAAGTTTGCACTCC |
| | | | TAGTATCTG |
| KKSC0314-R | 203 | 225 | 5'-CTAGATCTCGAGAAGC |
| | | | TTTTAGCCATAGTAGTTCT |
| | | | TGTCCCA |
| KKSC0862-F | 204 | 76 | 5'-ACACAACTGGGGATCC |
| | | | ACCATGGCAAAGCTCCTCA |
| | | | AG |
| KKSC0862-R | 205 | 76 | 5'-AGATCTCGAGAAGCTT |
| | | | ATTAGTGGGCGAAGACGTT |
| KKSC0819-F | 206 | 81 | 5-ACACAACTGGGGATCCA |
| | | | CCATGAAGCTTCTTTCCGC |
| | | | CCT |
| KKSC0819-R | 207 | 81 | 5'-AGATCTCGAGAAGCTT |
| | | | ATCACTGAGAGGCAAACTT |
| | | | GAC |
| KKSC0317-F | 208 | 84 | 5'-ACACAACTGGGGATCC |
| | | | ACCATGGCTTCCAGACTGA |
| | | | CCCT |
| KKSC0317-F | 209 | 84 | 5'-CTAGATCTCGAGAAGC |
| | | | TTTTAAGCTGCCACGCACT |
| | | | GGG |

*-F—forward primer;
-R—reverse primer

Bold letters represent coding sequence. The underlined sequence is homologous to the insertion sites of pDau109.

The PCR reaction (25 μl) was composed of 12.5 μl of 2× IPROOF™ HF Master Mix, 0.5 μl of appropriate forward primer (100 μM), 0.5 μl of the appropriate reverse primer (100 μM), 0.5 μl of genomic (100 ng/μl), and 11 μl of deionized water. The PCR reaction was incubated in a DYAD® Dual-Block Thermal Cycler (MJ Research Inc., Waltham, MA, USA) programmed for 1 cycle at 98° C. for 30 seconds; 30 cycles each at 98° C. for 10 seconds, 55° C. for 10 seconds, and 72° C. for 60 seconds; and 1 cycle at 72° C. for 10 minutes. Samples were cooled to 10° C. 10 before removal and further processing.

Five μl of the PCR reaction were analyzed by 1% agarose gel electrophoresis using TAE buffer where an DNA band of the appropriate size was observed. In the case of KKSC0317 for example, an approximately 860 base pair band was observed. The remaining PCR reactions were purified using an ILLUSTRA™ GFX™ PCR DNA and Gel Band Purification Kit according to the manufacturer's instructions.

The fragments were then cloned into BamHI and HindIII digested pDau109 using an IN-FUSION™ HD Plus EcoDry Cloning system kit (Takara, Kusatsu, Japan). Cloning of the GH25 lysozyme PCR inserts into BamHI-HindIII digested pDau109 resulted in the transcription of the cloned genes under the control of a NA2-tpi double promoter. NA2-tpi is a modified promoter from the gene encoding the Aspergillus niger neutral alpha-amylase in which the untranslated leader has been replaced by an untranslated leader from the gene encoding the Aspergillus nidulans triose phosphate isomerase. Isolated plasmids were sequenced with vector primers in order to determine a representative plasmid expression clone that was free of PCR errors.

### Example 17: Overlap Extension PCR Cloning (SEQ ID NO: 120, 123, 228, 231, 153 and

156)

pDAu724 plasmid was used as DNA template to amplify two PCR products (F1 and F3) in reactions composed of 10 μL of KAPA polymerase buffer 5×, 1 μL 10 mM KAPA PCR Nucleotide Mix, 1 μL of 10 μM of the appropriate forward primers (SEQ ID NO: 210 for F1 and SEQ ID NO: 212 for F3), 1 μL of 10 μM of the appropriate reverse primers (SEQ ID NO: 211 for F1 and SEQ ID NO: 213 for F3), 1 to 10 ng of pDAu724 plasmid, 1 μL of KAPA Biosystems polymerase KK2502 (1 unit) and PCR-grade water up to 50 μL.

PCR amplification reactions were carried out on a DYAD® Dual-Block Thermal Cycler (MJ Research Inc., Waltham, MA, USA) programmed for 2 min. at 98° C. and followed by 35 cycles of 10 sec. at 98° C. and 2 min. at 72° C. and one final cycle of 10 min. at 72° C.

Five μl of the PCR reaction were analyzed by 1% agarose gel electrophoresis using TAE buffer where DNA bands of the appropriate size were observed. The remaining PCR reactions were purified using an ILLUSTRAT GFX™ PCR DNA and Gel Band Purification Kit according to the manufacturer's instructions.

Forward primer F1: SEQ ID NO: 210:
GAATTCGAGCTCGGTACCTTGAAGTTC
Reverse primer F1: SEQ ID NO: 211:
GGTGGATCCCCAGTTGTGTATATAGAGGATT
Forward primer F3: SEQ ID NO: 212:
TGCGCGGCGCGGCTGGGTCGACTCTA
Reverse primer F3: SEQ ID NO: 213:
TTCACACAGGAAACAGCTATGACCATG

Overlap Extension PCR reaction for cloning lysozyme genes amplified from Gelasinospora cratophora, Metapochonia bulbillosa, Hamigera paravellanea, and Stropharia semiglobata (3 sequences) gDNAs were composed of 10 μL KAPA polymerase buffer (5×), 1 μL 10 mM KAPA PCR Nucleotide Mix, 50 ng of PCR fragment F1 and equimolar amounts of PCR fragment F3 and lysozyme genes encoding for SEQ ID NO: 120, 123, 228, 231, 153 and 156, 1 μl KAPA Biosystems polymerase KK2502 (1 unit) and PCR-grade water up to 48 μL. Reaction were incubated on a DYAD® Dual-Block Thermal Cycler (MJ Research Inc., Waltham, MA, USA) using a program composed of 2 min. at 98° C.; followed by 5 cycles each composed of 10 sec. at 98° C., 30 sec. at 68° C., and 5 min. at 72° C. and completed by a final extension of 8 min. at 72° C.

During the OE PCR reactions, annealing between fragment F1 and lysozyme genes encoding for SEQ ID NO: 120, 123, 228, 231, 153 and 156 respectively were ensured by overlap SEQ ID NO: 214 included in the forward cloning primers (C6ZGC-F; C6ZG7-F; C122PC-F; C122PA-F; C122P9-F and C122P5-F respectively, table 4a) and annealing between fragment F3 and the lysozyme genes encoding for SEQ ID NO: 120, 123, 228, 231, 153 and 156 respectively were ensured by the overlapping SEQ ID NO: 215 included in the reverse cloning primers (C6ZGC-R; C6ZG7-R; C122PC-R; C122PA-R; C122P9-R and C122P5-R respectively, table 4a).

TABLE 4a
Cloning primers

| | | Primer to | |
|---|---|---|---|
| | Primer | lysozyme | |
| | SEQ | SEQ | |
| Primer* | ID NO: | ID NO: | Sequence |
| C122P5-F | 176 | 156 | CTATATACACAACTGGGGA |
| | | | TCCACCATGAAGTCCTTCG |
| | | | TCCTCACGGC |
| C122P5-R | 177 | 156 | TAGAGTCGACCCAGCCGCG |
| | | | CCGGCCATTAAGATCCCTT |
| | | | AGCAAGAGCCTTAAGGC |
| C6ZGC-F | 178 | 120 | CTATATACACAACTGGGGA |
| | | | TCCACCATGAAGTCTGTTA |
| | | | CTTTCATCGCCAGTCT |
| C6ZGC-R | 179 | 120 | TAGAGTCGACCCAGCCGCG |
| | | | CCGGCCATTACGAAGCGTT |
| | | | AGCAATACGCTTAAGC |
| C6ZG7-F | 180 | 123 | CTATATACACAACTGGGGA |
| | | | TCCACCATGAAGGCTTCTT |
| | | | CCATCCTCTCCC |
| C6ZG7-R | 181 | 123 | TAGAGTCGACCCAGCCGCG |
| | | | CCGGCCATCATCCACTCGC |
| | | | CAACTTCTTCAAC |
| C122PC-F | 182 | 228 | CTATATACACAACTGGGGA |
| | | | TCCACCATGTTCTCTTTCG |
| | | | TCAAAGCGCTCA |
| C122PC-R | 183 | 228 | TAGAGTCGACCCAGCCGCG |
| | | | CCGGCCATTAATTTGCAAA |
| | | | GACATTGAGGTCGAATTGGC |
| C122PA-F | 184 | 231 | CTATATACACAACTGGGGA |
| | | | TCCACCATGTTCCCTTTCG |
| | | | TCAAAACGCTCA |
| C122PA-R | 185 | 231 | TAGAGTCGACCCAGCCGCG |
| | | | CCGGCCATTAATTTGCAAA |
| | | | CACATTGAGGTCGAATTGG |
| C122P9-F | 186 | 153 | CTATATACACAACTGGGGA |
| | | | TCCACCATGGTCAAAATCT |
| | | | TGAGCCTTCTAGCC |
| C122P9-R | 187 | 153 | TAGAGTCGACCCAGCCGCG |
| | | | CCGGCCACTATGCGAATAC |
| | | | GCTCAGGTCGAATTG |

One μL of 10 μM primer SEQ1 and 1 μL of 10 μM primer SEQ4 were added to the OE PCR reactions and the reactions were incubated a second time on a DYAD® Dual-Block Thermal Cycler (MJ Research Inc., Waltham, MA, USA) using a program composed of 2 min at 98° C.; followed by 25 cycles each composed of 10 sec. at 98° C., and 4 min. at 72° C. and completed by a final extension of 10 min. at 72° C.

Five μl of the PCR reactions were analyzed by 1% agarose gel electrophoresis using TAE buffer where an DNA bands of the appropriate size were observed. The remaining PCR reactions were up-concentrated to 20 μL by heating the tubes at 60° C. 10 μL of those reactions were used for Aspergillus oryzae DAu785 protoplasts transformation.

Primer bind forward SEQ ID NO: 214:
CTATATACACAACTGGGGATCCACC
Primer bind reverse SEQ ID NO: 215:
TAGAGTCGACCCAGCCGCGCCGGCCA

### Example 18: Preparation and Expression of Aspergillus Protoplasts (SEQ ID NO: 16, 19, 25, 31, 40, 73, 117, 120, 123, 228, 231, 153 and 156)

Protoplasts of Aspergillus oryzae MT3568 were prepared according to WO 95/002043. One hundred μl of protoplasts were mixed with 1-3 μg of the Aspergillus expression vectors or OE PCRs (for SEQ ID NO: 16, 19, 25, 31, 40, 73, 117, 120, 123, 228, 231, 153 and 156) and 250 μL of 60% PEG 4000 (Applichem, Darmstadt, Germany) (polyethylene glycol, molecular weight 4,000), 10 mM CaCl₂), and 10 mM Tris-HCl pH 7.5 and gently mixed. The mixtures were incubated at 37° C. for 30 minutes and the protoplasts were spread onto COVE plates for selection. After incubation for 4-7 days at 37° C. spores of four transformants were inoculated into 0.2 mL of YP+2% glucose or DAP4C-1 medium in 96 well microtiter plates. After 4 days cultivation at 30° C., the culture broths were analysed by SDS-PAGE to identify transformants producing the highest amounts of lysozyme enzymes.

Spores of the best transformants for each transformation were spread onto COVE plates containing 0.01% TRITON® X-100 in order to isolate single colonies. The spreading was repeated twice in total on COVE plates containing 10 mM sodium nitrate. Spores were then inoculated into 500 mL shake flasks containing 100 ml of YP+2% glucose and incubated for 4 days at 30° C. with shaking at 100 rpm.

Previously selected strains were inoculated in 250 mL shake flasks with baffle containing 100 to 150 mL of DAP4C-1 supplemented lactic acid and with diammonium phosphate or YP2% glucose medium and fermented during 4 days at a temperature of 30° C. under 150 rpm agitation. Culture broths were harvested by filtration using a 0.2 μm filter device.

The culture broths can be purified as described in Example 48.

### Example 19: Expression of GH25 Lysozymes in Aspergillus oryzae (SEQ ID NO: 4, 7, 22, 43, 46, 49, 52, 225, 76, 81 and 84)

Protoplasts of Aspergillus oryzae MT3568 were prepared according to WO 95/002043. One hundred μl of protoplasts were mixed with 1-3 μg of one of the following Aspergillus expression vectors: SEQ ID NO: 4, 7, 22, 43, 46, 49, 52, 225, 76, 81 and 84.

Six μl containing about 3.0 μg total DNA was used for the transformation. The DNA was gently added to 100 μl of A. oryzae MT3568 protoplasts and 250 μl of 60% PEG 4000 (Sigma-Aldrich cat. No. 95904). The 60% (W/V) PEG 4000 was prepared in the following manner: PEG 4000 powder was dissolved in double distilled H2O and then heated for 10-20 seconds in a microwave oven at 800 watt until dissolved. The dissolved solution was cooled down to room temperature and then then adjusted with CaCl₂ solution and Tris-HCl solution (pH 7.5) for a final concentration of 10 mM of each. After adding the 60% PEG 4000 solution, the tube was gently mixed and incubated at 37° C. for 30 minutes. The mix was added to 6 ml of top agar with 10 mM acetamide and plated onto COVE-sorbitol plates with 10 mM acetamide.

The plates were incubated at 37° C. for 3 or more days and then moved to 26° C. for two days. Spores from 4 to 8 individual colonies were picked by first dipping a white 10 μl inoculation pin (Nunc A/S, Denmark) in a 0.1% TWEEN® 80 solution, contacting the sporulating colony on the selection plate, and restreaking with the pin onto fresh COVE sorbitol plates containing 10 mM acetamide. After 5 days at 26° C., spores from the restreaked colonies were used to inoculate a 96 well deep dish plate (NUNC, cat. no. 260251, Thermoscientific, USA). The wells of the deep dish plate contained 500 uls of either YP+2% glucose or DAP4C media. The inoculated plate was sealed with gas permeable tape (89009-656, VWR.com). Plates were incubated stationary at 30 C for 5 days. Expression was verified by analysis of 20 uls of harvested culture fluid on SDS-PAGE using a NUPAGE® 10% Bis-Tris gel (Invitrogen, Carlsbad, CA, USA) and Coomassie blue staining. One transformant for each transformation experiment was selected for further work.

Spores of each designated transformant were inoculated into both YP+2% glucose medium and DAP-4C-1 medium (100 mls in 500 ml Erlenmeyer shake flask with baffles). The cultures were incubated at 26° C. and 150 rpm, 3 days and if necessary 4 days. An SDS gel was run as above to test protein amount.

After incubation for 4-7 days at 37° C. spores of four transformants were inoculated into 0.2 mL of YP+2% glucose or DAP4C-1 medium in 96 well microtiter plates. After 4 days cultivation at 30° C., the culture broths were analysed by SDS-PAGE to identify transformants producing the highest amounts of lysozyme enzymes.

The culture broths were purified as described in Example 48.

### Example 20: Purification of SEQ ID NO: 3

The culture supernatant of O23CD8 was firstly precipitated with ammonium sulfate (80% saturation), then the precipitation was added water to adjust conductance to about 170 mS/cm. The solution was filtered with 0.45 μm filter and then loaded into Phenyl Sepharose 6 Fast Flow column (GE Healthcare) equilibrated with 20 mM NaAc at pH5.0 with 1.5M (NH₄)₂SO₄ added. A gradient decrease of (NH₄)₂SO₄ concentration was applied as elution buffer from 1.5M to zero, and then elution fractions and flow-through fraction were collected to detect lysozyme activity. The fractions with lysozyme activity were analyzed by SDS-PAGE, and then concentrated, changed buffer by 20 mM PBS at pH6.0 for further evaluation. The protein concentration was determined by Qubit® Protein Assay Kit (Invitrogen, cat Q33212).

### Example 21: Purification of SEQ ID NO: 12

The culture supernatant of O23T24 was firstly precipitated with ammonium sulfate (80% saturation), then dialyzed with 20 mM NaAc at pH5.5. The solution was filtered with 0.45 μm filter and then loaded into SP Fast Flow column (GE Healthcare) equilibrated with 20 mM NaAc at pH5.5. A gradient increase of NaCl concentration was applied as elution buffer from zero to 1M, and then elution fractions and flow-through fraction were collected to detect lysozyme activity. The fractions with lysozyme activity were analyzed by SDS-PAGE, and then pooled and concentrated for further evaluation. The protein concentration was determined by Qubit® Protein Assay Kit (Invitrogen, cat Q33212).

### Example 22: Purification of SEQ ID NO: 15

The culture supernatant of O23T22 was firstly precipitated with ammonium sulfate (80% saturation), then dialyzed with 20 mM NaAc at pH4.5. The solution was filtered with 0.45 μm filter and then loaded into SP Fast Flow column (GE Healthcare) equilibrated with 20 mM NaAc at pH4.5. A gradient increase of NaCl concentration was applied as elution buffer from zero to 1M, and then elution fractions and flow-through fraction were collected to detect lysozyme activity. The fractions with lysozyme activity were analyzed by SDS-PAGE, and then pooled together.

Since the purified sample has more than two bands, the conductivity of sample was adjusted to 140 mS/cm, and then loaded into a Phenyl HP column (GE Healthcare), which was equilibrated by 20 mM PBS at pH6.0 with 1.2M (NH₄)₂SO₄ added. A gradient decrease of (NH₄)₂SO₄ concentration was applied as elution buffer from 1.2M to zero, and then elution fractions and flow-through fraction were collected to detect lysozyme activity. The fractions with lysozyme activity were analyzed by SDS-PAGE, and then pooled and concentrated. The buffer of sample was changed by 20 mM PBS at pH6.0 for further evaluation. The protein concentration was determined by Qubit® Protein Assay Kit (Invitrogen, cat Q33212).

### Example 23: Purification of SEQ ID NO: 30

The culture supernatant of O233Q7 was firstly precipitated with ammonium sulfate (80% saturation), then dialyzed with 20 mM NaAc at pH4.5. The solution was filtered with 0.45 μm filter and then loaded into SP Fast Flow column (GE Healthcare) equilibrated with 20 mM NaAc at pH4.5. A gradient increase of NaCl concentration was applied as elution buffer from zero to 1M, and then elution fractions and flow-through fraction were collected to detect lysozyme activity. The fractions with lysozyme activity were analyzed by SDS-PAGE, and then pooled and concentrated for further evaluation. The protein concentration was determined by Qubit® Protein Assay Kit (Invitrogen, cat Q33212).

### Example 24: Purification of SEQ ID NO: 36

The culture supernatant of O23CD9 was firstly precipitated with ammonium sulfate (80% saturation), then the precipitation was added water to adjust conductance to about 170 mS/cm. The solution was filtered with 0.45 μm filter and then loaded into Phenyl Sepharose 6 Fast Flow column (GE Healthcare) equilibrated with 20 mM PBS at pH7.5 with 1.5M (NH₄)₂SO₄ added. A gradient decrease of (NH₄)₂SO₄ concentration was applied as elution buffer from 1.5M to zero, and then elution fractions and flow-through fraction were collected to detect lysozyme activity.

The fractions with lysozyme activity were analyzed by SDS-PAGE, and then concentrated, changed buffer by 20 mM PBS at pH6.0 for further evaluation. The protein concentration was determined by Qubit® Protein Assay Kit (Invitrogen, cat Q33212).

### Example 25: Purification of SEQ ID NO: 39

The culture supernatant of O233Q9 was firstly precipitated with ammonium sulfate (80% saturation), then the precipitation was added water to adjust conductance to about 190 mS/cm. The solution was filtered with 0.45 μm filter and then loaded into Phenyl Sepharose 6 Fast Flow column (GE Healthcare) equilibrated with 20 mM PBS at pH6.5 with 1.8M (NH₄)₂SO₄ added. A gradient decrease of (NH₄)₂SO₄ concentration was applied as elution buffer from 1.8M to zero, and then elution fractions and flow-through fraction were collected to detect lysozyme activity.

The fractions with lysozyme activity were analyzed by SDS-PAGE, pooled together, and then concentrated. The buffer of final sample was changed by 20 mM PBS at pH6.0 for further evaluation. The protein concentration was determined by Qubit® Protein Assay Kit (Invitrogen, cat Q33212).

### Example 26: Purification of SEQ ID NO: 60

The culture supernatant of O234A2 was firstly precipitated with ammonium sulfate (80% saturation), then dialyzed with 20 mM NaAc at pH4.5. The solution was filtered with 0.45 μm filter and then loaded into Capto SP column (GE Healthcare) equilibrated with 20 mM NaAc at pH4.5. A gradient increase of NaCl concentration was applied as elution buffer from zero to 1M, and then elution fractions and flow-through fraction were collected to detect lysozyme activity. The fractions with lysozyme activity were analyzed by SDS-PAGE, pooled together, and then concentrated for further evaluation. The protein concentration was determined by Qubit® Protein Assay Kit (Invitrogen, cat Q33212).

### Example 27: Purification of SEQ ID NO: 63

The culture supernatant of O241RS was firstly precipitated with ammonium sulfate (80% saturation), then the precipitation was added water to adjust conductance to about 160 mS/cm. The solution was filtered with 0.45 μm filter and then loaded into Phenyl Sepharose 6 Fast Flow column (GE Healthcare) equilibrated with 20 mM NaAc at pH4.5 with 1.5M (NH₄)₂SO₄ added. A gradient decrease of (NH₄)₂SO₄ concentration was applied as elution buffer from 1.5M to zero, and then elution fractions and flow-through fraction were collected to detect lysozyme activity.

Since most lysozyme activity was kept in flow-through fraction, the conductivity of sample was adjusted to 180 mS/cm and loaded again into Phenyl Sepharose 6 Fast Flow column (GE Healthcare) equilibrated with 20 mM NaAc at pH4.5 with 1.8M (NH₄)₂SO₄ added. A gradient decrease of (NH₄)₂SO₄ concentration was applied as elution buffer from 1.8M to zero, and then elution fractions and flow-through fraction were collected to detect lysozyme activity. The fractions with lysozyme activity were analyzed by SDS-PAGE, but two major protein bands were found.

The fractions with lysozyme activity were pooled together and dialyzed with 20 mM NaAc at pH4.5. The sample was loaded into Capto SP column (GE Healthcare) equilibrated with 20 mM NaAc at pH4.5. A concentration gradient of NaCl was applied as elution buffer from zero to 1M, and then elution fractions and flow-through fraction were collected to detect lysozyme activity. The fractions with lysozyme activity were analyzed by SDS-PAGE and two bands also were found. The fractions were pooled together and loaded into SP High Performance column (GE Healthcare) for further purification, but the two bands still could not be separated. Finally, the fractions were pooled together, and then concentrated. The protein concentration was determined by Qubit® Protein Assay Kit (Invitrogen, cat Q33212).

### Example 28: Purification of SEQ ID NO: 66

The culture supernatant of O23T25 was firstly precipitated with ammonium sulfate (80% saturation), then dialyzed with 20 mM NaAc at pH4.5. The solution was filtered with 0.45 μm filter and then loaded into Capto SP column (GE Healthcare) equilibrated with 20 mM NaAc at pH4.5. A gradient increase of NaCl concentration was applied as elution buffer from zero to 1M, and then elution fractions and flow-through fraction were collected to detect lysozyme activity. The fractions with lysozyme activity were analyzed by SDS-PAGE, pooled together, and then concentrated for further evaluation. The protein concentration was determined by Qubit® Protein Assay Kit (Invitrogen, cat Q33212).

### Example 29: Purification of SEQ ID NO: 69

The culture supernatant of O23T23 was firstly precipitated with ammonium sulfate (80% saturation), then the precipitation was added water to adjust conductance to about 160 mS/cm. The solution was filtered with 0.45 μm filter and then loaded into Phenyl Sepharose 6 Fast Flow column (GE Healthcare) equilibrated with 20 mM NaAc at pH4.5 with 1.5M (NH₄)₂SO₄ added. A gradient decrease of (NH₄)₂SO₄ concentration was applied as elution buffer from 1.5M to zero, and then elution fractions and flow-through fraction were collected to detect lysozyme activity. The fractions with lysozyme activity were analyzed by SDS-PAGE, pooled together, and then concentrated. The buffer of final sample was changed by 20 mM NaAc at pH4.5 for further evaluation. The protein concentration was determined by Qubit® Protein Assay Kit (Invitrogen, cat Q33212).

### Example 30: Purification of SEQ ID NO: 72

The culture supernatant of O14PP6 was firstly precipitated with ammonium sulfate (80% saturation), then dialyzed with 20 mM NaAc at pH4.5. The solution was filtered with 0.45 μm filter and then loaded into SP Fast Flow column (GE Healthcare) equilibrated with 20 mM NaAc at pH4.5. A gradient increase of NaCl concentration was applied as elution buffer from zero to 1M, and then elution fractions and flow-through fraction were collected to detect lysozyme activity. The fractions with lysozyme activity were analyzed by SDS-PAGE, pooled together, and then concentrated for further evaluation. The protein concentration was determined by Qubit® Protein Assay Kit (Invitrogen, cat Q33212).

### Example 31: Purification of SEQ ID NO: 89

The culture supernatant of O24KKU was firstly precipitated with ammonium sulfate (80% saturation), then the precipitation was added water to adjust conductance to about 170 mS/cm. The solution was filtered with 0.45 μm filter and then loaded into Phenyl Sepharose High Performance column (GE Healthcare) equilibrated with 20 mM NaAc at pH5.0 with 1.5M (NH₄)₂SO₄ added. A gradient decrease of (NH₄)₂SO₄ concentration was applied as elution buffer from 1.5M to zero, and then elution fractions and flow-through fraction were collected to detect lysozyme activity. The fractions with lysozyme activity were analyzed by SDS-PAGE, pooled together, and then concentrated. The buffer of final sample was changed by 20 mM NaAc at pH5.0 for further evaluation. The protein concentration was determined by Qubit® Protein Assay Kit (Invitrogen, cat Q33212).

### Example 32: Purification of SEQ ID NO: 92

The culture supernatant of O24KKV was firstly precipitated with ammonium sulfate (80% saturation), then the precipitation was added water to adjust conductance to about 170 mS/cm. The solution was filtered with 0.45 μm filter and then loaded into Phenyl Sepharose High Performance column (GE Healthcare) equilibrated with 20 mM NaAc at pH4.5 with 1.5M (NH₄)₂SO₄ added. A gradient decrease of (NH₄)₂SO₄ concentration was applied as elution buffer from 1.5M to zero, and then elution fractions and flow-through fraction were collected to detect lysozyme activity. The fractions with lysozyme activity were analyzed by SDS-PAGE, pooled together, and then concentrated. The buffer of final sample was changed by 20 mM NaAc at pH4.5 for further evaluation. The protein concentration was determined by Qubit® Protein Assay Kit (Invitrogen, cat Q33212).

### Example 33: Purification of SEQ ID NO: 95

The culture supernatant of O24KKX was firstly precipitated with ammonium sulfate (80% saturation), then the precipitation was added water to adjust conductance to about 140 mS/cm. The solution was filtered with 0.45 μm filter and then loaded into Phenyl Sepharose High Performance column (GE Healthcare) equilibrated with 20 mM NaAc at pH4.5 with 1.2M (NH₄)₂SO₄ added. A gradient decrease of (NH₄)₂SO₄ concentration was applied as elution buffer from 1.2M to zero, and then elution fractions and flow-through fraction were collected to detect lysozyme activity. The fractions with lysozyme activity were analyzed by SDS-PAGE, pooled together, and then concentrated. The buffer of final sample was changed by 20 mM NaAc at pH4.5 for further evaluation. The protein concentration was determined by Qubit® Protein Assay Kit (Invitrogen, cat Q33212).

### Example 34: Purification of SEQ ID NO: 98

The culture supernatant of O33DRM was firstly precipitated with ammonium sulfate (80% saturation), then the precipitation was added water to adjust conductance to about 170 mS/cm. The solution was filtered with 0.45 μm filter and then loaded into Phenyl Sepharose 6 Fast Flow column (GE Healthcare) equilibrated with 20 mM Bis-Tris at pH6.5 with 1.5M (NH₄)₂SO₄ added. A gradient decrease of (NH₄)₂SO₄ concentration was applied as elution buffer from 1.5M to zero, and then elution fractions and flow-through fraction were collected to detect lysozyme activity. The fractions with lysozyme activity were analyzed by SDS-PAGE.

The fractions with lysozyme activity were pooled together and dialyzed with 20 mM Bis-Tris at pH6.5. The sample was loaded into Capto SP column (GE Healthcare) equilibrated with 20 mM Bis-Tris at pH6.5. A concentration gradient of NaCl was applied as elution buffer from zero to 1M, and then elution fractions and flow-through fraction were collected to detect lysozyme activity. The fractions with lysozyme activity were analyzed by SDS-PAGE. Finally, the fractions were pooled together, and then concentrated. The protein concentration was determined by Qubit® Protein Assay Kit (Invitrogen, cat Q33212).

### Example 35: Purification of SEQ ID NO: 101

The culture supernatant of O33DRQ was firstly precipitated with ammonium sulfate (80% saturation), then the precipitation was added water to adjust conductance to about 170 mS/cm. The solution was filtered with 0.45 μm filter and then loaded into Phenyl Sepharose 6 Fast Flow column (GE Healthcare) equilibrated with 20 mM PBS at pH7.0 with 1.5M (NH₄)₂SO₄ added. A gradient decrease of (NH₄)₂SO₄ concentration was applied as elution buffer from 1.5M to zero, and then elution fractions and flow-through fraction were collected to detect lysozyme activity. The fractions with lysozyme activity were analyzed by SDS-PAGE, pooled together, and then concentrated. The buffer of final sample was changed by 20 mM NaAc at pH5.0 for further evaluation. The protein concentration was determined by Qubit® Protein Assay Kit (Invitrogen, cat Q33212).

### Example 36: Purification of SEQ ID NO: 104

The culture supernatant of O33DRN was firstly precipitated with ammonium sulfate (80% saturation), then the precipitation was added water to adjust conductance to about 140 mS/cm. The solution was filtered with 0.45 μm filter and then loaded into Phenyl Sepharose High Performance column (GE Healthcare) equilibrated with 20 mM NaAc at pH5.5 with 1.2M (NH₄)₂SO₄ added. A gradient decrease of (NH₄)₂SO₄ concentration was applied as elution buffer from 1.2M to zero, and then elution fractions and flow-through fraction were collected to detect lysozyme activity. The fractions with lysozyme activity were analyzed by SDS-PAGE, pooled together, and then concentrated. The buffer of final sample was changed by 20 mM NaAc at pH5.0 for further evaluation. The protein concentration was determined by Qubit® Protein Assay Kit (Invitrogen, cat Q33212).

### Example 37: Purification of SEQ ID NO: 107

The culture supernatant of O33DRS was firstly precipitated with ammonium sulfate (80% saturation), then the precipitation was added water to adjust conductance to about 170 mS/cm. The solution was filtered with 0.45 μm filter and then loaded into Phenyl Sepharose High Performance column (GE Healthcare) equilibrated with 20 mM PBS at pH7.0 with 1.5M (NH₄)₂SO₄ added. A gradient decrease of (NH₄)₂SO₄ concentration was applied as elution buffer from 1.5M to zero, and then elution fractions and flow-through fraction were collected to detect lysozyme activity. The fractions with lysozyme activity were analyzed by SDS-PAGE, pooled together, and then concentrated. The buffer of final sample was changed by 20 mM NaAc at pH5.0 for further evaluation. The protein concentration was determined by Qubit® Protein Assay Kit (Invitrogen, cat Q33212).

### Example 38: Purification of SEQ ID NO: 110

The culture supernatant of O33DRT was firstly precipitated with ammonium sulfate (80% saturation), then the precipitation was added water to adjust conductance to about 150 mS/cm. The solution was filtered with 0.45 μm filter and then loaded into Phenyl Sepharose High Performance column (GE Healthcare) equilibrated with 20 mM NaAc at pH5.0 with 3M NaCl added. A gradient decrease of (NH₄)₂SO₄ concentration was applied as elution buffer from 1.2M to zero, and then elution fractions and flow-through fraction were collected to detect lysozyme activity. The fractions with lysozyme activity were analyzed by SDS-PAGE, pooled together, and then concentrated. The protein concentration was determined by Qubit® Protein Assay Kit (Invitrogen, cat Q33212).

### Example 39: Purification of SEQ ID NO: 113

The culture supernatant of O33DRU was firstly precipitated with ammonium sulfate (80% saturation), then the precipitation was added water to adjust conductance to about 185 mS/cm. The solution was filtered with 0.45 μm filter and then loaded into Phenyl Sepharose 6 Fast Flow column (GE Healthcare) equilibrated with 20 mM NaAc at pH5.0 with 1.8M (NH₄)₂SO₄ added. A gradient decrease of (NH₄)₂SO₄ concentration was applied as elution buffer from 1.8M to zero, and then elution fractions and flow-through fraction were collected to detect lysozyme activity. The fractions with lysozyme activity were analyzed by SDS-PAGE, pooled together, and then concentrated. The buffer of final sample was changed by 20 mM NaAc at pH5.0 for further evaluation. The protein concentration was determined by Qubit® Protein Assay Kit (Invitrogen, cat Q33212).

### Example 40: Purification of SEQ ID NO: 116

The culture supernatant of O33V2R was firstly precipitated with ammonium sulfate (80% saturation), then the precipitation was added water to adjust conductance to about 170 mS/cm. The solution was filtered with 0.45 μm filter and then loaded into Phenyl Sepharose 6 Fast Flow column (GE Healthcare) equilibrated with 20 mM NaAc at pH5.0 with 1.5M (NH₄)₂SO₄ added. A gradient decrease of (NH₄)₂SO₄ concentration was applied as elution buffer from 1.5M to zero, and then elution fractions and flow-through fraction were collected to detect lysozyme activity. The fractions with lysozyme activity were analyzed by SDS-PAGE, pooled together, and then concentrated. The buffer of final sample was changed by 20 mM NaAc at pH5.0 for further evaluation. The protein concentration was determined by Qubit® Protein Assay Kit (Invitrogen, cat Q33212).

### Example 41: Purification of SEQ ID NO: 128

The culture supernatant of O34A3R was firstly precipitated with ammonium sulfate (80% saturation), then dialyzed with 20 mM NaAc at pH5.5. The solution was filtered with 0.45 μm filter and then loaded into Capto SP column (GE Healthcare) equilibrated with 20 mM NaAc at pH5.5. A gradient increase of NaCl concentration was applied as elution buffer from zero to 1M, and then elution fractions and flow-through fraction were collected to detect lysozyme activity. The fractions with lysozyme activity were analyzed by SDS-PAGE, pooled together, and then concentrated for further evaluation. The protein concentration was determined by Qubit® Protein Assay Kit (Invitrogen, cat Q33212).

### Example 42: Purification of SEQ ID NO: 131

The culture supernatant of O34A3S was firstly precipitated with ammonium sulfate (80% saturation), then dialyzed with 20 mM NaAc at pH5.0. The solution was filtered with 0.45 μm filter and then loaded into Capto SP column (GE Healthcare) equilibrated with 20 mM NaAc at pH5.0. A gradient increase of NaCl concentration was applied as elution buffer from zero to 1M, and then elution fractions and flow-through fraction were collected to detect lysozyme activity. The fractions with lysozyme activity were analyzed by SDS-PAGE, pooled together, and then concentrated for further evaluation. The protein concentration was determined by Qubit® Protein Assay Kit (Invitrogen, cat Q33212).

### Example 43: Purification of SEQ ID NO: 134

The culture supernatant of O34A3T was firstly precipitated with ammonium sulfate (80% saturation), then dialyzed with 20 mM NaAc at pH5.5. The solution was filtered with 0.45 μm filter and then loaded into Capto SP column (GE Healthcare) equilibrated with 20 mM NaAc at pH5.5. A gradient increase of NaCl concentration was applied as elution buffer from zero to 1M, and then elution fractions and flow-through fraction were collected to detect lysozyme activity. The fractions with lysozyme activity were analyzed by SDS-PAGE, pooled together, and then concentrated for further evaluation. The protein concentration was determined by Qubit® Protein Assay Kit (Invitrogen, cat Q33212).

### Example 44: Purification of SEQ ID NO: 137

The culture supernatant of O34A3V was firstly precipitated with ammonium sulfate (80% saturation), then dialyzed with 20 mM NaAc at pH5.0. The solution was filtered with 0.45 μm filter and then loaded into Capto SP column (GE Healthcare) equilibrated with 20 mM NaAc at pH5.0. A gradient increase of NaCl concentration was applied as elution buffer from zero to 1M, and then elution fractions and flow-through fraction were collected to detect lysozyme activity. The fractions with lysozyme activity were analyzed by SDS-PAGE, but two bands were found.

The fractions with lysozyme activity were pooled together and adjusted pH to 4.5, then loaded into SP High Performance column (GE Healthcare) equilibrated with 20 mM NaAc at pH4.5. A gradient increase of NaCl concentration was applied as elution buffer from zero to 1M, and then elution fractions and flow-through fraction were collected to detect lysozyme activity. The fractions with lysozyme activity were analyzed by SDS-PAGE. Two bands with lysozyme activity were collected separately, then concentrated for further evaluation with two different purification IDs. The protein concentration was determined by Qubit® Protein Assay Kit (Invitrogen, cat Q33212).

### Example 45: Purification of SEQ ID NO: 140

The culture supernatant of O34A3W was firstly precipitated with ammonium sulfate (80% saturation), then dialyzed with 20 mM NaAc at pH4.5. The solution was filtered with 0.45 μm filter and then loaded into SP Fast Flow column (GE Healthcare) equilibrated with 20 mM NaAc at pH4.5. A gradient increase of NaCl concentration was applied as elution buffer from zero to 1M, and then elution fractions and flow-through fraction were collected to detect lysozyme activity. The fractions with lysozyme activity were analyzed by SDS-PAGE, pooled together, and then concentrated for further evaluation. The protein concentration was determined by Qubit® Protein Assay Kit (Invitrogen, cat Q33212).

### Example 46: Purification of SEQ ID NO: 143

The culture supernatant of O34A3X was firstly precipitated with ammonium sulfate (80% saturation), then the precipitation was added water to adjust conductance to about 200 mS/cm. The solution was filtered with 0.45 μm filter and then loaded into Phenyl Sepharose High Performance column (GE Healthcare) equilibrated with 20 mM PBS at pH6.0 with 2.0M (NH₄)₂SO₄ added. A gradient decrease of (NH₄)₂SO₄ concentration was applied as elution buffer from 2.0M to zero, and then elution fractions and flow-through fraction were collected to detect lysozyme activity and analyzed by SDS-PAGE. The fractions with lysozyme activity were pooled together and then dialyzed with 20 mM NaAc at pH5.0. The sample was loaded into Capto SP column (GE Healthcare) equilibrated with 20 mM NaAc at pH5.0. A gradient increase of NaCl concentration was applied as elution buffer from zero to 1M, and then elution fractions and flow-through fraction were collected to detect lysozyme activity. The fractions with lysozyme activity were analyzed by SDS-PAGE, pooled together, and then concentrated. The protein concentration was determined by Qubit® Protein Assay Kit (Invitrogen, cat Q33212).

### Example 47: Purification of SEQ ID NO: 146

The culture supernatant of O33X6Z was firstly precipitated with ammonium sulfate (80% saturation), then dialyzed with 20 mM NaAc at pH4.5. The solution was filtered with 0.45 μm filter and then loaded into Capto SP column (GE Healthcare) equilibrated with 20 mM NaAc at pH4.5. A gradient increase of NaCl concentration was applied as elution buffer from zero to 1M, and then elution fractions and flow-through fraction were collected to detect lysozyme activity. The fractions with lysozyme activity were analyzed by SDS-PAGE, pooled together, and then concentrated for further evaluation. The protein concentration was determined by Qubit® Protein Assay Kit (Invitrogen, cat Q33212).

### Example 48: Purification of GH25 Lysozymes

### General Purification Procedure

The fermentation supernatant with the lysozyme was filtered through a Fast PES Bottle top filter with a 0.22 μm cut-off. pH was adjusted to 4.5 with 10% acetic acid. After the pH-adjustment the solution became a little cloudy and this was removed by filtration through a Fast PES Bottle top filter with a 0.22 μm cut-off.

After pretreatment about 650 ml of the lysozyme containing solution was purified by chromatography on SP Sepharose, approximately 50 ml in a XK26 column, using as buffer A 50 mM Na-acetate pH 4.5, and as buffer B 50 mM Na-acetate+1 M NaCl pH 4.5. The fractions from the column were pooled based on the chromatogram (absorption at 280 and 254 nm) and SDS-PAGE analysis. The pooled fractions were buffer-changed into 50 mM Na-acetate, pH 5.5 and concentrated using Amicon spin filters with a 10 kDa cut-off.

### Purification of SEQ ID NO: 45, 48, 51 and 54

The fermentation broth biomass was separated by centrifugation. The sample filtration was then carried out by tangential flow filtration using hollow fiber 0.2 u cartridges mounted on a QuixStand® system and then loaded into a Hydrophobic column (TOYOPEARL® Phenyl-650M) equilibrated with 50 mM HEPES pH 8+1.5M Ammonium Sulphate at pH8.0. Step gradient decrease of ammonium sulphate concentration was applied as elution buffer from 1.5M to zero. The pooled elution peak fractions, wash and flow-through were collected and analysed on SDS gel. The peak with lysozyme band on gel was then buffer exchanged by 50 mM HEPES pH 8 and the protein concentration was determined by spectrophotometer (Agilent 8453 UV-visible Spectroscopy System).

### Purification of SEQ ID NO: 6, 18, 21, 24, 27, 33, 42, 227, 75, 86, 119, 122, 230, 233 and 155

The fermentation supernatant with the lysozyme was filtered through a Fast PES Bottle top filter with a 0.22 μm cut-off.

Pretreatment of the filtered broth may be necessary if the expression level is low and/or conductivity is high (in general >10S/m). Pretreatment can be performed using ultrafiltration on a 3-5 kDa cutoff membrane, buffer exchange on a G25 gel filtration column or dialysis. SEQ ID NO: 33, 42, 75 and 122 were pretreated using a G25 gel filtration column using 50 mM acetate pH4.5 whilst SEQ ID NO: 18, 21 and 27 were pretreated in a dialysis membrane (6-8 KDa) overnight in 50 mM scetate 4.5.

pH was adjusted to 4.5. If the solution became cloudy after the pH-adjustment, this was removed by filtration through a Fast PES Bottle top filter with a 0.22 μm cut-off. The lysozyme containing solution was purified by chromatography on SP Sepharose, approximately 50 ml in a XK26 column, using as buffer A 50 mM Na-acetate pH 4.5, and as buffer B 50 mM Na-acetate +1 M NaCl pH 4.5 and a 0-100% gradient over ca. 10 CV. The fractions from the column were pooled based on the chromatogram (absorption at 280 and 254 nm) and SDS-PAGE analysis.

The molecular weight was estimated from SDS-PAGE and the purity was >90%.

### Purification of SEQ ID NO: 9

The fermentation supernatant with the lysozyme was filtered through a Fast PES Bottle top filter with a 0.22 μm cut-off.

The pH adjusted to 7.5 and the sample was filtered through a Fast PES Bottle top filter with a 0.22 μm cut-off. The lysozyme containing solution was purified by chromatography on Q-Sepharose, approximately 70 ml in a XK26 column, using as buffer A 50 mM Tris pH 7.5 and as buffer B 50 mM Tris+1 M NaCl pH 7.5 and a 0-100% gradient over ca. 5 CV. The fractions from the column were pooled based on the chromatogram (absorption at 280 and 254 nm) and SDS-PAGE analysis.

The molecular weight was estimated from SDS-PAGE and the purity was >90%.

### Purification of SEQ ID NO: 78

The fermentation supernatant with the lysozyme was filtered through a Fast PES Bottle top filter with a 0.22 μm cut-off.

The sample was concentrated on a vivacelle (cut off 5000 kd). The sample was diluted 3× with Milli-Q water and pH adjusted to 7.5. The lysozyme containing solution was purified by chromatography on Q-Sepharose, approximately 30 ml in a XK26 column, using as buffer A 50 mM Hepes pH 7.5 and as buffer B 50 mM Hepes+1 M NaCl pH 7.5 and a 0-50% gradient over ca. 10 CV. The fractions from the column were pooled based on the chromatogram (absorption at 280 and 254 nm) and SDS-PAGE analysis.

The molecular weight was estimated from SDS-PAGE and the purity was >90%.

### Purification of SEQ ID NO: 83

The fermentation supernatant with the lysozyme was filtered through a Fast PES Bottle top filter with a 0.22 μm cut-off.

The sample was ultrafiltreted and diafiltrated on Sartorius UF-system 10 kDa. If the solution became cloudy after the pH-adjustment, this was removed by filtration through a Fast PES Bottle top filter with a 0.22 μm cut-off. The lysozyme containing solution was purified by chromatography on Q-Sepharose, approximately 50 ml in a XK26 column, using as buffer A 50 mM Hepes pH 7.0 and as buffer B 50 mM Hepes+1 M NaCl pH 7.0 and a 0-100% gradient over ca. 10 CV. The fractions from the column were pooled based on the chromatogram (absorption at 280 and 254 nm) and SDS-PAGE analysis.

The molecular weight was estimated from SDS-PAGE and the purity was >90%.

### Purification of SEQ ID NO: 125

The fermentation supernatant with the lysozyme was filtered through a Fast PES Bottle top filter with a 0.22 μm cut-off.

The sample was pretreated using a G25 gel filtration column using 50 mM Na-acetate pH and the elutant was purified by chromatography on Q-Sepharose, approximately 50 ml in a XK26 column, using as buffer A 50 mM Tris pH 8.0 and as buffer B 50 mM Tris+2 M NaCl pH 8.0 and a 0-100% gradient over ca. 10 CV. The fractions from the column were pooled based on the chromatogram (absorption at 280 and 254 nm) and SDS-PAGE analysis.

The molecular weight was estimated from SDS-PAGE and the purity was >90%.

### Purification of SEQ ID NO: 158

The fermentation supernatant with the lysozyme was filtered through a Fast PES Bottle top filter with a 0.22 μm cut-off.

The sample was diluted 50% with milli-Q water and the pH was adjusted to pH 4.5. The sample was purified by chromatography on Capto S, approximately 50 ml in a XK26 column, using as buffer A 50 mM acetate pH 9.0 and as buffer B 50 mM acetate+2 M NaCl pH 9.0. The fractions from the column were pooled based on the chromatogram (absorption at 280 and 254 nm) and SDS-PAGE analysis.

The molecular weight was estimated from SDS-PAGE and the purity was >90%.

### Example 49: Determination of Lysozyme Activity

Method for the Determination of Lysozyme Activity Against Micrococcus lysodeikticus.

The lysozymes of the application were tested according to Example 1 in the same plate as a prior art lysozyme (a GH25 lysozyme from Acremonium alcalophilum as described in WO 2013/076253, herein denoted SEQ ID NO: 159). The ratio of OD measurements of the lysozyme of the present disclosure to the lysozyme of SEQ ID NO: 159 was calculated and is shown in tables 5 to 32 below.

TABLE 5
OD Meaurement of SEQ ID NO: 24 and 227

| Lysozyme | OD Measurement | Ratio |
|---|---|---|
| SEQ ID NO: 159 | 0.41 | — |
| SEQ ID NO: 227 | 1.34 | 3.3 |
| SEQ ID NO: 24 | 1.99 | 4.9 |

TABLE 6
OD Measurement of SEQ ID NO: 6 and 9

| Lysozyme | OD Measurement | Ratio |
|---|---|---|
| SEQ ID NO: 159 | 0.37 | — |
| SEQ ID NO: 9 | 0.85 | 2.3 |
| SEQ ID NO: 6 | 1.78 | 4.8 |

TABLE 7
OD Measurement of SEQ ID NO: 72 and 83

| Lysozyme | OD Measurement | Ratio |
|---|---|---|
| SEQ ID NO: 159 | 0.49 | — |
| SEQ ID NO: 83 | 0.99 | 2.0 |
| SEQ ID NO: 72 | 2.22 | 4.5 |

TABLE 8
OD Measurement of SEQ ID NO: 86

| Lysozyme | OD Measurement | Ratio |
|---|---|---|
| SEQ ID NO: 159 | 0.65 | — |
| SEQ ID NO: 86 | 1.34 | 2.1 |

TABLE 9
OD Measurement of SEQ ID NO: 30 and 60

| Lysozyme | OD Measurement | Ratio |
|---|---|---|
| SEQ ID NO: 159 | 0.61 | — |
| SEQ ID NO: 60 | 2.32 | 3.5 |
| SEQ ID NO: 30 | 2.14 | 3.2 |

TABLE 10
OD Measurement of SEQ ID NO: 3, 36 and 39

| Lysozyme | OD Measurement | Ratio |
|---|---|---|
| SEQ ID NO: 159 | 0.52 | — |
| SEQ ID NO: 36 | 2.52 | 4.8 |
| SEQ ID NO: 3 | 2.60 | 5.0 |
| SEQ ID NO: 39 | 1.71 | 3.3 |

TABLE 11
OD Measurement of SEQ ID NO: 27 and 80

| Lysozyme | OD Measurement | Ratio |
|---|---|---|
| SEQ ID NO: 159 | 0.62 | — |
| SEQ ID NO: 80 | 1.86 | 3.0 |
| SEQ ID NO: 27 | 1.20 | 2.0 |

TABLE 12
OD Measurement of SEQ ID NO: 18 and 21

| Lysozyme | OD Measurement | Ratio |
|---|---|---|
| SEQ ID NO: 159 | 0.69 | — |
| SEQ ID NO: 18 | 1.76 | 2.6 |
| SEQ ID NO: 21 | 2.62 | 3.8 |

TABLE 13
OD Measurement of SEQ ID NO: 63, 66 and 69

| Lysozyme | OD Measurement | Ratio |
|---|---|---|
| SEQ ID NO: 159 | 0.63 | — |
| SEQ ID NO: 63 | 1.38 | 2.2 |
| SEQ ID NO: 66 | 1.72 | 2.7 |
| SEQ ID NO: 69 | 2.03 | 3.2 |

TABLE 14
OD Measurement of SEQ ID NO: 12 and 15

| Lysozyme | OD Measurement | Ratio |
|---|---|---|
| SEQ ID NO: 159 | 0.71 | — |
| SEQ ID NO: 15 | 1.79 | 2.5 |
| SEQ ID NO: 12 | 1.47 | 2.1 |

TABLE 15
OD Measurement of SEQ ID NO: 45, 51 and 54

| Lysozyme | OD Measurement | Ratio |
|---|---|---|
| SEQ ID NO: 159 | 0.50 | — |
| SEQ ID NO: 51 | 2.06 | 4.2 |
| SEQ ID NO: 54 | 1.98 | 4.0 |
| SEQ ID NO: 45 | 2.07 | 4.2 |

TABLE 16
OD Measurement of SEQ ID NO: 48

| Lysozyme | OD Measurement | Ratio |
|---|---|---|
| SEQ ID NO: 159 | 0.44 | — |
| SEQ ID NO: 48 | 0.93 | 2.1 |

TABLE 17
OD Measurement of SEQ ID NO: 33 and 75

| Lysozyme | OD Measurement | Ratio |
|---|---|---|
| SEQ ID NO: 159 | 0.56 | — |
| SEQ ID NO: 75 | 1.56 | 2.8 |
| SEQ ID NO: 33 | 2.33 | 4.2 |

TABLE 18
OD Measurement of SEQ ID NO: 21 and 42

| Lysozyme | OD Measurement | Ratio |
|---|---|---|
| SEQ ID NO: 159 | 0.72 | — |
| SEQ ID NO: 21 | 3.13 | 4.4 |
| SEQ ID NO: 42 | 1.94 | 2.7 |

TABLE 19
OD Measurement of SEQ ID NO: 24 and 125

| Lysozyme | OD Measurement | Ratio |
|---|---|---|
| SEQ ID NO: 159 | 0.44 | — |
| SEQ ID NO: 24 | 1.97 | 4.5 |
| SEQ ID NO: 125 | 1.74 | 2.9 |

TABLE 20
OD Measurement of SEQ ID NO: 98

| Lysozyme | OD Measurement | Ratio |
|---|---|---|
| SEQ ID NO: 159 | 0.55 | — |
| SEQ ID NO: 98 | 1.79 | 3.3 |

TABLE 21
OD Measurement of SEQ ID NO: 104 and 122

| Lysozyme | OD Measurement | Ratio |
|---|---|---|
| SEQ ID NO: 159 | 0.44 | — |
| SEQ ID NO: 122 | 2.09 | 4.8 |
| SEQ ID NO: 104 | 1.23 | 2.8 |

TABLE 22
OD Measurement of SEQ ID NO: 89

| Lysozyme | OD Measurement | Ratio |
|---|---|---|
| SEQ ID NO: 159 | 0.63 | — |
| SEQ ID NO: 89 | 2.11 | 3.4 |

TABLE 23
OD Measurement of SEQ ID NO: 92, 95, 128, 134 and 143

| Lysozyme | OD Measurement | Ratio |
|---|---|---|
| SEQ ID NO: 159 | 0.49 | — |
| SEQ ID NO: 92 | 1.30 | 2.6 |
| SEQ ID NO: 95 | 1.59 | 3.2 |
| SEQ ID NO: 128 | 2.38 | 4.8 |
| SEQ ID NO: 134 | 1.24 | 2.5 |
| SEQ ID NO: 143 | 2.59 | 5.2 |

TABLE 24
OD Measurement of SEQ ID NO: 146

| Lysozyme | OD Measurement | Ratio |
|---|---|---|
| SEQ ID NO: 159 | 0.38 | — |
| SEQ ID NO: 146 | 1.94 | 5.1 |

TABLE 25
OD Measurement of SEQ ID NO: 110 and 116

| Lysozyme | OD Measurement | Ratio |
|---|---|---|
| SEQ ID NO: 159 | 0.46 | — |
| SEQ ID NO: 110 | 1.53 | 3.3 |
| SEQ ID NO: 116 | 1.28 | 2.8 |

TABLE 26
OD Measurement of SEQ ID NO: 131

| Lysozyme | OD Measurement | Ratio |
|---|---|---|
| SEQ ID NO: 159 | 0.30 | — |
| SEQ ID NO: 131 | 2.35 | 7.8 |

TABLE 27
OD Measurement of SEQ ID NO: 110, 128 and 137

| Lysozyme | OD Measurement | Ratio |
|---|---|---|
| SEQ ID NO: 159 | 0.34 | — |
| SEQ ID NO: 137 | 1.87 | 5.5 |
| SEQ ID NO: 137 | 1.48 | 4.4 |
| SEQ ID NO: 128 | 2.09 | 6.2 |
| SEQ ID NO: 110 | 1.50 | 4.5 |

TABLE 28
OD Measurement of SEQ ID NO: 131

| Lysozyme | OD Measurement | Ratio |
|---|---|---|
| SEQ ID NO: 159 | 0.40 | — |
| SEQ ID NO: 131 | 2.42 | 6.0 |

TABLE 29
OD Measurement of SEQ ID NO: 140

| Lysozyme | OD Measurement | Ratio |
|---|---|---|
| SEQ ID NO: 159 | 0.61 | — |
| SEQ ID NO: 140 | 2.03 | 3.3 |

TABLE 30
OD Measurement of SEQ ID NO: 158

| Lysozyme | OD Measurement | Ratio |
|---|---|---|
| SEQ ID NO: 159 | 0.56 | — |
| SEQ ID NO: 158 | 1.65 | 3.0 |

TABLE 31
OD Measurement of SEQ ID NO: 230, 233 and 155

| Lysozyme | OD Measurement | Ratio |
|---|---|---|
| SEQ ID NO: 159 | 0.48 | — |
| SEQ ID NO: 230 | 1.34 | 2.8 |
| SEQ ID NO: 233 | 1.52 | 3.2 |
| SEQ ID NO: 155 | 1.01 | 2.1 |

TABLE 32
OD Measurement of SEQ ID NO: 101, 107, 113 and 119

| Lysozyme | OD Measurement | Ratio |
|---|---|---|
| SEQ ID NO: 159 | 0.53 | — |
| SEQ ID NO: 113 | 1.77 | 3.4 |
| SEQ ID NO: 101 | 1.91 | 3.6 |
| SEQ ID NO: 107 | 1.50 | 2.9 |
| SEQ ID NO: 119 | 2.40 | 4.6 |

The results show that the lysozymes of the present disclosure have increased activity compared to this prior art lysozyme.

### Example 50: Animal Feed and Animal Feed Additives Comprising a Lysozyme of the Present Disclosure

### Animal Feed Additive

A formulation of a lysozyme of the present disclosure (e.g. SEQ ID NO: 3, 6, 9, 12, 15, 18, 21, 24, 27, 30, 33, 36, 39, 42, 45, 48, 51, 54, 57, 60, 63, 66, 69, 72, 75, 80, 83, 86, 89, 92, 95, 98, 101, 104, 107, 110, 113, 116, 119, 122, 125, 128, 131, 134, 137, 140, 143, 146, 149, 152, 155, 158, 221, 224, 227, 230 or 233) containing 0.01 g to 10 g enzyme protein is added to the following premix (per kilo of premix):

| | | |
|---|---|---|
| 5000000 | IE | Vitamin A |
| 1000000 | IE | Vitamin D3 |
| 13333 | mg | Vitamin E |
| 1000 | mg | Vitamin K3 |
| 750 | mg | Vitamin B1 |
| 2500 | mg | Vitamin B2 |
| 1500 | mg | Vitamin B6 |
| 7666 | mcg | Vitamin B12 |
| 12333 | mg | Niacin |
| 33333 | mcg | Biotin |
| 300 | mg | Folic Acid |
| 3000 | mg | Ca-D-Panthothenate |
| 1666 | mg | Cu |
| 16666 | mg | Fe |
| 16666 | mg | Zn |
| 23333 | mg | Mn |
| 133 | mg | Co |
| 66 | mg | I |
| 66 | mg | Se |
| 5.8 | % | Calcium |
| 25 | % | Sodium |

### Animal Feed

This is an example of an animal feed (broiler feed) comprising the animal feed additive as described above:
62.55% Maize
33.8% Soybean meal (50% crude protein)
1.0% Soybean oil
0.2% DL-Methionine
0.22% DCP (dicalcium phosphate)
0.76% CaCO₃ (calcium carbonate)
0.32% Sand
0.15% NaCl (sodium chloride)
1% of the above Premix

The ingredients are mixed, and the feed is pelleted at the desired temperature, e.g. 60, 65, 75, 80, 85, 90 or even 95° C.

### Example 51: Cloning and Expression of Flammulina velutipes (SEQ ID NO: 221)

A GH25 lysozyme was identified from the downloaded genomic DNA assembly from the following project: Park Y J et al., “Whole genome and global gene expression analyses of the model mushroom Flammulina velutipes reveal a high capacity for lignocellulose degradation.”, PLOS One, 2014 Apr. 8; 9 (4): e93560.

A codon optimized, intronless version of the Flammulina velutipes GH25 lysozyme (SEQ ID NO: 217) was ordered from GeneArt, Invitrogen having SEQ ID NO: 219.

SEQ ID NO: 219—the underlined portions are restriction enzymes used for cloning into pDau109.

SEQ ID NO: 219
ACACAACTGGGGATCCACCATGCGAATCCTCTTGTTCATCGCAGT
CACAATCGCGTTGGGAGTCCATGCCAGGCTCAACGGCATCGATGT
CTCGGGATACCAGCCGAACGTCAACTGGGCCACGGTCAAAGCGAA
CGGCGTGTCCTTCGCGTACATCAAGGCAACCGAGGGCACCACGTA
TACAAACCCCTCGTTCTCCTCCCAGTACACCGGAGCAACAAAAGC
CGGATTGATCAGGGGCTCCTACCATTTCGCGCATCCTTCGTCGTC
CACAGGCGCAGCACAGGCACGATACTTCGTGGCACATGGCGGTGG
TTGGTCCGGTGATGGTATCACCTTGCCTGGTGCGCTCGATATCGA
GTATAACCCTTCGGGTGCGACATGTTACGGCCTCTCGACCTCGTC
CATGGTGAACTGGATCGCCGATTTCTCCAACACTTATCACTCGCT
CACAGGCAGGTACCCCGTCATTTACACCACTGCCGATTGGTGGCG
AACCTGTACCGGCAACTCCGCATCCTTCGCAAACAACTCGCCTCT
CTGGATTGCGCGTTACGCGTCGACTATCGGTACGCTCCCTGCCGG
ATGGTCGTACGCGACCTTCTGGCAGTATGCGGATTCGGGCTCCAA
CCCTGGCGATCAGGATTACTTCAACGGTGACGCAGCGGGTCTCAA
GCGTCTCGCGACATCGTAATAAGCTTCTCGAGATCT

The synthetic gene was cloned directly into pDau109 as described in the Example 16 as a ligation into the BamHI-HindIII site of the vector. The GH25 lysozyme having SEQ ID NO: 221 was expressed as described in Example 19 and purified as described in Example 52.

### Example 52: Purification of the GH25 Lysozyme from Flammulina velutipes (SEQ ID NO: 221)

The fermentation supernatant with the lysozyme was filtered through a Fast PES Bottle top filter with a 0.22 μm cut-off.

The sample was pretreated using a G25 gel filtration column using 50 mM acetate pH4.5 and the elutant was purified by chromatography on SP Sepharose, approximately 50 ml in a XK26 column, using as buffer A 50 mM Na-acetate pH 4.5, and as buffer B 50 mM Na-acetate+2 M NaCl pH 4.5 and a 0-100% gradient over ca. 10 CV. The fractions from the column were pooled based on the chromatogram (absorption at 280 and 254 nm) and SDS-PAGE analysis.

The molecular weight was estimated from SDS-PAGE and the purity was >90%.

### Example 53: PCR Amplification and InFusion Cloning of Lysozyme Encoding Sequences SEQ ID NO: 222

The InFusion cloning primers given in table 33 were used to generate an BamHI-HindIII flanked cloning cassette from the genomic DNA as described in Example 16.

TABLE 33
InFusion cloning primers

| | | Primer to | |
|---|---|---|---|
| | Primer | lysozyme | |
| | SEQ | SEQ | |
| Primer* | ID NO: | ID NO: | Sequence |
| C8VSE-F | 225 | 222 | 5'-ACACAACTGGGGATCCAC |
| | | | CATGCTTTTCGCAACCTTTCT |
| | | | TTGTCTTG |
| C8VSE-R | 226 | 222 | 5'-AGATCTCGAGAAGCTTAC |
| | | | GAGGAAAAGACGTTGAGGTCA |
| | | | AATTGA |

*-F—forward primer;
-R—reverse primer

Bold letters represent coding sequence. The underlined sequence is homologous to the insertion sites of pDau109.

The GH25 lysozymes were expressed in Aspergillus oryzae as described in Example 19 and purified as described in Example 54.

### Example 54: Purification of the GH25 Lysozyme from Deconica coprophila (SEQ ID NO: 222)

The fermentation supernatant with the lysozyme was filtered through a Fast PES Bottle top filter with a 0.22 μm cut-off.

The sample was pretreated using a G25 gel filtration column using 50 mM acetate pH4.5 and the elutant was purified by chromatography on SP Sepharose, approximately 50 ml in a XK26 column, using as buffer A 50 mM Na-acetate pH 4.5, and as buffer B 50 mM Na-acetate+2 M NaCl pH 4.5 and a 0-100% gradient over ca. 10 CV. The fractions from the column were pooled based on the chromatogram (absorption at 280 and 254 nm) and SDS-PAGE analysis.

The molecular weight was estimated from SDS-PAGE and the purity was >90%.

### Example 55: Determination of Lysozyme Activity

The lysozymes of the application were tested according to Example 1 in the same plate as a prior art lysozyme (a GH25 lysozyme from Acremonium alcalophilum as described in WO 2013/076253, herein denoted SEQ ID NO: 159). The ratio of OD measurement of the lysozyme of the present disclosure to the lysozyme of SEQ ID NO: 159 was calculated and is shown in Table 34 below.

TABLE 34
OD Measurement of SEQ ID NO: 221 and 224

| Lysozyme | OD Measurement | Ratio |
|---|---|---|
| SEQ ID NO: 159 | 0.51 | — |
| SEQ ID NO: 221 | 1.74 | 3.4 |
| SEQ ID NO: 224 | 1.80 | 3.5 |

The results show that the lysozymes of the present disclosure have increased activity compared to this prior art lysozyme.

### Example 56 Method for the Determination of Lysozyme Activity Against Lactobacillus johnsonii

### PGN Extraction:

Cultivation of Lactobacillus johnsonii:

### Materials

MRS broth, product number BD 288130, pH 6.3-6.7.
MRS agar plates, BD 288130; Agar Oxoid LP0011; pH 6.3-6.7.
0.9% NaCl, Merck 106404, Cas no. 7647145
jars, supplier Merck 116387, Anaerocult anaerobic jar 2.5 L
Anaerogen 2.5 L, ThermoScientific, catalogue no. AN0025A Lactobacillus johnsonii, DSM10533

### Procedure

L. johnsonii was streaked from freeze stock to MRS agar plate and incubated under anaerobic conditions for 2 days, anaerobic jar with Anaerogen 2.5 L, 30° C. Some colonies were inoculated into 500 mLMRS broth in a 500 mL blue cap bottle and placed in an anaerobic jar with Anaerogen 2.5 L for 72 hours at 30° C.

The culture was spun down (6000 rpm, 10 minutes) and the supernatant was poured off before another round of centrifugation was performed. The pellet was washed in 100 mL 0.9% NaCl and the suspension was mixed well and centrifuged at 6000 rpm for 10 minutes. The supernatant was poured off and the washing procedure in 0.9% NaCl was repeated to a total of three washes. Approximately 40 mL 0.9% NaCl was added to the pellet and the solution was transferred to a 50 mL falcon tube. The solution was centrifuged at 6000 rpm for 10 minutes and the supernatant was poured off. The pellet was stored at −18° C. until the extraction of the peptidoglycan was conducted.

### Extraction Procedure:

### Materials

Protease from Streptomyces griseus, Sigma-Aldrich P5147, CAS 9036-06-0
PBS pH 7.3:
NaCl: 8 g, Sigma-Aldrich 31434, CAS 7647-14-5
KCl: 0.2 g, Sigma-Aldrich P9333, CAS 7447-40-7
KH₂PO₄: 0.24 g, Sigma-Aldrich P5655, CAS 7778-77-0
Na₂HPO₄·2H₂O: 1.44 g, Sigma-Aldrich 30412, CAS 10028-24-7
Add Milli-Q water to 1000 mL
1% Triton-X 100 solution:
1 mL Triton X100, Sigma-Aldrich X100, CAS 9002-93-1
Add Milli Q water to 100 mL
500 mM sodium carbonate buffer, pH 9.3:
500 mM sodium carbonate is made from 21 g Na₂CO₃ (Sigma-Aldrich S7795, CAS 497-19-8) in 500 mL MQ water
500 mM sodium bicarbonate is made from 72 g NaHCO₃(Sigma-Aldrich S6014, CAS 144-55-8) in 500 mL MW water
The pH 9.3 buffer is made from 320 mL NaHCO₃ and 80 mL Na₂CO₃ and adjusting pH with HCl
Phenol solution with 10 mM Tris HCl, pH 8.0, 1 mM EDTA, Sigma-Aldrich P4557, CAS 108-95-2
Acetone, Sigma-Aldrich 32201-M, CAS 67-64-1
Ethanol, 96%, CCS Healthcare 1680643, CAS 64-17-5

### Procedure

L. johnsonii cell material was freeze dried. The freeze dried material (525 mg) was suspended in PBS (40 mL) in a 50 mL Falcon tube. The suspension was shaken for 2 h @ 700 rpm in a thermoshaker at room temperature. Streptomyces griseus protease (55 mg) was then added and the suspension was incubated 6 h @ 37 C in the thermoshaker. It was then centrifuged 20 min @ 1900 g at room temperature, and the supernatant was decanted. The pellet was re-suspended in 1% Triton X-100 (40 mL) and shaken overnight @ 37 C. After another centrifugation and decantation, the pellet was re-suspended in PBS (40 mL) and protease (55 mg) added again. The suspension was again incubated 6 h @ 37 C, centrifuged and decanted. The pellet was re-suspended in PBS (40 mL) and shaken overnight @ 37 C. This washing procedure was repeated once more with PBS (40 mL, 30 min agitation), then with 50% ethanol/water (40 mL, 30 min agitation). The pellet was then split into two Falcon tubes. To each tube was added phenol solution (15 mL) pre-heated to 40 C. The suspensions were shaken 10 min @ 40 C, and then added 96% ethanol (25 mL to each tube), centrifuged and decanted. The pellets were further washed with acetone (40 mL in each tube) and 96% ethanol (40 mL in each tube), before being freeze dried. Combining the pellets from the two tubes yielded 80 mg purified peptidoglycan as a white powder.

### Reducing End Assay

The lysozyme was diluted in phosphate dilution buffer (5 mM citrate, 5 mM K₂HPO₄, 0.01% TritonX-100, pH 5.0) to 200 or 50 μg/mL in polypropylene tubes, dependent on the strength of available stock solutions. The diluted lysozyme was further diluted in a 96-well polypropylene microtiter plate by preparing a two-fold dilution series down to a concentration of 6.3 μg/mL in phosphate dilution buffer (5 mM citrate, 5 mM K₂HPO₄, 0.01% TritonX-100, pH 5.0). A 50 mg/ml stock solution of L. johnsonii substrate in MillQ was prepared and diluted in phosphate buffer (50 mM citrate, 50 mM K₂HPO₄, pH 5.0) to 250 μg/ml. In a polypropylene deepwell plate 50 μL of the lysozyme dilution was mixed with 450 μL L. johnsonii solution and incubated at 40° C. with shaking (500 rpm) for 45 min. After incubation, the deepwell plate was centrifuged (3200 rpm, 7 min) to pellet insoluble material and 100 μL of the supernatant was mixed with 50 μL 3.2M HCl in a 96-well PCR plate and incubated at 95° C. for 80 min. 50 μL of 3.5 M NaOH was added to each well of the PCR plate, and 150 μL of each sample was transferred to a new PCR plate containing 75 μL/well 4-hydroxybenzhydrazide (PAHBAH) solution in K—Na tartrate/NaOH buffer (50 g/L K—Na tartrate+20 g/L NaOH). The plate was incubated at 95° C. for 10 min before 100 μL/sample was transferred to a clear flat-bottomed microtiter plate for optical density (OD) measurement at 405 nm. OD measurements were performed on three times diluted samples (50 μL sample diluted in 100 μL in Milli-Q water). The OD measurement values represent the difference after the original (background) reading was subtracted and represent the average of two OD measurement values. Results are shown in Table 35 and in FIGS. 3 and 4.

TABLE 35
Average OD405 measurements (background corrected)
in Reducing End Assay

| | Concentration of lysozyme in μg/mL | | | | | |
|---|---|---|---|---|---|---|
| Lysozyme | 20 | 10 | 5 | 2.5 | 1.25 | 0.63 |
| SEQ ID NO: 24 | 1.07 | 0.85 | 0.56 | 0.39 | 0.26 | 0.15 |
| SEQ ID NO: 227 | 0.19 | 0.12 | 0.10 | 0.07 | 0.01 | 0.01 |
| SEQ ID NO: 6 | 0.66 | 0.47 | 0.40 | 0.25 | 0.14 | 0.10 |
| SEQ ID NO: 9 | 0.03 | 0.01 | 0.19 | 0.02 | 0.01 | 0.00 |
| SEQ ID NO: 72 | 1.00 | 0.71 | 0.50 | 0.40 | 0.24 | 0.16 |
| SEQ ID NO: 83 | 0.29 | 0.13 | 0.08 | 0.09 | 0.02 | 0.03 |
| SEQ ID NO: 60 | 0.91 | 0.53 | 0.40 | 0.19 | 0.10 | 0.06 |
| SEQ ID NO: 30 | 0.33 | 0.24 | 0.12 | 0.04 | 0.00 | −0.04 |
| SEQ ID NO: 39 | 0.85 | 0.72 | 0.45 | 0.34 | 0.19 | 0.08 |
| SEQ ID NO: 3 | 0.90 | 0.66 | 0.49 | 0.29 | 0.18 | 0.07 |
| SEQ ID NO: 51 | 0.71 | 0.43 | 0.45 | 0.23 | 0.14 | 0.05 |
| SEQ ID NO: 54 | 0.58 | 0.48 | 0.27 | 0.16 | 0.12 | 0.02 |
| SEQ ID NO: 48 | 0.66 | 0.45 | 0.44 | 0.20 | 0.19 | 0.03 |
| SEQ ID NO: 18 | 0.98 | 0.75 | 0.55 | 0.33 | 0.16 | 0.28 |
| SEQ ID NO: 27 | −0.02 | 0.06 | −0.02 | −0.03 | −0.02 | −0.03 |
| SEQ ID NO: 224 | −0.06 | −0.07 | −0.04 | −0.06 | −0.07 | −0.07 |
| SEQ ID NO: 15 | 1.10 | 0.98 | 0.86 | 0.53 | 0.40 | 0.20 |
| SEQ ID NO: 69 | 0.45 | 0.27 | 0.16 | 0.05 | 0.17 | −0.04 |
| SEQ ID NO: 66 | −0.06 | −0.03 | 0.01 | −0.06 | −0.03 | −0.08 |
| SEQ ID NO: 63 | 0.15 | 0.04 | 0.03 | −0.04 | −0.05 | −0.06 |
| SEQ ID NO: 12 | −0.03 | −0.07 | −0.06 | −0.07 | −0.08 | 0.05 |
| SEQ ID NO: 45 | 0.98 | 0.74 | 0.61 | 0.26 | 0.27 | 0.10 |
| SEQ ID NO: 75 | 1.20 | 0.84 | 0.70 | 0.53 | 0.34 | 0.22 |
| SEQ ID NO: 119 | 1.23 | 1.05 | 0.84 | 0.75 | 0.59 | 0.27 |
| SEQ ID NO: 42 | 0.00 | 0.01 | −0.01 | 0.00 | 0.00 | −0.02 |
| SEQ ID NO: 21 | 0.86 | 0.43 | 0.31 | 0.21 | 0.12 | 0.06 |
| SEQ ID NO: 33 | 0.97 | 0.54 | 0.39 | 0.27 | 0.24 | 0.10 |
| SEQ ID NO: 89 | 0.46 | 0.31 | 0.28 | 0.12 | 0.07 | 0.03 |
| SEQ ID NO: 92 | 0.42 | 0.31 | 0.15 | 0.10 | 0.04 | 0.14 |
| SEQ ID NO: 95 | 0.94 | 0.81 | 0.62 | 0.49 | 0.24 | 0.17 |
| SEQ ID NO: 221 | 0.86 | 0.62 | 0.38 | 0.28 | 0.17 | 0.13 |
| SEQ ID NO: 125 | 0.19 | 0.08 | 0.11 | 0.03 | 0.02 | 0.01 |
| SEQ ID NO: 122 | 0.88 | 0.64 | 0.47 | 0.33 | 0.18 | 0.10 |
| SEQ ID NO: 101 | 0.79 | 0.57 | 0.54 | 0.42 | 0.13 | 0.07 |
| SEQ ID NO: 98 | 0.78 | 0.40 | 0.36 | 0.26 | 0.12 | 0.09 |
| SEQ ID NO: 104 | 0.39 | 0.27 | 0.17 | 0.06 | 0.12 | 0.06 |
| SEQ ID NO: 116 | 0.76 | 0.65 | 0.41 | 0.24 | 0.14 | 0.09 |
| SEQ ID NO: 146 | 0.85 | 0.61 | 0.50 | 0.41 | 0.19 | 0.19 |
| SEQ ID NO: 143 | 0.56 | 0.32 | 0.24 | 0.38 | 0.08 | 0.17 |
| SEQ ID NO: 110 | 0.51 | 0.32 | 0.19 | 0.07 | 0.05 | 0.00 |
| SEQ ID NO: 134 | 0.28 | 0.21 | 0.15 | 0.12 | 0.02 | 0.00 |
| SEQ ID NO: 140 | 0.00 | 0.00 | 0.02 | −0.02 | −0.02 | 0.09 |
| SEQ ID NO: 128 | −0.01 | 0.01 | 0.01 | −0.02 | −0.02 | −0.02 |
| SEQ ID NO: 137 | 0.95 | 0.68 | 0.51 | 0.36 | 0.21 | 0.13 |
| SEQ ID NO: 131 | 0.74 | 0.71 | 0.46 | 0.27 | 0.15 | 0.10 |
| SEQ ID NO: 86 | ND | ND | 0.05 | 0.03 | −0.01 | 0.03 |
| SEQ ID NO: 36 | ND | ND | 1.14 | 0.96 | 0.69 | 0.56 |
| SEQ ID NO: 107 | ND | ND | 0.14 | 0.08 | 0.28 | 0.00 |
| SEQ ID NO: 113 | ND | ND | 0.25 | 0.13 | 0.05 | 0.03 |
| SEQ ID NO: 158 | ND | ND | 0.04 | −0.02 | −0.09 | −0.02 |
| SEQ ID NO: 230 | ND | ND | 0.59 | 0.38 | 0.25 | 0.12 |
| SEQ ID NO: 233 | ND | ND | 0.58 | 0.30 | 0.16 | 0.07 |
| SEQ ID NO: 155 | ND | ND | 0.63 | 0.37 | 0.29 | 0.19 |
| SEQ ID NO: 80 | ND | ND | 1.62 | 0.98 | 0.57 | 0.39 |
| SEQ ID NO: 159 | 1.21 | 1.09 | 0.90 | 0.72 | 0.49 | 0.32 |
| HEWL | −0.02 | −0.02 | 0.00 | −0.01 | 0.00 | −0.02 |

ND: not determinded due to low concentration of enzyme stock solution

The present disclosure described and claimed herein is not to be limited in scope by the specific aspects herein disclosed, since these aspects are intended as illustrations of several aspects of the present disclosure. Any equivalent aspects are intended to be within the scope of this disclosure. Indeed, various modifications of the present disclosure in addition to those shown and described herein will become apparent to those skilled in the art from the foregoing description. Such modifications are also intended to fall within the scope of the appended claims. In the case of conflict, the present disclosure including definitions will control.

That which is claimed:

1. A method comprising administering a polypeptide having lysozyme activity and at least 80% sequence identity to SEQ ID NO: 9 to an animal.

2. The method of claim 1, wherein said polypeptide has at least 85% sequence identity to SEQ ID NO: 9.

3. The method of claim 1, wherein said polypeptide has at least 90% sequence identity to SEQ ID NO: 9.

4. The method of claim 1, wherein said polypeptide has at least 95% sequence identity to SEQ ID NO: 9.

5. The method of claim 1, wherein said polypeptide has at least 97% sequence identity to SEQ ID NO: 9.

6. The method of claim 1, wherein said polypeptide has at least 99% sequence identity to SEQ ID NO: 9.

7. The method of claim 1, wherein said polypeptide comprises the amino acid sequence of SEQ ID NO: 9.

8. The method of claim 1, wherein said polypeptide is a fragment of SEQ ID NO: 9 and comprises at least 180 amino acids.

9. The method of claim 1, wherein said animal is a ruminant animal.

10. The method of claim 1, wherein said animal is a cow.

11. The method of claim 1, wherein said polypeptide is administered to said animal in an amount effective to increase feed efficiency in said animal.

12. The method of claim 1, wherein said polypeptide is administered to said animal in an amount effective to increase body weight gain in said animal.

13. The method of claim 1, wherein said polypeptide is administered to said animal as part of an animal feed that comprises one or more plant materials.

14. The method of claim 13, wherein said one or more plant materials comprises forage.

15. A method comprising feeding a ruminant animal a composition that comprises a polypeptide having lysozyme activity and at least 80% sequence identity to SEQ ID NO: 9.

16. The method of claim 15, wherein said polypeptide has at least 95% sequence identity to SEQ ID NO: 9.

17. The method of claim 15, wherein said polypeptide has at least 97% sequence identity to SEQ ID NO: 9.

18. The method of claim 15, wherein said polypeptide has at least 99% sequence identity to SEQ ID NO: 9.

19. The method of claim 15, wherein said polypeptide comprises the amino acid sequence of SEQ ID NO: 9.

20. The method of claim 15, wherein said polypeptide is a fragment of SEQ ID NO: 9 and comprises at least 180 amino acids.


SEQUENCE LISTING (235 sequences)

SEQ ID NO: 1 - DNA, 858 bp, Sporormia fimetaria
  sig_peptide 1..51
  mat_peptide 52..855
  source 1..858: mol_type = genomic DNA
  CDS 1..141
  CDS 267..751
  CDS 810..855
atgaagtccgcattcgtctttctagctgcagtagcacagctagccagcgctgctgttcctggctttgacatctcccactaccaaagctcggtgaattacgccggtgcctactccagcggtgctcgctttgtcataataaaggtacgccccttctaattccttccctcgtcgcggacccgtattgtatacgttatttctccaagccgtctcccttcccgctcaatcaatgaaagtgacataaaaccgtcagtggctgacatttccaggccactgaaggaacaacctacatcgacccaaaattctctgaccattacattggcgccacaaatgccggactgattcgcggcgcgtaccattttgcgcgacccgccgcctctactggcgccgctcaggctaattacttcgtctcccacggcggaggttggtctgccgacggcatcactctgcccggaatgctcgacatggagtatgggtcgacatcggcctgccacgggctctcccagtccgcaatggttacctggatcacgagctttgtgaaccagtacaacagcctgacaggtcggtatccgatgatttacaccacggcagattggtggcaaacttgcacgggaaatagcgcggctttcaacaccaaatctcctctggtactggcgagatactcgagttctgcgggtacggtccctggaggctggccatattacacgatttggcaatttaatgatgcatatgcttatggaggggattcagatacgtaagtgcttttccatgcgcaattgatagtatgtattagggactaacaggacctatagttttaacggcgacctggccggcttaaagaggctcgcgaagggctcgtag

SEQ ID NO: 2 - protein, 224 aa, Sporormia fimetaria
MKSAFVFLAAVAQLASAAVPGFDISHYQSSVNYAGAYSSGARFVIIKATEGTTYIDPKFSDHYIGATNAGLIRGAYHFARPAASTGAAQANYFVSHGGGWSADGITLPGMLDMEYGSTSACHGLSQSAMVTWITSFVNQYNSLTGRYPMIYTTADWWQTCTGNSAAFNTKSPLVLARYSSSAGTVPGGWPYYTIWQFNDAYAYGGDSDTFNGDLAGLKRLAKGS

SEQ ID NO: 3 - protein, 207 aa, Sporormia fimetaria
AVPGFDISHYQSSVNYAGAYSSGARFVIIKATEGTTYIDPKFSDHYIGATNAGLIRGAYHFARPAASTGAAQANYFVSHGGGWSADGITLPGMLDMEYGSTSACHGLSQSAMVTWITSFVNQYNSLTGRYPMIYTTADWWQTCTGNSAAFNTKSPLVLARYSSSAGTVPGGWPYYTIWQFNDAYAYGGDSDTFNGDLAGLKRLAKGS

SEQ ID NO: 4 - DNA, 716 bp, Poronia punctata
  sig_peptide 1..51
  mat_peptide 52..713
  source 1..716: mol_type = genomic DNA
  CDS 1..141
  CDS 201..713
atgaagtcttacatcgcccccctcctcggcctcgcccaaggcgcgctcgccgccgtccagggcttcgacatttcgcactatcagtcgagcgtcaactttggcgccgccaagagctccggtgctcagtttgtcatcatcaaggtgagaaggacaagccataaaatgaggggaaagtcgtgtgtaactgacgtgaccaacaggcgactgagggtacctcctataccgaccccagtttcagctctcactataccggcgccaccaatgctggcttgatccgtggcggttaccacttcgcccacctcgattccagctctggtgctgcgcaggccaagtacttcctcgcccatggtggtggctggtctggagacggcatcaccctgcccggtatgctggatcttgagggcagctgcgtcctctcggctagcgctaccgtgtcttggatcaaggactttagcaacacctaccactcgtccacgggcgtgtaccctctaatttacacaaacccctcgtggtggtctagctgcaccggtaactccaaggcctttatcgacaccaatcctctcgtacttgcacggtacgcgtcaagcgctggcacccctcccggtggctggccttactataccatctggcagtacaacgatgcctacaagtacggaggtgactcggacgtcttcaacggagaccttgccggcctcaagagactggctaagggctaa

SEQ ID NO: 5 - protein, 218 aa, Poronia punctata
MKSYIAPLLGLAQGALAAVQGFDISHYQSSVNFGAAKSSGAQFVIIKATEGTSYTDPSFSSHYTGATNAGLIRGGYHFAHLDSSSGAAQAKYFLAHGGGWSGDGITLPGMLDLEGSCVLSASATVSWIKDFSNTYHSSTGVYPLIYTNPSWWSSCTGNSKAFIDTNPLVLARYASSAGTPPGGWPYYTIWQYNDAYKYGGDSDVFNGDLAGLKRLAKG

SEQ ID NO: 6 - protein, 201 aa, Poronia punctata
AVQGFDISHYQSSVNFGAAKSSGAQFVIIKATEGTSYTDPSFSSHYTGATNAGLIRGGYHFAHLDSSSGAAQAKYFLAHGGGWSGDGITLPGMLDLEGSCVLSASATVSWIKDFSNTYHSSTGVYPLIYTNPSWWSSCTGNSKAFIDTNPLVLARYASSAGTPPGGWPYYTIWQYNDAYKYGGDSDVFNGDLAGLKRLAKG

SEQ ID NO: 7 - DNA, 771 bp, Poronia punctata
  sig_peptide 1..48
  mat_peptide 49..768
  source 1..771: mol_type = genomic DNA
  CDS 1..138
  CDS 256..768
atgaagtatctcgttccccttttgggcttcgcccacggcgccctggcccaggttcaaggcttcgatatctcgagctaccagcccagcgtagactttgccggtgcttatgctgatggcgcccgattcgtcatcatcaaggtaagaaaaatactacaatccaagcccataaccataaccaaaaccccccgccccccattggattagatggatgatggactaggtaacctaacctgctctaacacaaaataatcataggccaccgaaggaactggctacatcgaccccaccttcagcgaccactacgtaggcgcaaccaacgccggcctgctccgcggcggttaccactacgcccacctggactcgacctcgggcgctacccaggcccagtacttcctcgccaacggcggtggctggtccggcgacggaatcaccctccccggcatgcttgatctcgagggtgactgcgtgctctcggccgccgacgccgtcgcctggatcaaggacttcagcgacacctaccacgccagcacgggcgtctaccccctgctgtacaccaacccttcgtggtgggcctcttgcaccggcgactccagcgccttcatcgacaccaaccccctcgtcctcgcccactacgccgacgccgccggcaccccccctggtggctggcccttctactccttctggcagtacaacgatgcctacccctacggtggcgactccgaggtctggaacggtgatatggacggtcttctccgccttgcttcgggctaa

SEQ ID NO: 8 - protein, 217 aa, Poronia punctata
MKYLVPLLGFAHGALAQVQGFDISSYQPSVDFAGAYADGARFVIIKATEGTGYIDPTFSDHYVGATNAGLLRGGYHYAHLDSTSGATQAQYFLANGGGWSGDGITLPGMLDLEGDCVLSAADAVAWIKDFSDTYHASTGVYPLLYTNPSWWASCTGDSSAFIDTNPLVLAHYADAAGTPPGGWPFYSFWQYNDAYPYGGDSEVWNGDMDGLLRLASG

SEQ ID NO: 9 - protein, 201 aa, Poronia punctata
QVQGFDISSYQPSVDFAGAYADGARFVIIKATEGTGYIDPTFSDHYVGATNAGLLRGGYHYAHLDSTSGATQAQYFLANGGGWSGDGITLPGMLDLEGDCVLSAADAVAWIKDFSDTYHASTGVYPLLYTNPSWWASCTGDSSAFIDTNPLVLAHYADAAGTPPGGWPFYSFWQYNDAYPYGGDSEVWNGDMDGLLRLASG

SEQ ID NO: 10 - DNA, 675 bp, Lecanicillium sp.
  sig_peptide 1..63
  mat_peptide 64..672
  source 1..675: mol_type = genomic DNA
  CDS 1..672
atgcgaccatttactgccctctttgtcgccttcgtcagcgccgccagtgcgctgacccacgccgtcgacagctcctccgaggtgagcgtcgccatttacaaaaaggccctcggccagggcttcacccgcgccatcttccgcggctaccaagaggcctgctcgcagggcggccgcgtcgacccgacctttgtgccgagctacaagaatgccgtcgcggccggctacaaggactttgacgcctacttcttcccctgcaccggcaagaccaacaagtgcaagccctacgccgcgcagctcgccgagctcctcgacaccatcaagggccagaagctggcgattcgccgcatctggctcgacattgagacggacagggtgtgcaacccgtttgactatggcgcacagggcaaccttgccgaggccaagaagctcgtggccgcgtttcgcgacgccaagcttgactggggcatctacacgtcgccgacgcagtgggagaccatctttggcgccaagaccgtcgagctggccaaggacgtgccgctatggtttgccaagtttgacaatgtcgagacgctggagctcaagacgccgtttggtggctggacaaaggcggatgcgaagcagtatactgaccagtcggccagcaacaagtttgacttgaacgtcttttctgcctaa

SEQ ID NO: 11 - protein, 224 aa, Lecanicillium sp.
MRPFTALFVAFVSAASALTHAVDSSSEVSVAIYKKALGQGFTRAIFRGYQEACSQGGRVDPTFVPSYKNAVAAGYKDFDAYFFPCTGKTNKCKPYAAQLAELLDTIKGQKLAIRRIWLDIETDRVCNPFDYGAQGNLAEAKKLVAAFRDAKLDWGIYTSPTQWETIFGAKTVELAKDVPLWFAKFDNVETLELKTPFGGWTKADAKQYTDQSASNKFDLNVFSA

SEQ ID NO: 12 - protein, 203 aa, Lecanicillium sp.
VDSSSEVSVAIYKKALGQGFTRAIFRGYQEACSQGGRVDPTFVPSYKNAVAAGYKDFDAYFFPCTGKTNKCKPYAAQLAELLDTIKGQKLAIRRIWLDIETDRVCNPFDYGAQGNLAEAKKLVAAFRDAKLDWGIYTSPTQWETIFGAKTVELAKDVPLWFAKFDNVETLELKTPFGGWTKADAKQYTDQSASNKFDLNVFSA

SEQ ID NO: 13 - DNA, 796 bp, Lecanicillium sp.
  sig_peptide 1..60
  mat_peptide 61..793
  source 1..796: mol_type = genomic DNA
  CDS 1..150
  CDS 199..381
  CDS 443..793
atgaagtcattctcatccattatcgccggcatcgccggccttgcctctgtcgcttctgccacggtgcagggcttcgatgtctctggctaccagcccactgtcaactggggtgcggcctacagcagcggtgctcgcttcgtcatgatcaaggtatgctgcagcggacggttcgaatcacagatgatgctgacaggctaggccaccgagggaactggttacatctcgtccagcttcggctcgcagtaccctggtgccaccaatgcgggctttatccgcggcggctaccactttgcgctgcccgaccggtcctctggctccgcacaggccgactactttctggcccacggcggcggctggagcggcgatggcatcactctaccggtaagtcccatcaccttccttgaatcgaagcgccatggtagtgctagtctgacgcatccagggcatgctggacattgagtataacccgtacggcgccacctgctacggcctctcgcagggcgccatggtcaactggatcagcgactttgtcgagcactacaaggccaggacgacgcagtaccccatcatctacacgacgaccgactggtggaagacgtgcacgggcaacagccctgcctttggccaaaagtgcccgctgagcctggcccggtactcgagcagcgtgggcgagatccccaacggctggccgttccagactttctggcagaacagcgacaagtatgcgtacggtggcgattcgcagattttcaacggcgcgtactctcagctgcagaagattgctcgcggtggttag

SEQ ID NO: 14 - protein, 228 aa, Lecanicillium sp.
MKSFSSIIAGIAGLASVASATVQGFDVSGYQPTVNWGAAYSSGARFVMIKATEGTGYISSSFGSQYPGATNAGFIRGGYHFALPDRSSGSAQADYFLAHGGGWSGDGITLPGMLDIEYNPYGATCYGLSQGAMVNWISDFVEHYKARTTQYPIIYTTTDWWKTCTGNSPAFGQKCPLSLARYSSSVGEIPNGWPFQTFWQNSDKYAYGGDSQIFNGAYSQLQKIARGG

SEQ ID NO: 15 - protein, 208 aa, Lecanicillium sp.
TVQGFDVSGYQPTVNWGAAYSSGARFVMIKATEGTGYISSSFGSQYPGATNAGFIRGGYHFALPDRSSGSAQADYFLAHGGGWSGDGITLPGMLDIEYNPYGATCYGLSQGAMVNWISDFVEHYKARTTQYPIIYTTTDWWKTCTGNSPAFGQKCPLSLARYSSSVGEIPNGWPFQTFWQNSDKYAYGGDSQIFNGAYSQLQKIARGG

SEQ ID NO: 16 - DNA, 734 bp, Onygena equina
  sig_peptide 1..54
  mat_peptide 55..731
  source 1..734: mol_type = genomic DNA
  CDS 1..144
  CDS 201..731
atgttgaaaacaattatctataccacccttgccgtcgctagcctggcgtcagcagccgttcccggtatcgacgtgtcgggctaccaaggcaacgtgaactgggcgaacgtcgccaacgctggaaagaagtttgcctacgtcaaggtatgcgtctccgtaatgagttatgaattggaactaatcaaatcaatcgggcataggccacggaacataccaactacatcaacccttacttcgcccagcagtacaatggcgcctacaaccagggcattattcgaggtgcataccactacgcccaccccaacggcgcaagcggagcttctcaggccaactacttccttgctcacggtggcggctggtctgctgatgggaaaacccttcctggtgccgtcgacctcgagtacggacccaatggcagcacttgctggggtatcagtcaatcggcgatgatcgcttggatccgtgacttctccaacacctaccgtgccaagaccggccggcctccagtcatctacaccagcacctcttggtggaagacctgcaccggtaactatggcggtttcggaaacgataatcccctttggattgctcgttattcaagcactgtcggcgaacttcctgctggctggcctttccacagcatctggcagaacaacgataacagcggtgttggaggggacggtgatatctggaacggtgacctggctggattgcaacgatttgcaaagggctaa

SEQ ID NO: 17 - protein, 225 aa, Onygena equina
MLKTIIYTTLAVASLASAAVPGIDVSGYQGNVNWANVANAGKKFAYVKATEHTNYINPYFAQQYNGAYNQGIIRGAYHYAHPNGASGASQANYFLAHGGGWSADGKTLPGAVDLEYGPNGSTCWGISQSAMIAWIRDFSNTYRAKTGRPPVIYTSTSWWKTCTGNYGGFGNDNPLWIARYSSTVGELPAGWPFHSIWQNNDNSGVGGDGDIWNGDLAGLQRFAKG

SEQ ID NO: 18 - protein, 207 aa, Onygena equina
AVPGIDVSGYQGNVNWANVANAGKKFAYVKATEHTNYINPYFAQQYNGAYNQGIIRGAYHYAHPNGASGASQANYFLAHGGGWSADGKTLPGAVDLEYGPNGSTCWGISQSAMIAWIRDFSNTYRAKTGRPPVIYTSTSWWKTCTGNYGGFGNDNPLWIARYSSTVGELPAGWPFHSIWQNNDNSGVGGDGDIWNGDLAGLQRFAKG

SEQ ID NO: 19 - DNA, 742 bp, Purpureocillium lilacinum
  sig_peptide 1..57: note = 739
  mat_peptide 58..739: note = 739
  source 1..742: mol_type = genomic DNA
  CDS 1..147
  CDS 206..739: note = 739
atgaagttcgcatccgtcgccgcctctgtgtccgccctctgcggcgtggcctctgccgctgtcaagggctttgacatttcccactaccagcccaacgtcgactttgccaaggcctatgccgatggcgcccgcttcgtgatgatcaaggtgcgttcacccagatgaagagcttcccccgaattccatctaacgttcacgtcggcaggccacggagggcaccacgtacacggaccccagcttcagctcgcactacacgggcgccaccaaggcgggcttcatccgcggcggctaccactttgcccgcccggcgtcctcgtccggtgccgcgcaggccaagtactttatcgcgcacggcggcggctggtccaaggacggcatcacgctgcctggcatgctcgacatggagtaccagtcgtcgagcagcgcgtgcggcgggctctcacagagcgccatggtcagctggatcaacgactttgtcaacacgtaccacgccgccacgggcgtctacccgctcatctacacctcgaccagctggtggacgcagtgcacgggcaacagcgccgcctttggcagcaagtgccctctcgtcgtcgcgcgctatgctagctccgtcggcacgctccctgctggctggggcttctacaccttctggcagtactcggacgcggcgccctggggtggtgatgcggatacctttaacggcgacattactgctctcaagaagattgccaacgccggttaa

SEQ ID NO: 20 - protein, 227 aa, Purpureocillium lilacinum
MKFASVAASVSALCGVASAAVKGFDISHYQPNVDFAKAYADGARFVMIKATEGTTYTDPSFSSHYTGATKAGFIRGGYHFARPASSSGAAQAKYFIAHGGGWSKDGITLPGMLDMEYQSSSSACGGLSQSAMVSWINDFVNTYHAATGVYPLIYTSTSWWTQCTGNSAAFGSKCPLVVARYASSVGTLPAGWGFYTFWQYSDAAPWGGDADTFNGDITALKKIANAG

SEQ ID NO: 21 - protein, 208 aa, Purpureocillium lilacinum
AVKGFDISHYQPNVDFAKAYADGARFVMIKATEGTTYTDPSFSSHYTGATKAGFIRGGYHFARPASSSGAAQAKYFIAHGGGWSKDGITLPGMLDMEYQSSSSACGGLSQSAMVSWINDFVNTYHAATGVYPLIYTSTSWWTQCTGNSAAFGSKCPLVVARYASSVGTLPAGWGFYTFWQYSDAAPWGGDADTFNGDITALKKIANAG

SEQ ID NO: 22 - DNA, 895 bp, Trichobolus zukalii
  sig_peptide 1..54
  mat_peptide 55..876
  source 1..895: mol_type = genomic DNA
  CDS 1..144
  CDS 293..750
  CDS 820..892
atgaagctcaccacttttatcacgggccttgtatcggccagcaccgctcttgctgccgttccaggattcgatatctcccactaccaaccgtccgttaactacgccggcgcctacaactccggcgctcgtttcgtcatcatcaaagtcctcttcccttactctcccttctccccttctccccttctccccttctccccttctccccttctccccttctccccttctccccttctccccttctccccttctccccttctcctctcaacatcccatcctaactcccttctcctaggcgaccgaaggtacaacctacactgaccccgtattctccactcactataccggagctaccaaagctggcttaatccggggaggctaccacttcgctcgtcccgcctcgtcgtccggctccgcccaagccgatttcttcttcaaaaacggaggcgggtggtctgctgacggtatcacacttcctggtatgctcgatatggaatatggcagcacttcatcatgccatggactttctcaaactgctatggtgaattggatcagcgatttcgtgaaccggtataaaacgttgagtgggaggtatccgatgatttatactggttactattggtgggtggagtgtacagggaactctaacaagtttgcaacgacttgtcctttggtgcttgcaaggtattcgagttccgtgggagagattccgggaggctgggggtatcaaacgatttggcagtttaatgataagtaagtggttttccgaaggtgagatgaagtgaggacgggaggatgtaggctaattagacaccgctgcaggtacgcttatggcggtgactcggatagtttcaatggctcgctcgatcgccttaaggcgcttgccaaggggacttaa

SEQ ID NO: 23 - protein, 225 aa, Trichobolus zukalii
MKLTTFITGLVSASTALAAVPGFDISHYQPSVNYAGAYNSGARFVIIKATEGTTYTDPVFSTHYTGATKAGLIRGGYHFARPASSSGSAQADFFFKNGGGWSADGITLPGMLDMEYGSTSSCHGLSQTAMVNWISDFVNRYKTLSGRYPMIYTGYYWWVECTGNSNKFATTCPLVLARYSSSVGEIPGGWGYQTIWQFNDKYAYGGDSDSFNGSLDRLKALAKGT

SEQ ID NO: 24 - protein, 207 aa, Trichobolus zukalii
AVPGFDISHYQPSVNYAGAYNSGARFVIIKATEGTTYTDPVFSTHYTGATKAGLIRGGYHFARPASSSGSAQADFFFKNGGGWSADGITLPGMLDMEYGSTSSCHGLSQTAMVNWISDFVNRYKTLSGRYPMIYTGYYWWVECTGNSNKFATTCPLVLARYSSSVGEIPGGWGYQTIWQFNDKYAYGGDSDSFNGSLDRLKALAKGT

SEQ ID NO: 25 - DNA, 672 bp, Penicillium citrinum
  sig_peptide 1..48
  mat_peptide 49..669
  source 1..672: mol_type = genomic DNA
  CDS 1..669
atgcgcccctccgtcatattgctcgcttttgccagcgcagccagcgctctcatccacgctgtcgacagctcctccgaagtatccgtcgacatttacaaaaaggccttgagcgagggcttctcgcgcgcaatcttccgcggttaccaggaggcctgctcccagggcggccgcgtcgacccaacctttttgcccagctacaagaatgcgcaaacagccggctacaaagactttgacgcctatttcttcccctgcacgggctccggaaacaaatgcaagccatacgacgtgcagattggcgagctcgtcgacgctattaaaaagaacaacatggccattcgccgcatctgggtcgactttgaaaaggacaagacctgcaacccgtttaactgggaccctaaacgcaacattgatgaggccaagaggattataggtgcagtgcgcaagacaaagttcgattttggcgtgtacacctcagcaacacagtggacttccatctttggctccaaggacgtagtcctggctaatgatgtgccgctttggtttgccaagtttgataatgtcgagaaccttgacctggcgcagccttttggaggctggacaaaggcggacggaaagcaatacaccgacaagtcagctagtaagaagtttgatttgaacgtgttttctgcttaa

SEQ ID NO: 26 - protein, 223 aa, Penicillium citrinum
MRPSVILLAFASAASALIHAVDSSSEVSVDIYKKALSEGFSRAIFRGYQEACSQGGRVDPTFLPSYKNAQTAGYKDFDAYFFPCTGSGNKCKPYDVQIGELVDAIKKNNMAIRRIWVDFEKDKTCNPFNWDPKRNIDEAKRIIGAVRKTKFDFGVYTSATQWTSIFGSKDVVLANDVPLWFAKFDNVENLDLAQPFGGWTKADGKQYTDKSASKKFDLNVFSA

SEQ ID NO: 27 - protein, 207 aa, Penicillium citrinum
LIHAVDSSSEVSVDIYKKALSEGFSRAIFRGYQEACSQGGRVDPTFLPSYKNAQTAGYKDFDAYFFPCTGSGNKCKPYDVQIGELVDAIKKNNMAIRRIWVDFEKDKTCNPFNWDPKRNIDEAKRIIGAVRKTKFDFGVYTSATQWTSIFGSKDVVLANDVPLWFAKFDNVENLDLAQPFGGWTKADGKQYTDKSASKKFDLNVFSA

SEQ ID NO: 28 - DNA, 690 bp, Cladorrhinum bulbillosum
  sig_peptide 1..66
  mat_peptide 67..687
  source 1..690: mol_type = genomic DNA
  CDS 1..687
atgaagctcctccccctttccaccaccttattgcccgtggccctcctcgccaccgaggcctcggcggcagtccaaggcttcgacatctcgcactaccaatcctcagtcaacttccaggcggcctacaactcgggcgcccgcttcgtcatcatcaaggcgacagagggcacgacctacatcgaccccaagttttcgtctcactacacgggagccaccaacgccgggctaatccggggcgggtaccactttgcccatccggactcgtcgaccggcgccgcgcaggcagattttttcctcgcccacggcggcggctggtccggcgacggcatcaccctgcccgggatgctcgacctcgaatccgtctccggaaaggcgacctgcttcgggctctcggcctcgtccatggtcgcctggatcaagtcgttctctgaccggtaccacacccggaccggacggtacccgatgctgtacaccaacccgtcttggtggaccacctgcaccggaaacagcaacgcgttcgtcaacacgaacccgctcgttctggctcggtacgccagcgcgcccgggaccatccccggtggatggccgtatcagaccatctggcagaattcggactcgtatacctacggaggggattcggatatttttaacggcgcgctgagtgggttgcaaaagttggccagcggttaa

SEQ ID NO: 29 - protein, 229 aa, Cladorrhinum bulbillosum
MKLLPLSTTLLPVALLATEASAAVQGFDISHYQSSVNFQAAYNSGARFVIIKATEGTTYIDPKFSSHYTGATNAGLIRGGYHFAHPDSSTGAAQADFFLAHGGGWSGDGITLPGMLDLESVSGKATCFGLSASSMVAWIKSFSDRYHTRTGRYPMLYTNPSWWTTCTGNSNAFVNTNPLVLARYASAPGTIPGGWPYQTIWQNSDSYTYGGDSDIFNGALSGLQKLASG

SEQ ID NO: 30 - protein, 207 aa, Cladorrhinum bulbillosum
AVQGFDISHYQSSVNFQAAYNSGARFVIIKATEGTTYIDPKFSSHYTGATNAGLIRGGYHFAHPDSSTGAAQADFFLAHGGGWSGDGITLPGMLDLESVSGKATCFGLSASSMVAWIKSFSDRYHTRTGRYPMLYTNPSWWTTCTGNSNAFVNTNPLVLARYASAPGTIPGGWPYQTIWQNSDSYTYGGDSDIFNGALSGLQKLASG

SEQ ID NO: 31 - DNA, 687 bp, Umbelopsis westeae
  sig_peptide 1..60
  mat_peptide 61..684
  source 1..687: mol_type = genomic DNA
  CDS 1..684
atgaagctcacctttgcctctctaactctcctatcttccgctcttgttggagtgtcagctaaattgaaaggattggatgttagcggttaccaaccaaatgtagcttggtcaaccgtcaaggccaatggtgcatcctttgcttatatcaaagctactgaaggcactaattataaaaacccatcgtttgctcaacagtacaacggagcatataatgctggcttgattcgcggctcgtaccattttgctcagccttcctcctctaccggtgctgctcaagccaactacttccttgcgcacggaggtggctggtctcctgatggcaagactcttcccggtgctttggatatggaatataaccctcatggctctacatgctatggcttatccaaggatgctatggtaaagtggattaaggatttcagtaatacctaccactctgccactggccgttatcccgtcatttacactaccactagttggtggacgacttgcaccggtaacagtgctgcatttggcgctaccaaccctctttggatcgctagatactcctctacggctgggaacttgcccaatggttgggcattctactctttctggcaaaatgccgacagtggcatcttccctggtgatcaagatatttggaacggtgatgctgctgctttgtctaggatggccaaaggagcctaa

SEQ ID NO: 32 - protein, 228 aa, Umbelopsis westeae
MKLTFASLTLLSSALVGVSAKLKGLDVSGYQPNVAWSTVKANGASFAYIKATEGTNYKNPSFAQQYNGAYNAGLIRGSYHFAQPSSSTGAAQANYFLAHGGGWSPDGKTLPGALDMEYNPHGSTCYGLSKDAMVKWIKDFSNTYHSATGRYPVIYTTTSWWTTCTGNSAAFGATNPLWIARYSSTAGNLPNGWAFYSFWQNADSGIFPGDQDIWNGDAAALSRMAKGA

SEQ ID NO: 33 - protein, 208 aa, Umbelopsis westeae
KLKGLDVSGYQPNVAWSTVKANGASFAYIKATEGTNYKNPSFAQQYNGAYNAGLIRGSYHFAQPSSSTGAAQANYFLAHGGGWSPDGKTLPGALDMEYNPHGSTCYGLSKDAMVKWIKDFSNTYHSATGRYPVIYTTTSWWTTCTGNSAAFGATNPLWIARYSSTAGNLPNGWAFYSFWQNADSGIFPGDQDIWNGDAAALSRMAKGA

SEQ ID NO: 34 - DNA, 684 bp, Zygomycetes sp.
  sig_peptide 1..57
  mat_peptide 58..681
  source 1..684: mol_type = genomic DNA
  CDS 1..681
atgaaagcaatcgtaacagcattagcattatccttgttatgggcgggtgcccatgcaactttgcccggcttagacgtcagcagctaccaaggtaacgtcaattggggaacagtggcgagtcaaggagcaaaatttgcttacgtcaaggctaccgagggtacgacctacacgaatccctattttgcgtcccaatacgacggatcctacaacgcgggcctaattcgcggtgcctatcactttgcccatcccgattcttcctctggagctacccaagcaaactatttccttgctcatggtggcggctggtccgctgacggaaagaccttacctggtgcgctagatattgagtacaatcctaacggcgctgaatgctacggcttgtctcaattggccatgattagctggattcaagacttcagcaacacctatcactcccacacgggcagatatccggtcatttacacgactacggactggtggaccacctgcacgggtaacagcgcagcctttggaaccaacaaccctctctggattgctcggtattcgtcttcggtgggcaccctgcctgcaggttggggctacgagagcttctggcagaaggcatcttcgggtacgttccctggagaccaagatatctggaatggcgatgctgctggactctccagattcgccaccggcaaatga

SEQ ID NO: 35 - protein, 227 aa, Zygomycetes sp.
MKAIVTALALSLLWAGAHATLPGLDVSSYQGNVNWGTVASQGAKFAYVKATEGTTYTNPYFASQYDGSYNAGLIRGAYHFAHPDSSSGATQANYFLAHGGGWSADGKTLPGALDIEYNPNGAECYGLSQLAMISWIQDFSNTYHSHTGRYPVIYTTTDWWTTCTGNSAAFGTNNPLWIARYSSSVGTLPAGWGYESFWQKASSGTFPGDQDIWNGDAAGLSRFATGK

SEQ ID NO: 36 - protein, 208 aa, Zygomycetes sp.
TLPGLDVSSYQGNVNWGTVASQGAKFAYVKATEGTTYTNPYFASQYDGSYNAGLIRGAYHFAHPDSSSGATQANYFLAHGGGWSADGKTLPGALDIEYNPNGAECYGLSQLAMISWIQDFSNTYHSHTGRYPVIYTTTDWWTTCTGNSAAFGTNNPLWIARYSSSVGTLPAGWGYESFWQKASSGTFPGDQDIWNGDAAGLSRFATGK

SEQ ID NO: 37 - DNA, 747 bp, Chaetomium cupreum
  sig_peptide 1..51
  mat_peptide 52..744
  source 1..747: mol_type = genomic DNA
  CDS 1..141
  CDS 217..744
atgaaatccgccatcctcgcctgtgtcggttttgctgccgcggtgcaagccacggtccagggctttgacatctccggctatcagccaaatgtcaactttgccgccgcctatgccgcaggcgcccgcttcgtcatcatcaaggtgagcagcccaagtcatcacagtcacccactacctgaggtacggtagtcaaggcaatcagctaacctctgccaggccaccgagggcaccagctacatcagcccctccttctcctcgcagtacacgggcgccaccaacgccggcttcatccgcggcggctaccacttcgcccacccgggcgcgtcgtccggcaccacccaggccgactacttcatcgcgcacggcggcggctggactcctgacggcatcacgctgccgggcatgctggacctcgagtccgagtcgagtggcacgtgctggggcctgtcggcgagcgccatggtggcctggatcaaggacttcagcgaccactaccactcgcggatgggcgtctacccgctgctgtacacgaacccgtcgtggtgggaggagtgcacgggcaactccaacgccttcgtcgacaccaacccgctcgtgctggcgcactacagcagcagcgtcgggacgatccccggcgggtggccgtatgagacgatctggcagaactcggactcgtatgcgtacggcggtgactcggatgtgtttaatggggatctggctgggctgcagagacttgcgaggggttaa

SEQ ID NO: 38 - protein, 223 aa, Chaetomium cupreum
MKSAILACVGFAAAVQATVQGFDISGYQPNVNFAAAYAAGARFVIIKATEGTSYISPSFSSQYTGATNAGFIRGGYHFAHPGASSGTTQADYFIAHGGGWTPDGITLPGMLDLESESSGTCWGLSASAMVAWIKDFSDHYHSRMGVYPLLYTNPSWWEECTGNSNAFVDTNPLVLAHYSSSVGTIPGGWPYETIWQNSDSYAYGGDSDVFNGDLAGLQRLARG

SEQ ID NO: 39 - protein, 206 aa, Chaetomium cupreum
TVQGFDISGYQPNVNFAAAYAAGARFVIIKATEGTSYISPSFSSQYTGATNAGFIRGGYHFAHPGASSGTTQADYFIAHGGGWTPDGITLPGMLDLESESSGTCWGLSASAMVAWIKDFSDHYHSRMGVYPLLYTNPSWWEECTGNSNAFVDTNPLVLAHYSSSVGTIPGGWPYETIWQNSDSYAYGGDSDVFNGDLAGLQRLARG

SEQ ID NO: 40 - DNA, 675 bp, Cordyceps cardinalis
  sig_peptide 1..51
  mat_peptide 52..672
  source 1..675: mol_type = genomic DNA
  CDS 1..672
atgcgcgcctttattccagtctttctcgccctagccggcgcagccaacgccctcattcacgccgtcgacagctcctccgaggtgtccgtcgacgtctacaaaaaggccctcgccgagggcttcacacgcgccatcttccgcggctaccaggaggcctgctcgcagggcggccgcgtcgaccctaccttcctgcccagctacaagaacgcccagaaggcgggctacaaggactttgacgcctacttcttcccgtgcaccggctccggcaacaagtgcaagccctacgcgaagcagattggcgagctcgtcgacgccatcgagggcaatcagctggccatccgccgcatctggatcgacatcgagacggacaaggtctgcaacgcgtttaactggggcgccgagggcaacatccaagaggccaagaagctcatcgccgccgtgcgaggcacaaagcgcgactttggcatctacacgtcggcgacgcaatgggagaacatctttggctccaggactgtggaactggccaaggacgtgccgctgtggtttgccaagtttgacaatgttgagacgctggagctgaagacgccttttggcggctggacaaaggccgacgcgaagcagtatactgacaagtcggctagcaagaaatttgatctcaacgttttctctgcctaa

SEQ ID NO: 41 - protein, 224 aa, Cordyceps cardinalis
MRAFIPVFLALAGAANALIHAVDSSSEVSVDVYKKALAEGFTRAIFRGYQEACSQGGRVDPTFLPSYKNAQKAGYKDFDAYFFPCTGSGNKCKPYAKQIGELVDAIEGNQLAIRRIWIDIETDKVCNAFNWGAEGNIQEAKKLIAAVRGTKRDFGIYTSATQWENIFGSRTVELAKDVPLWFAKFDNVETLELKTPFGGWTKADAKQYTDKSASKKFDLNVFSA

SEQ ID NO: 42 - protein, 207 aa, Cordyceps cardinalis
LIHAVDSSSEVSVDVYKKALAEGFTRAIFRGYQEACSQGGRVDPTFLPSYKNAQKAGYKDFDAYFFPCTGSGNKCKPYAKQIGELVDAIEGNQLAIRRIWIDIETDKVCNAFNWGAEGNIQEAKKLIAAVRGTKRDFGIYTSATQWENIFGSRTVELAKDVPLWFAKFDNVETLELKTPFGGWTKADAKQYTDKSASKKFDLNVFSA

SEQ ID NO: 43 - DNA, 761 bp, Penicillium sp.
  sig_peptide 1..51
  mat_peptide 52..758
  source 1..761: mol_type = genomic DNA
  CDS 1..165
  CDS 225..758
atgaagactacgggtgtctctcttctgcttgcagcaggtactgcctacgcatcgacaatccagcctcgggcaagcggcgtccagggattcgatatctcaagctaccaaggcaccgtcaactttgccggcgcctacggagccggtgcacgattcgtcatgatcaaggtgagcctcggcataaacttgtgaccggcgagttgtttggactaactgaaacgcgctaggcgactgaaggcaccacctacatagattccaccttctccagccactatgacggtgctaccagcgccggcttgatccgcggggcttaccacttcgcccacccggactccagctctggcgctacccaggccgagtacttcctggctcacggaggtggctggaccaacgatggcatcaccttgcccggcatgctggacatcgaatacaacccctcgggctctacctgctacggtctgagtgcttccgccatggtctcctggatcaaggacttcggagagacctacaacagcaagactggtcggtaccctatgatctacagcacggccgattggtggagcacctgcacaggagacagcacatccttcagtagtgactaccctctggtgcttgctcagtatgctagctccattagcaccgtccccggaggctggccttaccagagcttctggcagaacgcggactcatacagctatggcggtgattctgatctgtggaatggtagcgaggactctctgaagacctttgccaagggttcttaa

SEQ ID NO: 44 - protein, 233 aa, Penicillium sp.
MKTTGVSLLLAAGTAYASTIQPRASGVQGFDISSYQGTVNFAGAYGAGARFVMIKATEGTTYIDSTFSSHYDGATSAGLIRGAYHFAHPDSSSGATQAEYFLAHGGGWTNDGITLPGMLDIEYNPSGSTCYGLSASAMVSWIKDFGETYNSKTGRYPMIYSTADWWSTCTGDSTSFSSDYPLVLAQYASSISTVPGGWPYQSFWQNADSYSYGGDSDLWNGSEDSLKTFAKGS

SEQ ID NO: 45 - protein, 216 aa, Penicillium sp.
STIQPRASGVQGFDISSYQGTVNFAGAYGAGARFVMIKATEGTTYIDSTFSSHYDGATSAGLIRGAYHFAHPDSSSGATQAEYFLAHGGGWTNDGITLPGMLDIEYNPSGSTCYGLSASAMVSWIKDFGETYNSKTGRYPMIYSTADWWSTCTGDSTSFSSDYPLVLAQYASSISTVPGGWPYQSFWQNADSYSYGGDSDLWNGSEDSLKTFAKGS

SEQ ID NO: 46 - DNA, 781 bp, Aspergillus sp.
  sig_peptide 1..54
  mat_peptide 55..778
  source 1..781: mol_type = genomic DNA
  CDS 1..174
  CDS 245..778
atgaagttcactaccattgccactaccgcggccatctctggccttgccacagccttgcctaccaaactggctgctcgatatagcactgtgcaaggatttgatgtgtccaattaccaaccgaacgtggacttctctgccgcaaagagtgctggtgccgaatttgtcattatcaaggtacatccagctttatcccttttctacccagtaacatggtacagataagttaacaagtcattcataacaggccaccgaaggaaccgattacaaagacacctatttcaactcccactacaccggcgccaccaacgctggcctcatccgcggtggctatcatttcgcgcgccctgataaatcttccggtaccgcgcaagccgagtatttcctcgcgcacggtggcggctggagcaaagatggcagaaccctccctggcatgctagacatcgagtataacccgtacggcgccacctgctacggtctcagtcactccgcgatggtctcatgggtcaacgaattcctcaacgaatatcacagcaagacgggtgtttatccgttgctctataccacggcagattggtggaatcagtgcacagggaatgctcatggatttggtaacaagagccctcttgttctggcttcatatagcagtgagtcacctcggactgtgcctggagattggcagacgtggactatctggcagaacgcggataagtataagtatgggggtgattccgatattttcaatggcgatctcacgcagttgaagaagattgtcgaggggtag

SEQ ID NO: 47 - protein, 236 aa, Aspergillus sp.
MKFTTIATTAAISGLATALPTKLAARYSTVQGFDVSNYQPNVDFSAAKSAGAEFVIIKATEGTDYKDTYFNSHYTGATNAGLIRGGYHFARPDKSSGTAQAEYFLAHGGGWSKDGRTLPGMLDIEYNPYGATCYGLSHSAMVSWVNEFLNEYHSKTGVYPLLYTTADWWNQCTGNAHGFGNKSPLVLASYSSESPRTVPGDWQTWTIWQNADKYKYGGDSDIFNGDLTQLKKIVEG

SEQ ID NO: 48 - protein, 218 aa, Aspergillus sp.
LPTKLAARYSTVQGFDVSNYQPNVDFSAAKSAGAEFVIIKATEGTDYKDTYFNSHYTGATNAGLIRGGYHFARPDKSSGTAQAEYFLAHGGGWSKDGRTLPGMLDIEYNPYGATCYGLSHSAMVSWVNEFLNEYHSKTGVYPLLYTTADWWNQCTGNAHGFGNKSPLVLASYSSESPRTVPGDWQTWTIWQNADKYKYGGDSDIFNGDLTQLKKIVEG

SEQ ID NO: 49 - DNA, 747 bp, Paecilomyces sp.
  sig_peptide 1..57
  mat_peptide 58..744
  source 1..747: mol_type = genomic DNA
  CDS 1..135
  CDS 211..744
atgaagtctgttgctgtctttgccggtctggcctccatggtcagcattgccacagccaccgttgcaggcttcgatatttccaactaccaaccttcggtcaactttgcaaaagcatacgcggacggtgcacgcttcgtcattatcaaggcaagcaggccagaccagcgcctgccagctcaaacaagaccagattgctaactctcttctcaggccaccgaaggcaccacctacatcgaccccagtttcagctcccattacaccggggccactaacgccggtctcatccgcggaggctaccattttgcccatccgggatccagcaccggcgccgctcaggccacctacttccttgcccacggcggcggctggtccaaggacggcatcacgctccctggcatgatcgacctcgagtacaaccccagtggcgcgacctgctatggcctctcgaccagcgccatggtcagctggatctccgactttgtcgagacgtaccacagcaagacgggcgtctacccgctcatttatacctcgacaagctggtggaaccagtgtaccggcagcagcaccgcctttgccagcaagtgtcctcttgtggttgctcgctacgccagcagcgttggcactcttcctgccggttggggctaccagaccatctggcagaatagcgatagctcgccctggggcggtgacaatgatattttcaacggcagtctggaccagctcaagcgcattgcgaatgcttcttag

SEQ ID NO: 50 - protein, 223 aa, Paecilomyces sp.
MKSVAVFAGLASMVSIATATVAGFDISNYQPSVNFAKAYADGARFATEGTTYIDPSFSSHYTGATNAGLIRGGYHFAHPGSSTGAAQATYFLAHGGGWSKDGITLPGMIDLEYNPSGATCYGLSTSAMVSWISDFVETYHSKTGVYPLIYTSTSWWNQCTGSSTAFASKCPLVVARYASSVGTLPAGWGYQTIWQNSDSSPWGGDNDIFNGSLDQLKRIANAS

SEQ ID NO: 51 - protein, 204 aa, Paecilomyces sp.
TVAGFDISNYQPSVNFAKAYADGARFATEGTTYIDPSFSSHYTGATNAGLIRGGYHFAHPGSSTGAAQATYFLAHGGGWSKDGITLPGMIDLEYNPSGATCYGLSTSAMVSWISDFVETYHSKTGVYPLIYTSTSWWNQCTGSSTAFASKCPLVVARYASSVGTLPAGWGYQTIWQNSDSSPWGGDNDIFNGSLDQLKRIANAS

SEQ ID NO: 52 - DNA, 669 bp, Paecilomyces sp.
  sig_peptide 1..57
  mat_peptide 58..666
  source 1..669: mol_type = genomic DNA
  CDS 1..666
atgaagctcacgagtgtgttgaccctggttggctgtgccgtcacaggcacatctgccgccgtgcaaggacacgacgtcagccattggcagggtaacatcaactggggcgcggtcaaggcagccggcgtcaagtttacatacattaaagcaacagagtcaaccaactacatcgaccccagcttcaacgcaaattatgtcggcgccaccaataccggactgatacgcggcgcataccactttgcccggccaggggattcatcaggtgccgcgcaggcaaattattttgtcagccatggtggtgggtggtccgcagacgggagaactttgcctggcgctcttgatcttgaggcgggctgtagcggattgtcgcaatcagcaatgacggcctggatccgggacttcagcaacacctatcacgcgcggactggtcggttccccgtcatttacacaactaccagctggtggaagacttgcaccggcaatgcgtccggatttcagaacgaccatccgctttggattgcgcgatggggcccttcacctggggagttgccggcaggatatggctttcacaccttttggcagtatgcggacaagggacctcttccaggcgaccaggacaactttaatggcgatgaggccggtcttgcaaggcttgctagaggttcgtaa

SEQ ID NO: 53 - protein, 222 aa, Paecilomyces sp.
MKLTSVLTLVGCAVTGTSAAVQGHDVSHWQGNINWGAVKAAGVKFTYIKATESTNYIDPSFNANYVGATNTGLIRGAYHFARPGDSSGAAQANYFVSHGGGWSADGRTLPGALDLEAGCSGLSQSAMTAWIRDFSNTYHARTGRFPVIYTTTSWWKTCTGNASGFQNDHPLWIARWGPSPGELPAGYGFHTFWQYADKGPLPGDQDNFNGDEAGLARLARGS

SEQ ID NO: 54 - protein, 203 aa, Paecilomyces sp.
AVQGHDVSHWQGNINWGAVKAAGVKFTYIKATESTNYIDPSFNANYVGATNTGLIRGAYHFARPGDSSGAAQANYFVSHGGGWSADGRTLPGALDLEAGCSGLSQSAMTAWIRDFSNTYHARTGRFPVIYTTTSWWKTCTGNASGFQNDHPLWIARWGPSPGELPAGYGFHTFWQYADKGPLPGDQDNFNGDEAGLARLARGS

SEQ ID NO: 55 - DNA, 944 bp, Rhizomucor pusillus
  sig_peptide 1..60
  mat_peptide 61..941
  source 1..944: mol_type = genomic DNA
  CDS 1..184
  CDS 241..242
  CDS 244..484
  CDS 555..688
  CDS 757..825
  CDS 885..941
atgaagtttgcactcctagtatctgctatcgcaggccttgcagcaaccgccgtccaagcctacgaaactggcgttgatgtctctgccttgacttccacctccgcctggagctgtgcaaagaaactcggttacgatcacgctattgtccgctgctacattgaggcatacggaggcaaccctgcaagtatcatgcatcagagaagacgactactacgttctagcgtgctaacaatgtactagggtggtaaaattgacagcaactgcttccagaactacaagaacgccaaagcaggtggattcaccagcgttgacatttacatgttcccctgcactggccgctccacatgcaagtcgcccgctgctcaggtcaaggaggtcgttgactacgttggctccaacaagatgactgttggacgtctctggctcgacgtcgaaatcgatccttctgctaacaactggccatccgccagtagcgctcgcagcagtaagaaagaggacaagaaaccaattcaggatgctcacaatgttgctaacattttttattacttgttaagccttgaagtccttcaagtctgctcttgactcgactggctggaaatacggtatctattcttctgcttcccaatggtcgcagattaccggatcctcctcctgggagcttgattcctcgttgccactttggtacgctgtaagtagctctcacgttattccgtatattgtgggagaacgtggctgatctgaatttattaccacaagcactacgacgcttctctcagcttcagcgacttttcgccatttggtggctggactaagcctaccatcaaggtaaatattcaggtcatccggtgcttgacgcatatgtactcaattaaatcgccatttagcaatatgctggctctgtaagcttctgctccgctggctgggacaagaactactatggctaa

SEQ ID NO: 56 - protein, 229 aa, Rhizomucor pusillus
MKFALLVSAIAGLAATAVQAYETGVDVSALTSTSAWSCAKKLGYDHAIVRCYIEAYGGNPARGKIDSNCFQNYKNAKAGGFTSVDIYMFPCTGRSTCKSPAAQVKEVVDYVGSNKMTVGRLWLDVEIDPSANNWPSASSARSTLKSFKSALDSTGWKYGIYSSASQWSQITGSSSWELDSSLPLWYAHYDASLSFSDFSPFGGWTKPTIKQYAGSVSFCSAGWDKNYYG

SEQ ID NO: 57 - protein, 209 aa, Rhizomucor pusillus
YETGVDVSALTSTSAWSCAKKLGYDHAIVRCYIEAYGGNPARGKIDSNCFQNYKNAKAGGFTSVDIYMFPCTGRSTCKSPAAQVKEVVDYVGSNKMTVGRLWLDVEIDPSANNWPSASSARSTLKSFKSALDSTGWKYGIYSSASQWSQITGSSSWELDSSLPLWYAHYDASLSFSDFSPFGGWTKPTIKQYAGSVSFCSAGWDKNYYG

SEQ ID NO: 58 - DNA, 887 bp, Pycnidiophora cf.
  sig_peptide 1..51
  mat_peptide 52..884
  source 1..887: mol_type = genomic DNA
  CDS 1..144
  CDS 205..265
  CDS 358..781
  CDS 839..884
atgaagtccgtactcgccctgctcgccgctggcgcccaacttgccagtgcggccgtctccggcatggatatctcccactaccaagggacaaactacaactttgccggcgcatactcctctggtgctcgctttgtcatcatcaaggtacatgcccattctagcttaaggatttgaccccaatgttcggcctttaacgcatcctaggccaccgaaggaaccacatataccgatcctcagttctccgctaactacattggtgccacgagtaagtagatttccccatgaggtagctggtcgttagtcttctcctctcctaccacactgtccatgctggggagacccattaacactgagtagatgctggttttatccgcggcgcgtaccatttcgcccgccctgccgcctctactggggccgtccaagcctcctacttcgtctcccatggcggtgggtggtcatcggatggcatcacactccccggcatgcttgacatggaatatggctctacctccacttgccacggcctttctgtctccgccatgaacacgtggatcgcctccttcgtgaaccagtacaagagtttgacgggcgcataccccatgatttacaccacagccgattggtggaagacttgcacaggagacagcacggcttggaacaccaagtgccctttggtattggccaggtactctagttctgtggggacgattcctggaggttggccgtatcataccatttggcaatattccgattcctatgcgtacggtggggattcggatacgtaagttcgacatgctcaagcgaatggaacggatggagtggctaacgggatggacagattcaatggcgacttggctggcttaaagaggcttgcgaagggcagctaa

SEQ ID NO: 59 - protein, 225 aa, Pycnidiophora cf.
MKSVLALLAAGAQLASAAVSGMDISHYQGTNYNFAGAYSSGARFVIIKATEGTTYTDPQFSANYIGATNAGFIRGAYHFARPAASTGAVQASYFVSHGGGWSSDGITLPGMLDMEYGSTSTCHGLSVSAMNTWIASFVNQYKSLTGAYPMIYTTADWWKTCTGDSTAWNTKCPLVLARYSSSVGTIPGGWPYHTIWQYSDSYAYGGDSDTFNGDLAGLKRLAKGS

SEQ ID NO: 60 - protein, 208 aa, Pycnidiophora cf.
AVSGMDISHYQGTNYNFAGAYSSGARFVIIKATEGTTYTDPQFSANYIGATNAGFIRGAYHFARPAASTGAVQASYFVSHGGGWSSDGITLPGMLDMEYGSTSTCHGLSVSAMNTWIASFVNQYKSLTGAYPMIYTTADWWKTCTGDSTAWNTKCPLVLARYSSSVGTIPGGWPYHTIWQYSDSYAYGGDSDTFNGDLAGLKRLAKGS

SEQ ID NO: 61 - DNA, 974 bp, Thermomucor indicae
  sig_peptide 1..66
  mat_peptide 67..971
  source 1..974: mol_type = genomic DNA
  CDS 1..186
  CDS 250..493
  CDS 579..712
  CDS 777..851
  CDS 915..971
atgaagttttcaatctttgctattgctgctgctgctgctgccgccgtggccaccttggttcaaggctaccaaaccggtctggatgtatctgccttgacctcgacatcgtcctttagctgtgcgaaaaaccttggatatgaccatgtgattgctcgatgctacatggaagcctacggcaacaacccggtaaaaccaacatcgaactcaaaggaagatggaaatgaaataacaaaaaagggttatacgcagggtggcaaagtggatcccaactgttatagcaattacaagaacgccaaggcagctggattcacgtcggtggatatctacatgttcccatgtaccggccgatccacctgcaagtcgccagcgactcaagtccaagaaatcgtggattacgtcggggcccacaagatgattgtcggaactttgtggttggatgtcgaggtcgattcggctgctaataactggccgtccacgtccgaagcccggtccagtaagttcccccgtccagagctgtctatctatctatctatcaattgtatgaaccgttcgtcataactcacggtgcgcatcgttagcactccgagctttcaagacggcgttggataagtccggatggaaatggggcgtttattccagcaagtcgcaatggacgcgtatcaccggatcagcttcatgggttttagacccgtcggtgcctttgtggtattcggtaagttgttacataacagctgcgatttccgtgccatgatctttcttatctgtttcatacgtagcactacgacgatacccttagtttcagtgactatccatcgcacgctttcggtggctggtccaaaccaacaatcaaggtacgttagcatttttttcaaaataaaatagttctctgccgtattaatcctctccatgctcagcaatatacgggagatgcttccttttgctctgccagttgggacaagaactactatggttag

SEQ ID NO: 62 - protein, 232 aa, Thermomucor indicae
MKFSIFAIAAAAAAAVATLVQGYQTGLDVSALTSTSSFSCAKNLGYDHVIARCYMEAYGNNPGGKVDPNCYSNYKNAKAAGFTSVDIYMFPCTGRSTCKSPATQVQEIVDYVGAHKMIVGTLWLDVEVDSAANNWPSTSEARSTLRAFKTALDKSGWKWGVYSSKSQWTRITGSASWVLDPSVPLWYSHYDDTLSFSDYPSHAFGGWSKPTIKQYTGDASFCSASWDKNYYG

SEQ ID NO: 63 - protein, 210 aa, Thermomucor indicae
YQTGLDVSALTSTSSFSCAKNLGYDHVIARCYMEAYGNNPGGKVDPNCYSNYKNAKAAGFTSVDIYMFPCTGRSTCKSPATQVQEIVDYVGAHKMIVGTLWLDVEVDSAANNWPSTSEARSTLRAFKTALDKSGWKWGVYSSKSQWTRITGSASWVLDPSVPLWYSHYDDTLSFSDYPSHAFGGWSKPTIKQYTGDASFCSASWDKNYYG

SEQ ID NO: 64 - DNA, 675 bp, Isaria farinosa
  sig_peptide 1..51
  mat_peptide 52..672
  source 1..675: mol_type = genomic DNA
  CDS 1..672
atgcgcgcctctacagccctctttatcgcctttgccggcgtggccactgccctgacccacgccgtcgacagctcctccgaggtgagcgtcgacatttacaaaaaggcgctcggccagggcttcacgcgcgccatcttccgcggctaccaggaggcgtgctcgctcggcggccgcgtcgacccgacctttgtgcccagctacaaaaacgccgtcgcggccggctacaaggactttgacgcctacttcttcccctgcaccggcacgaccaacaagtgcaagccgtacgcgacgcagctcgccgagctgctcgacaccatcagcagccagaagctggcgatccgccgcatctggctcgacattgagacggaccaggtctgcagcccgttcgactatggcgcgcagggcaacattgccgaggccaagaagctcgtcgccgcgtttcgcgccgcaaagcacgactggggcatctacacgtcgccgacgcagtgggagactatctttggctccaagacattcgtgctggccaatgatgtgccgctctggtttgccaagtttgacaatgtcgagacgctggacctgaagacgccgtttggcggctggacaaaggccgacgcaaaacagtacacggaccagtcggctagcaagaagtttgacttgaacgtcttctctgcataa

SEQ ID NO: 65 - protein, 224 aa, Isaria farinosa
MRASTALFIAFAGVATALTHAVDSSSEVSVDIYKKALGQGFTRAIFRGYQEACSLGGRVDPTFVPSYKNAVAAGYKDFDAYFFPCTGTTNKCKPYATQLAELLDTISSQKLAIRRIWLDIETDQVCSPFDYGAQGNIAEAKKLVAAFRAAKHDWGIYTSPTQWETIFGSKTFVLANDVPLWFAKFDNVETLDLKTPFGGWTKADAKQYTDQSASKKFDLNVFSA

SEQ ID NO: 66 - protein, 207 aa, Isaria farinosa
LTHAVDSSSEVSVDIYKKALGQGFTRAIFRGYQEACSLGGRVDPTFVPSYKNAVAAGYKDFDAYFFPCTGTTNKCKPYATQLAELLDTISSQKLAIRRIWLDIETDQVCSPFDYGAQGNIAEAKKLVAAFRAAKHDWGIYTSPTQWETIFGSKTFVLANDVPLWFAKFDNVETLDLKTPFGGWTKADAKQYTDQSASKKFDLNVFSA

SEQ ID NO: 67 - DNA, 829 bp, Lecanicillium sp.
  sig_peptide 1..57
  mat_peptide 58..826
  source 1..829: mol_type = genomic DNA
  CDS 1..147
  CDS 210..393
  CDS 480..826
atgaagtcttttggcctgttcgcctcactcgcctctctggctggcatcgccagcgcctcggtccagggttttgacatttcccactaccagagctctgtcaactttggcgcggcctacgctgacggcgctcgcttcgtcattatcaaggtgcgattttgtcgcctggcgttttctcaaccctggctacctactcacaaccttatcattaggcaaccgagggaacgacgtaccgcgaccccaagttcagcgagcactacggcggcgccaccaaggccggcttcatccgcggcggctatcactttgcccagcctgcctcatcctctggcgccgcgcaggccaactttttcctcgctcacggcggcggctggagcggcgacggcatcaccctgcccggtaagcctcgcgcttatacacgcatgcttttcacaccccagctcgacaaagaaccctcatttctgattaaactttttgtggcttaggtatgctggatctcgagtatggcccgaacgggaacacctgctacggcctcggcccggcgtccatgcggagctggatcagcgactttgtcgagacgtaccacgccaagacgggccgctaccccctcatctacacgtcgacgagctggtggaagacgtgcacgggcaacacgtccctctttgccgacaagtgcccgctggtcgtcgcgcgctataacagccaggtcggcgagctccctgccggctggggcttctacactttctggcagttcaacgatcactacaagcatggcggcgactcggacgtgttcaacggcgcctactctcagcttcagaagattgccactggttag

SEQ ID NO: 68 - protein, 226 aa, Lecanicillium sp.
MKSFGLFASLASLAGIASASVQGFDISHYQSSVNFGAAYADGARFVIIKATEGTTYRDPKFSEHYGGATKAGFIRGGYHFAQPASSSGAAQANFFLAHGGGWSGDGITLPGMLDLEYGPNGNTCYGLGPASMRSWISDFVETYHAKTGRYPLIYTSTSWWKTCTGNTSLFADKCPLVVARYNSQVGELPAGWGFYTFWQFNDHYKHGGDSDVFNGAYSQLQKIATG

SEQ ID NO: 69 - protein, 207 aa, Lecanicillium sp.
SVQGFDISHYQSSVNFGAAYADGARFVIIKATEGTTYRDPKFSEHYGGATKAGFIRGGYHFAQPASSSGAAQANFFLAHGGGWSGDGITLPGMLDLEYGPNGNTCYGLGPASMRSWISDFVETYHAKTGRYPLIYTSTSWWKTCTGNTSLFADKCPLVVARYNSQVGELPAGWGFYTFWQFNDHYKHGGDSDVFNGAYSQLQKIATG

SEQ ID NO: 70 - DNA, 742 bp, Zopfiella sp.
  sig_peptide 1..51
  mat_peptide 52..739
  source 1..742: mol_type = genomic DNA
  CDS 1..141
  CDS 206..739
atgaaatccatcctcctctcagctgcttccctcatcggggccgcccaagccgcggtccagggattcgacgtctcccattggcagtccagcgtcaactttgccgccgcctacaactcgggggctcggtttgtgatcatcaaggtctttttttttccttcattcattcaccaataactacctactgacccgtgagatgtcatgacaggccaccgagagcaacaactacatcgaccccaagttcaacacgtactatccggccgccaccagcgccggtctgatccggggcgggtaccacttcgcccacccgggggagaccacgggcgccgtgcaggcggactacttcatcgcgcacggcgggggttggtccagcgacggcatcactctcccgggaatgctcgacctggagaacgcaagcggctacccggcctgctggggcctgtcgcagagcgccatggtctcgtggatcaaggccttcagcgaccggtacaaggcccgcaccggcgtctaccccatgctctacaccaacccgtcctggtggaccagctgcaccggcaactccaacgccttcgtcaacaccaaccccctcgtcctggcccgctacgccagctcccccggcaccatccccggcggctggccctaccagaccatctggcagaactcggactcgtatacctacggcggcgactcggacatctttaatggggatctggctggtctcaagaggttggccaagggttcttaa

SEQ ID NO: 71 - protein, 225 aa, Zopfiella sp.
MKSILLSAASLIGAAQAAVQGFDVSHWQSSVNFAAAYNSGARFVIIKATESNNYIDPKFNTYYPAATSAGLIRGGYHFAHPGETTGAVQADYFIAHGGGWSSDGITLPGMLDLENASGYPACWGLSQSAMVSWIKAFSDRYKARTGVYPMLYTNPSWWTSCTGNSNAFVNTNPLVLARYASSPGTIPGGWPYQTIWQNSDSYTYGGDSDIFNGDLAGLKRLAKGS

SEQ ID NO: 72 - protein, 208 aa, Zopfiella sp.
AVQGFDVSHWQSSVNFAAAYNSGARFVIIKATESNNYIDPKFNTYYPAATSAGLIRGGYHFAHPGETTGAVQADYFIAHGGGWSSDGITLPGMLDLENASGYPACWGLSQSAMVSWIKAFSDRYKARTGVYPMLYTNPSWWTSCTGNSNAFVNTNPLVLARYASSPGTIPGGWPYQTIWQNSDSYTYGGDSDIFNGDLAGLKRLAKGS

SEQ ID NO: 73 - DNA, 767 bp, Malbranchea flava
  sig_peptide 1..54
  mat_peptide 55..764
  source 1..767: mol_type = genomic DNA
  CDS 1..174
  CDS 234..764
atgaagctgtctctcctccttattgttgctgcatcactggccgtggccagtgcaggccccaaggagttcgagtcacgcgcgtcgggcgtccagggctttgacatctctggttggcagtccaacgtcaattttgcaggtgcatacaattctggcgcacgcttcgtcatgatcaaggtacatttgagtgaattcgtttctcctggtataataccctgactaatgtaaagatccaggctagcgagggtaccaccttcaaggaccgtcaattcagcaatcattacattggcgccaccaaggctggctttatccgtggcggctaccactttgcgttgccagacgtcagcagcgccactgcccaagtgaaccatttcctggccagcggtggtggctggagcagagacggcatcacgctgccgggcatgctggacatcgagagcaacccgtatggcgcccagtgctacggccttgacgctggtcgtatggttgcctggatccgggagtttgttgacgcgtacaagcgcgcaactggacggtatcctctgatctacacgtctcccagctggtggcagacttgcacgggcaatagcaatgcctttatagacaagtgcccgcttgtgttggcacggtgggcgagtagccctggcactccgcctggtgggtggccgttccacagtttttggcagtacgccgattcctatcaattcggtggtgacgcccaggtattcaatggcgatgaggctgggttgaagagaatggccctaggttaa

SEQ ID NO: 74 - protein, 235 aa, Malbranchea flava
MKLSLLLIVAASLAVASAGPKEFESRASGVQGFDISGWQSNVNFAGAYNSGARFVMIKASEGTTFKDRQFSNHYIGATKAGFIRGGYHFALPDVSSATAQVNHFLASGGGWSRDGITLPGMLDIESNPYGAQCYGLDAGRMVAWIREFVDAYKRATGRYPLIYTSPSWWQTCTGNSNAFIDKCPLVLARWASSPGTPPGGWPFHSFWQYADSYQFGGDAQVFNGDEAGLKRMALG

SEQ ID NO: 75 - protein, 217 aa, Malbranchea flava
GPKEFESRASGVQGFDISGWQSNVNFAGAYNSGARFVMIKASEGTTFKDRQFSNHYIGATKAGFIRGGYHFALPDVSSATAQVNHFLASGGGWSRDGITLPGMLDIESNPYGAQCYGLDAGRMVAWIREFVDAYKRATGRYPLIYTSPSWWQTCTGNSNAFIDKCPLVLARWASSPGTPPGGWPFHSFWQYADSYQFGGDAQVFNGDEAGLKRMALG

SEQ ID NO: 76 - DNA, 935 bp, Hypholoma polytrichi
  sig_peptide 1..60
  mat_peptide 61..932
  source 1..935: mol_type = genomic DNA
  CDS 1..396
  CDS 454..552
  CDS 620..703
  CDS 770..798
  CDS 857..932
atggctaagctcttgaaacaactcgtgctccttcctttcctcgcgctcgcggcgcacgcgctcgtctacggcgtggactcctccagcctcgtccccgtcgcgacgtaccagaaggcgctcggcgagggcttcacgaaggcggttatccgcgggtacgaggaggcgtgcggcgtggggggcgaggtcgaccccaacttcgtcccgtcgtacaagaacgcgcgcgcggcggggtacacggacattgacatgtactggttcccgtgcaacggctcgacgcacagctgcaagtcgtatgcggcgcagctcgccgcgatcgcggccgcgttctctgcgaatgcgatgaagatcggcacgatctggatcgacatcgagaaggacgcggcgatctgcaacaacgtgagtggtccgtcgtgttgtgcggcgcacggcgtgagttgaccgtttggcacacagtgggactatggcacggcggggaacctcgcgcaggcgaaagcgctgatcgcggctgcgaaggcgtcgggattcaattttggcatctacagtagccctggggtatgtgtggtgctgcattagcctgccaatggatcttcactgaaaatttggattctcgtatacacaggagtggagcacaattttcggctcgacgagtgtcgtcgtcgacaactcggcaccgctctggtttgctacttacaacaacgtccaagtacgtgctcacattaattgactgctatctgctgcttttcgttgcttacctctcacttcgtggcagacccttactttgggaacaccgttcggcgggtaagtcatgcgcccaacgtcaattctgattctgtgattctcaaattgtgcgccttagctggagtacggctgtgggccaccaatatactgacgtctcggcgtcggggttattcgatctcaacgtttttgcccactaa

SEQ ID NO: 77 - protein, 228 aa, Hypholoma polytrichi
MAKLLKQLVLLPFLALAAHALVYGVDSSSLVPVATYQKALGEGFTKAVIRGYEEACGVGGEVDPNFVPSYKNARAAGYTDIDMYWFPCNGSTHSCKSYAAQLAAIAAAFSANAMKIGTIWIDIEKDAAICNNWDYGTAGNLAQAKALIAAAKASGFNFGIYSSPGEWSTIFGSTSVVVDNSAPLWFATYNNVQTLTLGTPFGGWSTAVGHQYTDVSASGLFDLNVFAH

SEQ ID NO: 78 - DNA, 687 bp, Synthetic construct
  sig_peptide 1..60
  mat_peptide 61..684
  source 1..687: mol_type = other DNA
  CDS 1..684
atggcaaagctcctcaagcagttggtgttgctcccgttcctcgcgttggcagcacacgcattggtctacggagtcgattcgtcctcgttggtccctgtggcgacgtatcagaaggcattgggagaaggcttcacaaaggccgtcattaggggctacgaagaggcctgtggagtcggaggagaggtcgatcccaacttcgtcccctcctacaaaaacgcacgagcggcaggatacacagacatcgatatgtactggttcccctgtaacggctccactcattcgtgtaaatcgtatgccgcacagttggcagccattgccgcagccttctcggcgaacgccatgaagatcggtactatttggatcgacatcgaaaaagatgcagccatctgtaacaactgggattacggcactgcaggtaacttggcccaggcgaaggcattgattgccgcagcgaaggcatccggtttcaacttcggcatctactcgtcgcctggagagtggtcgaccatcttcggctcgacctcggtcgtcgtcgacaactccgcaccgctctggttcgcgacctataacaacgtccagaccctcacgctcggcactcctttcggaggctggtcgacagccgtcggtcatcagtataccgatgtgtccgcctccggactcttcgacctcaacgtcttcgcccactaa

SEQ ID NO: 79 - protein, 228 aa, Synthetic construct; identical to SEQ ID NO: 77
  REGION 1..228: note = Synthetic construct

SEQ ID NO: 80 - protein, 208 aa, Hypholoma polytrichi
LVYGVDSSSLVPVATYQKALGEGFTKAVIRGYEEACGVGGEVDPNFVPSYKNARAAGYTDIDMYWFPCNGSTHSCKSYAAQLAAIAAAFSANAMKIGTIWIDIEKDAAICNNWDYGTAGNLAQAKALIAAAKASGFNFGIYSSPGEWSTIFGSTSVVVDNSAPLWFATYNNVQTLTLGTPFGGWSTAVGHQYTDVSASGLFDLNVFAH

SEQ ID NO: 81 - DNA, 701 bp, Aspergillus deflectus
  sig_peptide 1..45
  mat_peptide 46..698
  source 1..701: mol_type = genomic DNA
  CDS 1..135
  CDS 186..698
atgaagcttctttccgcccttgcgctccctggtctggcctacgccgcagtccaaggcttcgacatttcgcactaccagtcgagtgtcgactatgccggcgcctactcctcgggtgcccgcttcgtcatgatcaaggtaccctcctagcctctaccagcagaaacaagggactaagagagagaaaggccaccgaaggaacgacgtatacggacccggcttttagtactcactatacgggtgctacaaacgctggcctgatccgtggcggttatcactttgctcgccccggttctagttctggtgccgcccaagcagaatatttccttgcccatggcggtgggtggactggtgatggaatcacccttcctggtatgttggaccttgaggctggatgctctggtctgtcggcctcagccatggtctcctggatccaggactttggcgagacctacaaggcaagcacgggacggtatccaatgatctataccacaacaagctggtggagttcctgtacaggcaacaatggtggctttggcgattatcctctggtccttgcgagatgggcttcatcgccaggcgagcttccgaacggctggtcggtgcactctttctggcaaaatgccgatacttatgagtatgggggtgattccgagatttggaacggaagtcaagaaaatcttgtcaagtttgcctctcagtga

SEQ ID NO: 82 - protein, 216 aa, Aspergillus deflectus
MKLLSALALPGLAYAAVQGFDISHYQSSVDYAGAYSSGARFVMIKATEGTTYTDPAFSTHYTGATNAGLIRGGYHFARPGSSSGAAQAEYFLAHGGGWTGDGITLPGMLDLEAGCSGLSASAMVSWIQDFGETYKASTGRYPMIYTTTSWWSSCTGNNGGFGDYPLVLARWASSPGELPNGWSVHSFWQNADTYEYGGDSEIWNGSQENLVKFASQ

SEQ ID NO: 83 - protein, 201 aa, Aspergillus deflectus
AVQGFDISHYQSSVDYAGAYSSGARFVMIKATEGTTYTDPAFSTHYTGATNAGLIRGGYHFARPGSSSGAAQAEYFLAHGGGWTGDGITLPGMLDLEAGCSGLSASAMVSWIQDFGETYKASTGRYPMIYTTTSWWSSCTGNNGGFGDYPLVLARWASSPGELPNGWSVHSFWQNADTYEYGGDSEIWNGSQENLVKFASQ

SEQ ID NO: 84 - DNA, 820 bp, Ascobolus stictoideus
  sig_peptide 1..48
  mat_peptide 49..817
  source 1..820: mol_type = genomic DNA
  CDS 1..138
  CDS 230..675
  CDS 748..817
atgaagtggcttggtctcatcaccctcctcgtcggaacagcctcggctgcagtccctggcttcgacatctcccactaccaaagcaccgtcaatttcgccgatgcctactcctcaggagctcgattcgtcatcatcaaggtaatattcctcctcttaccccttcctcctatcgacctcaagccattatcaattccatcatgatcgatcagtgctaaccttctctctccaggccaccgaaggcacaacctacaaagacccaaaattctcctcccactacaccggcgcaaccaacgccggtctaattcgcggcggctaccatttcgcccgtcccgcctccagcaccggcgccgtgcaagcccaatactttgtcagtaacggtggtggttggtccggcgatggccttactctccccggcatgcttgatcttgagggagattgtgccggattatcacaagccggtatggtatcatggattacatcattcgtcaacaagtacaaggctttgactacaagatatccaatgatttatactaccaattcttggtggaatacttgtacggggaatagtcaggcttttagtgcgaattgtcccttggttattgcgaggtataattcggttgttggaactattcctggaggctggccttattatactatttggcagtttaatgatgcgtaagttactccccatgagcctgtttttggatgaggggggagtagagtttggctaactaggtggctttataggtactcctatggtggtgattctgataccttcaatggggcttactcgcagcttgttaagctcgctactggttaa

SEQ ID NO: 85 - protein, 218 aa, Ascobolus stictoideus
MKWLGLITLLVGTASAAVPGFDISHYQSTVNFADAYSSGARFVIIKATEGTTYKDPKFSSHYTGATNAGLIRGGYHFARPASSTGAVQAQYFVSNGGGWSGDGLTLPGMLDLEGDCAGLSQAGMVSWITSFVNKYKALTTRYPMIYTTNSWWNTCTGNSQAFSANCPLVIARYNSVVGTIPGGWPYYTIWQFNDAYSYGGDSDTFNGAYSQLVKLATG

SEQ ID NO: 86 - protein, 202 aa, Ascobolus stictoideus
AVPGFDISHYQSTVNFADAYSSGARFVIIKATEGTTYKDPKFSSHYTGATNAGLIRGGYHFARPASSTGAVQAQYFVSNGGGWSGDGLTLPGMLDLEGDCAGLSQAGMVSWITSFVNKYKALTTRYPMIYTTNSWWNTCTGNSQAFSANCPLVIARYNSVVGTIPGGWPYYTIWQFNDAYSYGGDSDTFNGAYSQLVKLATG

SEQ ID NO: 87 - DNA, 734 bp, Coniochaeta sp.
  sig_peptide 1..51
  mat_peptide 52..731
  source 1..734: mol_type = genomic DNA
  CDS 1..141
  CDS 201..731
atgaagtccacattcatcaccgctctaggcctagcaggcgtcgcgcaggcgactgtccagggcttcgacatctcgcactaccagcccacggtcaactatgccggtgcttataacgcaggtgcacgcttcgtcatcatcaaggtcagtcacctcggttgaaccttctgcttcacacatgtcaaagattaacatcaagccaggccaccgagggaacaacttacaccgacccttccttcagcacccactacaacggcgcaaccaaggccggtctcatccgcggcggctaccacttcgcccaccccggcgtcaccaccggcgccgcggaggccaacttcttcctcgcccacggcggcggctggtcgggcgacggcatcaccctccccggcatgctcgacctcgagtccgagggctccaacccgcagtgctggggcctgtccacctccggcatggtcgcgtggatcaagtccttcagcgaccggtaccacaccgtcaccggccggtatcccatgctctacaccaacccgtcctggtggagcacctgcaccggcaacagcaacgctttcgtcaacaccaacccgctcgtcctggcgcggtatgcttccgcgcccggcaccatccccggcggctggccgtatcagaccatctggcagaacagcgactcgtactcttatggcggtgattcggacatcttcaacgggaacttggctagcttgcagaagttggctactggttag

SEQ ID NO: 88 - protein, 224 aa, Coniochaeta sp.
MKSTFITALGLAGVAQATVQGFDISHYQPTVNYAGAYNAGARFVIIKATEGTTYTDPSFSTHYNGATKAGLIRGGYHFAHPGVTTGAAEANFFLAHGGGWSGDGITLPGMLDLESEGSNPQCWGLSTSGMVAWIKSFSDRYHTVTGRYPMLYTNPSWWSTCTGNSNAFVNTNPLVLARYASAPGTIPGGWPYQTIWQNSDSYSYGGDSDIFNGNLASLQKLATG

SEQ ID NO: 89 - protein, 207 aa, Coniochaeta sp.
TVQGFDISHYQPTVNYAGAYNAGARFVIIKATEGTTYTDPSFSTHYNGATKAGLIRGGYHFAHPGVTTGAAEANFFLAHGGGWSGDGITLPGMLDLESEGSNPQCWGLSTSGMVAWIKSFSDRYHTVTGRYPMLYTNPSWWSTCTGNSNAFVNTNPLVLARYASAPGTIPGGWPYQTIWQNSDSYSYGGDSDIFNGNLASLQKLATG

SEQ ID NO: 90 - DNA, 815 bp, Daldinia fissa
  sig_peptide 1..60
  mat_peptide 61..812
  source 1..815: mol_type = genomic DNA
  CDS 1..150
  CDS 226..712
  CDS 784..812
atgaagaccttcattacaagttgtctaggcctcgctggcctcgctagccttgccctcgctgctgtcccaggcttcgacatttcccattatcagtcgagcgtagactttggggcagcatatagttctggagcccgtttcgttatcatcaaagtgagtcctacctaggtacctacctatatctaccaaccaaatacgtccccctcgaaacttaccatatgagaccaggctacagagggcaccacctaccaggaccccaaattcagcagccactacgccggcgctaccaacgccggcctcatccgcggcggctaccacttcgcgcgccccgcgtcgtcctccggcgccgcccaagcaacgttcttcctcgcgcacggtggcgggtggtcgggcgatgggatcacgctgccgggaatgctggacttagaaggtgattgtgcgggcttgtcgaccagcgccatggtctcgtggatcagggactttagcgatacgtatcacggcaagacggggcggtatcccctgctgtacacgaatccgtcgtggtggtcgagttgtacgggcgggtcgagcgcgttcgtgaatacgaatccgctcgtgcttgcgcggtatgctagtagcccgggggcgttgccggggggctggccgtattataccatttggcagtttaatgatgcgtataagtatgggggcgactcggatacgtttaatggcgatcgtgagtaacctagttttacttgtctttctctgttcctttttttgtccccttttgctaacgtagtgttgcagtcacgcagttgaagaaattggcttcggggtaa

SEQ ID NO: 91 - protein, 222 aa, Daldinia fissa
MKTFITSCLGLAGLASLALAAVPGFDISHYQSSVDFGAAYSSGARFVIIKATEGTTYQDPKFSSHYAGATNAGLIRGGYHFARPASSSGAAQATFFLAHGGGWSGDGITLPGMLDLEGDCAGLSTSAMVSWIRDFSDTYHGKTGRYPLLYTNPSWWSSCTGGSSAFVNTNPLVLARYASSPGALPGGWPYYTIWQFNDAYKYGGDSDTFNGDLTQLKKLASG

SEQ ID NO: 92 - protein, 202 aa, Daldinia fissa
AVPGFDISHYQSSVDFGAAYSSGARFVIIKATEGTTYQDPKFSSHYAGATNAGLIRGGYHFARPASSSGAAQATFFLAHGGGWSGDGITLPGMLDLEGDCAGLSTSAMVSWIRDFSDTYHGKTGRYPLLYTNPSWWSSCTGGSSAFVNTNPLVLARYASSPGALPGGWPYYTIWQFNDAYKYGGDSDTFNGDLTQLKKLASG

SEQ ID NO: 93 - DNA, 732 bp, Delitschia sp.
  sig_peptide 1..51
  mat_peptide 52..725
  source 1..732: mol_type = genomic DNA
  CDS 1..141
  CDS 217..729
atgaaggctactctcgcttccctcctcggtcttgccaacggcgccctcgcggctgtccagggcttcgacatctcccactaccagtcgagcgtcaacttcgccggtgcgtatagcgccggtgcccgtttcgtcatcatcaaggtgagacccgtcgacgcacatcagcccacgagaaagaaaaaaccagtcaggagaactaacccggcgcgtgcacaggcgacagagggcacgtcctacatcgaccccaagttcagctcccactacatcggtgccacgaatgccggcctgatccgcggtggctaccacttcgcccacctgggctcgagctccggtgcggcccaggctaactacttcctcgcccacggcggtggctggtccggcgacggcatcaccctccccggcatgctcgacctcgagggcgactgcgtgctctcggccagcggcgccgtggcctggatcaaggacttcagcgacacctaccactccaagaccggtgtgtaccccctgctctacaccaacccctcgtggtggtcgtcctgcacgggcaactccaacgccttcgtcaacaccaaccccctggtgctcgcccgctacagctccagcgccggcacccctcccggtggctggccctactacaccatctggcagtacaacgacgcctacgcgtacggtggtgactcggacgtgttcaacggcgacatggctggcctcctccgccttgccaagggataa

SEQ ID NO: 94 - protein, 218 aa, Delitschia sp.
MKATLASLLGLANGALAAVQGFDISHYQSSVNFAGAYSAGARFVIIKATEGTSYIDPKFSSHYIGATNAGLIRGGYHFAHLGSSSGAAQANYFLAHGGGWSGDGITLPGMLDLEGDCVLSASGAVAWIKDFSDTYHSKTGVYPLLYTNPSWWSSCTGNSNAFVNTNPLVLARYSSSAGTPPGGWPYYTIWQYNDAYAYGGDSDVFNGDMAGLLRLAKG

SEQ ID NO: 95 - protein, 201 aa, Rosellinia sp.
AVQGFDISHYQSSVNFAGAYSAGARFVIIKATEGTSYIDPKFSSHYIGATNAGLIRGGYHFAHLGSSSGAAQANYFLAHGGGWSGDGITLPGMLDLEGDCVLSASGAVAWIKDFSDTYHSKTGVYPLLYTNPSWWSSCTGNSNAFVNTNPLVLARYSSSAGTPPGGWPYYTIWQYNDAYAYGGDSDVFNGDMAGLLRLAKG

SEQ ID NO: 96 - DNA, 784 bp, Ascobolus sp.
  sig_peptide 1..48
  mat_peptide 49..781
  source 1..784: mol_type = genomic DNA
  CDS 1..138
  CDS 195..640
  CDS 712..781
atgaagtggctcggtctcgtcaccctccttgtcggcgcggcgcaagctgcagtccctggtttcgacatctcccactggcagagcagtgtcaactttgcctctgcctactcctctggtgcccgtttcgtcatcatcaaggtagtccaacacctccatccaccctagagtccgagtattaaccaccttctctccaggctaccgaaggcacgacctacaaggacccaaagttctcctcccactacactggcgccaccaaggccggtttcatccgcggaggctaccactttgcccgccctgcgtccagcactggcgccgcgcaagcccaattcttcgccagtaacggcggcgggtggtccggcgatggcatcacgctccccggcatgttggatcttgagggagactgtgctggtctttctcagtccggcatggtgtcatggattagctctttcgtcaacaagtacaggtcgctcacaggcagatacccaatgatctacaccaccaactcgtggtgggttacctgcactggtaacagcaaagctttcagctcaaactgcccgttggtcattgctagatacaactcggtggttggaactattccgggaggttggccttattataccatttggcagtacaacgatgcgtaagttttcctccgtgctgggtgtgtgggagacattgggaacattgactaactctggttttcctttttagctataagtatggtggtgattcggatactttcaacggtgcttactctcagctcgtcaagctcgccactggttaa

SEQ ID NO: 97 - protein, 218 aa, Ascobolus sp.
MKWLGLVTLLVGAAQAAVPGFDISHWQSSVNFASAYSSGARFVIIKATEGTTYKDPKFSSHYTGATKAGFIRGGYHFARPASSTGAAQAQFFASNGGGWSGDGITLPGMLDLEGDCAGLSQSGMVSWISSFVNKYRSLTGRYPMIYTTNSWWVTCTGNSKAFSSNCPLVIARYNSVVGTIPGGWPYYTIWQYNDAYKYGGDSDTFNGAYSQLVKLATG

SEQ ID NO: 98 - protein, 202 aa, Ascobolus sp.
AVPGFDISHWQSSVNFASAYSSGARFVIIKATEGTTYKDPKFSSHYTGATKAGFIRGGYHFARPASSTGAAQAQFFASNGGGWSGDGITLPGMLDLEGDCAGLSQSGMVSWISSFVNKYRSLTGRYPMIYTTNSWWVTCTGNSKAFSSNCPLVIARYNSVVGTIPGGWPYYTIWQYNDAYKYGGDSDTFNGAYSQLVKLATG

SEQ ID NO: 99 - DNA, 956 bp, Curreya sp.
  sig_peptide 1..54
  mat_peptide 55..953
  source 1..956: mol_type = genomic DNA
  CDS 1..214
  CDS 275..331
  CDS 385..450
  CDS 506..648
  CDS 700..821
  CDS 884..953
atgaatctctctggtcttctcgcaatcgcgtcctaccttgctctttcggcagcaaccgtgcctgggttcgacatttcccactaccaaggcactgtgaacttcgctggtgcttactcctcaggggcgcgctttgtgatcatcaaagcaactgagggcaccacttatacggacccaaacttctccaacaactatgttggtgcgacgaatgctaaatgtatgcaagcccttcggcataactccgatacctgcctgctaacggtgcatgcacatccagttattcgcggtgcttatcatttcgctcgaccagatggcggatcgggctccacccaaggttcgctattctttggtcaaacttacattggagtcacccctaatgaagctcagcacaattctttcattctcatggtggtgggtggtctggcgatggcatcaccctgcctgggatgttgggtaagtgtgctcaagctcatgggattttcccttgactaacgtcctgggtacgaagacatcgaatatggccctacatcgacatgctatggcctctccacgtctgctatggtcacatggattactgattttgtcaacgagtaccatgctttgacaggtcgctaccctctcatctacaccaccaatgactggtggaacactgtgagtatactgtaccgtcaccgtctccggagctctttctcacagtaatagtgcaccggaaacacaaacaagtttagcactacttgcccgcttgtcctcgcacggtacagcagctccgtggggaccatccccggaggttggccgttccagacaatctggcaattcaatgacaagtaagtgcttcaccttagttgtaacgaagaaattggacttcgctgatgcagttgtattgcagttacgcttatggtggcgactctgatacatttaacggagatctggcgggattgaagaagcttgcgaccggataa

SEQ ID NO: 100 - protein, 224 aa, Curreya sp.
MNLSGLLAIASYLALSAATVPGFDISHYQGTVNFAGAYSSGARFVIIKATEGTTYTDPNFSNNYVGATNAKFIRGAYHFARPDGGSGSTQAQFFHSHGGGWSGDGITLPGMLDIEYGPTSTCYGLSTSAMVTWITDFVNEYHALTGRYPLIYTTNDWWNTCTGNTNKFSTTCPLVLARYSSSVGTIPGGWPFQTIWQFNDNYAYGGDSDTFNGDLAGLKKLATG

SEQ ID NO: 101 - protein, 206 aa, Curreya sp.
TVPGFDISHYQGTVNFAGAYSSGARFVIIKATEGTTYTDPNFSNNYVGATNAKFIRGAYHFARPDGGSGSTQAQFFHSHGGGWSGDGITLPGMLDIEYGPTSTCYGLSTSAMVTWITDFVNEYHALTGRYPLIYTTNDWWNTCTGNTNKFSTTCPLVLARYSSSVGTIPGGWPFQTIWQFNDNYAYGGDSDTFNGDLAGLKKLATG

SEQ ID NO: 102 - DNA, 814 bp, Coniothyrium sp
  sig_peptide 1..48
  mat_peptide 49..811
  source 1..814: mol_type = genomic DNA
  CDS 1..231
  CDS 389..811
atgaagtgtctttcattccttccattgttggctgcaacagctcatggcgcggtccaaggcttcgacatctcccattatcaagcatccgtcaatttcgctgcagcctactcgggcggcctacgcttcgtatacatcaaagctacagaaggcacgacataccaggatccagccttctcctcgcattacagtggcgccacgtctgcagggttcatccgcggcggctaccacttcgtaagctctccccatacacaattgccaatcgatgaagcatgagatgttcgagtccctggagctcataaacagaatgcaaaatttacccccgatcttttccgcaacgttttcatcctcatgcgaaagacaaccactaacctgtaccgcacactcacaggcacgccccgcctcctccaccggcgcagcccaagcctcctacttcgtcgcgcacggcggcgggtggtcgaacgacggcataacgctcccagggatgctcgacctggaaggcgactgcgcgggcctctctaccgcctccatggtatcctggatcagcagcttctccaaccagtaccacagcctgacgggccgctggcccgtcatctacacgacgaacagctggtggacgacgtgcacggggaacagcgcggcgttcaacgcgaacagcccgctgatgctggcgcggtggggaagcacggcgggcacgatcccggggggctggccttattatacgatttggcagtacaaggatagcaatacgtatgggggtgatagcgatgtgttcaacggtgatgcgacgcagttgaagaaattggcgacaggttaa

SEQ ID NO: 103 - protein, 218 aa, Coniothyrium sp
MKCLSFLPLLAATAHGAVQGFDISHYQASVNFAAAYSGGLRFVYIKATEGTTYQDPAFSSHYSGATSAGFIRGGYHFARPASSTGAAQASYFVAHGGGWSNDGITLPGMLDLEGDCAGLSTASMVSWISSFSNQYHSLTGRWPVIYTTNSWWTTCTGNSAAFNANSPLMLARWGSTAGTIPGGWPYYTIWQYKDSNTYGGDSDVFNGDATQLKKLATG

SEQ ID NO: 104 - protein, 202 aa, Coniothyrium sp
AVQGFDISHYQASVNFAAAYSGGLRFVYIKATEGTTYQDPAFSSHYSGATSAGFIRGGYHFARPASSTGAAQASYFVAHGGGWSNDGITLPGMLDLEGDCAGLSTASMVSWISSFSNQYHSLTGRWPVIYTTNSWWTTCTGNSAAFNANSPLMLARWGSTAGTIPGGWPYYTIWQYKDSNTYGGDSDVFNGDATQLKKLATG

SEQ ID NO: 105 - DNA, 802 bp, Hypoxylon sp.
  sig_peptide 1..51
  mat_peptide 52..799
  source 1..802: mol_type = genomic DNA
  CDS 1..141
  CDS 210..696
  CDS 771..799
atgaaagctttcattactacctgtctaagcttgagcagccttgccctcgctgctgtccctggctttgatatttctcactaccagtcgaacgtagacttcggggcagcatatagttccggggcacgattcgttatcatcaaagtgagcttaacctacctatatctataactcaaatatatcccccgcgaaactcaccagatgaaaaacaggctacagaaggcaccacttaccaagaccccagcttcagcacccactacaccggcgccaccaaagccggcctcatccgcggcagctaccacttcgcgcgccccggatcgtcctccggcgctgcccaagcgacgtacttcctcgcgcacggcggcgggtggtcgggcgacgggatcacgctgccggggatgctggacctagaaggcgattgcgcgggcctgtcgaccaacgccatggtcgcgtggatcagggactttagcgatacgtaccacggcaggacgggacggtatccgctgctgtacacgaacccgtcgtggtggtcgggttgcgcgggcgggtcggccgcgttcgtcggtacgaatccgcttgtgcttgcgcggtatgctgggagcccgggggcgttgccgggagggtggccgtattatacgatttggcagtttgacgatgcgtataagtatgggggcgattcggatacgtttaatggcgatcgtgagtacctatcgttgcttaatttttcttttctattattttcttgtcctttggctaacgtggtggtgttgcagtcacgcagttaaagaaattggcttctggctag

SEQ ID NO: 106 - protein, 219 aa, Hypoxylon sp.
MKAFITTCLSLSSLALAAVPGFDISHYQSNVDFGAAYSSGARFVIIKATEGTTYQDPSFSTHYTGATKAGLIRGSYHFARPGSSSGAAQATYFLAHGGGWSGDGITLPGMLDLEGDCAGLSTNAMVAWIRDFSDTYHGRTGRYPLLYTNPSWWSGCAGGSAAFVGTNPLVLARYAGSPGALPGGWPYYTIWQFDDAYKYGGDSDTFNGDLTQLKKLASG

SEQ ID NO: 107 - protein, 202 aa, Hypoxylon sp.
AVPGFDISHYQSNVDFGAAYSSGARFVIIKATEGTTYQDPSFSTHYTGATKAGLIRGSYHFARPGSSSGAAQATYFLAHGGGWSGDGITLPGMLDLEGDCAGLSTNAMVAWIRDFSDTYHGRTGRYPLLYTNPSWWSGCAGGSAAFVGTNPLVLARYAGSPGALPGGWPYYTIWQFDDAYKYGGDSDTFNGDLTQLKKLASG

SEQ ID NO: 108 - DNA, 837 bp, Xylariaceae sp.
  sig_peptide 1..51
  mat_peptide 52..834
  source 1..837: mol_type = genomic DNA
  CDS 1..141
  CDS 199..685
  CDS 806..834
atgaagctcttcattgccacgtgtttgggcctgggtagcatcgccctcgcagctgtcccaggattcgatatctcccactatcaatcgagcgtggattttgcggcagcttacagtgctggagctcgcttcgttattatcaaggtagcactaaaccccaaaattgaagccattgaaaggagaactaacaagtaatactaggctacagagggtaccacctacattgacccgagtttcagcagtcactacaccggtgccaccaacgccgggctcatccgcggcggctaccacttcgcacatcccggttcctcttcgggtgccacgcaggccaactatttcctagcacatggcggaggctggtcaggagacggaattactctcccaggcatgttggacttagagggtgactgcgcaggcctctcaacaagcgccatggtctcctggatcaaagacttcagcaatgcatatcacagcaaaacgggacgctacccactcctctacacaaacccgtcgtggtggtccagttgcactggtagctcaagtgcttttgtcaatacgaaccctcttgtccttgcacggtatagcagcagtgctggaacacctcctggtggctggccgtactacacgatttggcagttcaatgatgcgtacaagtatggtggcgactcggatactttcaatggagaatgtaagtcaacctaagtcatttcctgcttctttgtcttgatctgcgagtggcagatccctaatggcagtgcaaactttctcagtgctgagcaataactctggctaatacacggtgttgtagatgcttcattgcaaaagctggctactggttaa

SEQ ID NO: 109 - protein, 219 aa, Xylariaceae sp.
MKLFIATCLGLGSIALAAVPGFDISHYQSSVDFAAAYSAGARFVIIKATEGTTYIDPSFSSHYTGATNAGLIRGGYHFAHPGSSSGATQANYFLAHGGGWSGDGITLPGMLDLEGDCAGLSTSAMVSWIKDFSNAYHSKTGRYPLLYTNPSWWSSCTGSSSAFVNTNPLVLARYSSSAGTPPGGWPYYTIWQFNDAYKYGGDSDTFNGEYASLQKLATG

SEQ ID NO: 110 - protein, 202 aa, Xylariaceae sp.
AVPGFDISHYQSSVDFAAAYSAGARFVIIKATEGTTYIDPSFSSHYTGATNAGLIRGGYHFAHPGSSSGATQANYFLAHGGGWSGDGITLPGMLDLEGDCAGLSTSAMVSWIKDFSNAYHSKTGRYPLLYTNPSWWSSCTGSSSAFVNTNPLVLARYSSSAGTPPGGWPYYTIWQFNDAYKYGGDSDTFNGEYASLQKLATG

SEQ ID NO: 111 - DNA, 835 bp, Hypoxylon sp.
  sig_peptide 1..51
  mat_peptide 52..832
  source 1..835: mol_type = genomic DNA
  CDS 1..141
  CDS 201..687
  CDS 804..832
atgaagttcttcgttgccactttcttaggtctaagcagcgttgccctcgcagcagtgccaggattcgatatttctcactaccaatcgagtgtggattttgcagcagcctacaattccggggcgcgctttgttattatcaaggtaaccctgaatcctaaagagaaaagtcgttcgaaatagatttaacccgttccgaataggctactgagggcaccacgtacatcgacccaagtttcagtagtcactataccggtgctaccaaggctggattcatccgcggtggctatcattttgcacatcccggttcttcgtcgggagcggcgcaagccaactatttcctagcacatggcggtggctggtcgggcgacggaatcactttcccaggcatgttggatttagagggtgactgcgcgggcctctcgacaagtgctatggtctcttggatcaaggacttcagcgatacatatcacagtaagacgggacgctaccctctcctctacacaaacccttcgtggtggtcgagctgcaccggtgattcgagtgcctttgtcaatacgaaccccctcgtcctcgcacgatatagcagcagcgctggaacgccgcccggcggctggccgtattacacgatctggcagtttaacgacgcatacaaatatggtggtgactcggatactttcaatggaaactgtaagtgaacttgcgatcgttttggccaccatcgaaacctataaatgcgaggtcgtcgatggcagtgcaaactctacctactcttagcaacaatttcagctaatatgatattttagatgcttcgctgcaaaagttagctactggttag

SEQ ID NO: 112 - protein, 219 aa, Hypoxylon sp.
MKFFVATFLGLSSVALAAVPGFDISHYQSSVDFAAAYNSGARFVIIKATEGTTYIDPSFSSHYTGATKAGFIRGGYHFAHPGSSSGAAQANYFLAHGGGWSGDGITFPGMLDLEGDCAGLSTSAMVSWIKDFSDTYHSKTGRYPLLYTNPSWWSSCTGDSSAFVNTNPLVLARYSSSAGTPPGGWPYYTIWQFNDAYKYGGDSDTFNGNYASLQKLATG

SEQ ID NO: 113 - protein, 202 aa, Hypoxylon sp.
AVPGFDISHYQSSVDFAAAYNSGARFVIIKATEGTTYIDPSFSSHYTGATKAGFIRGGYHFAHPGSSSGAAQANYFLAHGGGWSGDGITFPGMLDLEGDCAGLSTSAMVSWIKDFSDTYHSKTGRYPLLYTNPSWWSSCTGDSSAFVNTNPLVLARYSSSAGTPPGGWPYYTIWQFNDAYKYGGDSDTFNGNYASLQKLATG

SEQ ID NO: 114 - DNA, 748 bp, Yunnania penicillata
  sig_peptide 1..54
  mat_peptide 55..743
  source 1..748: mol_type = genomic DNA
  CDS 1..144
  CDS 218..745
atgaagtccattaccgccgccgctcttctgggcctcgccgccactgtccaggccgatgtcgacggctttgacatctcccactaccaggagactgtcgactatgccggtgcctacggcgctggtgcccgcttcgtcatcatcaaggtgagcacaccacatcaccaaacagtggatgtgtcttatcgagacaagtgacagtgactaaccccgtccacaggcgactgagggcacgaactacatcgactcctctttcaacacgcactacgctggtgccaccgacgcgggcctcatccgcggcggctaccacttcgcgcacccgggcgagacgacgggcgcggagcaggccgactacttcatcgcgcacggcggcaactggtccaacgacggcatcacgctgccgggcatgctggacctcgagggcgagggcagcaccacgtgctgggacctgagcgccgccgacatggtcgcctggatcaaggccttcagcgaccggtaccaggaggtcacgagccggtaccccctgctctacaccaacccgtcgtggtggtccgagtgcacgggcaactcggacgccttcgtggacaccaacccgctggtgcttgcgcggtacgcgagctcgcctggcgagatccccggcggctggccggcgcagaccatctggcagaactcggacagctactcgttcggtggagactcggacatcttcaacggcgacgaggccggcctgaagaagcttgcgagcggttaa

SEQ ID NO: 115 - protein, 224 aa, Yunnania penicillata
MKSITAAALLGLAATVQADVDGFDISHYQETVDYAGAYGAGARFVIIKATEGTNYIDSSFNTHYAGATDAGLIRGGYHFAHPGETTGAEQADYFIAHGGNWSNDGITLPGMLDLEGEGSTTCWDLSAADMVAWIKAFSDRYQEVTSRYPLLYTNPSWWSECTGNSDAFVDTNPLVLARYASSPGEIPGGWPAQTIWQNSDSYSFGGDSDIFNGDEAGLKKLASG

SEQ ID NO: 116 - protein, 206 aa, Yunnania penicillata
DVDGFDISHYQETVDYAGAYGAGARFVIIKATEGTNYIDSSFNTHYAGATDAGLIRGGYHFAHPGETTGAEQADYFIAHGGNWSNDGITLPGMLDLEGEGSTTCWDLSAADMVAWIKAFSDRYQEVTSRYPLLYTNPSWWSECTGNSDAFVDTNPLVLARYASSPGEIPGGWPAQTIWQNSDSYSFGGDSDIFNGDEAGLKKLASG

SEQ ID NO: 117 - DNA, 782 bp, Engyodontium album
  sig_peptide 1..60
  mat_peptide 61..779
  source 1..782: mol_type = genomic DNA
  CDS 1..150
  CDS 200..383
  CDS 433..779
atgaagtcttttggtgttattgctaccggtttggccacccttgtgggtgttgcctctgccagagtccaaggtttcgacatctcccactatcagcccagcgtcgacttcaatgcggcctatgctgacggagctcgctttgtgatcatcaaggtataacaaaccataacttggcttatgaacaccatctaatgtattgcaggcaaccgagggtaccacctacaaagatcccaagttcagccagcactacatcggtgctaccaacgccggattcatccgcggtggctaccactttgctcagcctgcttcctcttctggtgcagcgcaggcagactatttcctcaagaacggaggtggttggtctagcgatggaattactctcccaggtgagcaaagtcacaaacgttcgagggcagttcactaatatcgtggcaggtatgcttgatatggagtacaaccccaatggcagtgcttgctacggtctttcccaggcttccatgcgcaactggatcaacgactttgtcaacacctaccactcccgcacgggtgtctaccctctcctttacaccaccaccagctggtggaaaacctgcacgggtaacactgccatgtttgccgacaagtgccctctcgtcatcgctcgctacaacagcgtagtcggagagctccccgctggttggtctttctggacaatttggcagtacaacgaccactacaagcatggtggtgactcagacgcttttaacggagactactctcagcttcagagaatcgccagaggctaa

SEQ ID NO: 118 - protein, 227 aa, Engyodontium album
MKSFGVIATGLATLVGVASARVQGFDISHYQPSVDFNAAYADGARFVIIKATEGTTYKDPKFSQHYIGATNAGFIRGGYHFAQPASSSGAAQADYFLKNGGGWSSDGITLPGMLDMEYNPNGSACYGLSQASMRNWINDFVNTYHSRTGVYPLLYTTTSWWKTCTGNTAMFADKCPLVIARYNSVVGELPAGWSFWTIWQYNDHYKHGGDSDAFNGDYSQLQRIARG

SEQ ID NO: 119 - protein, 207 aa, Engyodontium album
RVQGFDISHYQPSVDFNAAYADGARFVIIKATEGTTYKDPKFSQHYIGATNAGFIRGGYHFAQPASSSGAAQADYFLKNGGGWSSDGITLPGMLDMEYNPNGSACYGLSQASMRNWINDFVNTYHSRTGVYPLLYTTTSWWKTCTGNTAMFADKCPLVIARYNSVVGELPAGWSFWTIWQYNDHYKHGGDSDAFNGDYSQLQRIARG

SEQ ID NO: 120 - DNA, 741 bp, Metapochonia bulbillosa
  sig_peptide 1..57
  mat_peptide 58..738
  source 1..741: mol_type = genomic DNA
  CDS 1..147
  CDS 205..738
atgaagtctgttactttcatcgccagtctcgcctccattgtgagcgtagccaccgccaccgttgctgggtttgacatttcaaactaccaacccaccgttgactttaaaaaggcctacgcagacggtgctcgcttcgtcattatcaaagtaagacaaatagcacagtgtacaaaagtgaaaacaatactctaacaaaaccaccaggccaccgaaggcacaacatacaccgatcccagcttcagctcccactacactggcgccacgcaggccggcctcatccggggaggctaccacttcgcacaccccggatcaggcaccggcgccgcccaggcgaattacttcctcgcccacggcggcggctggtccaaggatggcatcacgctcccgggcatgattgatctcgagtacaaccccagcggtgctacgtgctacggtctctcggccagcggcatggtcagctggatctctgactttgtcgagacgtaccatagcaagactggggtttatcctcttatttacacgtcgacgagctggtggaaccagtgtacgggtagcagtactgcctttggtaataagtgcccgcttgtggttgcgcggtatgctagctctgttggggcgttgcctgctggttggggatttcagactatttggcagaatagcgataagtcgccttggggtggtgacaatgatattttcaacggtagccttgatcagcttaagcgtattgctaacgcttcgtaa

SEQ ID NO: 121 - protein, 227 aa, Metapochonia bulbillosa
MKSVTFIASLASIVSVATATVAGFDISNYQPTVDFKKAYADGARFVIIKATEGTTYTDPSFSSHYTGATQAGLIRGGYHFAHPGSGTGAAQANYFLAHGGGWSKDGITLPGMIDLEYNPSGATCYGLSASGMVSWISDFVETYHSKTGVYPLIYTSTSWWNQCTGSSTAFGNKCPLVVARYASSVGALPAGWGFQTIWQNSDKSPWGGDNDIFNGSLDQLKRIANAS

SEQ ID NO: 122 - protein, 208 aa, Metapochonia bulbillosa
TVAGFDISNYQPTVDFKKAYADGARFVIIKATEGTTYTDPSFSSHYTGATQAGLIRGGYHFAHPGSGTGAAQANYFLAHGGGWSKDGITLPGMIDLEYNPSGATCYGLSASGMVSWISDFVETYHSKTGVYPLIYTSTSWWNQCTGSSTAFGNKCPLVVARYASSVGALPAGWGFQTIWQNSDKSPWGGDNDIFNGSLDQLKRIANAS

SEQ ID NO: 123 - DNA, 756 bp, Hamigera paravellanea
  sig_peptide 1..54
  mat_peptide 55..753
  source 1..756: mol_type = genomic DNA
  CDS 1..168
  CDS 223..753
atgaaggcttcttccatcctctccctgctggcctccctggccctcaccagcgcagctcctctcgaggcgcgcgccggcagcgtgcaaggcttcgacatctcgcactaccaagcgaaggtcgacttcgccgccgcataccgcagcggtgctcgcttcgtcatcatcaaggtacttactcctcgtgctcgtcctcacagacacagacactgaccacagaacaaggccaccgaaggaaccacgtacaccgacccggccttctcctcgcactacacctccgccaccaacgccggcttcatccggggcggctaccacttcgcacacccggactccagctcgggcgcagcgcaagccacctacttccttgcccacggcggcggctggtccggcgacggcatcacgctgcccggaatgctggacctcgagtacaacccctcgggggcgacgtgctacgggctcagcgatgcggccatggtcgcctggatccaggacttcgtggacacgtaccacgcccgcacggggcgctacccgatgatctacaccacggccgactggtggaacacctgcacgggcaacagcagcaagttcagccagacgtgtccgttggtgctggcccggtacgccagcagcgtcggcaccgttcccggcggctgggggtatcagaccatctggcagaattcggatagctatgcgtatggcggggattcggatatttttaacggggatgagacgcagttgaagaagttggcgagtggatga

SEQ ID NO: 124 - protein, 233 aa, Hamigera paravellanea
MKASSILSLLASLALTSAAPLEARAGSVQGFDISHYQAKVDFAAAYRSGARFVIIKATEGTTYTDPAFSSHYTSATNAGFIRGGYHFAHPDSSSGAAQATYFLAHGGGWSGDGITLPGMLDLEYNPSGATCYGLSDAAMVAWIQDFVDTYHARTGRYPMIYTTADWWNTCTGNSSKFSQTCPLVLARYASSVGTVPGGWGYQTIWQNSDSYAYGGDSDIFNGDETQLKKLASG

SEQ ID NO: 125 - protein, 215 aa, Hamigera paravellanea
APLEARAGSVQGFDISHYQAKVDFAAAYRSGARFVIIKATEGTTYTDPAFSSHYTSATNAGFIRGGYHFAHPDSSSGAAQATYFLAHGGGWSGDGITLPGMLDLEYNPSGATCYGLSDAAMVAWIQDFVDTYHARTGRYPMIYTTADWWNTCTGNSSKFSQTCPLVLARYASSVGTVPGGWGYQTIWQNSDSYAYGGDSDIFNGDETQLKKLASG

SEQ ID NO: 126 - DNA, 774 bp, Metarhizium iadini
  sig_peptide 1..60
  mat_peptide 61..771
  source 1..774: mol_type = genomic DNA
  CDS 1..180
  CDS 241..771
atgaagacttcaggcgccatttcccttggcctcgccgctctcgtcagctcagcagccgcctcccccgtggagctcgagcagcgcgccgccagcgtcaagggcttcgacatctcgggataccagcccaacgttgacttcaacaaggcctatgccgacggcgctcgcttcgtcatcatcaaggtaagttcgcacctggacacatgtctgaccttggcagcacagccgtctaaccacgtcaaggccaccgagggaaccacctacatcgacaagaccttctccaagcactacacgggcgcaaccaaggccaagctcatccgcggcgcgtaccacttcgcccacccggggcagaacaaggcctcggccgaggctgacttcttcgtccagcacggcggcaactggtccaaggacgccatcaccctgcccggcatggtcgatctagagtccgaaaagggccaccccccgtgctgggggttgtcgcattccgcaatggtcgcttggatcagcgagtttgtcgccgcttaccacaagaaaacgacccgatatcccatgctgtacaccaacccgtcgtggtggtccgcttgcaccggcaacagcaaggccttcaaggatacctgccctctggtcctggccagatacgccagctcgcccggcgccattcctggtggctggcctgctcagaccatctggcaaaacagcgataagagcccgtggggtggtgactctgacatgttcaacggcgacttggcccagttgaagaagcttgctaccggttag

SEQ ID NO: 127 - protein, 237 aa, Metarhizium iadini
MKTSGAISLGLAALVSSAAASPVELEQRAASVKGFDISGYQPNVDFNKAYADGARFVIIKATEGTTYIDKTFSKHYTGATKAKLIRGAYHFAHPGQNKASAEADFFVQHGGNWSKDAITLPGMVDLESEKGHPPCWGLSHSAMVAWISEFVAAYHKKTTRYPMLYTNPSWWSACTGNSKAFKDTCPLVLARYASSPGAIPGGWPAQTIWQNSDKSPWGGDSDMFNGDLAQLKKLATG

SEQ ID NO: 128 - protein, 217 aa, Metarhizium iadini
SPVELEQRAASVKGFDISGYQPNVDFNKAYADGARFVIIKATEGTTYIDKTFSKHYTGATKAKLIRGAYHFAHPGQNKASAEADFFVQHGGNWSKDAITLPGMVDLESEKGHPPCWGLSHSAMVAWISEFVAAYHKKTTRYPMLYTNPSWWSACTGNSKAFKDTCPLVLARYASSPGAIPGGWPAQTIWQNSDKSPWGGDSDMFNGDLAQLKKLATG

SEQ ID NO: 129 - DNA, 747 bp, Thermoascus aurantiacus
  sig_peptide 1..48
  mat_peptide 49..746
  source 1..747: mol_type = genomic DNA
  CDS 1..159
  CDS 214..744
atgaagatcgctcctctcctcctcgccggcctcgccgccgccgcccccgagctcgacaagcgcgcccgcggcgtccagggctttgatatctcgcactaccagcccaacgtcgacttcaagggcgcgtacaacagcggcgcccgcttcgtcatcatcaaggtatgcctctctctctctctctatcttcttcgcacctctactgatgcaggccaggccaccgagggcacgacgtacaaggacccggccttctccaagcactacatcggcgccaccgaggccggcctcatccgcggcggataccacttcgcccacccggacaagagcagcggcgccgcgcaggccaacttcttcctcgcccacggcggcggctggtccggcgacggcatcacgctgcccggcatggtcgacctcgagtacaacccctcgggcgacgcctgctacggcctgtccgactcgcagatggtctcctggatccgggactttgtcaacacctaccacgcgcacacgggacgctaccccatgatctacaccacggccgactggtggaagaggtgcaccggcgacagccacgccttctcgaccacctgcccgctcgtcctggcgcggtacaacagctcaccgggcacggtgcccggcggctggccctaccacaccatctggcagaactcggacaagtaccgcttcggcggcgactcggacatcttcaacggcgacctggcggggctgaagaggctggccaagggctga

SEQ ID NO: 130 - protein, 230 aa, Thermoascus aurantiacus
MKIAPLLLAGLAAAAPELDKRARGVQGFDISHYQPNVDFKGAYNSGARFVIIKATEGTTYKDPAFSKHYIGATEAGLIRGGYHFAHPDKSSGAAQANFFLAHGGGWSGDGITLPGMVDLEYNPSGDACYGLSDSQMVSWIRDFVNTYHAHTGRYPMIYTTADWWKRCTGDSHAFSTTCPLVLARYNSSPGTVPGGWPYHTIWQNSDKYRFGGDSDIFNGDLAGLKRLAKG

SEQ ID NO: 131 - protein, 214 aa, Thermoascus aurantiacus
ELDKRARGVQGFDISHYQPNVDFKGAYNSGARFVIIKATEGTTYKDPAFSKHYIGATEAGLIRGGYHFAHPDKSSGAAQANFFLAHGGGWSGDGITLPGMVDLEYNPSGDACYGLSDSQMVSWIRDFVNTYHAHTGRYPMIYTTADWWKRCTGDSHAFSTTCPLVLARYNSSPGTVPGGWPYHTIWQNSDKYRFGGDSDIFNGDLAGLKRLAKG

SEQ ID NO: 132 - DNA, 753 bp, Clonostachys rossmaniae
  sig_peptide 1..54
  mat_peptide 55..750
  source 1..753: mol_type = genomic DNA
  CDS 1..144
  CDS 217..750
atgaagagttctctgtaccttgcttttgttgcacttgcgacggtggccgtagcagcagtgccaggatttgatatatccgggtggcagaaatccactgactttgctaagtcatacgcaaacggagatcgttttgtgtacatcaaggtaaaacccaagtcatatttttcactggaacattccccgtttagttcaactgagcaggctaacaccttgtaggctacagagggtacaacctttaagaatcctcttttctcgaagcagtataccggtgctacaaatgcccgtctcattcgtggtgcctatcactttgcccagccggcatcttcatccggggcctcacaggctcgtttctttgttgcaaatggcggaggttggtccaacgatggcatcaccttgcctggtgcggtggatatggagtacaaccccagtggcgccacatgctacgggctgtccaaaaccgccatggtgaactggatcgaggactttgtgtcaacataccaggctttgaccgggcgctggcctgtcgtttataccacgcttgactggtggacccaatgcactggcaacagcgccaagtttggggatcggtgcccgctgtgggtggcacgatatgcaagtgctgtcggccaaatccccgcaggttggagctttcacactatatggcaatataacgcgaaataccctgaaggtggagactctgatatattcaatggcgacgagactcgccttaaagctttagcttccggggcctga

SEQ ID NO: 133 - protein, 226 aa, Clonostachys rossmaniae
MKSSLYLAFVALATVAVAAVPGFDISGWQKSTDFAKSYANGDRFVYIKATEGTTFKNPLFSKQYTGATNARLIRGAYHFAQPASSSGASQARFFVANGGGWSNDGITLPGAVDMEYNPSGATCYGLSKTAMVNWIEDFVSTYQALTGRWPVVYTTLDWWTQCTGNSAKFGDRCPLWVARYASAVGQIPAGWSFHTIWQYNAKYPEGGDSDIFNGDETRLKALASGA

SEQ ID NO: 134 - protein, 208 aa, Clonostachys rossmaniae
AVPGFDISGWQKSTDFAKSYANGDRFVYIKATEGTTFKNPLFSKQYTGATNARLIRGAYHFAQPASSSGASQARFFVANGGGWSNDGITLPGAVDMEYNPSGATCYGLSKTAMVNWIEDFVSTYQALTGRWPVVYTTLDWWTQCTGNSAKFGDRCPLWVARYASAVGQIPAGWSFHTIWQYNAKYPEGGDSDIFNGDETRLKALASGA

SEQ ID NO: 135 - DNA, 669 bp, Simplicillium obclavatum
  sig_peptide 1..57
  mat_peptide 58..666
  source 1..669: mol_type = genomic DNA
  CDS 1..666
atgaagcttgccaccgtgctaaacgttcttgcacttgctgtttctagcgttatcggcgcaccgaaagggattgacgtcagccattggcagggatccatcaactggggtgcagtgaaggcgaacggtatcgaatgggcatacatcaaagccacagaatccacgaactacaaagacccaaacttcaatgcaaactacgttggtgctaccaatgcggggctcatccgcggcgcataccattttgctagacccggcgattcttcaggtgcggcccaggcaaattattttgctagcaacggcggcggttggtcagcagacggcataactctgcctggtgccgttgatctcgaagcaggatgttctggactgtcccagtccgcaatgactgcttggataaaggacttcagcaacacctatcacgcgcgcaccggccggtacccagctatttacaccaccacgagctggtggaaacaatgcactggcaatgcatctggtttccaaaacaataacccgctatggattgctcgctgggcttcttccgtaggagagttgcctgcgggttacagctatcacacattttggcaatatgcggaccacggaccgaacccaggcgaccaggacgttttcaatggtgattctgctggcctcaagaggatggccaagggtagttag

SEQ ID NO: 136 - protein, 222 aa, Simplicillium obclavatum
MKLATVLNVLALAVSSVIGAPKGIDVSHWQGSINWGAVKANGIEWAYIKATESTNYKDPNFNANYVGATNAGLIRGAYHFARPGDSSGAAQANYFASNGGGWSADGITLPGAVDLEAGCSGLSQSAMTAWIKDFSNTYHARTGRYPAIYTTTSWWKQCTGNASGFQNNNPLWIARWASSVGELPAGYSYHTFWQYADHGPNPGDQDVFNGDSAGLKRMAKGS

SEQ ID NO: 137 - protein, 203 aa, Simplicillium obclavatum
APKGIDVSHWQGSINWGAVKANGIEWAYIKATESTNYKDPNFNANYVGATNAGLIRGAYHFARPGDSSGAAQANYFASNGGGWSADGITLPGAVDLEAGCSGLSQSAMTAWIKDFSNTYHARTGRYPAIYTTTSWWKQCTGNASGFQNNNPLWIARWASSVGELPAGYSYHTFWQYADHGPNPGDQDVFNGDSAGLKRMAKGS

SEQ ID NO: 138 - DNA, 771 bp, Aspergillus inflatus
  sig_peptide 1..51
  mat_peptide 52..768
  source 1..771: mol_type = genomic DNA
  CDS 1..165
  CDS 235..768
atgaagttctctgccattgctctcctggcttctgcctctgccgtggccgcggcccctcttgaggctcgtgccaacactgtccaggggttcgacatctccagcttccagcccaatgttgactttgccgctgcctacaaggccggtgcccgttttgtcatgatgaaggtcggtcccattgaattcatcgacgcagatatagatcaaccgaaatcactggtactgactgcgcgctaggccacccaaaacaccaactacgttgacaagacctttaacgcacactacgagggtgccaccaaggctggcctcatccgtggtggttaccatttcgccattcccaacggcccctccggcgctgcccaggccgagtacttcctcgcccatggtggcgggtggtccgacgatggcaagaccctgcccggcatgatcgatcttgagtacaacccctatggccaaacctgctacgacctctccgccgcgaagatggtcgactggatcaaggacttctccaacacctaccacgccaagaccaagcgctaccccatgatctacactaccgccaactggtggaaggagtgcactggcgacagcaaagagttcagccagaccaaccccttggttctggctcgctactccagctccgcgggcactgtccctggtggctggcccgcctactctttctggcagaacgccgacaagtacaagttcggaggagactctgacatctggaacggcagtgaggacaacctcaagaagtttgccaagggtgcttaa

SEQ ID NO: 139 - protein, 233 aa, Aspergillus inflatus
MKFSAIALLASASAVAAAPLEARANTVQGFDISSFQPNVDFAAAYKAGARFVMMKATQNTNYVDKTFNAHYEGATKAGLIRGGYHFAIPNGPSGAAQAEYFLAHGGGWSDDGKTLPGMIDLEYNPYGQTCYDLSAAKMVDWIKDFSNTYHAKTKRYPMIYTTANWWKECTGDSKEFSQTNPLVLARYSSSAGTVPGGWPAYSFWQNADKYKFGGDSDIWNGSEDNLKKFAKGA

SEQ ID NO: 140 - protein, 216 aa, Aspergillus inflatus
APLEARANTVQGFDISSFQPNVDFAAAYKAGARFVMMKATQNTNYVDKTFNAHYEGATKAGLIRGGYHFAIPNGPSGAAQAEYFLAHGGGWSDDGKTLPGMIDLEYNPYGQTCYDLSAAKMVDWIKDFSNTYHAKTKRYPMIYTTANWWKECTGDSKEFSQTNPLVLARYSSSAGTVPGGWPAYSFWQNADKYKFGGDSDIWNGSEDNLKKFAKGA

SEQ ID NO: 141 - DNA, 738 bp, Paracremonium inflatum
  sig_peptide 1..63
  mat_peptide 64..735
  source 1..738: mol_type = genomic DNA
  CDS 1..153
  CDS 205..735
atgaaaactttcggctatttgggccttggcatggtgtcccttgcgatcacctcggtgtctgcaaaggtgttgggttttgatattagtcattaccaggccactgttgacttcaacgctgctaaagacgcgggagcccgatttgtgatcatcaaggtagactacctacccaaccagatggcgacgttcccgagttaataagaccaggccacagagggaacaacctacaaagacccagcgttcagcaaacattatacgggggccacaaaggcaggcctgatccgaggcggctatcacttcgctcagccagcttcctcgtctggcgctgctcaggcgactttcttcctcgcacacggcggcggttggtcgtctgatggcataacattgccgggcatgctggatttagagtacaaccccagtggctcgacttgctacgggctgtcacaaagcagcatggttcagtggatttccgacttcatcgacacataccactccaaaacagggcgatatccgttgatatatacatctaccagctggtggaaaacttgcacgggaaactcaagcaagtttgcagcgaactgcccgctcgtcgttgctcgctactcgagttctgtcggagagctccctgctggctggacgtattacacgatctggcaaaattcggatagctataaatacgggggtgattcggatatcttcaatggagacgagtcgcaactccagaaacttgccaagggttaa

SEQ ID NO: 142 - protein, 228 aa, Paracremonium inflatum
MKTFGYLGLGMVSLAITSVSAKVLGFDISHYQATVDFNAAKDAGARFVIIKATEGTTYKDPAFSKHYTGATKAGLIRGGYHFAQPASSSGAAQATFFLAHGGGWSSDGITLPGMLDLEYNPSGSTCYGLSQSSMVQWISDFIDTYHSKTGRYPLIYTSTSWWKTCTGNSSKFAANCPLVVARYSSSVGELPAGWTYYTIWQNSDSYKYGGDSDIFNGDESQLQKLAKG

SEQ ID NO: 143 - protein, 207 aa, Paracremonium inflatum
KVLGFDISHYQATVDFNAAKDAGARFVIIKATEGTTYKDPAFSKHYTGATKAGLIRGGYHFAQPASSSGAAQATFFLAHGGGWSSDGITLPGMLDLEYNPSGSTCYGLSQSSMVQWISDFIDTYHSKTGRYPLIYTSTSWWKTCTGNSSKFAANCPLVVARYSSSVGELPAGWTYYTIWQNSDSYKYGGDSDIFNGDESQLQKLAKG

SEQ ID NO: 144 - DNA, 872 bp, Westerdykella sp.
  sig_peptide 1..51
  mat_peptide 52..869
  source 1..872: mol_type = genomic DNA
  CDS 1..144
  CDS 199..259
  CDS 342..765
  CDS 824..869
atgaagtctgtactcgccctcctcgccgctggcgcccagtttgccagcgctgccgtctccggcatggacatctctcattaccaaggcacgaactacaacttcgccggcgcatactcgtctggtgctcgtttcgtcatcatcaaggtacatattcgtcaacgcataggacccaaagtccagccattgaacccatcttaggcaaccgaagggaccacctacacggatccccaattttctgctaactatattgcggccaccagtacgtaatacctctatgtgccgtcgactttctgtgccccaatcacgctgcatgttcacggacatcaactaacacctagcagatgcgggcttcatcagggggggctaccatttcgcccgccctgcggactctaccggcgccgcccaagccaaatacttcgtctcccacggcggtggctggtcttccgatggtatcactctccccggaatgctcgacctcgagtacggctcatcgtccgcatgccacgggctttccgtatcggccatgaacacatggatcgcctcgttcatcaaccagtataggagcttgacgggcgcgtatcccatgatctacactacggcggattggtggaagacgtgcaccggagatagccaggcttggaacaccaagtgccctctggtattggcccggtactccagctctgttgggacgatcccaggcggctggccttatcagactatctggcagttcaacgattcgtataagtatggcggggattcggacacgtgagttctcccaaattgcggagtcgccaattgatgaatgtgctgacggaacggatagattcaatggtgacttggctggcttgaagaggctcgccaaaggcagctga

SEQ ID NO: 145 - protein, 225 aa, Westerdykella sp.
MKSVLALLAAGAQFASAAVSGMDISHYQGTNYNFAGAYSSGARFVIIKATEGTTYTDPQFSANYIAATNAGFIRGGYHFARPADSTGAAQAKYFVSHGGGWSSDGITLPGMLDLEYGSSSACHGLSVSAMNTWIASFINQYRSLTGAYPMIYTTADWWKTCTGDSQAWNTKCPLVLARYSSSVGTIPGGWPYQTIWQFNDSYKYGGDSDTFNGDLAGLKRLAKGS

SEQ ID NO: 146 - protein, 208 aa, Westerdykella sp.
AVSGMDISHYQGTNYNFAGAYSSGARFVIIKATEGTTYTDPQFSANYIAATNAGFIRGGYHFARPADSTGAAQAKYFVSHGGGWSSDGITLPGMLDLEYGSSSACHGLSVSAMNTWIASFINQYRSLTGAYPMIYTTADWWKTCTGDSQAWNTKCPLVLARYSSSVGTIPGGWPYQTIWQFNDSYKYGGDSDTFNGDLAGLKRLAKGS

SEQ ID NO: 147 - DNA, 909 bp, Stropharia semiglobata
  sig_peptide 1..60
  mat_peptide 61..906
  source 1..909: mol_type = genomic DNA
  CDS 1..393
  CDS 447..545
  CDS 608..691
  CDS 744..772
  CDS 831..906
atgttctctttcgtcaaagcgctcatcctcctcccggcccttgtgacttccgcgtatgccctcgtatatggcgtagactcgtctaccttggtttctactgcaacctacaagaaagccaagagcgagggcttcactaaggctatcatccgcgggtatcaggaagcatgtggcagtggtggacgcgtcgaccccaacttcgtcgcgacctacaagaatgcacgggccgcgggtatcaccgatatcgacatgtactggttcccatgcaacgggtccggtaactcatgcaagtcgtacgctaagcagctctccgagatcgcgaatgtcttcagtgcgaatagcatgaaaattgggacaatttggattgattttgagaaggattctggttgcaacaacgtgagtttcatggacattatgtccttcataataaaatgacggattttatcaagtggaactacggcaccacgggtaatctcaaccatgcaaaggcactcatctccgcgattaaagcgactgggtttaaattcggtatctacagctcacctggcgtaaggctttttacctttcgtatccgcttcatcaagttaacttagtccgcgactcgttttaggaatggggtaccctgtttgggtccaccggggtagtactcgatagctcggcccccctttggttcgctacgtggaacaatgtaaaggtatgtgaccccgcctactacagaaataacatctctcaaagtttaaccatagactcttaccttgggaacgcatttcggggggtaagtgcaatatatatacatcgaaatatgggatattgaatactgacgttgcttgaaggtggactaaagccgttggccaccagtatacagatgtgtccgcctctggccaattcgacctcaatgtctttgcaaattaa

SEQ ID NO: 148 - protein, 227 aa, Stropharia semiglobata
MFSFVKALILLPALVTSAYALVYGVDSSTLVSTATYKKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTKAVGHQYTDVSASGQFDLNVFAN

SEQ ID NO: 149 - protein, 207 aa, Stropharia semiglobata
LVYGVDSSTLVSTATYKKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTKAVGHQYTDVSASGQFDLNVFAN

SEQ ID NO: 150 - DNA, 909 bp, Stropharia semiglobata
  sig_peptide 1..60
  mat_peptide 61..906
  source 1..909: mol_type = genomic DNA
  CDS 1..393
  CDS 447..545
  CDS 608..691
  CDS 743..771
  CDS 831..906
atgttccctttcgtcaaaacgctcatactactcccgacccttgtgacctccgcatacgcccttgtatatggcgtagactcgtccactctagtctctactgcaacgtacagtaaagccaagagcgagggcttcactaaggctatcatccgcgggtatcaggaagcatgtggcagtggtggacgcgtcgaccccaacttcgtcgcgacctacaagaatgcacgggccgcgggcatcaccgatatcgacatgtactggttcccatgcaacgggtccggtaactcttgcaagtcgtacgctacacagctctccgagatcgcgaatgcgttcagtgcgaatagcatgaaaattgggacaatttggattgattttgagaaggattctacctgcaacaacgtgagtttcatggacattatgtccttcataataaattgacggattttatcaagtggaactacggcaccacgggtaatctcaaccatgcaaaggcactcatctccgcgattaaagcgactgggtttaaattcggtatctacagctcacctggcgtaaggctttttacctttcgtatccgcttcatcaagttaacttagtccgcgactcgttttaggaatggggtaccctgtttgggtccaccggggtagtactcgatagctcggcccccctttggttcgctacgtggaacaatgtaaaggtatgtgaccccgcctactacagaaataacatctctcaaagtttaacatagactcttaccttgggaacgcatttcggggggtaagtgcaatatatatacatcgaaagatggaataattaaatactgatctcgcttaaaggtggactacagccgcgggtcaccagtacacagacgtgtcttcctctggccaattcgacctcaatgtgtttgcaaattaa

SEQ ID NO: 151 - protein, 227 aa, Stropharia semiglobata
MFPFVKTLILLPTLVTSAYALVYGVDSSTLVSTATYSKAKSEGFTKAIIRGYQEACGSGGRVDPNFVATYKNARAAGITDIDMYWFPCNGSGNSCKSYATQLSEIANAFSANSMKIGTIWIDFEKDSTCNNWNYGTTGNLNHAKALISAIKATGFKFGIYSSPGEWGTLFGSTGVVLDSSAPLWFATWNNVKTLTLGTHFGGWTTAAGHQYTDVSSSGQFDLNVFAN

SEQ ID NO: 152 - protein, 207 aa, Stropharia semiglobata
LVYGVDSSTLVSTATYSKAKSEGFTKAIIRGYQEACGSGGRVDPNFVATYKNARAAGITDIDMYWFPCNGSGNSCKSYATQLSEIANAFSANSMKIGTIWIDFEKDSTCNNWNYGTTGNLNHAKALISAIKATGFKFGIYSSPGEWGTLFGSTGVVLDSSAPLWFATWNNVKTLTLGTHFGGWTTAAGHQYTDVSSSGQFDLNVFAN

SEQ ID NO: 153 - DNA, 917 bp, Stropharia semiglobata
  sig_peptide 1..57
  mat_peptide 58..914
  source 1..917: mol_type = genomic DNA
  CDS 1..393
  CDS 464..562
  CDS 617..700
  CDS 757..785
  CDS 842..914
atggtcaaaatcttgagccttctagccctccttcccctgctcacgcagtcctatgcgctcacgtacgccgtggactcgtccacgctcgtctcggtcgcgacgtacaccaaggcaaagagccagggcttcaccaaggccattatccgcggctaccaggaggcgtgcggctccgggggggcagtcgatcccaacttcgtccaaacttacaagaacgcgcgcgcagcaggctacaccgacatcgacatgtactggttcccatgcaacgggtccacgcacaactgcaagtcgtacgcgacgcagattgccgctatagccgcgacgttcagcgcgaactcgatgaagatcggcaggatctggatcgacatcgagaaggacgctgccgtctgcaataacgtacgtgctgtggtgtactgactggggagtgagcgcactgatgacacgcgaatgacgcctcggtttgcagtggaattatggcacggccggtaatctatcccaggcgaaagcactgatctcggccattaaggcttcgggtttcgtgtacggcatctacagcagtcctggggtatgtttctttttttatcgttttttttagggtactcaccgttaaattttgtaggaatggggcaatatcttcggttccacaagcgtcgttgttgacaactctgccccactctggtttgctacatggaacaacgttcaggtacctaaattcacaatcatttttgcttctccactgtttctgatgataattctcagactctgactatgggaaccaagtttggagggtgtgtatccctgtactttttattattgtttcactcaataacgacgcgttcttcaggtggacttcggcaatgggccatcaatacacggacgtctctgcctctggacaattcgacctgagcgtattcgcatag

SEQ ID NO: 154 - protein, 226 aa, Stropharia semiglobata
MVKILSLLALLPLLTQSYALTYAVDSSTLVSVATYTKAKSQGFTKAIIRGYQEACGSGGAVDPNFVQTYKNARAAGYTDIDMYWFPCNGSTHNCKSYATQIAAIAATFSANSMKIGRIWIDIEKDAAVCNNWNYGTAGNLSQAKALISAIKASGFVYGIYSSPGEWGNIFGSTSVVVDNSAPLWFATWNNVQTLTMGTKFGGWTSAMGHQYTDVSASGQFDLSVFA

SEQ ID NO: 155 - protein, 207 aa, Stropharia semiglobata
LTYAVDSSTLVSVATYTKAKSQGFTKAIIRGYQEACGSGGAVDPNFVQTYKNARAAGYTDIDMYWFPCNGSTHNCKSYATQIAAIAATFSANSMKIGRIWIDIEKDAAVCNNWNYGTAGNLSQAKALISAIKASGFVYGIYSSPGEWGNIFGSTSVVVDNSAPLWFATWNNVQTLTMGTKFGGWTSAMGHQYTDVSASGQFDLSVFA

SEQ ID NO: 156 - DNA, 765 bp, Gelasinospora cratophora
  sig_peptide 1..54
  mat_peptide 55..762
  source 1..765: mol_type = genomic DNA
  CDS 1..144
  CDS 229..762
atgaagtccttcgtcctcacggccttcgccggcctcatcggcgctgctcaggctaccgtccagggcttcgatatctcccactaccagagcagcgtcaactttgccggcgcttactcttccggtgcccgcttcgtcatcatcaaggtgcgtcttttcttgtctcagagcaagccccatatcttcacaggacctccatccatccatactaacccacgaccccgctcacaggccaccgaaggaaccacctacatcgactcctcgttctcctcccactacaccggcgccacctccgccggcctgatccgcggcggctaccacttcgcgcacccggactcctccaccggcgccgctcaggccgactacttcctcgcgcacggcggcggctggtccgccgacggcatcaccttgcccggcatgatcgacctggagtccgtctcgggcaaagccacttgcttcggcctgtcgacttcggccatggtctcgtggatcaagtccttctcggatcgctactacgccaagacgggccgctaccccatgatctacaccaactactcgtggtggaaccagtgcacgggtaactctgccagctttgccgcgaccaacccgctggtgctggcgaggtggtcgagcacggtgggcaccctgccggggggttggagtgttcagactatttggcagaatgcggatacgtatacttatggtggtgactcggatgtttttaacggtagccttgatcgccttaaggctcttgctaagggatcttaa

SEQ ID NO: 157 - protein, 226 aa, Gelasinospora cratophora
MKSFVLTAFAGLIGAAQATVQGFDISHYQSSVNFAGAYSSGARFVIIKATEGTTYIDSSFSSHYTGATSAGLIRGGYHFAHPDSSTGAAQADYFLAHGGGWSADGITLPGMIDLESVSGKATCFGLSTSAMVSWIKSFSDRYYAKTGRYPMIYTNYSWWNQCTGNSASFAATNPLVLARWSSTVGTLPGGWSVQTIWQNADTYTYGGDSDVFNGSLDRLKALAKGS

SEQ ID NO: 158 - protein, 208 aa, Gelasinospora cratophora
TVQGFDISHYQSSVNFAGAYSSGARFVIIKATEGTTYIDSSFSSHYTGATSAGLIRGGYHFAHPDSSTGAAQADYFLAHGGGWSADGITLPGMIDLESVSGKATCFGLSTSAMVSWIKSFSDRYYAKTGRYPMIYTNYSWWNQCTGNSASFAATNPLVLARWSSTVGTLPGGWSVQTIWQNADTYTYGGDSDVFNGSLDRLKALAKGS

SEQ ID NO: 159 - protein, 208 aa, Acremonium alcalophilum
RIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 160 - protein, 4 aa, synthetic construct
  REGION 1..4: note = Conserved motif YKNA
YKNA

SEQ ID NO: 161 - protein, 5 aa, synthetic construct
  REGION 1..5: note = Conserved motif FGGW[S/T].
  SITE 5: note = MISC_FEATURE - The amino acid in position 1 of the conserved motif is either serine (Ser, S) or threonine (T, Thr).
FGGWX

SEQ ID NO: 162 - DNA, 52 bp, synthetic construct
  misc_feature 1..52: note = Primer
  source 1..52: mol_type = other DNA
acacaactggggatccaccatgttgaaaacaattatctataccacccttgcc

SEQ ID NO: 163 - DNA, 41 bp, synthetic construct
  misc_feature 1..41: note = Primer
  source 1..41: mol_type = other DNA
agatctcgagaagcttagccctttgcaaatcgttgcaatcc

SEQ ID NO: 164 - DNA, 40 bp, synthetic construct
  misc_feature 1..40: note = Primer
  source 1..40: mol_type = other DNA
acacaactggggatccaccatgaagttcgcatccgtcgcc

SEQ ID NO: 165 - DNA, 38 bp, synthetic construct
  misc_feature 1..38: note = Primer
  source 1..38: mol_type = other DNA
agatctcgagaagcttaaccggcgttggcaatcttctt

SEQ ID NO: 166 - DNA, 41 bp, synthetic construct
  misc_feature 1..41: note = Primer
  source 1..41: mol_type = other DNA
acacaactggggatccaccatgcgcccctccgtcatattgc

SEQ ID NO: 167 - DNA, 50 bp, synthetic construct
  misc_feature 1..50: note = Primer
  source 1..50: mol_type = other DNA
agatctcgagaagcttaagcagaaaacacgttcaaatcaaacttcttact

SEQ ID NO: 168 - DNA, 46 bp, synthetic construct
  misc_feature 1..46: note = Primer
  source 1..46: mol_type = other DNA
acacaactggggatccaccatgaagctcacctttgcctctctaact

SEQ ID NO: 169 - DNA, 40 bp, synthetic construct
  misc_feature 1..40: note = Primer
  source 1..40: mol_type = other DNA
agatctcgagaagcttaggctcctttggccatcctagaca

SEQ ID NO: 170 - DNA, 42 bp, synthetic construct
  misc_feature 1..42: note = Primer
  source 1..42: mol_type = other DNA
acacaactggggatccaccatgcgcgcctttattccagtctt

SEQ ID NO: 171 - DNA, 48 bp, synthetic construct
  misc_feature 1..48: note = Primer
  source 1..48: mol_type = other DNA
agatctcgagaagcttaggcagagaaaacgttgagatcaaatttcttg

SEQ ID NO: 172 - DNA, 48 bp, synthetic construct
  misc_feature 1..48: note = Primer
  source 1..48: mol_type = other DNA
acacaactggggatccaccatgaagctgtctctcctccttattgttgc

SEQ ID NO: 173 - DNA, 41 bp, synthetic construct
  misc_feature 1..41: note = Primer
  source 1..41: mol_type = other DNA
agatctcgagaagcttaacctagggccattctcttcaaccc

SEQ ID NO: 174 - DNA, 48 bp, synthetic construct
  misc_feature 1..48: note = Primer
  source 1..48: mol_type = other DNA
acacaactggggatccaccatgaagtcttttggtgttattgctaccgg

SEQ ID NO: 175 - DNA, 39 bp, synthetic construct
  misc_feature 1..39: note = Primer
  source 1..39: mol_type = other DNA
agatctcgagaagcttagcctctggcgattctctgaagc

SEQ ID NO: 176 - DNA, 48 bp, synthetic construct
  misc_feature 1..48: note = Primer
  source 1..48: mol_type = other DNA
ctatatacacaactggggatccaccatgaagtccttcgtcctcacggc

SEQ ID NO: 177 - DNA, 55 bp, synthetic construct
  misc_feature 1..55: note = Primer
  source 1..55: mol_type = other DNA
tagagtcgacccagccgcgccggccattaagatcccttagcaagagccttaaggc

SEQ ID NO: 178 - DNA, 54 bp, synthetic construct
  misc_feature 1..54: note = Primer
  source 1..54: mol_type = other DNA
ctatatacacaactggggatccaccatgaagtctgttactttcatcgccagtct

SEQ ID NO: 179 - DNA, 54 bp, synthetic construct
  misc_feature 1..54: note = Primer
  source 1..54: mol_type = other DNA
tagagtcgacccagccgcgccggccattacgaagcgttagcaatacgcttaagc

SEQ ID NO: 180 - DNA, 50 bp, synthetic construct
  misc_feature 1..50: note = Primer
  source 1..50: mol_type = other DNA
ctatatacacaactggggatccaccatgaaggcttcttccatcctctccc

SEQ ID NO: 181 - DNA, 51 bp, synthetic construct
  misc_feature 1..51: note = Primer
  source 1..51: mol_type = other DNA
tagagtcgacccagccgcgccggccatcatccactcgccaacttcttcaac

SEQ ID NO: 182 - DNA, 50 bp, synthetic construct
  misc_feature 1..50: note = Primer
  source 1..50: mol_type = other DNA
ctatatacacaactggggatccaccatgttctctttcgtcaaagcgctca

SEQ ID NO: 183 - DNA, 58 bp, synthetic construct
  misc_feature 1..58: note = Primer
  source 1..58: mol_type = other DNA
tagagtcgacccagccgcgccggccattaatttgcaaagacattgaggtcgaattggc

SEQ ID NO: 184 - DNA, 50 bp, synthetic construct
  misc_feature 1..50: note = Primer
  source 1..50: mol_type = other DNA
ctatatacacaactggggatccaccatgttccctttcgtcaaaacgctca

SEQ ID NO: 185 - DNA, 57 bp, synthetic construct
  misc_feature 1..57: note = Primer
  source 1..57: mol_type = other DNA
tagagtcgacccagccgcgccggccattaatttgcaaacacattgaggtcgaattgg

SEQ ID NO: 186 - DNA, 52 bp, synthetic construct
  misc_feature 1..52: note = Primer
  source 1..52: mol_type = other DNA
ctatatacacaactggggatccaccatggtcaaaatcttgagccttctagcc

SEQ ID NO: 187 - DNA, 53 bp, synthetic construct
  misc_feature 1..53: note = Primer
  source 1..53: mol_type = other DNA
tagagtcgacccagccgcgccggccactatgcgaatacgctcaggtcgaattg

SEQ ID NO: 188 - DNA, 37 bp, synthetic construct
  misc_feature 1..37: note = Primer
  source 1..37: mol_type = other DNA
acacaactggggatccaccatgaagactacgggtgtc

SEQ ID NO: 189 - DNA, 35 bp, synthetic construct
  misc_feature 1..35: note = Primer
  source 1..35: mol_type = other DNA
ccctctagatctcgagttaagaacccttggcaaag

SEQ ID NO: 190 - DNA, 39 bp, synthetic construct
  misc_feature 1..39: note = Primer
  source 1..39: mol_type = other DNA
acacaactggggatccaccatgaagttcactaccattgc

SEQ ID NO: 191 - DNA, 34 bp, synthetic construct
  misc_feature 1..34: note = Primer
  source 1..34: mol_type = other DNA
ccctctagatctcgagctacccctcgacaatctt

SEQ ID NO: 192 - DNA, 38 bp, synthetic construct
  misc_feature 1..38: note = Primer
  source 1..38: mol_type = other DNA
acacaactggggatccaccatgaagtctgttgctgtct

SEQ ID NO: 193 - DNA, 36 bp, synthetic construct
  misc_feature 1..36: note = Primer
  source 1..36: mol_type = other DNA
ccctctagatctcgagctaagaagcattcgcaatgc

SEQ ID NO: 194 - DNA, 37 bp, synthetic construct
  misc_feature 1..37: note = Primer
  source 1..37: mol_type = other DNA
acacaactggggatccaccatgaagctcacgagtgtg

SEQ ID NO: 195 - DNA, 35 bp, synthetic construct
  misc_feature 1..35: note = Primer
  source 1..35: mol_type = other DNA
ccctctagatctcgagttacgaacctctagcaagc

SEQ ID NO: 196 - DNA, 40 bp, synthetic construct
  misc_feature 1..40: note = Primer
  source 1..40: mol_type = other DNA
acacaactggggatccaccatgaagtcttacatcgccccc

SEQ ID NO: 197 - DNA, 39 bp, synthetic construct
  misc_feature 1..39: note = Primer
  source 1..39: mol_type = other DNA
gtgcggccgcaagcttaatcagaggcttcctcccatagg

SEQ ID NO: 198 - DNA, 43 bp, synthetic construct
  misc_feature 1..43: note = Primer
  source 1..43: mol_type = other DNA
acacaactggggatccaccatgaagtatctcgttccccttttg

SEQ ID NO: 199 - DNA, 38 bp, synthetic construct
  misc_feature 1..38: note = Primer
  source 1..38: mol_type = other DNA
agatctcgagaagcttattagcccttagccagtctctt

SEQ ID NO: 200 - DNA, 43 bp, synthetic construct
  misc_feature 1..43: note = Primer
  source 1..43: mol_type = other DNA
acacaactggggatccaccatgaagctcaccacttttatcacg

SEQ ID NO: 201 - DNA, 38 bp, synthetic construct
  misc_feature 1..38: note = Primer
  source 1..38: mol_type = other DNA
ctagatctcgagaagcttttaagtccccttggcaagcg

SEQ ID NO: 202 - DNA, 44 bp, synthetic construct
  misc_feature 1..44: note = Primer
  source 1..44: mol_type = other DNA
acacaactggggatccaccatgaagtttgcactcctagtatctg

SEQ ID NO: 203 - DNA, 42 bp, synthetic construct
  misc_feature 1..42: note = Primer
  source 1..42: mol_type = other DNA
ctagatctcgagaagcttttagccatagtagttcttgtccca

SEQ ID NO: 204 - DNA, 37 bp, synthetic construct
  misc_feature 1..37: note = Primer
  source 1..37: mol_type = other DNA
acacaactggggatccaccatggcaaagctcctcaag

SEQ ID NO: 205 - DNA, 35 bp, synthetic construct
  misc_feature 1..35: note = Primer
  source 1..35: mol_type = other DNA
agatctcgagaagcttattagtgggcgaagacgtt

SEQ ID NO: 206 - DNA, 39 bp, synthetic construct
  misc_feature 1..39: note = Primer
  source 1..39: mol_type = other DNA
acacaactggggatccaccatgaagcttctttccgccct

SEQ ID NO: 207 - DNA, 38 bp, synthetic construct
  misc_feature 1..38: note = Primer
  source 1..38: mol_type = other DNA
agatctcgagaagcttatcactgagaggcaaacttgac

SEQ ID NO: 208 - DNA, 39 bp, synthetic construct
  misc_feature 1..39: note = Primer
  source 1..39: mol_type = other DNA
acacaactggggatccaccatggcttccagactgaccct

SEQ ID NO: 209 - DNA, 38 bp, synthetic construct
  misc_feature 1..38: note = Primer
  source 1..38: mol_type = other DNA
ctagatctcgagaagcttttaagctgccacgcactggg

SEQ ID NO: 210 - DNA, 27 bp, synthetic construct
  misc_feature 1..27: note = Primer
  source 1..27: mol_type = other DNA
gaattcgagctcggtaccttgaagttc

SEQ ID NO: 211 - DNA, 31 bp, synthetic construct
  misc_feature 1..31: note = Primer
  source 1..31: mol_type = other DNA
ggtggatccccagttgtgtatatagaggatt

SEQ ID NO: 212 - DNA, 26 bp, synthetic construct
  misc_feature 1..26: note = Primer
  source 1..26: mol_type = other DNA
tgcgcggcgcggctgggtcgactcta

SEQ ID NO: 213 - DNA, 27 bp, synthetic construct
  misc_feature 1..27: note = Primer
  source 1..27: mol_type = other DNA
ttcacacaggaaacagctatgaccatg

SEQ ID NO: 214 - DNA, 25 bp, synthetic construct
  misc_feature 1..25: note = Primer
  source 1..25: mol_type = other DNA
ctatatacacaactggggatccacc

SEQ ID NO: 215 - DNA, 26 bp, synthetic construct
  misc_feature 1..26: note = Primer
  source 1..26: mol_type = other DNA
tagagtcgacccagccgcgccggcca

SEQ ID NO: 216 - DNA, 10767 bp, synthetic construct
  misc_feature 1..10767: note = Synthetic DNA construct of plasmid pDAu770.
  source 1..10767: mol_type = other DNA
taggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggcctccatggccgcggccgcgctttgctaaaactttggttgatggaaggtatctggcgataaactccgacgacgtctagaagcaacaatcttatgcaaacgctcattggttcttttcgaccgcaacatccatcatgaaactggtattttgtctgtgtcagcagtctagaaccccttgccgggtattttagcatttcatttttctataaaaaggtaccagcatgtatggatcgtatcttccgtaccgtggttattaaatcccagcagaggccgataggcttaagaagtgaacatggcatggttaaggaagaagccattactgagtatatatggctagaataatcgctgggaaagatttatgcttccaagaggcgtaggacggtataccatacagtacggtatttatgaacaattcgataataccactccccaaagcgggagataggacacccgcctcaggcaccaaccaccccctttttcaactgtcagtggtgcacgtttccatcgagcataagcttggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggttgaagttcctattccgagttcctattcttcaaatagtataggaacttcattagtttaaacgtacgattttgacatttgctccattgtcgaggatggatggaacgagcggcgtgcgccacgaaagtgaggctattgcctatcagctctttgctacattccggaaacaaacatccctttttgtgaattatctacgcaacttagatggcgtgaacgcatcttcaaagtctttcggcaggtccggcacgacttttgcatccagagaagcgcctacatgtgtattcgaccacctcctagcgcgcttggatatgaggaaatattactgagagtcgaaaacaagctccaccgcaccagctcttcttggagttttatattaaagaatattcccagctcgttgtattattctttttctaccgtgctaatgtatcaaggactttggtacctattaacgttattattcgtgtgctattcccaaacataaccctgtatatgtttcgaacgccgttatgacccatgtcttacatactcattaagtcattcccttggataatctcgactcagatgcggcggttgatgtaggaggagaggtaatcgaggacctcctgggagatgatgccgttccaggcggggtagcggatggagccctcggcggagcccttgagctgctcgatatgctgccactcctcgatggggttggtctcatccttgagggcgatcatctccttggagatgggatcgtaggcgtagtagcgggagactagtgcgaagtaatgatcggggatggcggtgatctgatgggtgtaggtggtgcgggcgacggcggaggcgcgcttatcggaccagttgccgacgacgttggtgagctcggtgaggcccttcatggagaggaaggaggtcatgagatggcggccgatatgggacttggggccgttcttgatggcgaagatggagtagggggcgttcttcttgagggccttgttgtaggagcggacgaggttatccttgaggagctggtactcctgcttgttggaggaggagttgccggtgcggttgacgcgcttgaggacgggctcggagttgcggaggaactcatcgaggtagacgaggggatcgatgcggccgcgggcggagaagaagtagatatggcgggagacggaggtcttggtctcggtgacgaggcactggatgatgacgccgaggtacttgttctggacgagcttgaaggacttgggatcgacgttcttgatatcggagaagcggccgcagttgatgaaggtggcgaggaagaggaactggtagagggtcttggtcttggtgaagcgggaggtgtactcgaaggagttgaggatcttctcggtgatctcccagatggactcgccctcggagaggagggccttgagcatcttcttggaatgggagttgcccttatcggcctcctcggaggactcgaactggagctggagggaggagacgatatcggtgatatcggactgatgcttctggccgtagtaggggatgatggtgaactcccaggcggggatgagcttcttgagggaggcctccaggatggtggccttctgggtcttgtacttgaactggagggacttgttgacgatatcgaaggagagggagttggagatgatggtgttgtaggacatgaaggtggcgcgcttgatggcggtgccgttatgggtgatcatccagcagaggtaggtgagctcggcggcgcagagggcgatcttctcgccggaggggcgctcgaagcgctcgacgaactggcggacgaggaccttggggggggtcttgcagaggatatcgaactggggcatggtgctcagatactacggctgatcgcgtagaggtactgagcaaaacagatgtcagtaaggagaagagttgaatgaatggaagaagagtaggaaaggaggtatgggggaaagatatacgtactgatgcggacgaagagagaaagaaggaaaaaagttgtgggaggggaaggagggggaatccttatatggaggggcaagcgagaaggcgaattagtgggcgggcttaagccctcgaccgccgcccttatcattggacatggaggggtaatgcccccaccacgcatgtgcgggaccgacgcagaatctgcacggcggagtctcttccagactgttgacttttgggcgatgactcttgttgctgcggccttttgggtacaccaacctcgttgatcttgtttccttggttctctttcgctcggagacccgaccatgaccccaccatcagtcactatcctgcctcgtcgataaaaattttttcttccctctgattgttacatagtatgtttccacctttccggtggatttcggacagtcaaactgggcatcaacgcagtggtgggctgcttcgtttgctgcgtgttgtacttgtttgcatttgaaccccgcggtcgttcgagtccttaattggtccgctcccggtcaacacccaagcagctgtggcccggccgagtggcgcctgtctggtccacagttaattaaaggagagagttgaacctggacgccgcgcaaaaagcaaagacgcgcctcgtgggcggtggatcaatgatcggatttagtggcagatggcatcacaggcggccaatgaccaccgggccaactggccccgacattccagcaatactgcctaattgactccaccatgcatctcggctattattgaactgggtttgatggatggggaccctcttggaattgtcaaagattttgaagcgaagacgatctattggacggtagagatatactcttgatttagtcgttgggaggcccctggggaaagcaatgatggggaatgttgctgctccactgtggacctcggctatggaattacgtgcttggatctaagatgagctcatggctatgcattgaatgacagtgatatcagcagagcaagcagagaaggatggaatgctaattttctagtgctttgtgcaagggtaaatcagggactgtctgtctggtcttctacacgaaggaaagaccatggctttcacggtgtctgtatttccggatatcctcaattccgtcggtcgattacaatcacatgacttggcttccatttcactactattatgcacacccactacatacatgatcatataaccaattgccctcatccccatcctttaactatagcgaaatggattgattgtctaccgccaggtgtcagtcaccctctagatctcgagctcgctagagtcgacctatggagtcaccacatttcccagcaacttccccacttcctctgcaatcgccaacgtcctctcttcactgagtctccgtccgataacctgcactgcaaccggtgccccatggtacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggaatcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccatggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatggagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatcaccgatttctttgggaaatcaataacgctgtcttccgcaggcagcgtctggactttccattcatcagggatggtttttgcgaggcgggcgcgcttatcagcggccagttcttcccaggattgaggcatgtgcatgcaatgtgtgtttatgtggaagtaagatacgacgagtttgattgagaaaagacagggtgattgtcaagttcagtatggaagaaagagtagaagaagatcagacgacagggaagagcgatgacataaaaggtggaagacggaagaaaaacgaaccaaatcaatcccactctatggcgggggttggactgcctgaggccggcactggtggggcttatcgataagttctcgtcaccggatgcaatgcgctgtcaactgctgacttggccctgaacatcctgtcctctacagatccatactatacaatgatcccagttatagtgcggtaaggtgcatatcatatctcattctcatgactcattcgacttttttttagagaaagtacatacgtggaacatacactaaacgcaacaggtcgcgacaacactggtatacaaaacggtccccggtgaatgacgttattagtgtctatcccccactcacacccgaaaagaataatagaaactaacagaaaaagcggcccgaggataagaggaacattcaaacagaaggggaatcataaaaaccgaaaaatgcaaggaaaagagaactcaaatcaataattttcataatactgtcgagagtaatacggaccagcgtctctcagggacatgcgtcggcgcaaggcatcatccaatctctcatctaacacatccagcattcgtgttcgatagtctaactgcttctctcggcgctcaagtcttgcttcccgatcatcgagttaattaagaagttcctatactttctagagaataggaactcggaataggaacttcaaggtaccgagctctatcctcaataccctattttccacgattccattgtcatatccaattccgttttcttttcttgttttcccctcatccaatcccgtccatcatttactcctttttcttgtgaatgcaagtggcactaagaaatccaacccccagacaaattttcctactcaggaacacaaaaacctcgtttctgctcccttctcgtacttcattcctatcgtctcggaatttcctcaacaaccctttccgactttgcgacagcgtcgcgattccagacttatgtgttctcgttcctactgtcgttaccagtctatttattccgaaacctctgatcgctgaatttcacacacaacacccccccgttgatgctggtggagaatccgtagcgtcaagagttgaattcactccatgttgtaacgaagtccacgaattgagacgattgatgattacaaccccgcgatcgcctatcgacgattcgacgagatgccattctcatcctcctcatcctcctccacccccgaggtgtctaccaccccgctcgcagattacttctggatcgcaggtgtcgatggcgcggaaatcttagagactttccaaagactcggcgacgaatacagggcaaacagtgccaccgctcctggccccgctcttgcggacacgatcgaggaagatgcggacgcggaggaggcacacgacccccgtctggactccctctctcgacccaattccatggctgggggccgcaattccttccagcggttctcaatgcgctcaggagactccagtgagtccagtgggaatggtaccagcagcaaccggagcagtctgaccatcaagggtaatcagtcgcccagagggtcgtcgtttctagaagatttcgactttgacaaggccctgttcaagtttgcaaacgagcgggagtcgttcctgtcggatctgagtctcagtgccggagcaatcactcccacctcccgtcctaggtccaggttacgtacacagaagattgtctccgaggaaagtccctcccagccatccagcttgcttcgatcaggcattggtagtgtgcggcgtcatatggcattcagagacatgaatagtatgaaacggcagccgtcagttgctcgtcgcggccgcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttttcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaa

SEQ ID NO: 217 - DNA, 1052 bp, Flammulina velutipes
  sig_peptide 1..51
  mat_peptide 52..1049
  source 1..1052: mol_type = genomic DNA
  CDS 1..157
  CDS 213..347
  CDS 401..451
  CDS 504..600
  CDS 659..666
  CDS 715..838
  CDS 895..981
  CDS 1037..1049
atgcgcatcttactcttcatcgctgtgaccattgcactcggcgtccatgctcgtctcaatggcatcgacgtttccgggtatcagccgaacgtcaactgggccactgtcaaggctaatggcgtctcattcgcgtatatcaaggccaccgaaggcactagtaagttcacttcctatatccttttggtccagtgctgatatcatcgacaacccagcttatacgaacccgtcgttctcatcgcaatacaccggagctaccaaagctggactcattcgaggatcgtaccattttgcgcacccaagtagtagcacgggcgctgcacaggccagatactttgtcgcgcatggcggtaggaattgatccatcttgtgtggcgtcccccattgacacaatccttgataggtggctggtcgggagacggaatcactctgcctggggcgctggatatagaatgtaatgatcagtcagatctattacgttttgagtcacattgacaacttcttagacaatccaagtggcgcaacttgctacggcctgagtacctcatcaatggtcaattggattgccgacttctctaacacttaccattccctcactggaaggttaggcttcgtaaacacaagccatgtactgccgtagtaacgctaatctccatttcagataccctggtacaccaatcccacaatcattttgagtctgctcatctcccctgacagtaatttacaccaccgcagactggtggaggacatgtactggtaacagtgcatcctttgccaacaacagtcctctctggattgcgagatatgccagtaccatcggtacactccctgctggatggaggtgcgacccttctacttattcgatgactctgttctgaaccaagtgttcattctaagctacgccacattctggcagtacgctgactcgggtagtaatcccggagatcaggattatttcaacggggatgctgcaggtctcaaacggtaaattgatatctttttatatatcttcacctgcgagactaatccatgtttataggcttgccaccagttga

SEQ ID NO: 218 - protein, 224 aa, Flammulina velutipes
MRILLFIAVTIALGVHARLNGIDVSGYQPNVNWATVKANGVSFAYIKATEGTTYTNPSFSSQYTGATKAGLIRGSYHFAHPSSSTGAAQARYFVAHGGGWSGDGITLPGALDIEYNPSGATCYGLSTSSMVNWIADFSNTYHSLTGRYPVIYTTADWWRTCTGNSASFANNSPLWIARYASTIGTLPAGWSYATFWQYADSGSNPGDQDYFNGDAAGLKRLATS

SEQ ID NO: 219 - DNA, 711 bp, synthetic construct
  misc_feature 1..711: note = Codon optimised sequence
  sig_peptide 20..70
  mat_peptide 71..691
  source 1..711: mol_type = other DNA
  CDS 20..691
acacaactggggatccaccatgcgaatcctcttgttcatcgcagtcacaatcgcgttgggagtccatgccaggctcaacggcatcgatgtctcgggataccagccgaacgtcaactgggccacggtcaaagcgaacggcgtgtccttcgcgtacatcaaggcaaccgagggcaccacgtatacaaacccctcgttctcctcccagtacaccggagcaacaaaagccggattgatcaggggctcctaccatttcgcgcatccttcgtcgtccacaggcgcagcacaggcacgatacttcgtggcacatggcggtggttggtccggtgatggtatcaccttgcctggtgcgctcgatatcgagtataacccttcgggtgcgacatgttacggcctctcgacctcgtccatggtgaactggatcgccgatttctccaacacttatcactcgctcacaggcaggtaccccgtcatttacaccactgccgattggtggcgaacctgtaccggcaactccgcatccttcgcaaacaactcgcctctctggattgcgcgttacgcgtcgactatcggtacgctccctgccggatggtcgtacgcgaccttctggcagtatgcggattcgggctccaaccctggcgatcaggattacttcaacggtgacgcagcgggtctcaagcgtctcgcgacatcgtaataagcttctcgagatct

SEQ ID NO: 220 - protein, 224 aa, synthetic construct; identical to SEQ ID NO: 218
  REGION 1..224: note = Synthetic Construct

SEQ ID NO: 221 - protein, 207 aa, Flammulina velutipes
RLNGIDVSGYQPNVNWATVKANGVSFAYIKATEGTTYTNPSFSSQYTGATKAGLIRGSYHFAHPSSSTGAAQARYFVAHGGGWSGDGITLPGALDIEYNPSGATCYGLSTSSMVNWIADFSNTYHSLTGRYPVIYTTADWWRTCTGNSASFANNSPLWIARYASTIGTLPAGWSYATFWQYADSGSNPGDQDYFNGDAAGLKRLATS

SEQ ID NO: 222 - DNA, 921 bp, Deconica coprophila
  sig_peptide 1..57
  mat_peptide 58..918
  source 1..921: mol_type = genomic DNA
  CDS 1..390
  CDS 449..547
  CDS 600..683
  CDS 751..779
  CDS 843..918
atgcttttcgcaacctttctttgtcttgcgagctacgcgtttggatcggcctatgcactcgtgcatgccgtagactcttcttccctcgtctccaccgccactttctccaaggcaaagtctgaaggctttaccaaggcagtcatccgcgggtaccaggaggcttgcggaagtggtggacgcgtcgaccccaactttgttcagacatacaagaacgcaagggccgcaggaattaccaacatcgacacctactggtatccatgcaatggatccggcaacagctgcaagtcctacgcaaagcaaattgcgggtatcagcgccacgttcaacgcgcattccatgaagatcggcaggatttggatcgacatcgagaaagattctatttgcaacaacgtaagtggggttgtcatacttttatctacccacctctaaaaactgcgttaccactcagtggaactacggcacttctggaaatagggaccatgccaagaagttgattaccgccatcaagaactccggcttcaaatatggaatctacagctctcctggggtaagtgcatccatgccctggaacgcgaccgacacagtaactccctatacaggaatggagcactatcttcggctccgagagcttcgacctcgatagcggggcgccactgtggtttgcgacatggaacaacgttcaggtacgcaggttatgactcgtaactctaggaggtggaatcactcatacgatatattatgcatacacagactctgacactcggcacgcactttggagggtaagtctctaggtttaaagggttcgatatagatccatagactaatccacgttactctaataggtggacaagcgctcatggacaccagtacacggataagtcagcctcgggtcaatttgacctcaacgtcttttcctcgtaa

SEQ ID NO: 223 - protein, 226 aa, Deconica coprophila
MLFATFLCLASYAFGSAYALVHAVDSSSLVSTATFSKAKSEGFTKAVIRGYQEACGSGGRVDPNFVQTYKNARAAGITNIDTYWYPCNGSGNSCKSYAKQIAGISATFNAHSMKIGRIWIDIEKDSICNNWNYGTSGNRDHAKKLITAIKNSGFKYGIYSSPGEWSTIFGSESFDLDSGAPLWFATWNNVQTLTLGTHFGGWTSAHGHQYTDKSASGQFDLNVFSS

SEQ ID NO: 224 - protein, 207 aa, Deconica coprophila
LVHAVDSSSLVSTATFSKAKSEGFTKAVIRGYQEACGSGGRVDPNFVQTYKNARAAGITNIDTYWYPCNGSGNSCKSYAKQIAGISATFNAHSMKIGRIWIDIEKDSICNNWNYGTSGNRDHAKKLITAIKNSGFKYGIYSSPGEWSTIFGSESFDLDSGAPLWFATWNNVQTLTLGTHFGGWTSAHGHQYTDKSASGQFDLNVFSS

SEQ ID NO: 225 - DNA, 944 bp, Rhizomucor pusillus
  sig_peptide 1..60
  mat_peptide 61..941
  source 1..944: mol_type = genomic DNA
  CDS 1..181
  CDS 242..484
  CDS 555..688
  CDS 757..825
  CDS 885..941
atgaagtttgcactcctagtatctgctatcgcaggccttgcagcaaccgccgtccaagcctacgaaactggcgttgatgtctctgccttgacttccacctccgcctggagctgtgcaaagaaactcggttacgatcacgctattgtccgctgctacattgaggcatacggaggcaaccctgcaagtatcatgcatcagagaagacgactactacgttctagcgtgctaacaatgtactagggtggtaaaattgacagcaactgcttccagaactacaagaacgccaaagcaggtggattcaccagcgttgacatttacatgttcccctgcactggccgctccacatgcaagtcgcccgctgctcaggtcaaggaggttgttgactacgttggctccaacaagatgactgttggacgtctctggctcgacgtcgaaatcgatccttctgctaacaactggccatccgccagtagcgctcgcagcagtaagaaagaggacaagaaaccaattcaggatgctcacaatgttgctaacattttttattacttgttaagccttgaagtccttcaagtctgctcttgactcgactggctggaaatacggtatctattcttctgcttcccaatggtcgcagatcaccggatcctcctcctgggagcttgattcctcgttgccactttggtacgctgtaagtagctctcacgttattccgtatattgtgggagaacgtggctgatctgaatttattaccacaagcactacgacgcttctctcagcttcagcgacttttcgccatttggtggctggactaagcctaccatcaaggtaaatattcaggtcatccggtgcttgacgcatatgtactcaattaaattgccatttagcaatatgctggctctgtaagcttctgctccgctggctgggacaagaactactatggctaa

SEQ ID NO: 226 - protein, 228 aa, Rhizomucor pusillus
MKFALLVSAIAGLAATAVQAYETGVDVSALTSTSAWSCAKKLGYDHAIVRCYIEAYGGNPGGKIDSNCFQNYKNAKAGGFTSVDIYMFPCTGRSTCKSPAAQVKEVVDYVGSNKMTVGRLWLDVEIDPSANNWPSASSARSTLKSFKSALDSTGWKYGIYSSASQWSQITGSSSWELDSSLPLWYAHYDASLSFSDFSPFGGWTKPTIKQYAGSVSFCSAGWDKNYYG

SEQ ID NO: 227 - protein, 208 aa, Rhizomucor pusillus
YETGVDVSALTSTSAWSCAKKLGYDHAIVRCYIEAYGGNPGGKIDSNCFQNYKNAKAGGFTSVDIYMFPCTGRSTCKSPAAQVKEVVDYVGSNKMTVGRLWLDVEIDPSANNWPSASSARSTLKSFKSALDSTGWKYGIYSSASQWSQITGSSSWELDSSLPLWYAHYDASLSFSDFSPFGGWTKPTIKQYAGSVSFCSAGWDKNYYG

SEQ ID NO: 228 - DNA, 909 bp, Stropharia semiglobata
  sig_peptide 1..60
  mat_peptide 61..906
  source 1..909: mol_type = genomic DNA
  CDS 1..393
  CDS 447..545
  CDS 608..691
  CDS 744..772
  CDS 831..906
atgttctctttcgtcaaagcgctcatactactcccgacccttgtgacctccgcatacgcccttgtatatggcgtagactcgtccactctagtctctactgcaacgtacagtaaagccaagagcgagggcttcactaaggctatcatccgcgggtatcaggaagcatgtggcagtggtggacgcgtcgaccccaacttcgtcgcgacctacaagaatgcacgggccgcgggtatcaccgatatcgacatgtactggttcccatgcaacgggtccggtaactcatgcaagtcgtacgctaagcagctctccgagatcgcgaatgtcttcagtgcgaatagcatgaaaattgggacaatttggattgattttgagaaggattctggttgcaacaacgtgagtttcatggacattatgtccttcataataaattgacggattttatcaagtggaactacggcaccacgggtaatctcaaccatgcaaaggcactcatctccgcgattaaagcgactgggtttaaattcggtatctacagctcacctggcgtaaggctttttacctttcgtatccgcttcatcaagttaacttagtccgcgactcgttttaggaatggggtaccctgtttgggtccaccggggtagtactcgatagctcggcccccctttggttcgctacgtggaacaatgtaaaggtatgtgaccccgcctactacagaaataacatctctcaaagtttaaccatagactcttaccttgggaacgcatttcggggggtaagtgcaatatatatacatcgaaatatgggatattgaatactgacgttgcttgaaggtggactaaagccgttggccaccagtatacagatgtgtccgcctctggccaattcgacctcaatgtctttgcaaattaa

SEQ ID NO: 229 - protein, 227 aa, Stropharia semiglobata
MFSFVKALILLPTLVTSAYALVYGVDSSTLVSTATYSKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTKAVGHQYTDVSASGQFDLNVFAN

SEQ ID NO: 230 - protein, 207 aa, Stropharia semiglobata
LVYGVDSSTLVSTATYSKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTKAVGHQYTDVSASGQFDLNVFAN

SEQ ID NO: 231 - DNA, 910 bp, Stropharia semiglobata
  sig_peptide 1..60
  mat_peptide 61..907
  source 1..910: mol_type = genomic DNA
  CDS 1..393
  CDS 447..545
  CDS 608..691
  CDS 744..772
  CDS 832..907
atgttccctttcgtcaaaacgctcatcctcctcccggcccttgtgacttccgcgtatgccctcgtatatggcgtagactcgtctaccttggtttctactgcaacctacaagaaagccaagagcgagggcttcactaaggctatcatccgcgggtatcaggaagcatgtggcagtggtggacgcgtcgaccccaacttcgtcgcgacctacaagaatgcacgggccgcgggtatcaccgatatcgacatgtactggttcccatgcaacgggtccggtaactcatgcaagtcgtacgctaagcagctctccgagatcgcgaatgtcttcagtgcgaatagcatgaaaattgggacaatttggattgattttgagaaggattctggttgcaacaacgtgagtttcatggacattatgtccttcataataaaatgacggattttatcaagtggaactacggcaccacgggtaatctcaaccatgcaaaggcactcatctccgcgattaaagcgactgggtttaaattcggtatctacagctcacctggcgtaaggctttttacctttcgtatccgcttcatcaagttaacttagtccgcgactcgttttaggaatggggtaccctgtttgggtccaccggggtagtactcgatagctcggcccccctttggttcgctacgtggaacaatgtaaaggtatgtgaccccgcctactacagaaataacatctctcaaagtttaaccatagactcttaccttgggaacgcatttcggggggtaagtgcaatatatatacatcgaaagatggaataattaaatactgatctcgcttaaaggtggactacagccgcgggtcaccagtacacagacgtgtcttcctctggccaattcgacctcaatgtgtttgcaaattaa

SEQ ID NO: 232 - protein, 227 aa, Stropharia semiglobata
MFPFVKTLILLPALVTSAYALVYGVDSSTLVSTATYKKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTTAAGHQYTDVSSSGQFDLNVFAN

SEQ ID NO: 233 - protein, 207 aa, Stropharia semiglobata
LVYGVDSSTLVSTATYKKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTTAAGHQYTDVSSSGQFDLNVFAN

SEQ ID NO: 234 - DNA, 47 bp, synthetic construct
  misc_feature 1..47: note = SEQ ID NO: 225 is the forward primer C8VSE-F.
  source 1..47: mol_type = other DNA
acacaactggggatccaccatgcttttcgcaacctttctttgtcttg

SEQ ID NO: 235 - DNA, 45 bp, synthetic construct
  misc_feature 1..45: note = SEQ ID NO: 226 is the reverse primer C8VSE-R.
  source 1..45: mol_type = other DNA
agatctcgagaagcttacgaggaaaagacgttgaggtcaaattga